(19) United States
(12) Patent Application Publication
(10) Pub. No.: US 2023/0138100 A1
(43) Pub. Date: May 4, 2023

(54) COMPOUNDS FOR ELECTRONIC DEVICES

(71) Applicant: Merck Patent GmbH, Darmstadt (DE)

(72) Inventors: Amir Hossain PARHAM, Darmstadt (DE); Elvira MONTENEGRO, Darmstadt (DE)

(21) Appl. No.: 17/785,635

(22) PCT Filed: Dec. 17, 2020
(86) PCT No.: PCT/EP2020/086602

§ 371 Date: Jun. 15, 2022

(30) Foreign Application Priority Data
Dec. 19, 2019 (EP) 19218239.2

(51) Int. Cl. C07D 409/04; H01L 51/00; C07D 409/14; C07D 405/10; C07D 405/14; C07D 409/10; C07D 405/04; C07D 495/04; H01L 51/50

(52) U.S. Cl. CPC C07D 409/04; H01L 51/0074; H01L 51/0067; C07D 409/14; H01L 51/0073; H01L 51/0054; C07D 405/10; C07D 405/14; C07D 409/10; C07D 405/04; C07D 495/04; H01L 51/0071; H01L 51/0072; H01L 51/0064; H01L 51/5072

(57) ABSTRACT

The present application relates to a compound of a formula (I), to the use thereof in electronic devices, to processes for preparing the compound, and electronic devices comprising the compound.

The present application relates to fluorene derivatives and to spirobifluorene derivatives in which one of the benzene rings has been exchanged for a heteroaryl ring. The compounds are suitable for use in electronic devices.

Electronic devices in the context of this application are understood to mean what are called organic electronic devices, which contain organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

In electronic devices, especially OLEDs, there is great interest in an improvement in the performance data. In these aspects, it has not yet been possible to find any entirely satisfactory solution.

A great influence on the performance data of electronic devices is possessed by emission layers and layers having an electron-transporting function. There is a continuing search for novel compounds for use in these layers, especially electron-transporting compounds and compounds that can serve as electron-transporting matrix material, especially for phosphorescent emitters, in an emitting layer, and lead in this use to electronic devices that simultaneously have high efficiency, long lifetime and low operating voltage. For this purpose, there is a particular search for compounds that have a high glass transition temperature, high stability, and high electron conductivity. A high stability of the compound is a prerequisite for achieving a long lifetime of the electronic device.

The prior art especially discloses heterofluorene and heterospirobifluorene derivatives having fused-on phenyl groups and bipolar heterofluorene and heterospirobifluorene derivatives especially having N-carbazole substitution as electron transport materials and electron-transporting matrix materials for electronic devices.

However, there is still a need for alternative compounds suitable for use in electronic devices, especially for compounds having one or more of the abovementioned advantageous properties. There is still a need for improvement in the performance data achieved when the compounds are used in electronic devices, especially in respect of lifetime, operating voltage and efficiency of the devices.

It has now been found that particular fluorene derivatives and spirobifluorene derivatives in which one of the benzene rings has been exchanged for a heteroaryl ring are of excellent suitability for use in electronic devices. They are especially suitable for use in OLEDs, and in these especially for use as electron transport materials and for use as electron-transporting matrix materials, especially for phosphorescent emitters. The compounds found lead to high lifetime, high efficiency and low operating voltage of the electronic devices. Further preferably, the compounds found have a high glass transition temperature, high stability and high conductivity for electrons.

The present application thus provides compounds of the following formula (I):

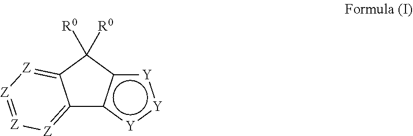

where:

Y is the same or different at each instance and is selected from O, S, NAr⁰ and CAr¹, where there must be at least one Y present which is selected from O, S and NAr⁰;

Z is the same or different at each instance and is selected from N and CR¹;

Ar⁰ is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;

Ar¹ is the same or different at each instance and is selected from H, D, F, Cl, Br, I, C(═O)R², CN, Si(R²)₃, P(═O)(R²)₂, OR², S(═O)R², S(═O)₂R², straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R² radicals; and where one or more CH₂ groups in the alkyl, alkoxy, alkenyl and alkynyl groups mentioned may be replaced by —R²C═CR²—, —C≡C—, Si(R²)₂, C═O, C═NR², —C(═O)O—, —C(═O)NR²—, NR², P(═O)(R²), —O—, —S—, SO or SO₂, with exclusion of joining of two Ar¹ groups to one another to form a mono- or polycyclic, aliphatic or aromatic ring system;

R⁰ is the same or different at each instance and is selected from H, D, F, Cl, Br, I, C(═O)R¹, CN, Si(R¹)₃, N(R¹)₂, P(═O)(R¹)₂, OR¹, S(═O)R¹, S(═O)₂R¹, straight-chain alkyl, alkoxy or thioalkyl groups having 1 to 20 carbon atoms, branched or cyclic alkyl, alkoxy or thioalkyl groups having 3 to 20 carbon atoms, alkenyl or alkynyl groups having 2 to 20 carbon atoms, aromatic ring systems having 6 to 40 aromatic ring atoms, heteroaromatic ring systems having 5 to 40 aromatic ring atoms, aryloxy or heteroaryloxy groups having 5 to 40 aromatic ring atoms, and aralkyl groups having 5 to 40 aromatic ring atoms; where the two R⁰ radicals may be joined to one another and may form an aliphatic or heteroaliphatic ring, so as to form a spiro compound in position 8 of the fluorene derivative, especially a spirobifluorene derivative; where the alkyl, alkoxy, thioalkyl, alkenyl, alkynyl, aryloxy, heteroaryloxy and aralkyl groups mentioned and the aromatic ring systems and heteroaromatic ring systems mentioned are each substituted by R¹ radicals; and where one or more CH₂ groups in the alkyl, alkoxy, thioalkyl, alkenyl, alkynyl, aryloxy, heteroaryloxy and aralkyl groups mentioned may be replaced by —R¹C═CR¹—, —C≡C—, Si(R¹)₂, C═O, C═NR¹, —C(═O)O—, —C(═O)NR¹—, NR¹, P(═O)(R¹), —O—, —S—, SO or SO₂;

R¹ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form an aliphatic or heteroaliphatic ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═0, C═NR⁵, —O(═O)O—, —C(═O)NR⁵—, NR⁵, P(═O)(R⁵), —O—, —S—, SO or SO₂;

R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;

R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;

R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and ON; and in the formula (I), at least one R¹ radical, one Ar⁰ group or one Ar¹ group is substituted by an A group conforming to a formula (A):

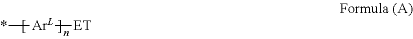

where:

* denotes to bond to the structure of the formula (I);

Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;

ET is the same or different at each instance and is an electron-transporting group selected from electron-deficient heteroaromatic groups to which one or more Ar² groups are bonded;

Ar² is the same or different at each instance and is selected from H, D, F, Cl, Br, I, C(═O)R³, CN, Si(R³)₃, P(═O)(R³)₂, OR³, S(═O)R³, S(═O)₂R³, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R³ radicals; where one or more CH₂ groups in the alkyl, alkoxy, alkenyl and alkynyl groups mentioned may be replaced by —R³C═CR³—, —C═C—, Si(R³)₂, C═O, C═NR³, —C(═O)O—, —C(═O)NR³—, NR³, P(═O)(R³), —O—, —S—, SO or SO₂, and where two or more adjacent Ar² groups together may form a mono- or polycyclic, aliphatic or aromatic ring system;

R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂; and

n is 0 or 1, where, in the case that n=0, the Ar^{L} group is absent and the FG group is bonded directly to the structure of the formula (I) via a single bond.

Consequently, the compounds of the formula (I), by definition, always contain at least one substituent of the formula (A) having an electron-transporting group ET.

The manner of counting in the heterofluorene derivatives according to the present application is fixed as follows:

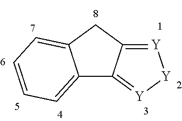

The manner of counting in the heterospirobifluorene derivatives according to the present application is fixed as follows:

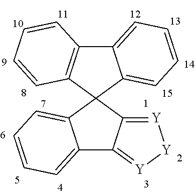

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atom, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aryloxy group as defined in the present invention is understood to mean an aryl group as defined above bonded via an oxygen atom. An analogous definition applies to heteroaryloxy groups.

An aromatic ring system in the context of this invention is a system which does not necessarily contain solely aryl groups, but which may additionally contain one or more non-aromatic rings fused to at least one aryl group. These non-aromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more non-aromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a non-aromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be replaced by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

In a preferred embodiment of the invention, in formula (I), exactly two Y are selected from NAr⁰, O and S, more preferably exactly one Y, and the other two Y are CAr¹. More preferably, exactly one Y is selected from O and S, most preferably from S, and the two other Y are CAr¹.

In a preferred embodiment of the invention, the compound of the formula (I) is accordingly selected from the compounds of the following formulae (II), (III) and (IV):

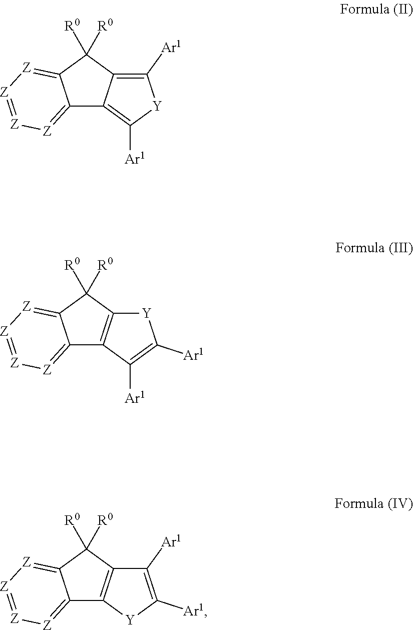

where there is at least one A group per formula.

Among the abovementioned formulae, preference is given to the formulae (II) and (IV), especially formula (II), where Y is preferably O or S; more preferably, Y is S.

Preferably not more than two Z groups in formula (I) are N; more preferably not more than one Z group in formula (I) is N; very preferably, no Z group is N. The remaining Z groups are correspondingly CR¹. It is additionally preferable that no adjacent Z groups in one ring are N.

Ar⁰ is preferably the same or different at each instance and is selected from aromatic ring systems which have 6 to 40 aromatic ring atoms and are each substituted by R² radicals. More preferably, Ar⁰ is the same or different at each instance and is selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R² radicals. Very particular preference is given to phenyl, biphenyl, terphenyl, quaterphenyl, naphthyl, fluorenyl, especially 9,9′-dimethylfluorenyl and 9,9′-diphenylfluorenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R² radicals. Most preferably, Ar⁰ is phenyl substituted by R² radicals, where R² is preferably H.

Ar¹ is preferably the same or different at each instance and is selected from H, D, straight-chain alkyl or alkoxy groups having 1 to 20 carbon atoms, branched or cyclic alkyl or alkoxy groups having 3 to 20 carbon atoms, aromatic ring atoms having 6 to 40 aromatic ring atoms, and heteroaromatic ring systems having 5 to 40 aromatic ring atoms, where the alkyl groups, alkoxy groups, aromatic ring systems and heteroaromatic ring systems are each substituted by R² radicals, with exclusion of joining of two Ar¹ groups to one another to form an aliphatic or heteroaromatic ring; Ar¹ is more preferably the same or different at each instance and is selected from H and aromatic ring systems which have 6 to 40 aromatic ring atoms and are substituted by R² radicals. Even more preferably, Ar¹ is the same or different at each instance and is selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R² radicals, and H. Even greater preference is given to phenyl, biphenyl, terphenyl, quaterphenyl, naphthyl, fluorenyl, especially 9,9′-dimethylfluorenyl and 9,9′-diphenylfluorenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R² radicals, where R² is preferably H, and H.

Preferred Ar¹ groups are shown in the following table:

| | |
|---|---|
| 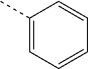 | Ar¹-1 |
| 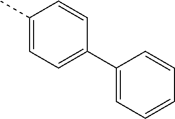 | Ar¹-2 |
| 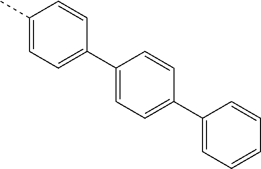 | Ar¹-3 |
| 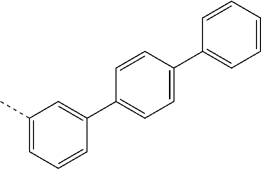 | Ar¹-4 |
| 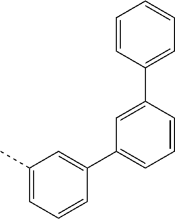 | Ar¹-5 |
| 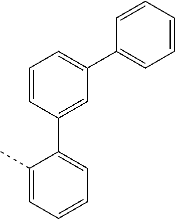 | Ar¹-6 |
| 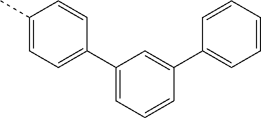 | Ar¹-7 |
| 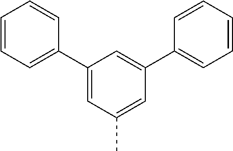 | Ar¹-8 |
| 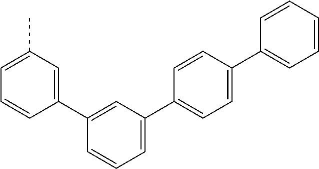 | Ar¹-9 |
| 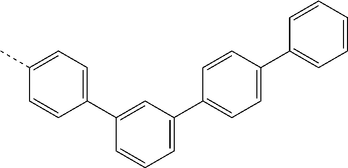 | Ar¹-10 |
| 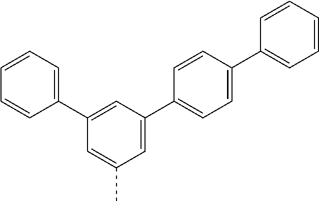 | Ar¹-11 |
| 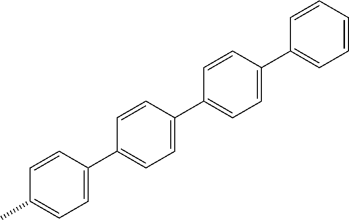 | Ar¹-12 |
| 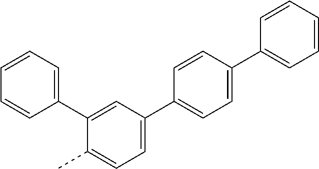 | Ar¹-13 |
| 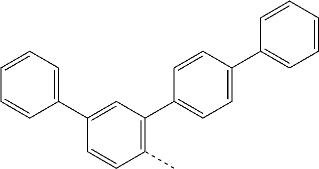 | Ar¹-14 |
| 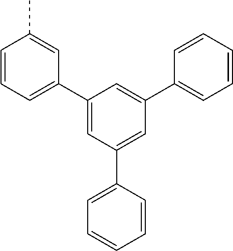 | Ar¹-15 |
| 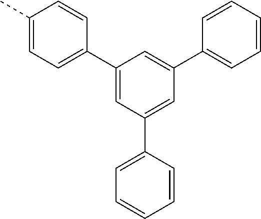 | Ar¹-16 |
| 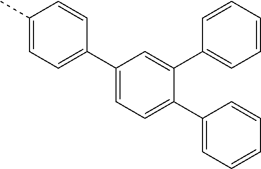 | Ar¹-17 |
| 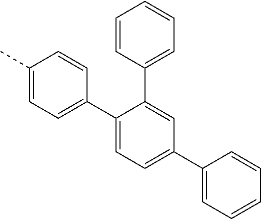 | Ar¹-18 |
| 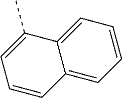 | Ar¹-19 |
| 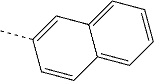 | Ar¹-20 |
| 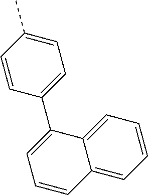 | Ar¹-21 |
| 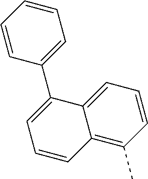 | Ar¹-22 |
| 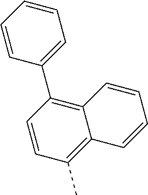 | Ar¹-23 |
| 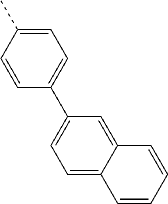 | Ar¹-24 |
| 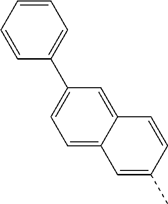 | Ar¹-25 |
| 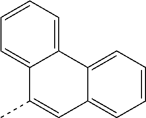 | Ar¹-26 |
| 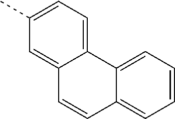 | Ar¹-27 |
| 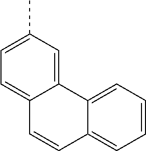 | Ar¹-28 |
| 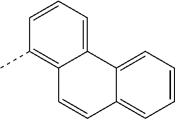 | Ar¹-29 |
| 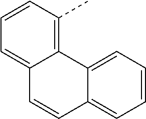 | Ar¹-30 |
| 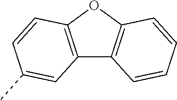 | Ar¹-31 |
| 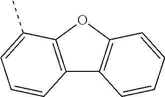 | Ar¹-32 |
| 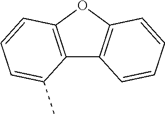 | Ar¹-33 |
| 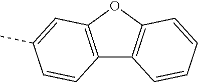 | Ar¹-34 |
| 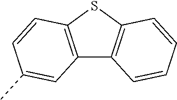 | Ar¹-35 |
| 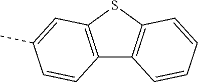 | Ar¹-36 |
| 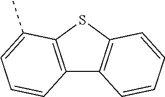 | Ar¹-37 |
| 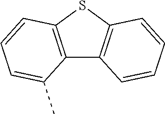 | Ar¹-38 |
| 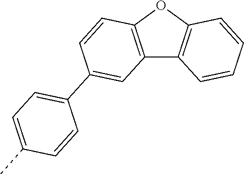 | Ar¹-39 |
| 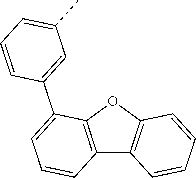 | Ar¹-40 |
| 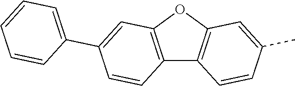 | Ar¹-41 |
| 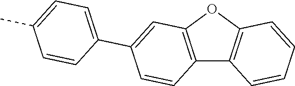 | Ar¹-42 |
| 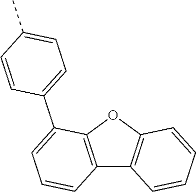 | Ar¹-43 |
| 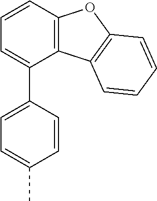 | Ar¹-44 |
| 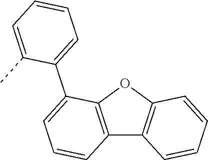 | Ar¹-45 |
| 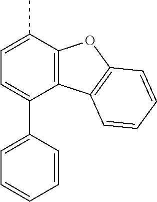 | Ar¹-46 |
| 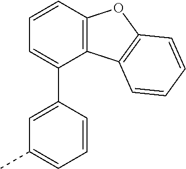 | Ar¹-47 |
| 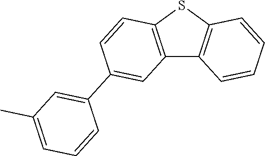 [SMILES: Cc1cccc(-c2ccc3sc4ccccc4c3c2)c1] | Ar¹-48 |
| 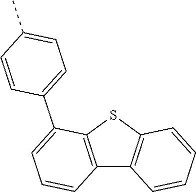 | Ar¹-49 |
| 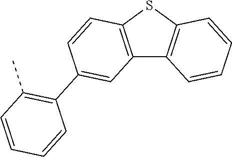 | Ar¹-50 |
| 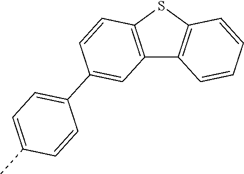 | Ar¹-51 |
| 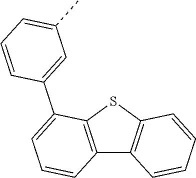 | Ar¹-52 |
| 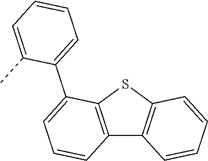 | Ar¹-53 |
| 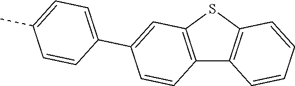 | Ar¹-54 |
| 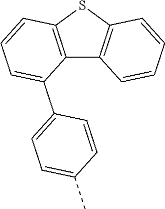 | Ar¹-55 |
| 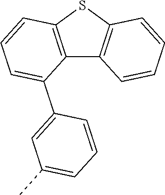 | Ar¹-56 |
| 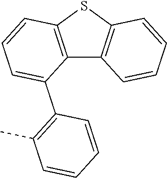 | Ar¹-57 |
| 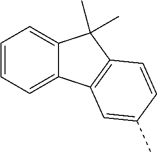 | Ar¹-82 |
| 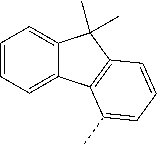 | Ar¹-83 |
| 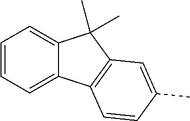 | Ar¹-84 |
| 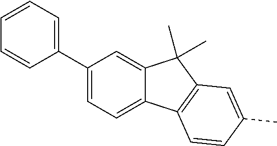 | Ar¹-85 |
| 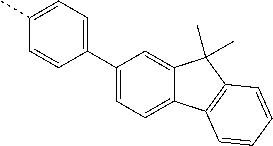 | Ar¹-86 |
| 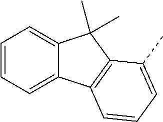 | Ar¹-87 |
| 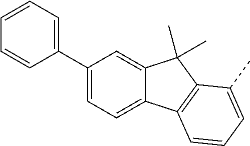 | Ar¹-88 |
| 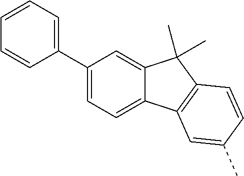 | Ar¹-89 |
| 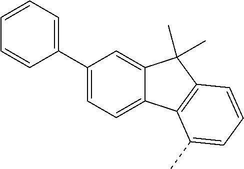 | Ar¹-90 |
| 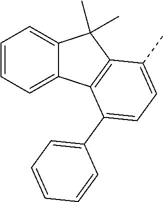 | Ar¹-91 |
| 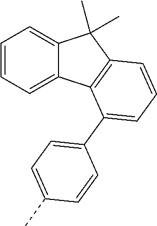 | Ar¹-92 |
| 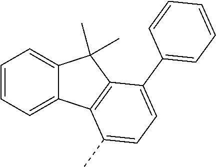 | Ar¹-93 |
| 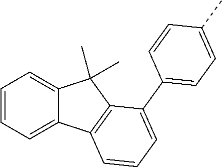 | Ar¹-94 |
| 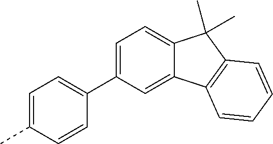 | Ar¹-95 |
| 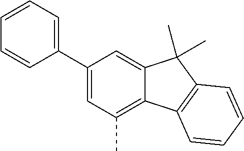 | Ar¹-96 |
| 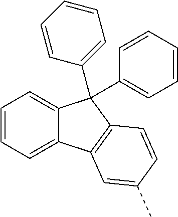 | Ar¹-97 |
| 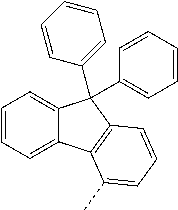 | Ar¹-98 |
| 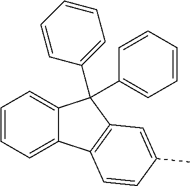 | Ar¹-99 |
| 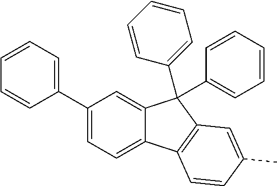 | Ar¹-100 |
| 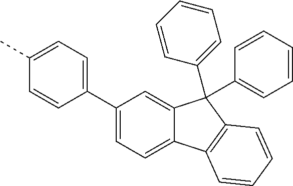 | Ar¹-101 |
| 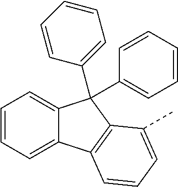 | Ar¹-102 |
| 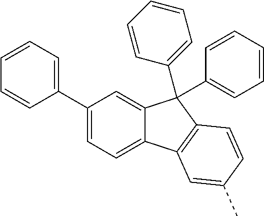 | Ar¹-103 |
| 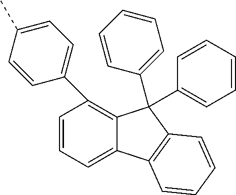 | Ar¹-104 |
| 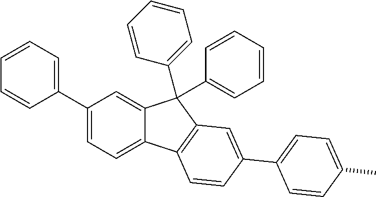 | Ar¹-105 |
| 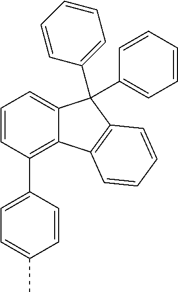 | Ar¹-106 |
| 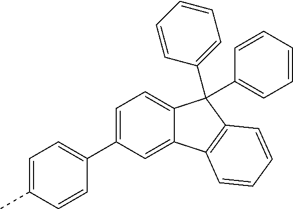 | Ar¹-107 |
| 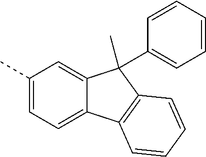 | Ar¹-108 |
| 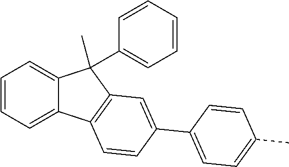 | Ar¹-109 |
| 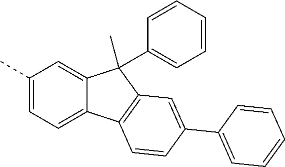 | Ar¹-110 |
| 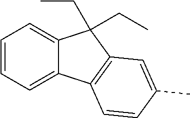 | Ar¹-111 |
| 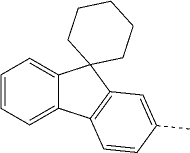 | Ar¹-112 |
| 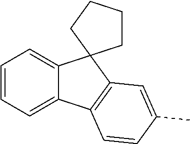 | Ar¹-113 |
| 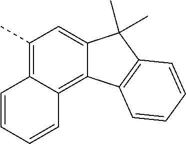 | Ar¹-114 |
| 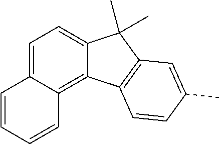 | Ar¹-115 |
| 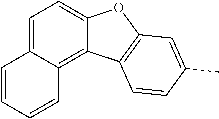 | Ar¹-116 |
| 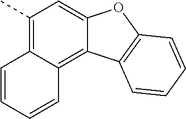 | Ar¹-117 |
| 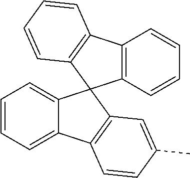 | Ar¹-118 |
| 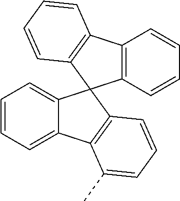 | Ar¹-119 |
| 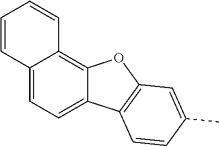 | Ar¹-120 |
| 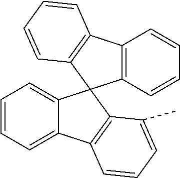 | Ar¹-121 |
| 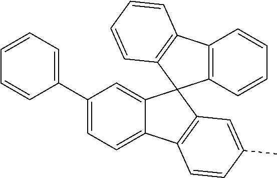 | Ar¹-122 |
| 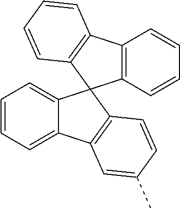 | Ar¹-123 |
| 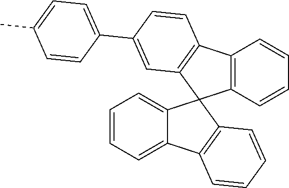 | Ar¹-124 |
| 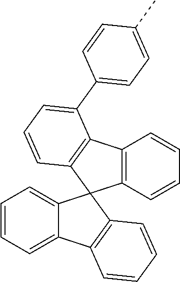 | Ar¹-125 |
| 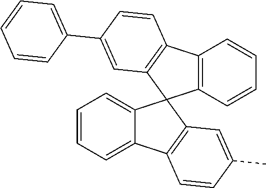 | Ar¹-126 |
| 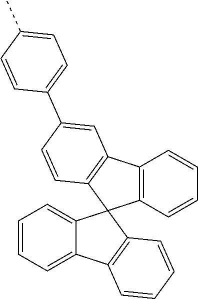 | Ar¹-127 |
| 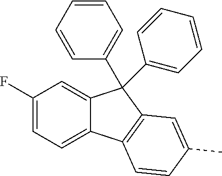 | Ar¹-128 |
| 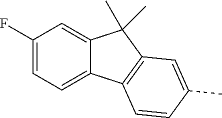 | Ar¹-129 |
| 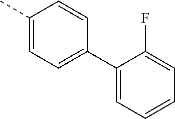 | Ar¹-130 |
| 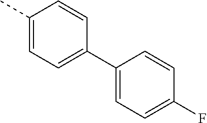 | Ar¹-131 |
| 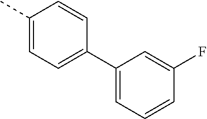 | Ar¹-132 |
| 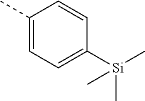 | Ar¹-133 |
| 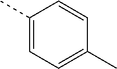 | Ar¹-134 |
| 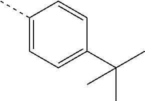 | Ar¹-135 |
| 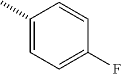 | Ar¹-136 |
| 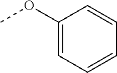 | Ar¹-137 |
| 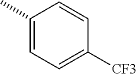 | Ar¹-138 |
| 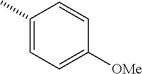 | Ar¹-139 |
| 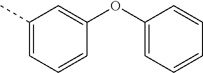 | Ar¹-140 |
| 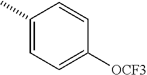 | Ar¹-141 |
| 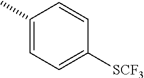 | Ar¹-142 |
| —F | Ar¹-143 |
| —Cl | Ar¹-144 |
| —Br | Ar¹-145 |
| —I | Ar¹-146 |
| —H | Ar¹-147 |
| —CH₃ | Ar¹-148 |
| 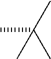 | Ar¹-149 |
| —CH₂CH₃ | Ar¹-150 |
| 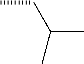 | Ar¹-151 |
| —CF₃ | Ar¹-152 |
| —CF₂CF₃ | Ar¹-153 |
| —OCF₃ | Ar¹-154 |
| —SCF₃ | Ar¹-155 |
| —SF₅ | Ar¹-156 |
| —OCF₂CF₃ | Ar¹-157 |
| —SCF₂CF₃ | Ar¹-158 |
| 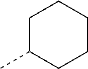 | Ar¹-159 |
| 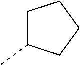 | Ar¹-160 |
| 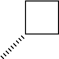 | Ar¹-161 |
| 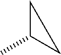 | Ar¹-162 |
| —CN | Ar¹-163 |
| —SCN | Ar¹-164 |
| —OCH₂CH₃ | Ar¹-165 |
| —OCH₃ | Ar¹-166 |
| —SCH₃ | Ar¹-167 |
| —Si(CH₃)₃ | Ar¹-168 |
| —Si(CH₃)₂t-Bu | Ar¹-169 |
| —Si(iPr)₃ | Ar¹-170 |
| —Si(CH₃)₂Ph | Ar¹-171 |
| Si—(Ph)₃ | Ar¹-172 |
| C—(Ph)₃ | Ar¹-173 |
| —D | Ar¹-174 |
| —CD₃ | Ar¹-175 |
| —CD₂—(CD₃)₂ | Ar¹-176 |
| —C(CD₃)₃ | Ar¹-177 |
| —CD₂—(CD₃)₂ | Ar¹-178 |
| —OCD₃ | Ar¹-179 |
| —SCD₃ | Ar¹-180 |
| —Si(CD₃)₃ | Ar¹-181 |
| 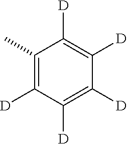 | Ar¹-182 |
| 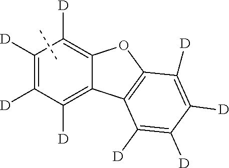 | Ar¹-183 |
| 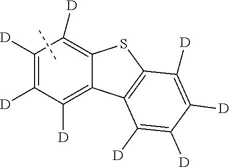 | Ar¹-184 |
| 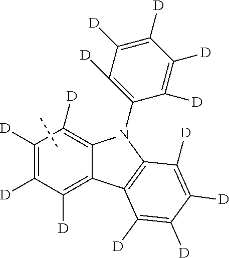 | Ar¹-185 |
| —CD₂(CH₃)₂ | Ar¹-186 |
| —CH₂(CH₃)₃ | Ar¹-187 |
| 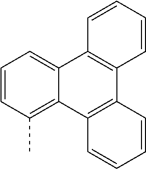 | Ar¹-188 |
| 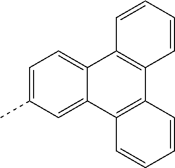 | Ar¹-189 |
| 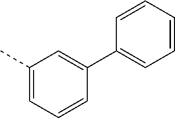 | Ar¹-190 |
| 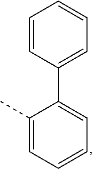 | Ar¹-191 |

where the dotted lines represent the bonds to the rest of the formula (I). Particular preference is given here to the (Ar¹-1), (Ar¹-2), (Ar¹-8), (Ar¹-28), (Ar¹-30), (Ar¹-31), (Ar¹-34), (Ar¹-34), (Ar¹-43), (Ar¹-44), (Ar¹-144), (Ar¹-163), (Ar¹-189) and (Ar¹-190) groups.

R¹ is preferably the same or different at each instance and is selected from H, D, F, CN, Si(R⁵)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—. More preferably, R¹ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H. Even more preferably, R¹ is H.

It is preferably ruled out here that R¹, and also Ar⁰ and Ar¹, denote a carbazolyl group bonded to the rest of the formula (I) via its nitrogen atom.

Preferred R¹ groups are shown in the following table:

| | |
|---|---|
| 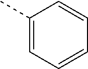 | R¹-1 |
| 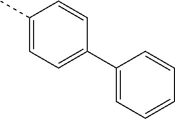 | R¹-2 |
| 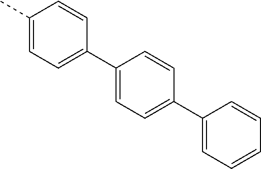 | R¹-3 |
| 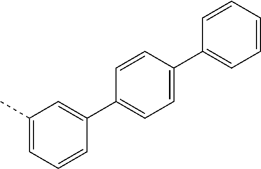 | R¹-4 |
| 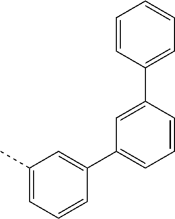 | R¹-5 |
| 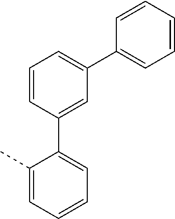 | R¹-6 |
| 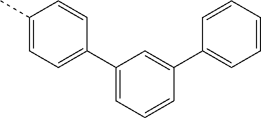 | R¹-7 |
| 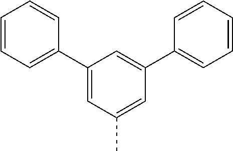 | R¹-8 |
| 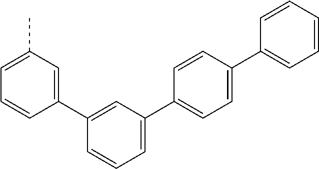 | R¹-9 |
| 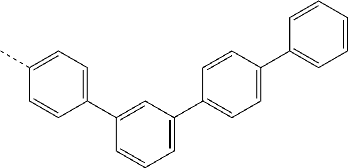 | R¹-10 |
| 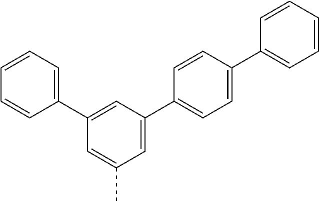 | R¹-11 |
| 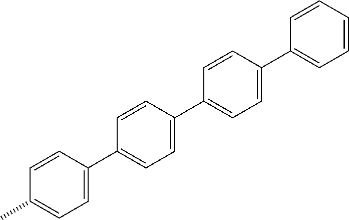 | R¹-12 |
| 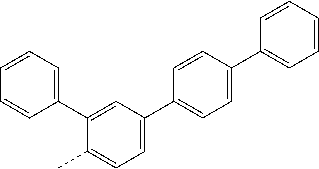 | R¹-13 |
| 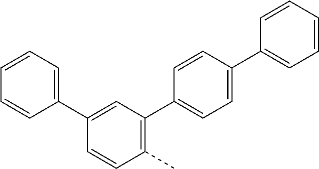 | R¹-14 |
| 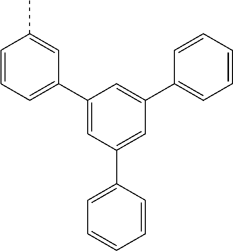 | R¹-15 |
| 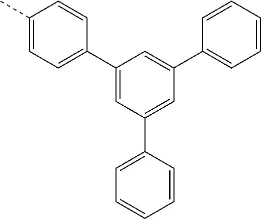 | R¹-16 |
| 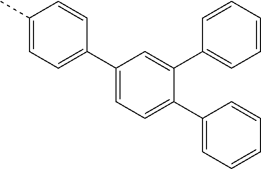 | R¹-17 |
| 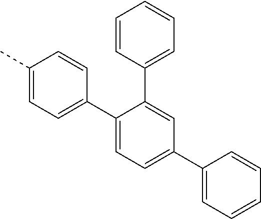 | R¹-18 |
| 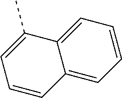 | R¹-19 |
| 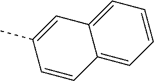 | R¹-20 |
| 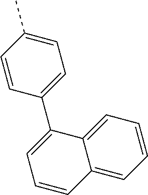 | R¹-21 |
| 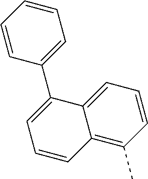 | R¹-22 |
| 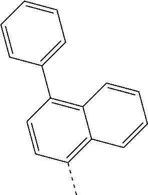 | R¹-23 |
| 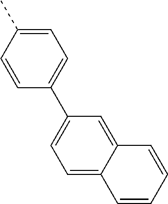 | R¹-24 |
| 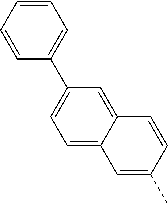 | R¹-25 |
| 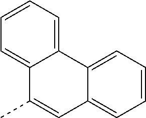 | R¹-26 |
| 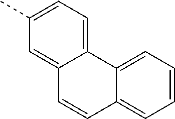 | R¹-27 |
| 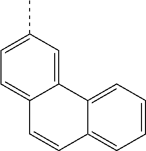 | R¹-28 |
| 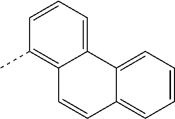 | R¹-29 |
| 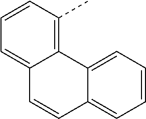 | R¹-30 |
| 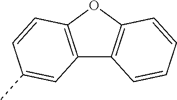 | R¹-31 |
| 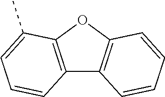 | R¹-32 |
| 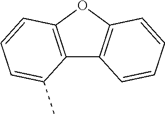 | R¹-33 |
| 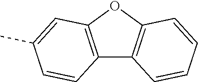 | R¹-34 |
| 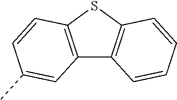 | R¹-35 |
| 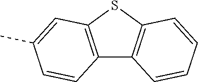 | R¹-36 |
| 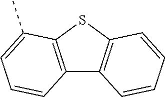 | R¹-37 |
| 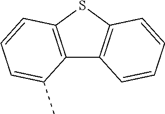 | R¹-38 |
| 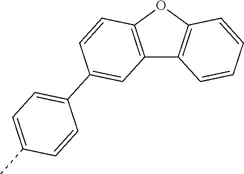 | R¹-39 |
| 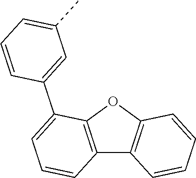 | R¹-40 |
| 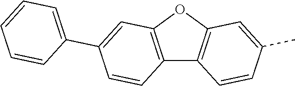 | R¹-41 |
| 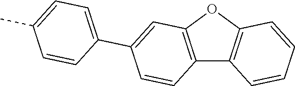 | R¹-42 |
| 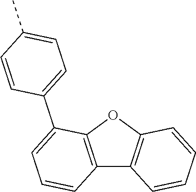 | R¹-43 |
| 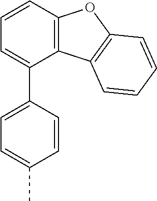 | R¹-44 |
| 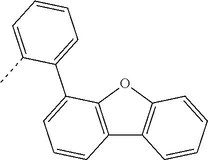 | R¹-45 |
| 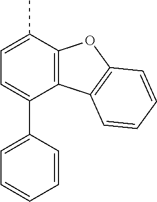 | R¹-46 |
| 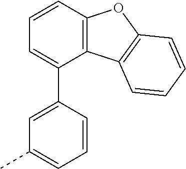 | R¹-47 |
| 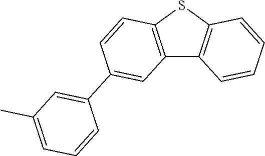 [SMILES: Cc1cccc(-c2ccc3sc4ccccc4c3c2)c1] | R¹-48 |
| 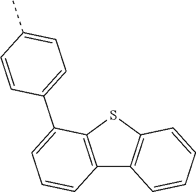 | R¹-49 |
| 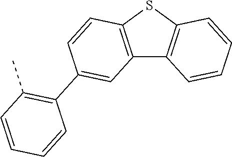 | R¹-50 |
| 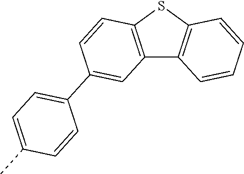 | R¹-51 |
| 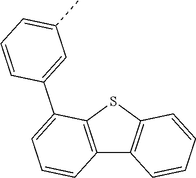 | R¹-52 |
| 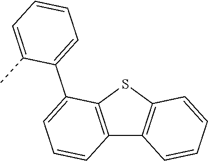 | R¹-53 |
| 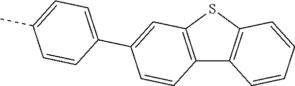 | R¹-54 |
| 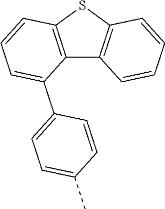 | R¹-55 |
| 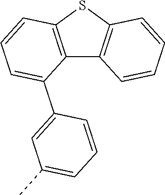 | R¹-56 |
| 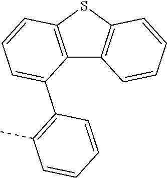 | R¹-57 |
| 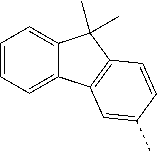 | R¹-82 |
| 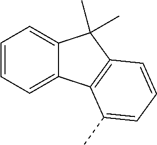 | R¹-83 |
| 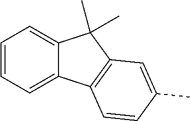 | R¹-84 |
| 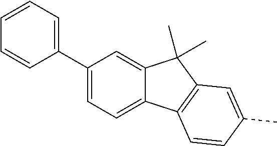 | R¹-85 |
| 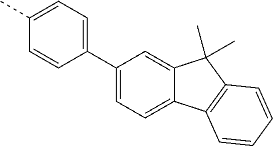 | R¹-86 |
| 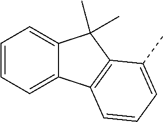 | R¹-87 |
| 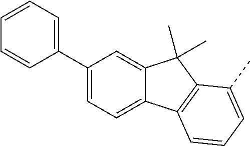 | R¹-88 |
| 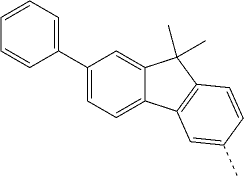 | R¹-89 |
| 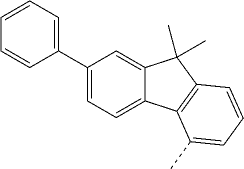 | R¹-90 |
| 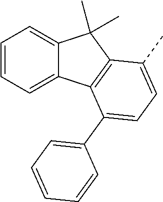 | R¹-91 |
| 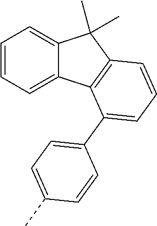 | R¹-92 |
| 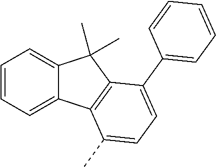 | R¹-93 |
| 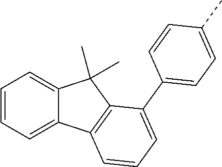 | R¹-94 |
| 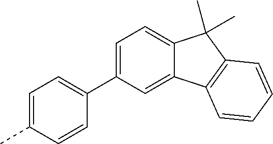 | R¹-95 |
| 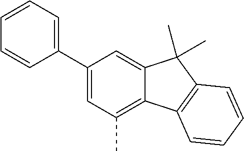 | R¹-96 |
| 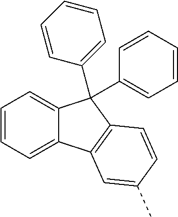 | R¹-97 |
| 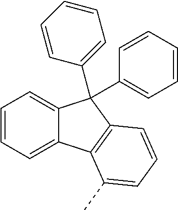 | R¹-98 |
| 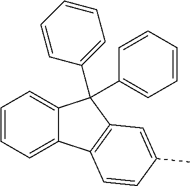 | R¹-99 |
| 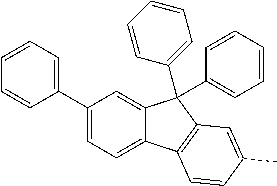 | R¹-100 |
| 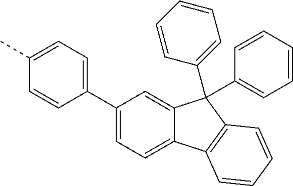 | R¹-101 |
| 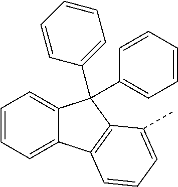 | R¹-102 |
| 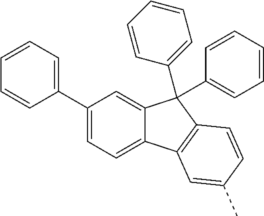 | R¹-103 |
| 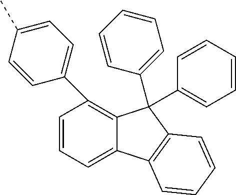 | R¹-104 |
| 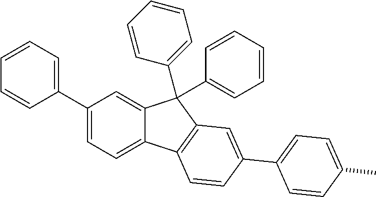 | R¹-105 |
| 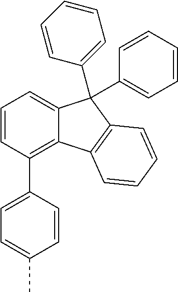 | R¹-106 |
| 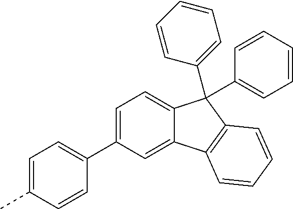 | R¹-107 |
| 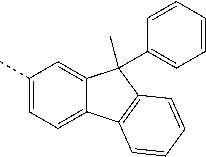 | R¹-108 |
| 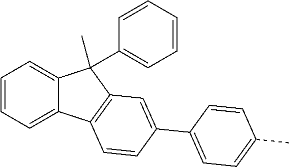 | R¹-109 |
| 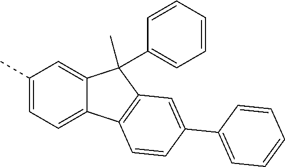 | R¹-110 |
| 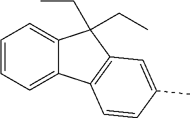 | R¹-111 |
| 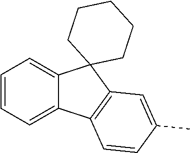 | R¹-112 |
| 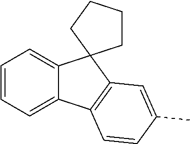 | R¹-113 |
| 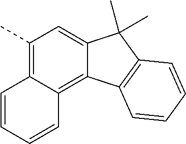 | R¹-114 |
| 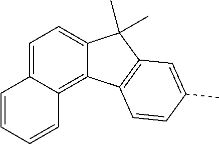 | R¹-115 |
| 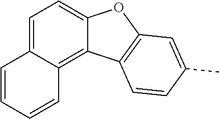 | R¹-116 |
| 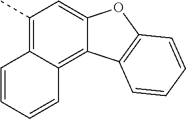 | R¹-117 |
| 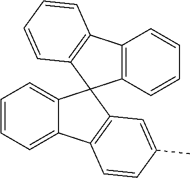 | R¹-118 |
| 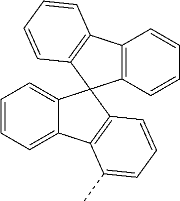 | R¹-119 |
| 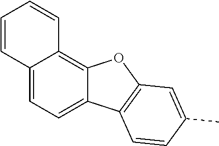 | R¹-120 |
| 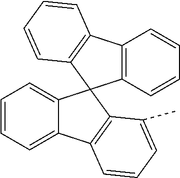 | R¹-121 |
| 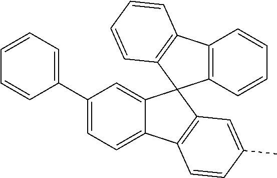 | R¹-122 |
| 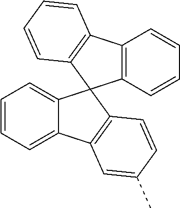 | R¹-123 |
| 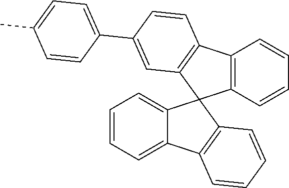 | R¹-124 |
| 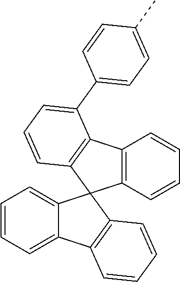 | R¹-125 |
| 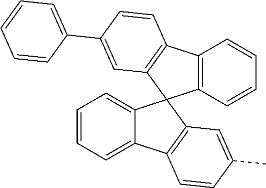 | R¹-126 |
| 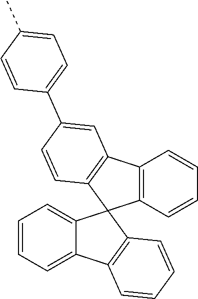 | R¹-127 |
| 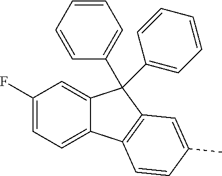 | R¹-128 |
| 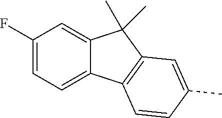 | R¹-129 |
| 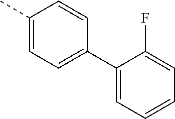 | R¹-130 |
| 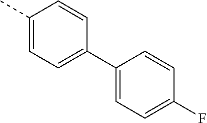 | R¹-131 |
| 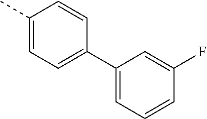 | R¹-132 |
| 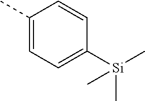 | R¹-133 |
| 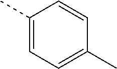 | R¹-134 |
| 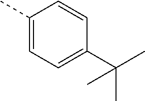 | R¹-135 |
| 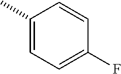 | R¹-136 |
| 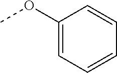 | R¹-137 |
| 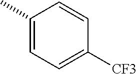 | R¹-138 |
| 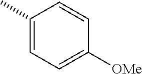 | R¹-139 |
| 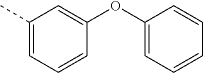 | R¹-140 |
| 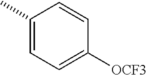 | R¹-141 |
| 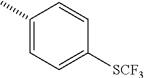 | R¹-142 |
| —F | R¹-143 |
| —Cl | R¹-144 |
| —Br | R¹-145 |
| —I | R¹-146 |
| —H | R¹-147 |
| —CH₃ | R¹-148 |
| 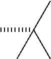 | R¹-149 |
| —CH₂CH₃ | R¹-150 |
| 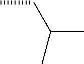 | R¹-151 |
| —CF₃ | R¹-152 |
| —CF₂CF₃ | R¹-153 |
| —OCF₃ | R¹-154 |
| —SCF₃ | R¹-155 |
| —SF₅ | R¹-156 |
| —OCF₂CF₃ | R¹-157 |
| —SCF₂CF₃ | R¹-158 |
| 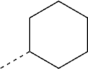 | R¹-159 |
| 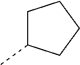 | R¹-160 |
| 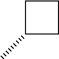 | R¹-161 |
| 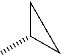 | R¹-162 |
| —CN | R¹-163 |
| —SCN | R¹-164 |
| —OCH₂CH₃ | R¹-165 |
| —OCH₃ | R¹-166 |
| —SCH₃ | R¹-167 |
| —Si(CH₃)₃ | R¹-168 |
| —Si(CH₃)₂t-Bu | R¹-169 |
| —Si(iPr)₃ | R¹-170 |
| —Si(CH₃)₂Ph | R¹-171 |
| Si—(Ph)₃ | R¹-172 |
| C—(Ph)₃ | R¹-173 |
| —D | R¹-174 |
| —CD₃ | R¹-175 |
| —CD₂—(CD₃)₂ | R¹-176 |
| —C(CD₃)₃ | R¹-177 |
| —CD₂—(CD₃)₂ | R¹-178 |
| —OCD₃ | R¹-179 |
| —SCD₃ | R¹-180 |
| —Si(CD₃)₃ | R¹-181 |
| 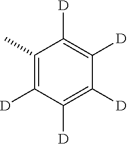 | R¹-182 |
| 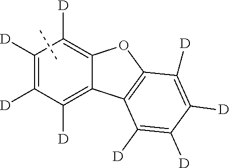 | R¹-183 |
| 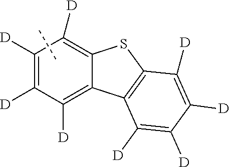 | R¹-184 |
| 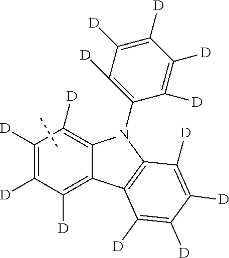 | R¹-185 |
| —CD₂(CH₃)₂ | R¹-186 |
| —CH₂(CH₃)₃ | R¹-187 |
| 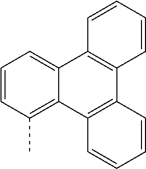 | R¹-188 |
| 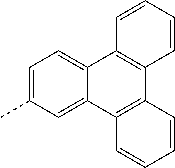 | R¹-189 |
| 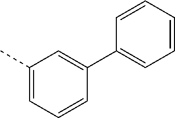 | R¹-190 |
| 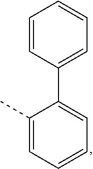 | R¹-191 |

where the dotted lines represent the bonds to the rest of the formula (I). Particular preference is given here to the (Ar¹-1), (Ar¹-2), (Ar¹-8), (Ar¹-28), (Ar¹-30), (Ar¹-31), (Ar¹-34), (Ar¹-34), (Ar¹-43), (Ar¹-44), (Ar¹-144), (Ar¹-163), (Ar¹-189) and (Ar¹-190) groups.

R² is preferably the same or different at each instance and is selected from H, D, F, CN, Si(R⁵)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—. More preferably, R² is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H. Even more preferably, R² is H.

Preferably, only one or two A groups are present in formula (I); more preferably, exactly one A group is present in formula (I).

When two A groups are present in formula (I), they are preferably both bonded to the heteroaromatic five-membered ring of the formula (I).

The bridging Ar^{L} group is preferably the same or different at each instance and is selected from aromatic ring systems which have 6 to 20 aromatic ring atoms and are substituted by R² radicals, and heteroaromatic ring systems which have 5 to 20 aromatic ring atoms and are substituted by R² radicals. Particularly preferred Ar^{L} groups are the same or different at each instance and are selected from divalent groups derived from benzene, biphenyl, terphenyl, naphthalene, fluorene, indenofluorene, indenocarbazole, spirobifluorene, dibenzofuran and dibenzothiophene, each of which are substituted by R² radicals. Even more preferably, Ar^{L} is a divalent group derived from benzene, biphenyl, dibenzofuran or dibenzothiophene, each of which is substituted by one or more R² radicals, where the R² radicals in this case are preferably H.

Preferably, n is 0.

Preferred —(Ar^{L})ₙ— groups in the case that n=1 conform to the following formulae:

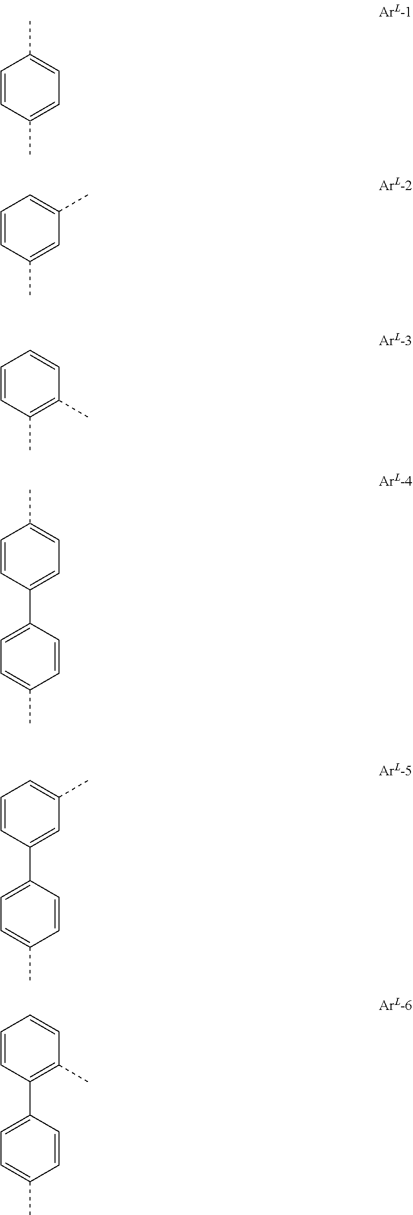

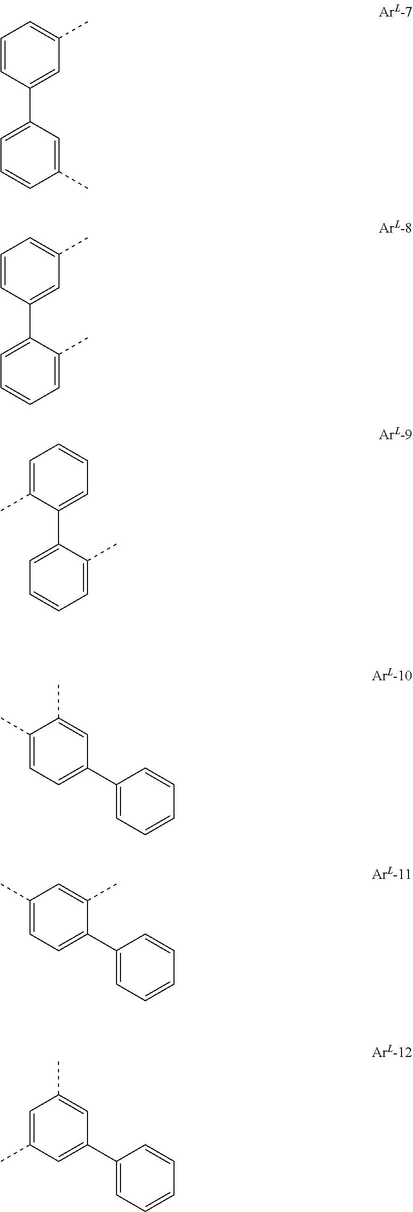

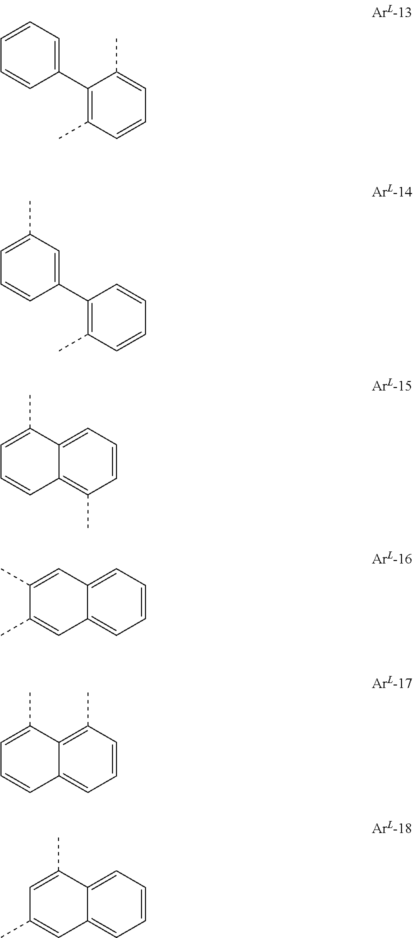

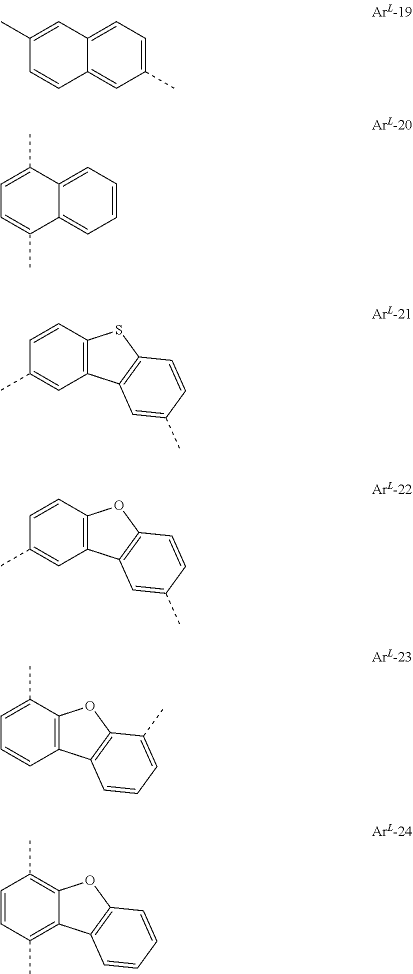

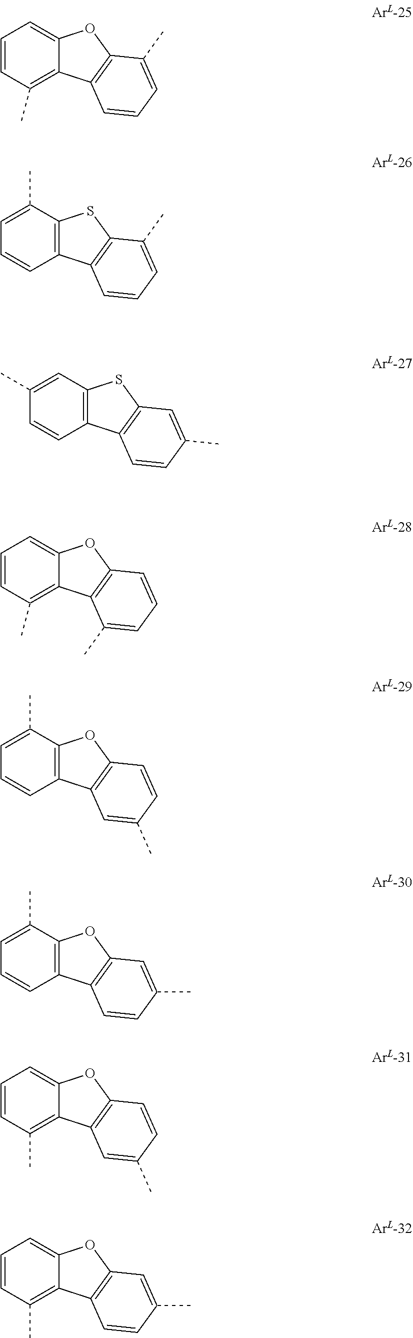

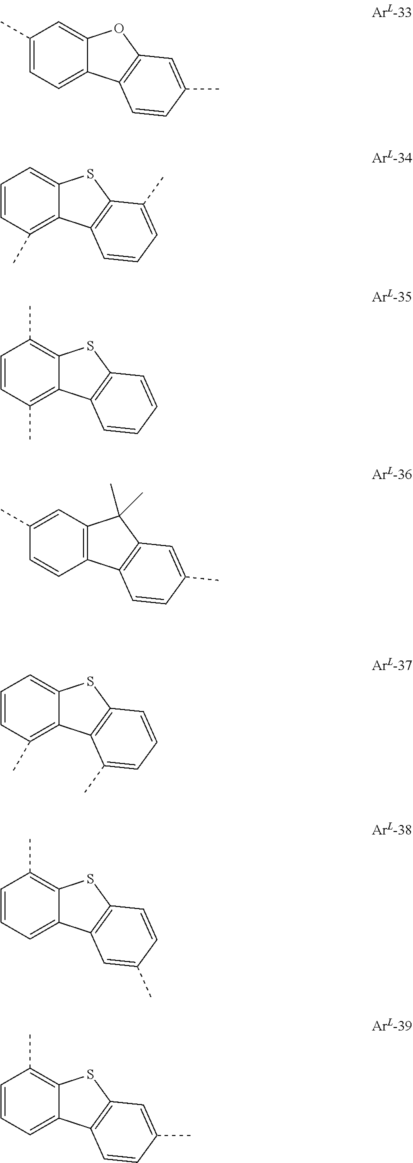

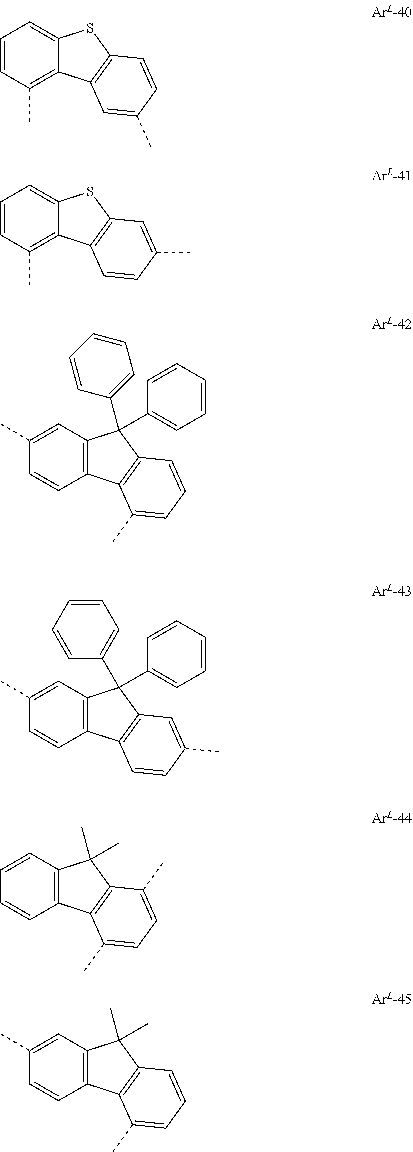

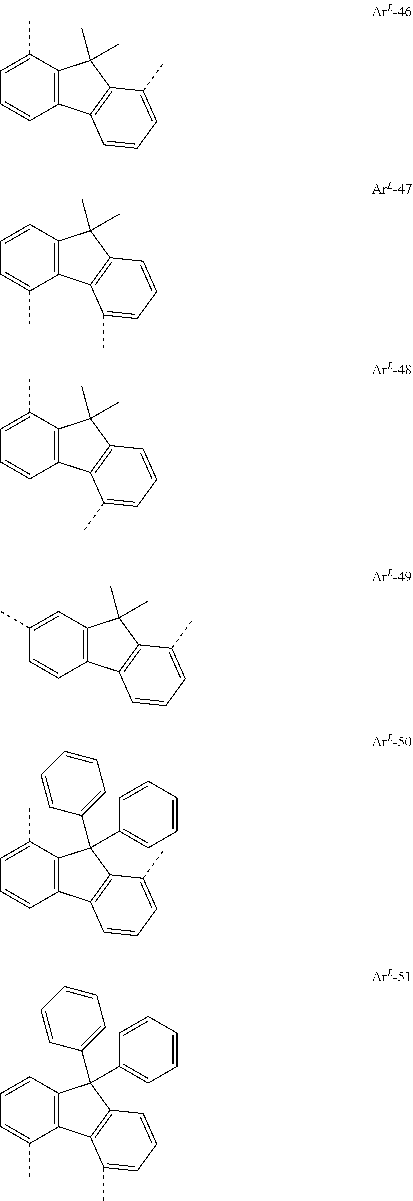

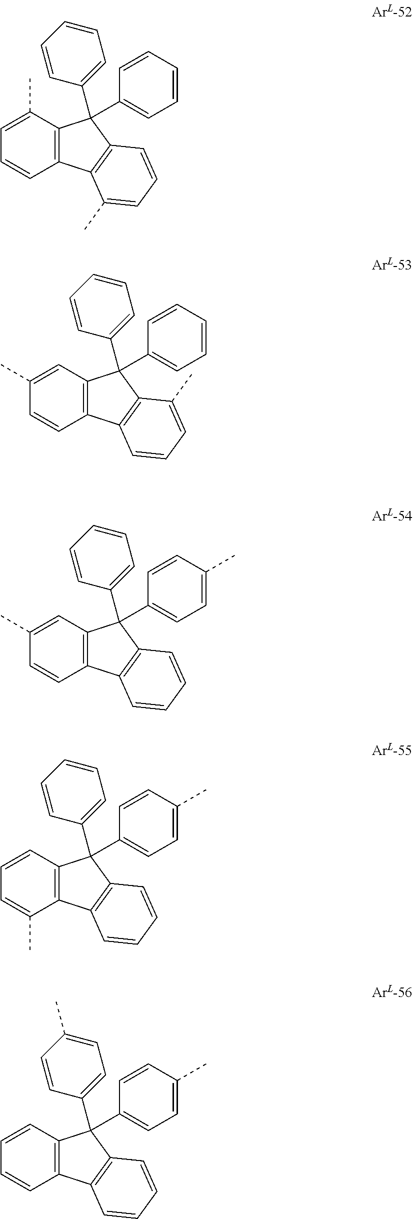

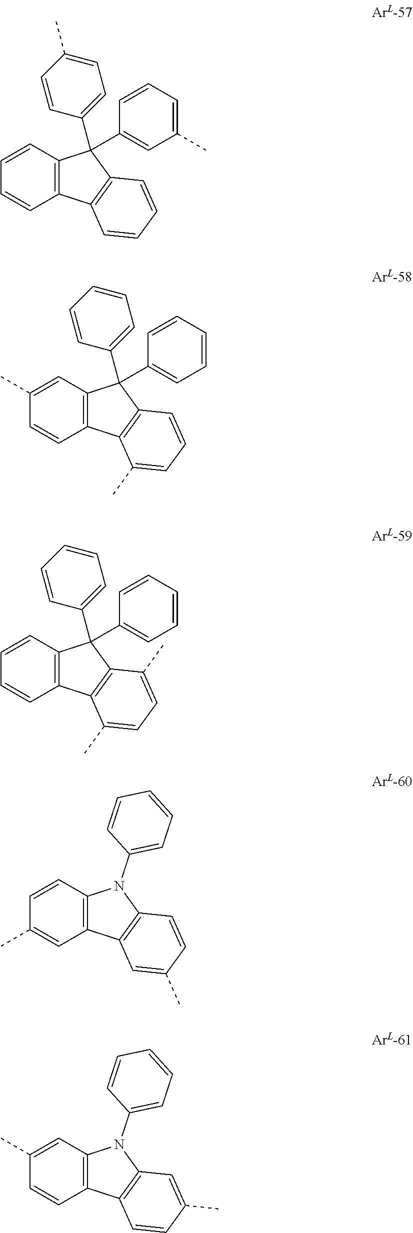

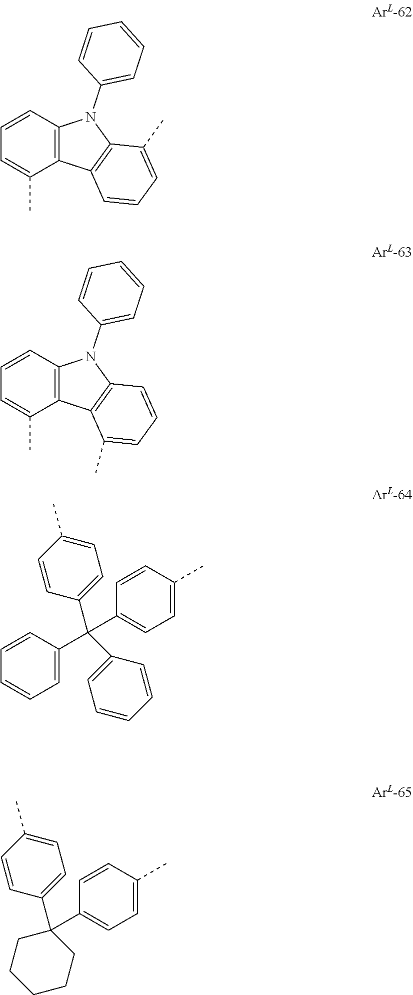

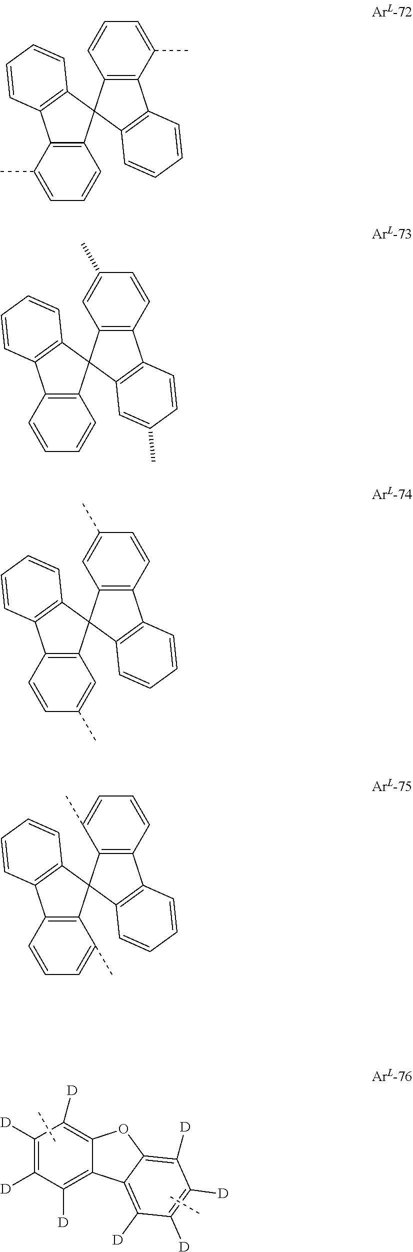

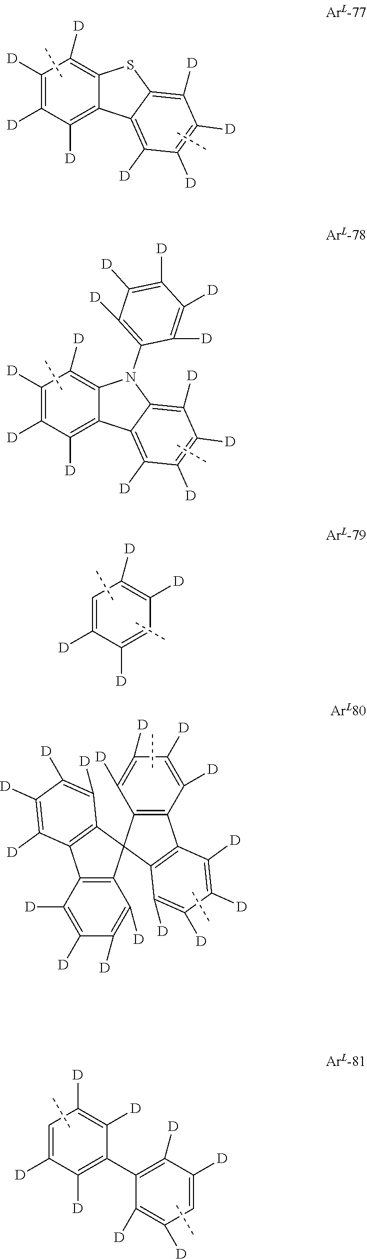

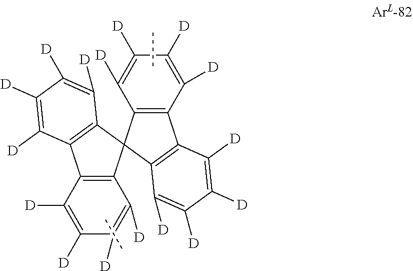

where the dotted lines represent the bonds to the rest of the formula (I), and where the groups at the positions shown as unsubstituted are each substituted by R² radicals, where the R² radicals in these positions are preferably H.

Among the abovementioned formulae, particular preference is given to the formulae (Ar^{L}-1), (Ar^{L}-2), (Ar^{L}-12), (Ar^{L}-20), (Ar^{L}-31).

The electron-transporting ET group is preferably the same or different at each instance and is selected from heteroaryl groups having 5 to 40 aromatic ring atoms, of which at least one, preferably at least two and very preferably at least three are heteroatoms that are preferably selected from N, O and/or S, and are most preferably N, where the heteroaryl groups are each substituted by Ar² groups.

It is preferable that each ET group is substituted by at least one Ar² group that is not H, more preferably at least two Ar² groups that are not H.

Further preferably, the ET group is the same or different at each instance and is selected from the groups of the formulae (ET-1) to (ET-11) and (ET-11a) to (ET-11c):

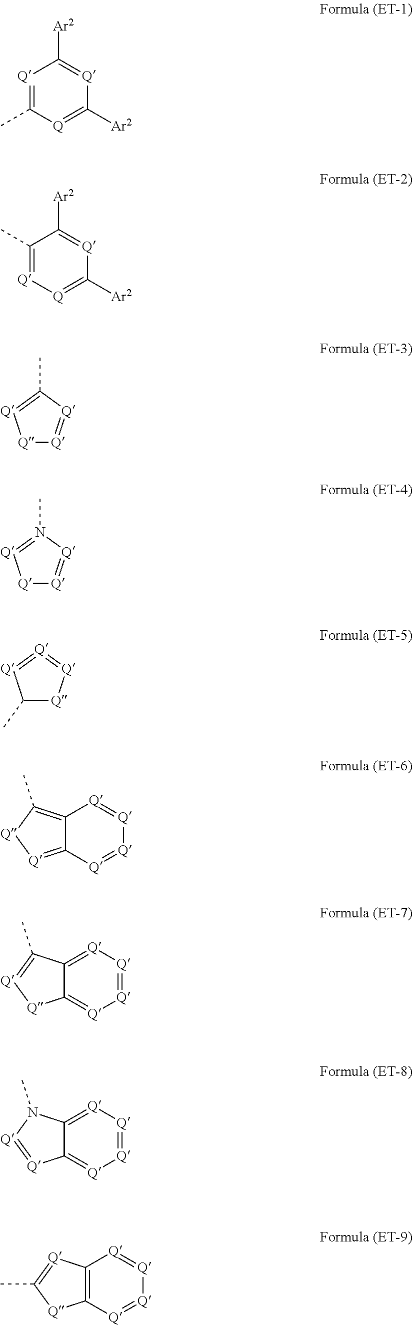

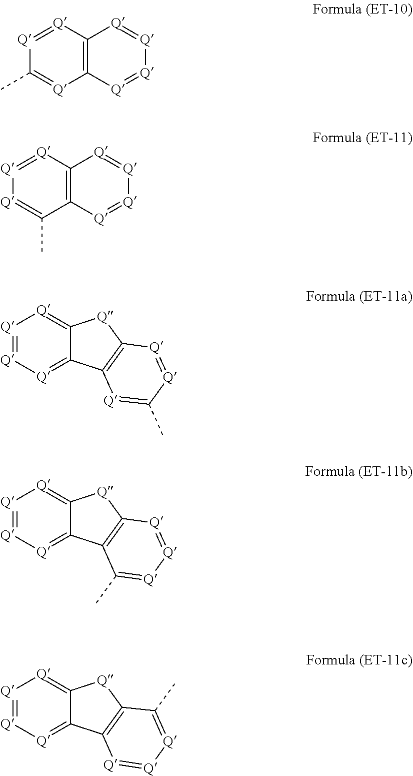

where

Q′ is the same or different at each instance and is selected from CAr² or N;

Q″ is the same or different at each instance and is selected from NR², O or S;

the dotted line in each case marks the attachment position to the rest of the formula (I), and R² and Ar² are as defined above; and

where at least one Q′ is N.

Particularly preferred ET groups are the same or different at each instance and are selected from groups derived from pyridine, pyrazine, pyrimidine, pyridazine, 1,2,4-triazine, 1,3,5-triazine, quinoline, isoquinoline, quinoxaline, quinazoline, pyrazole, imidazole, benzimidazole, thiazole, benzothiazole, oxazole, oxadiazole and benzoxazole, each of which are substituted by Ar² groups. The ET group is most preferably a pyridine, pyrazine, pyrimidine, pyridazine, 1,3,5-triazine, benzimidazole and quinazoline substituted by one or more Ar² groups.

Very particularly preferred heteroaromatic electron-transporting ET groups are selected from the formulae (ET-12) to (ET-35)

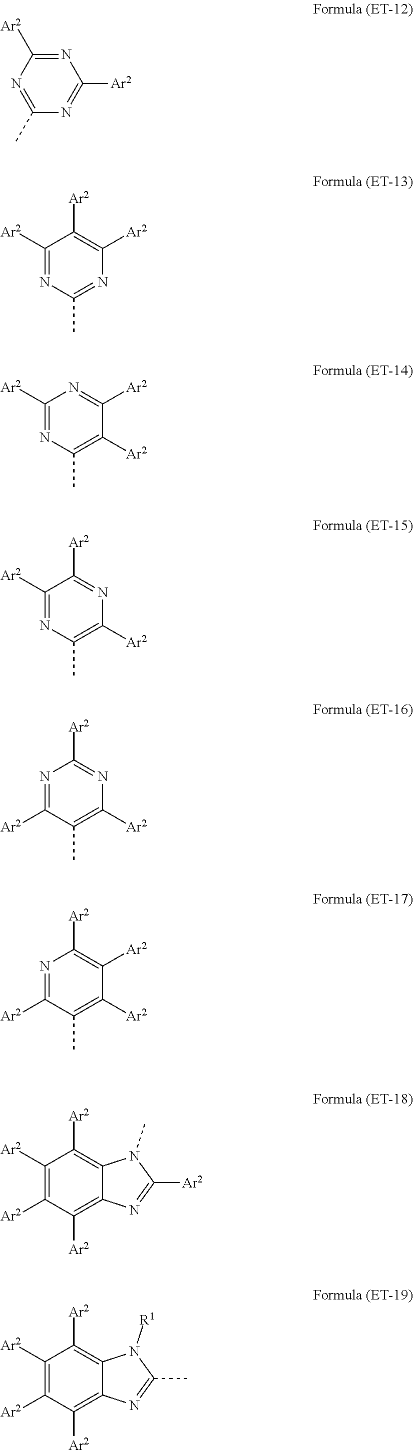

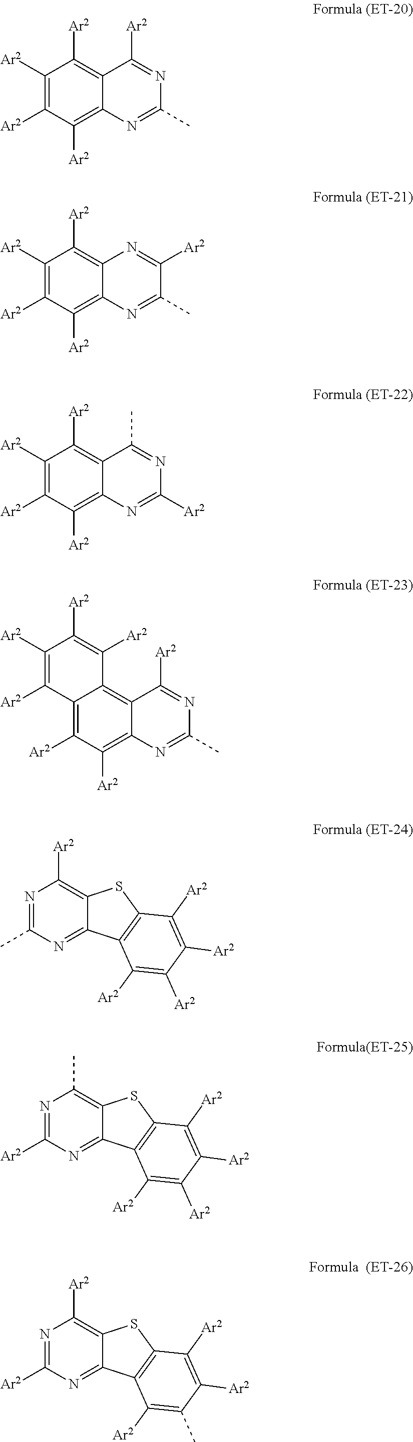

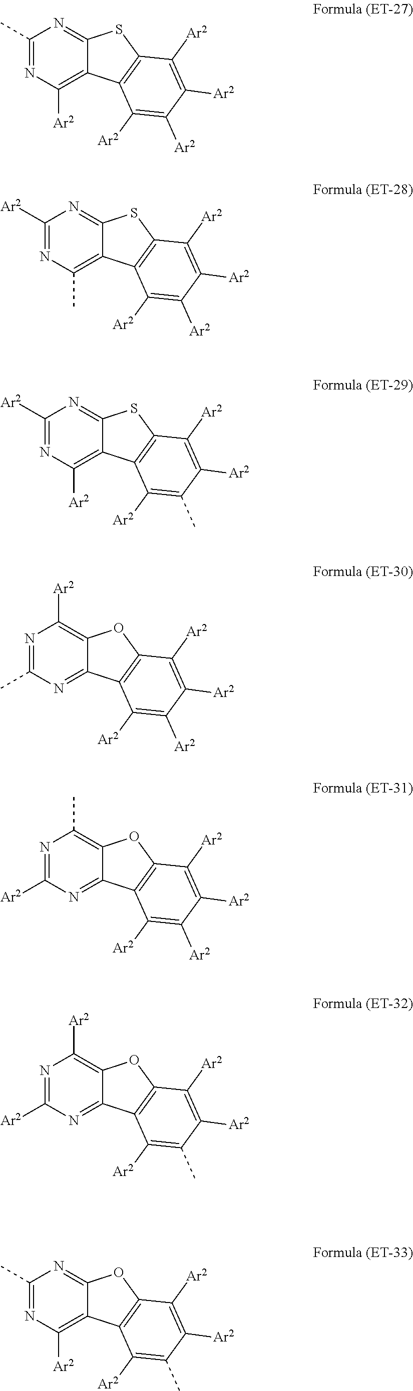

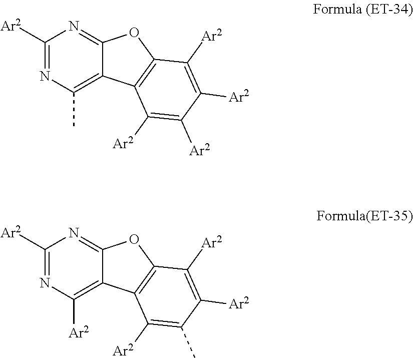

where the dotted lines represent the bonds to the rest of the formula (I), very particular preference being given to groups of the formula (ET-12), (ET-13), (ET-14), (ET-20), (ET-21) and (ET-22), and greatest preference to groups of the formula (ET-12).

Preferably, Ar² is the same or different at each instance and is selected from

H, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms, where the alkyl groups, alkoxy groups, aromatic ring systems and heteroaromatic ring systems may each be substituted by R³ radicals, and where two or more adjacent Ar² groups together may form a mono- or polycyclic, aliphatic or aromatic ring system.

Ar² is more preferably the same or different at each instance and is selected from H and aromatic ring systems which have 6 to 40 aromatic ring atoms and are substituted by R³ radicals. More particularly preferably, Ar² is the same or different at each instance and is selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R³ radicals, and H. Very particularly preferred Ar² groups are the same or different at each instance and are selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R³ radicals, and H.

Very particularly preferred Ar² groups are the same or different and are selected from H and the following formulae:

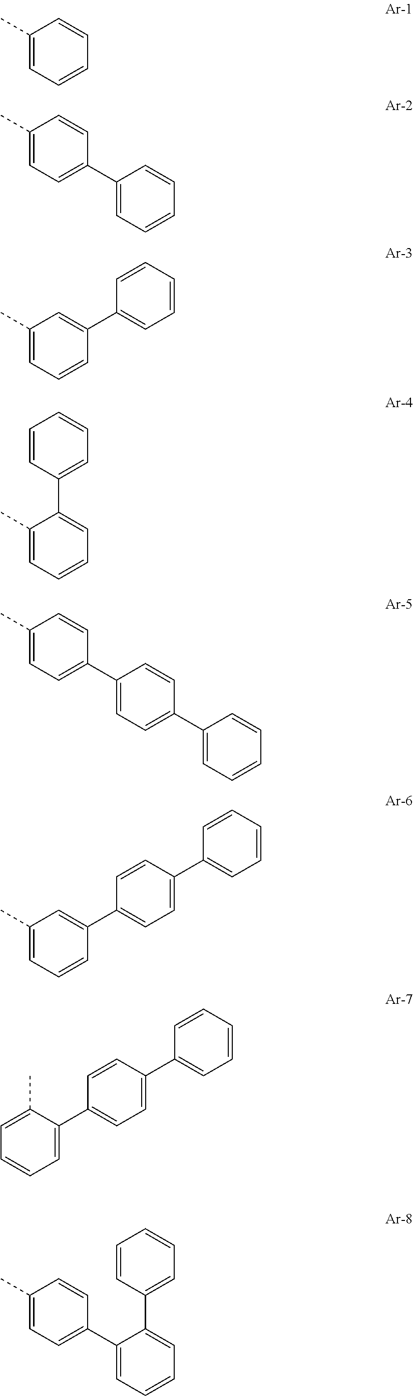

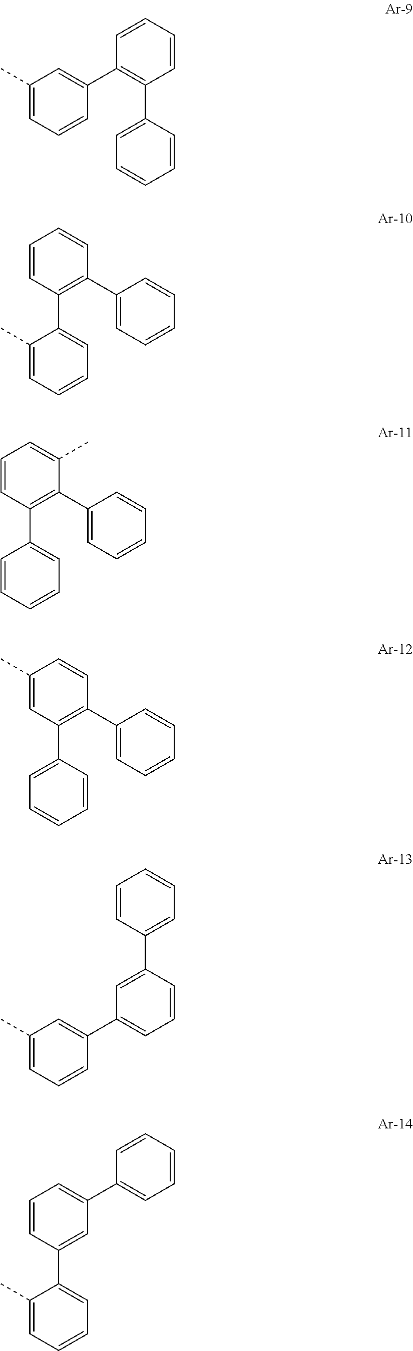

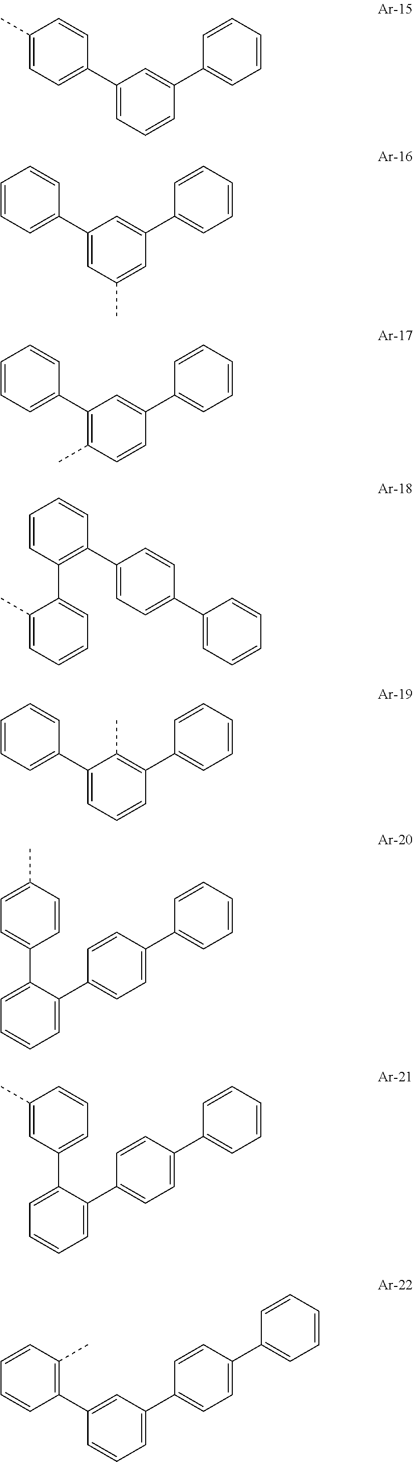

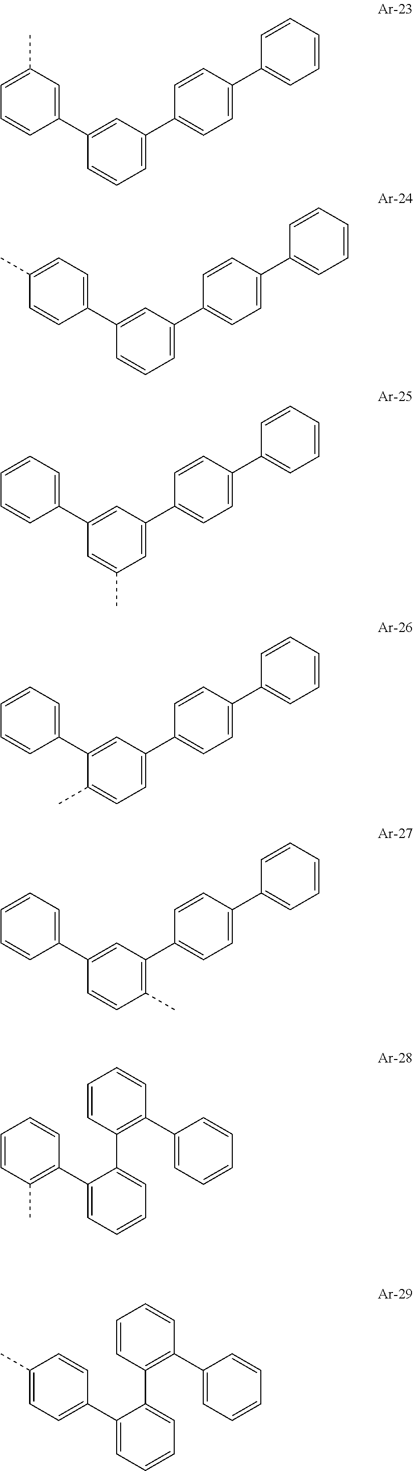

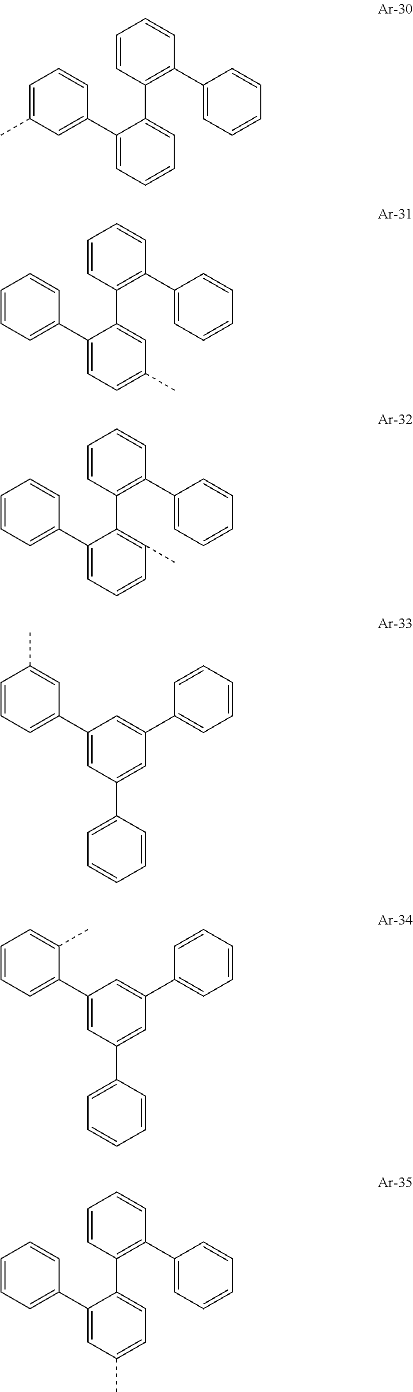

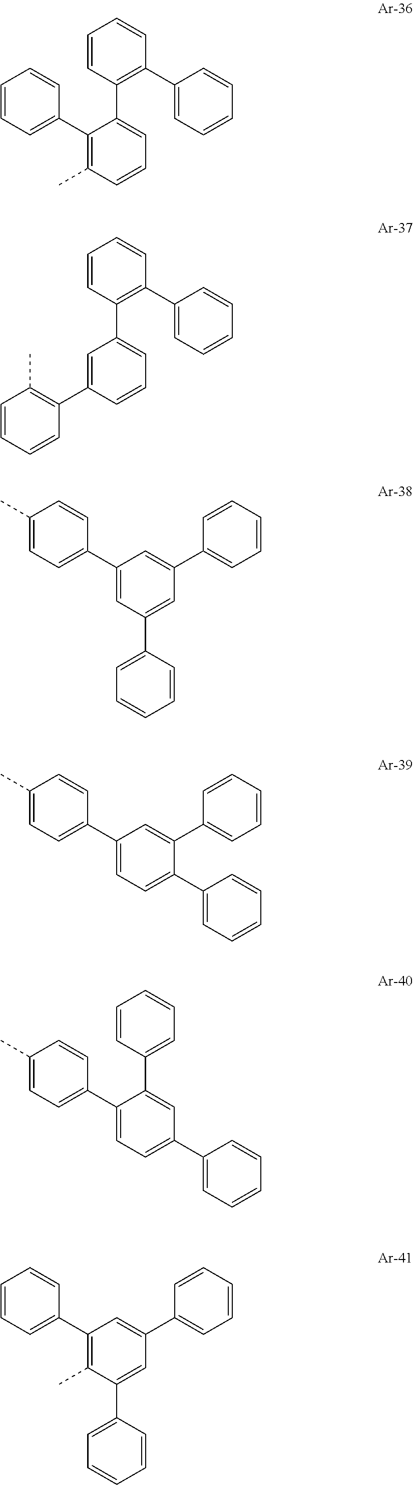

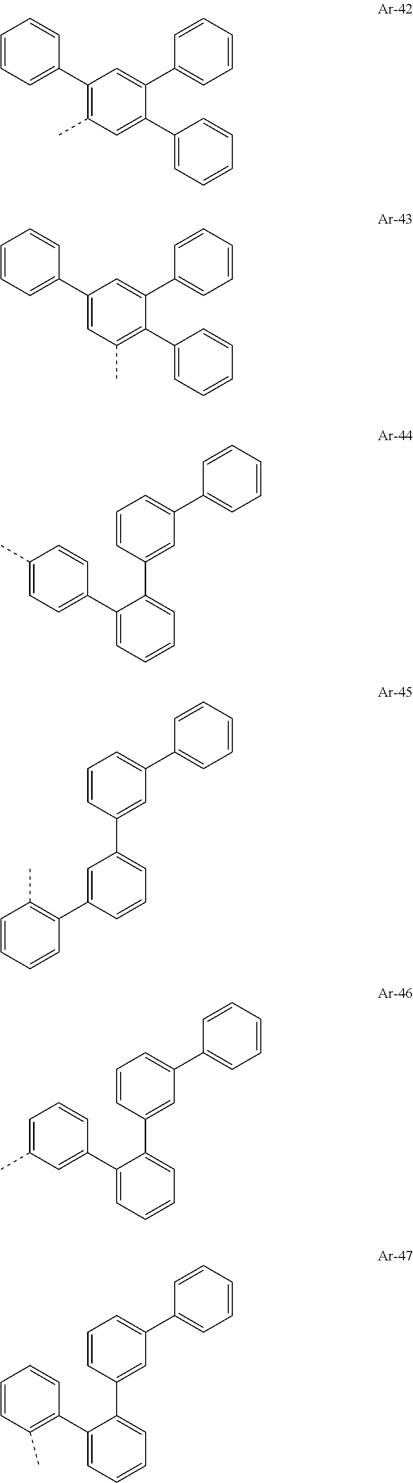

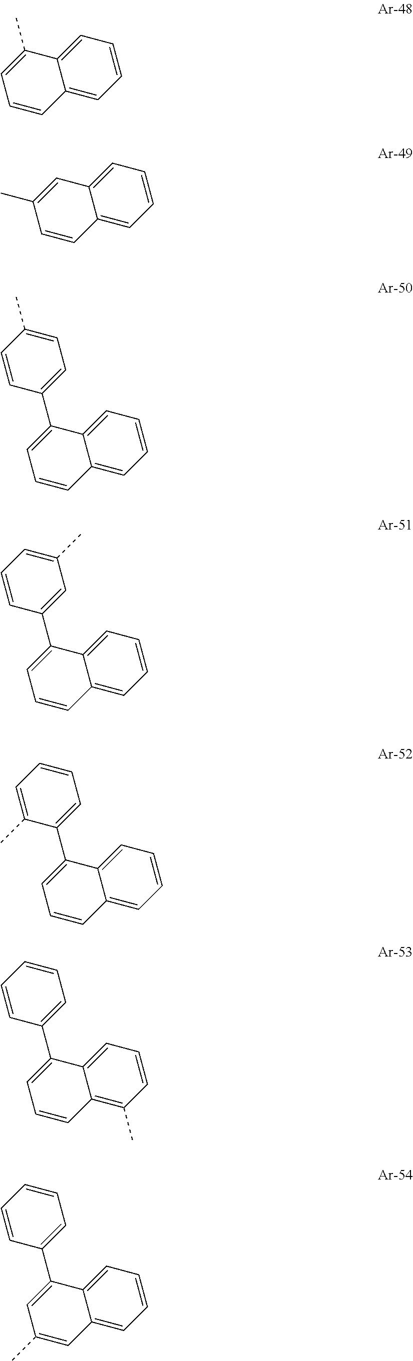

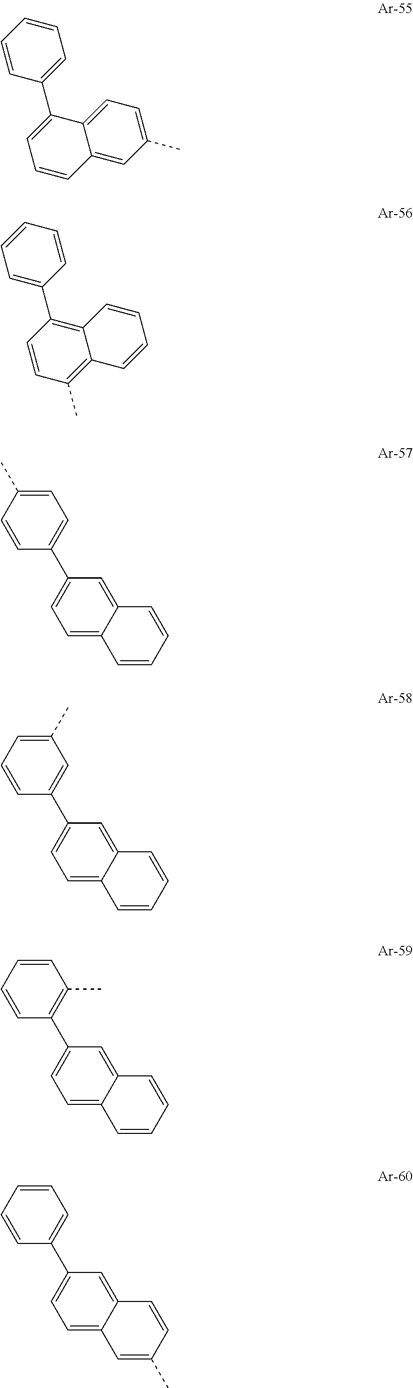

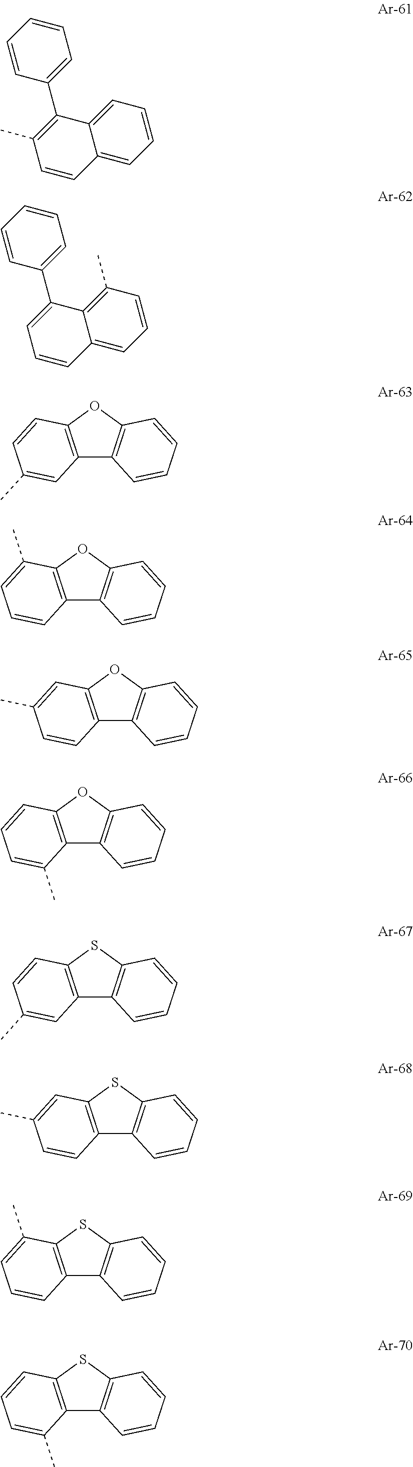

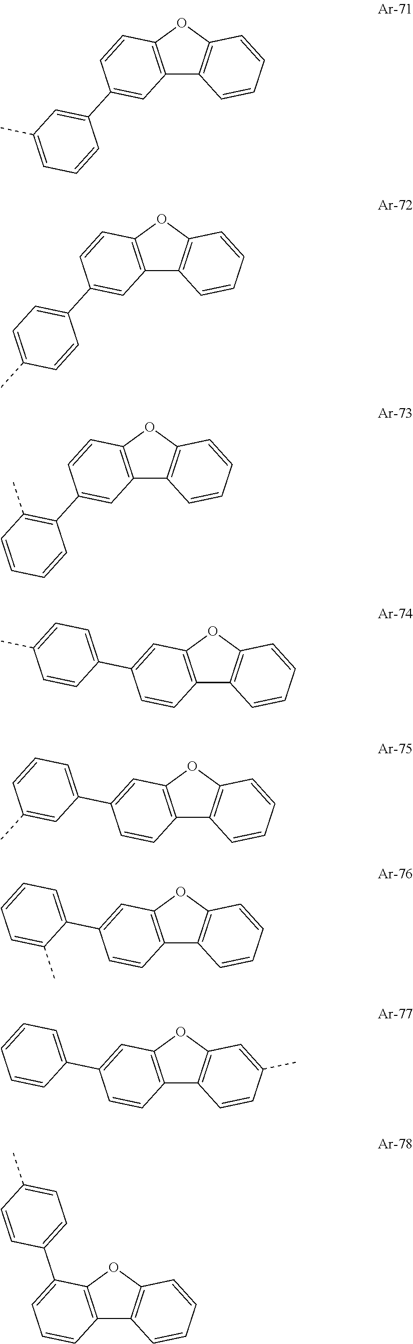

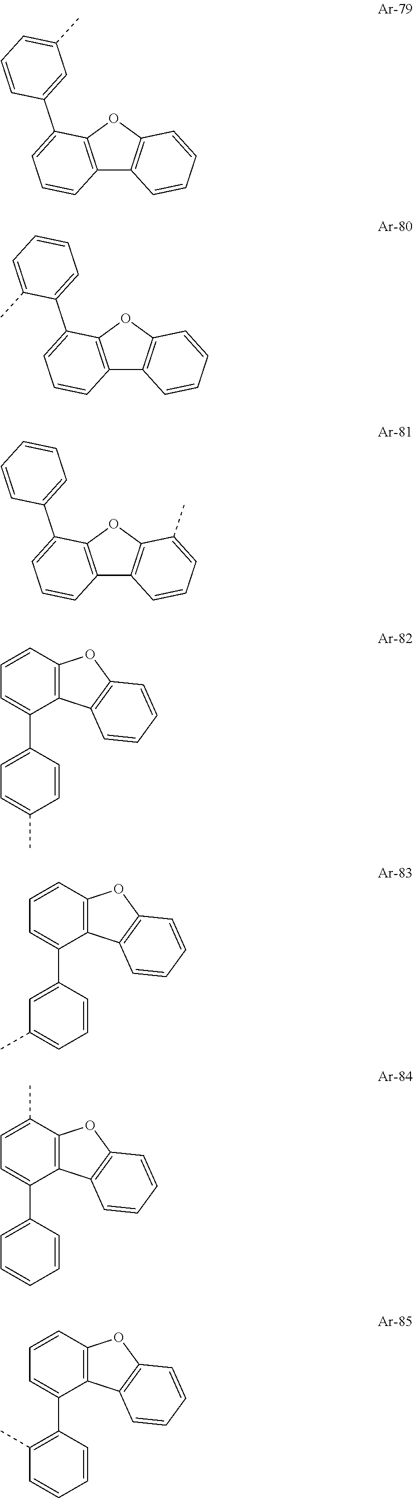

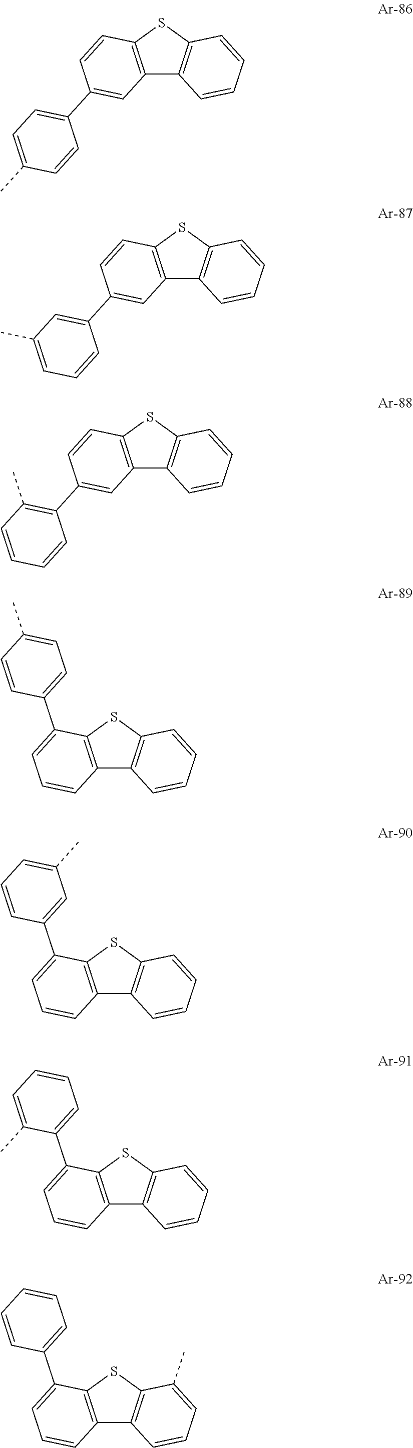

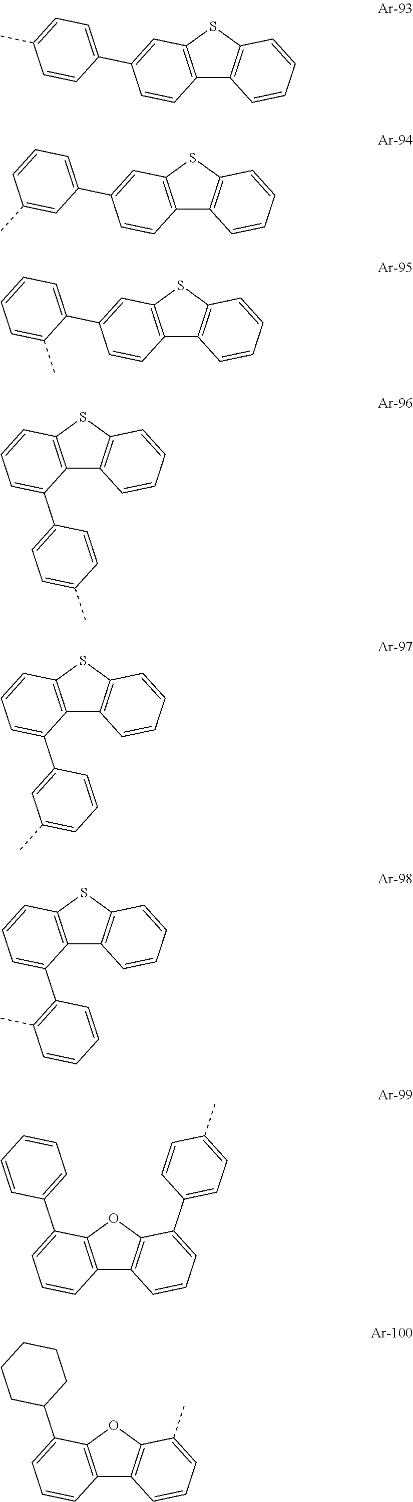

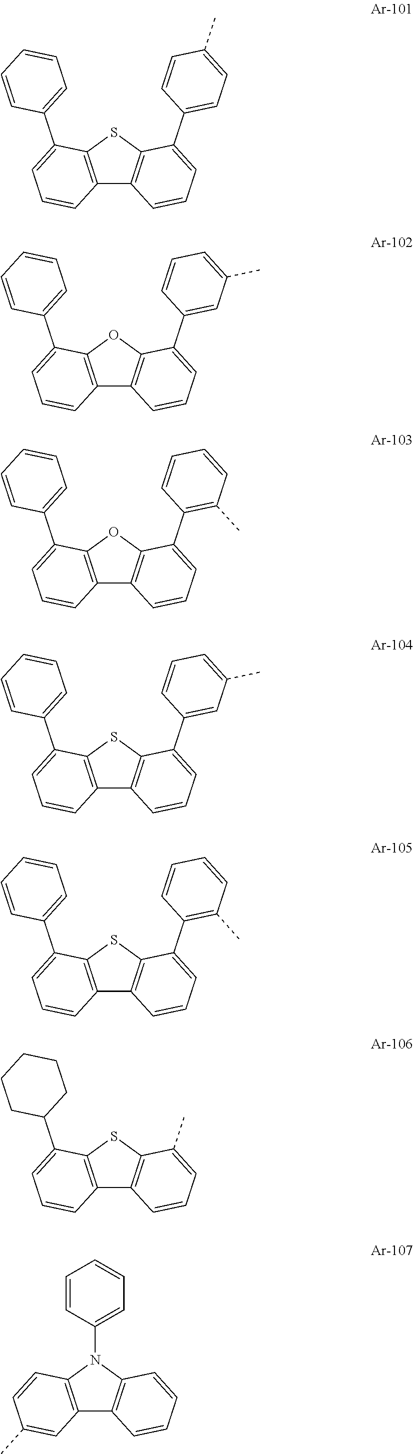

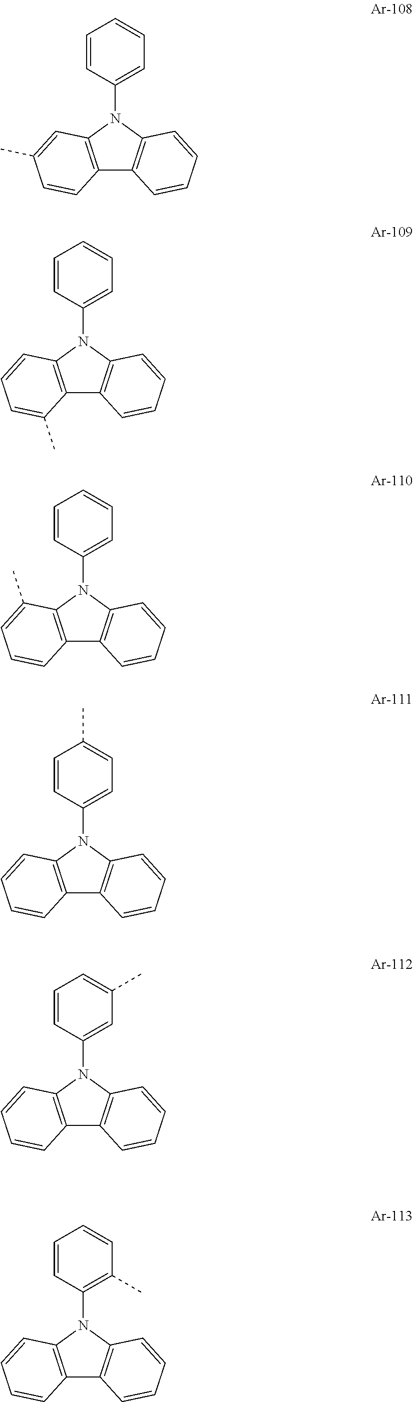

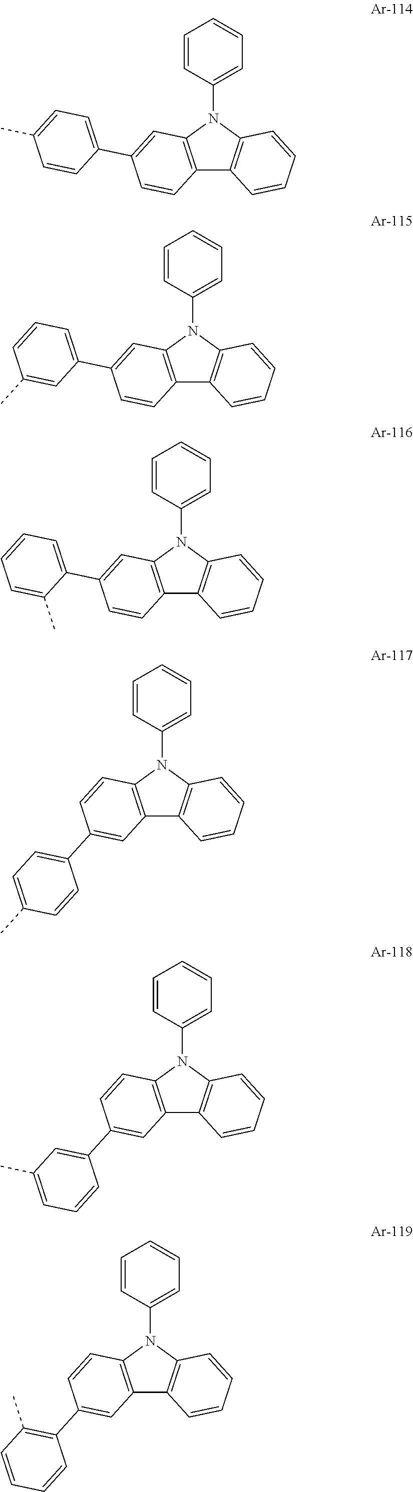

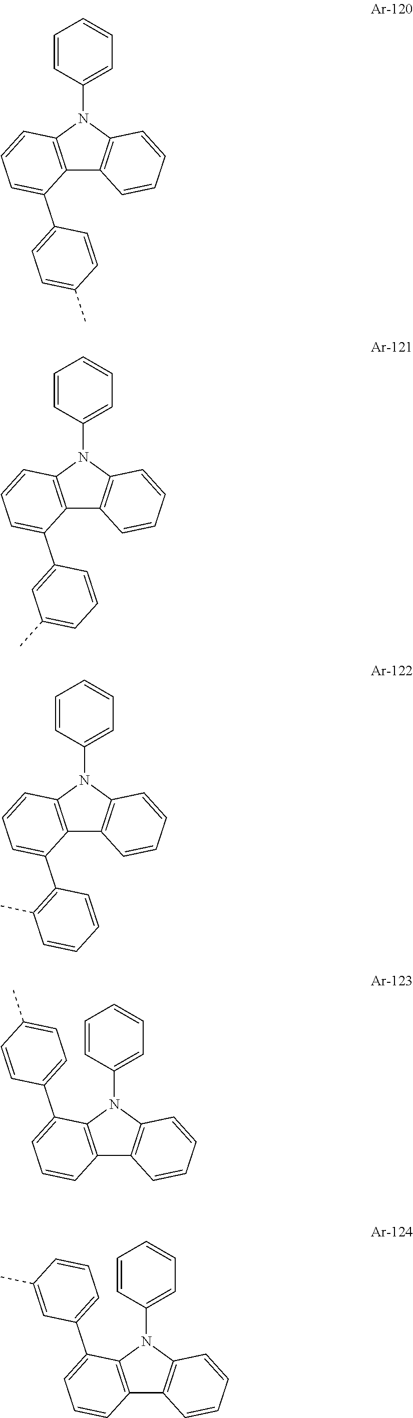

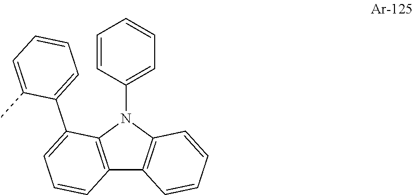

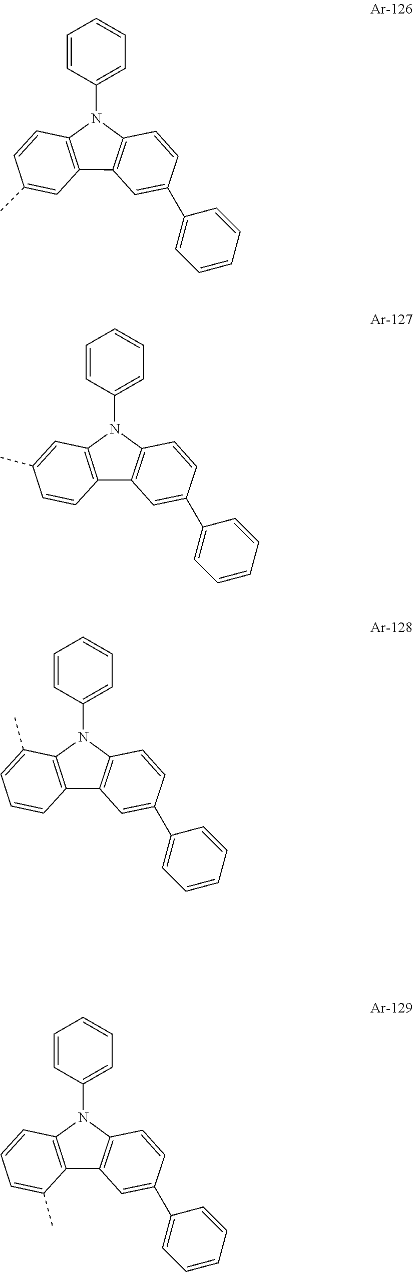

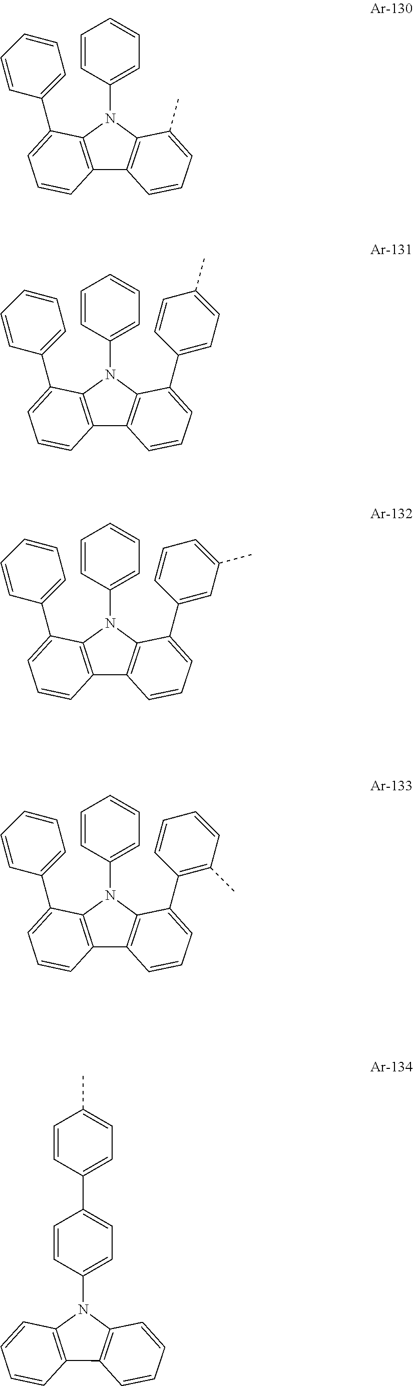

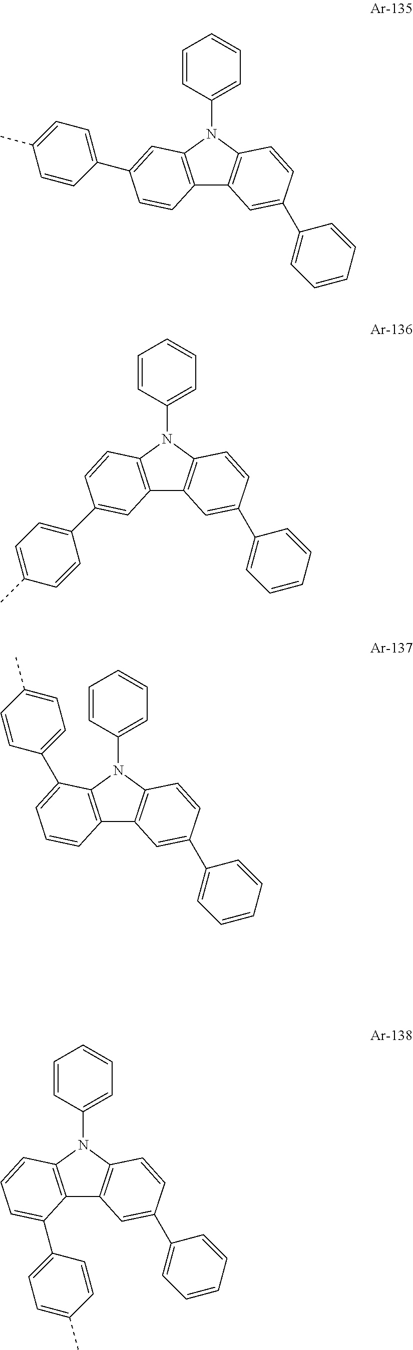

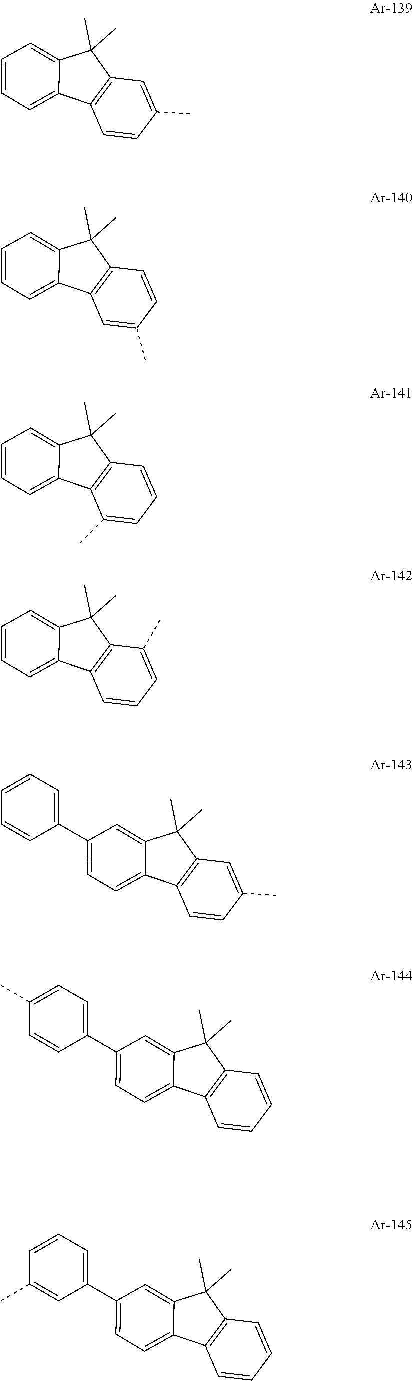

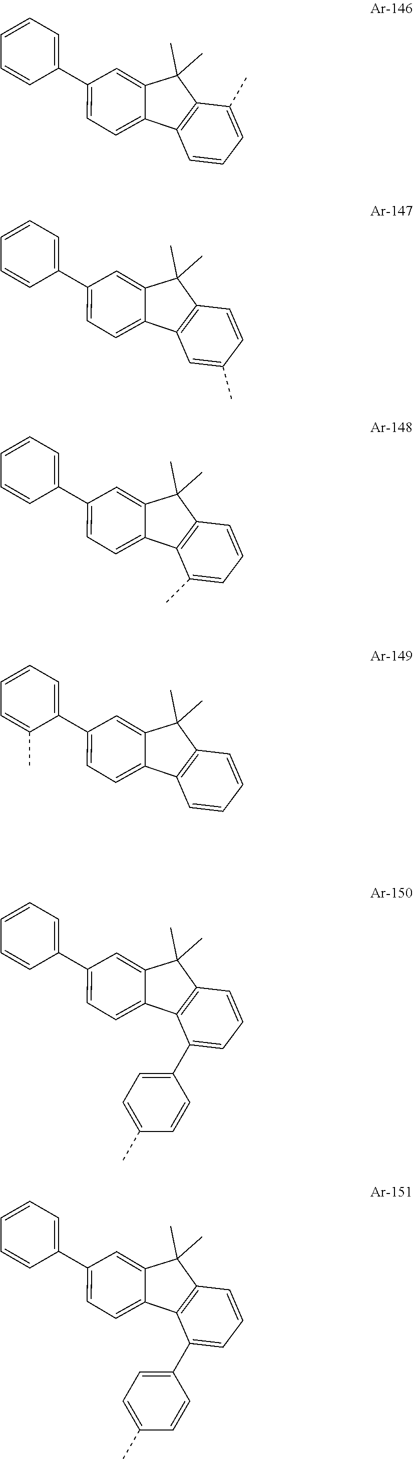

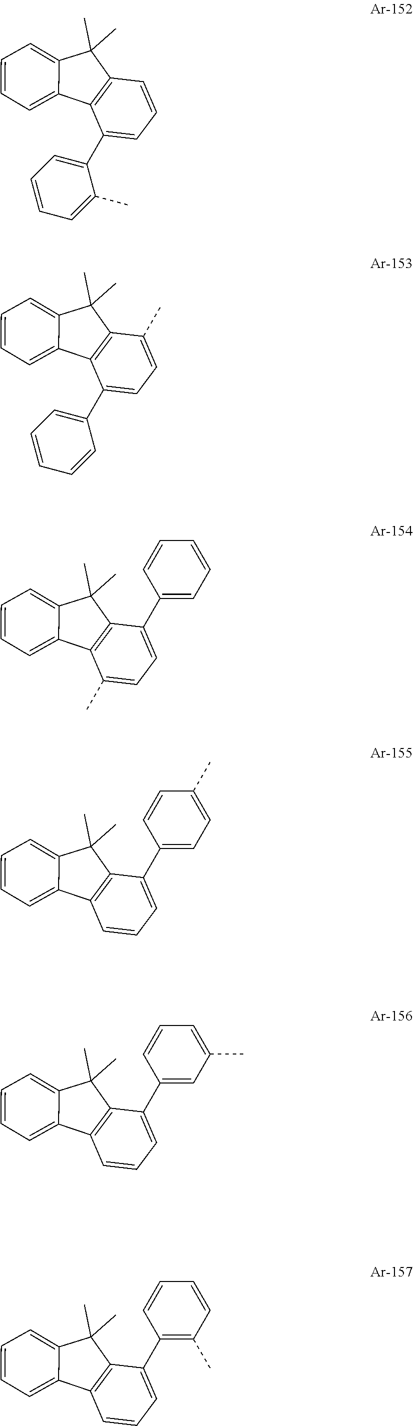

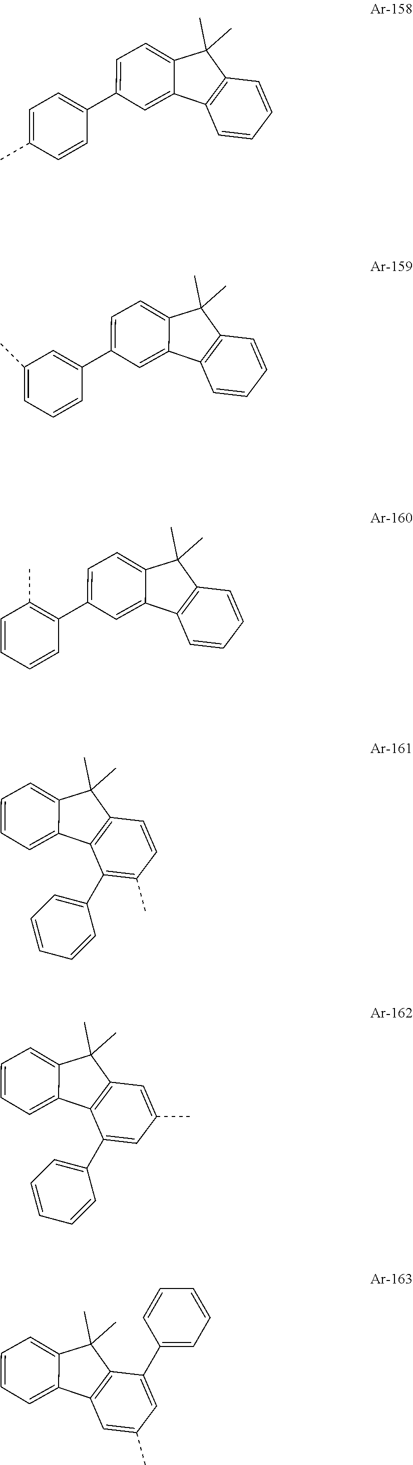

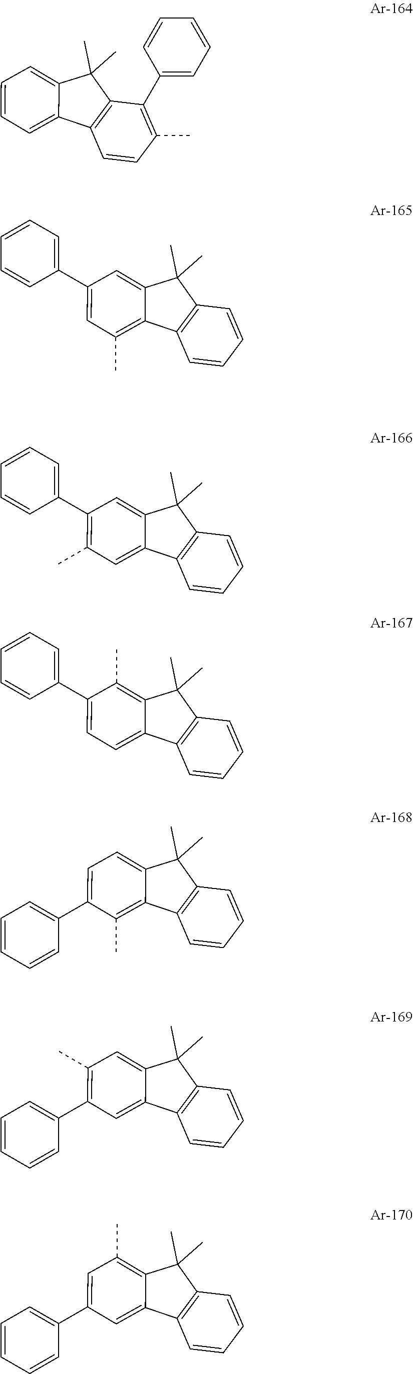

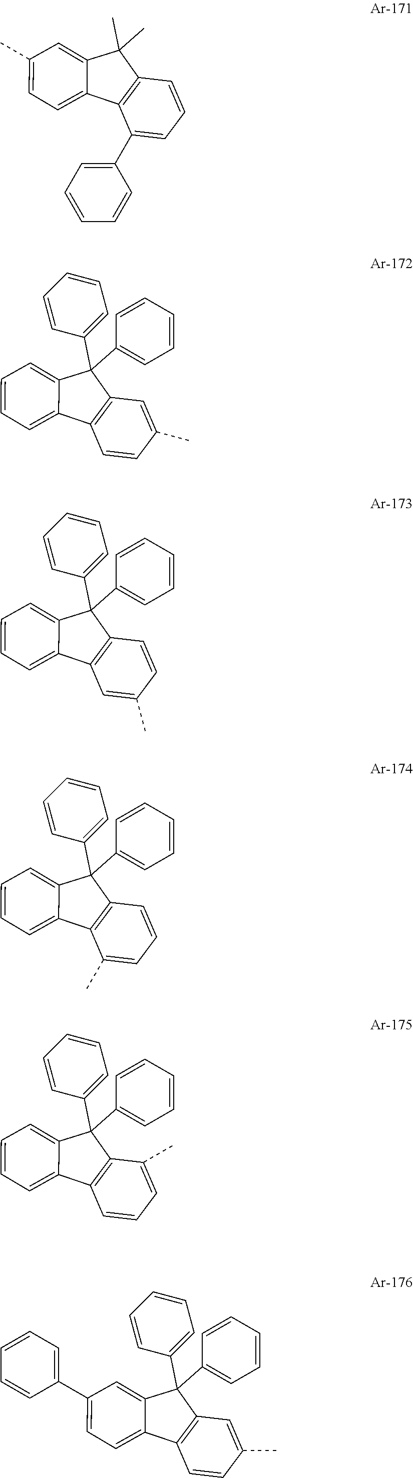

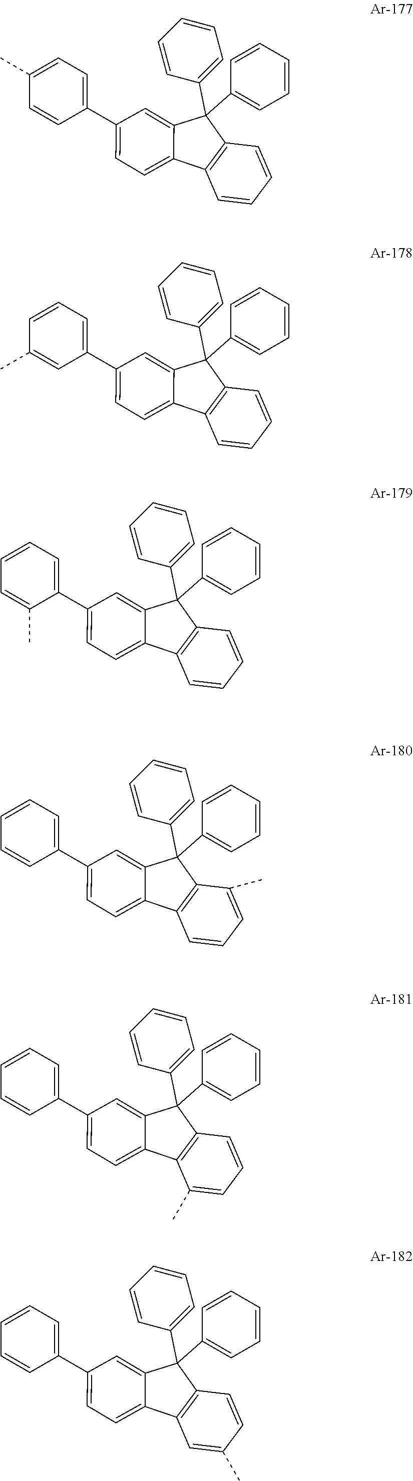

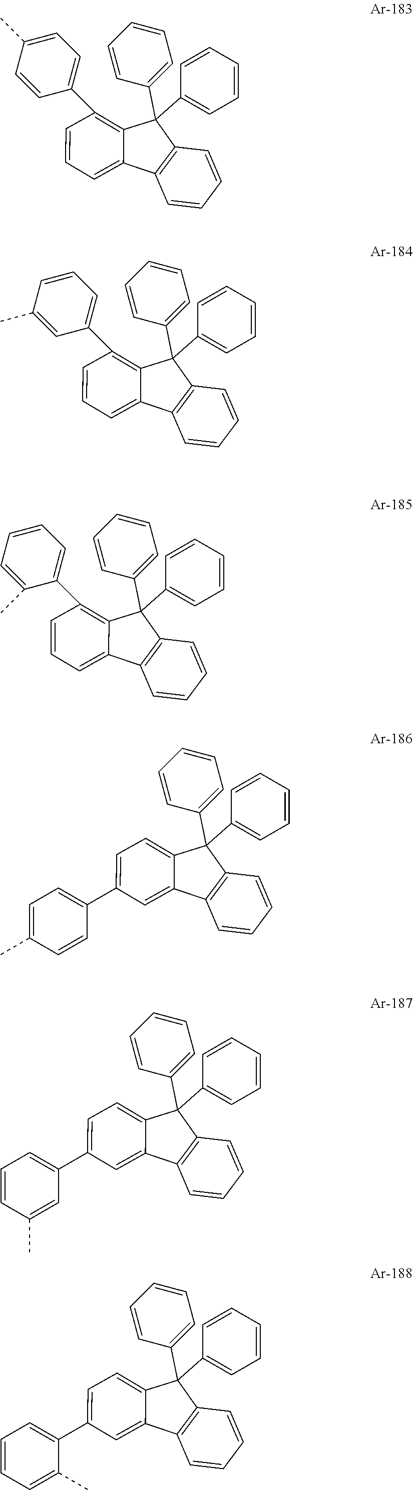

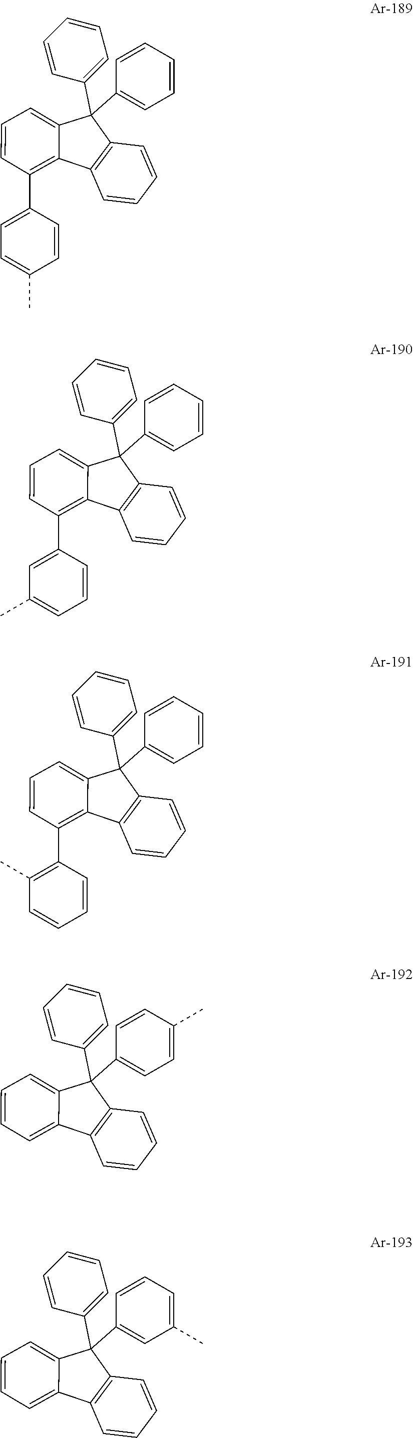

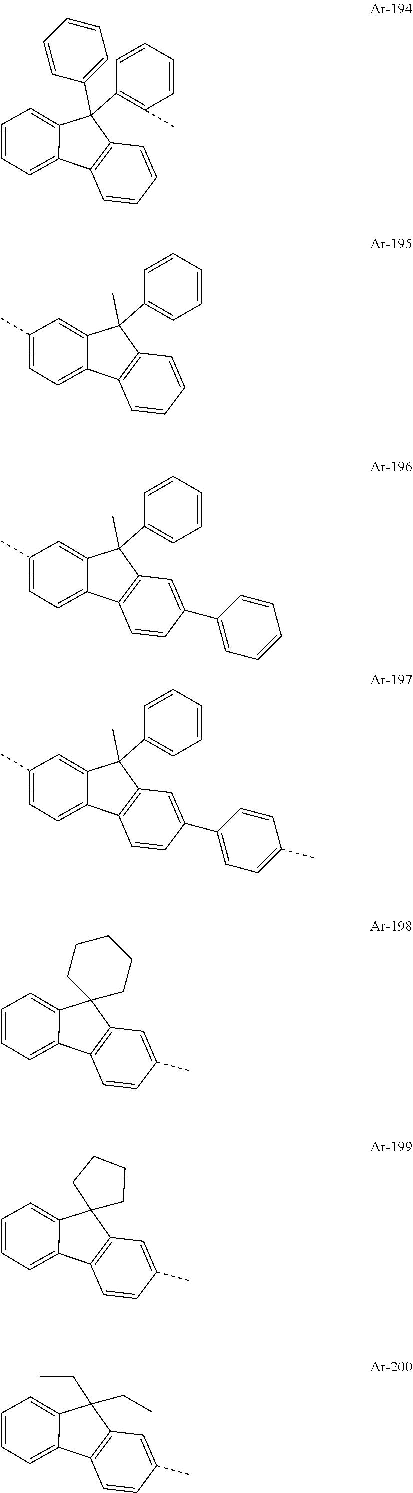

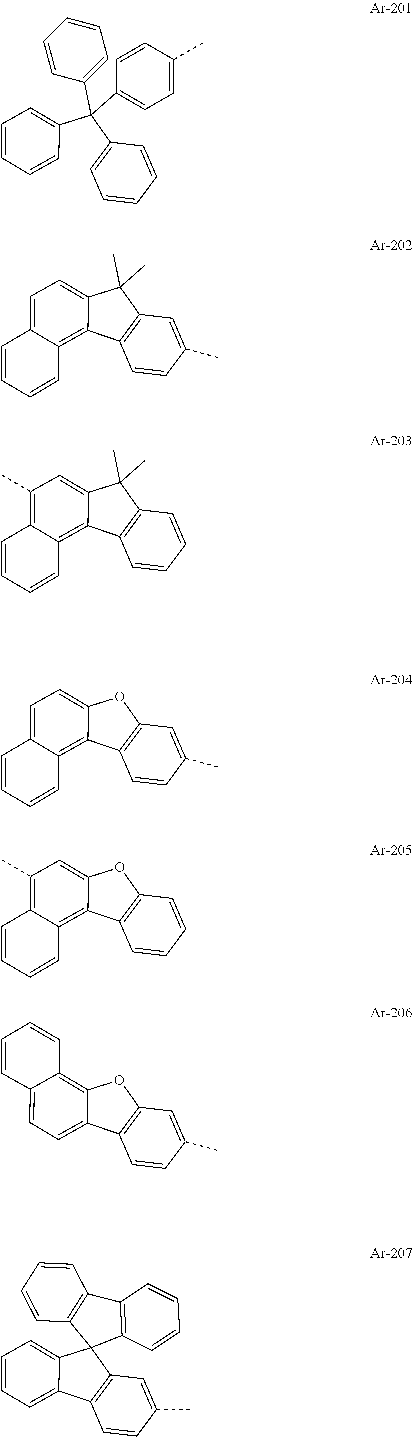

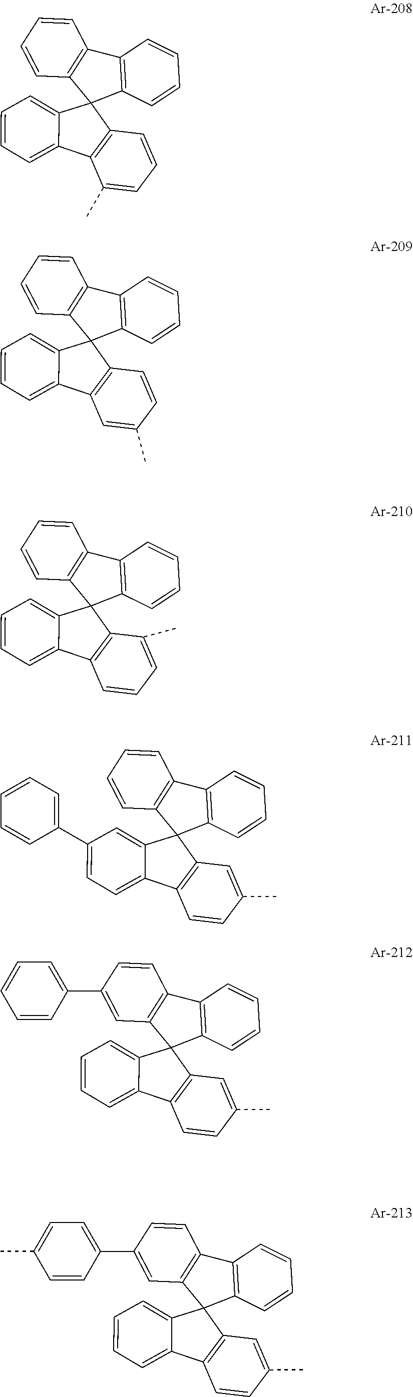

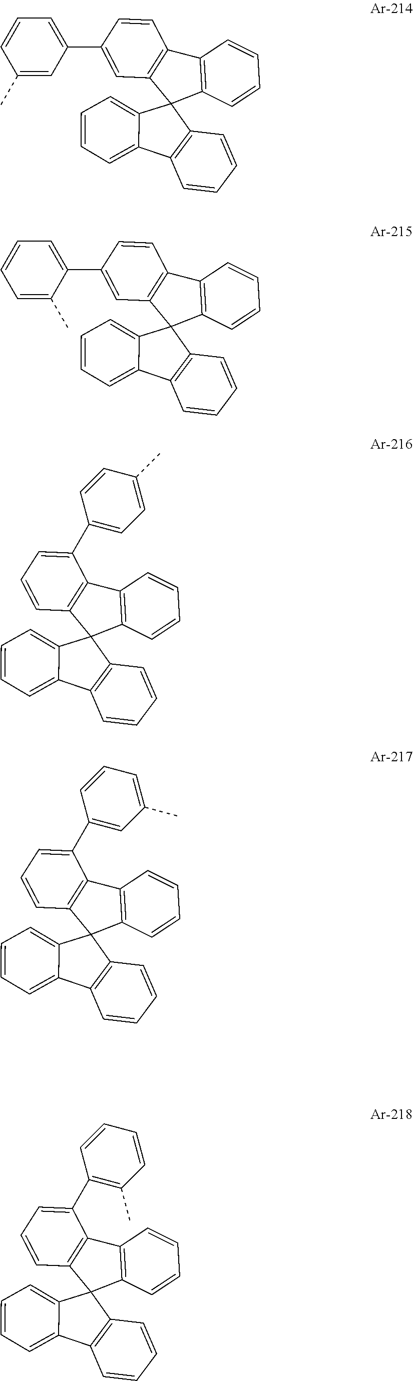

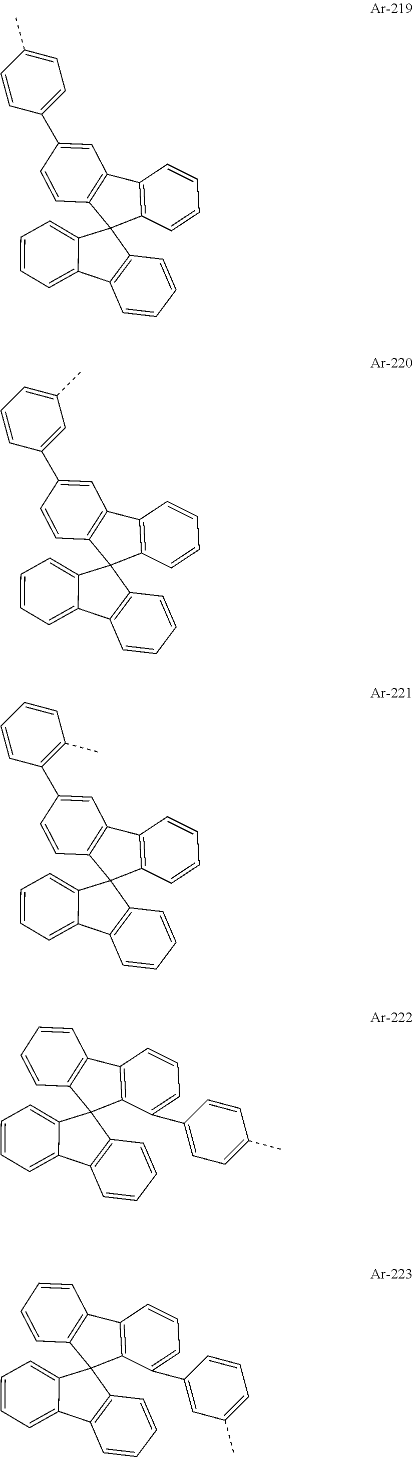

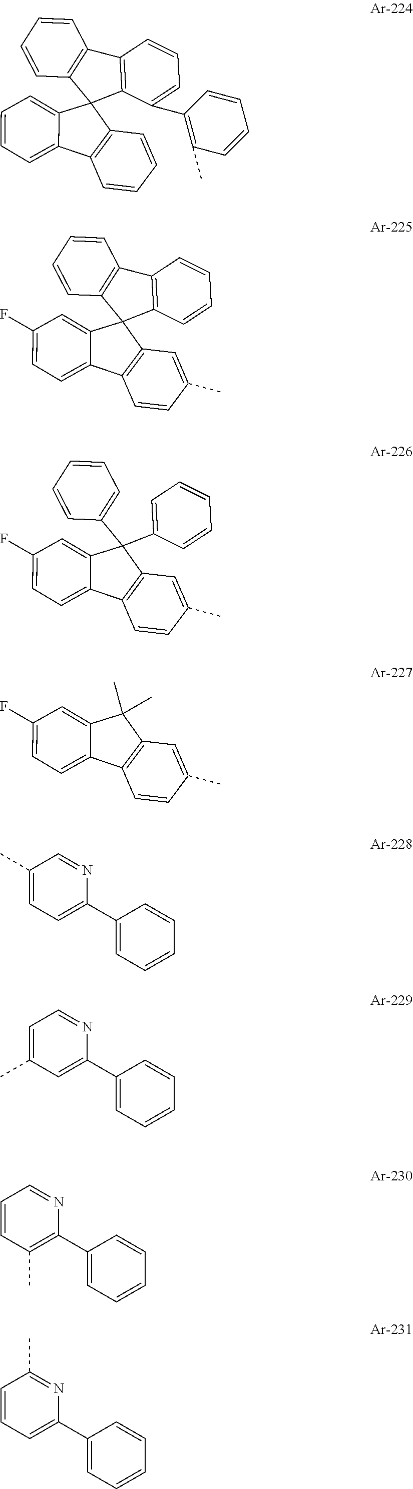

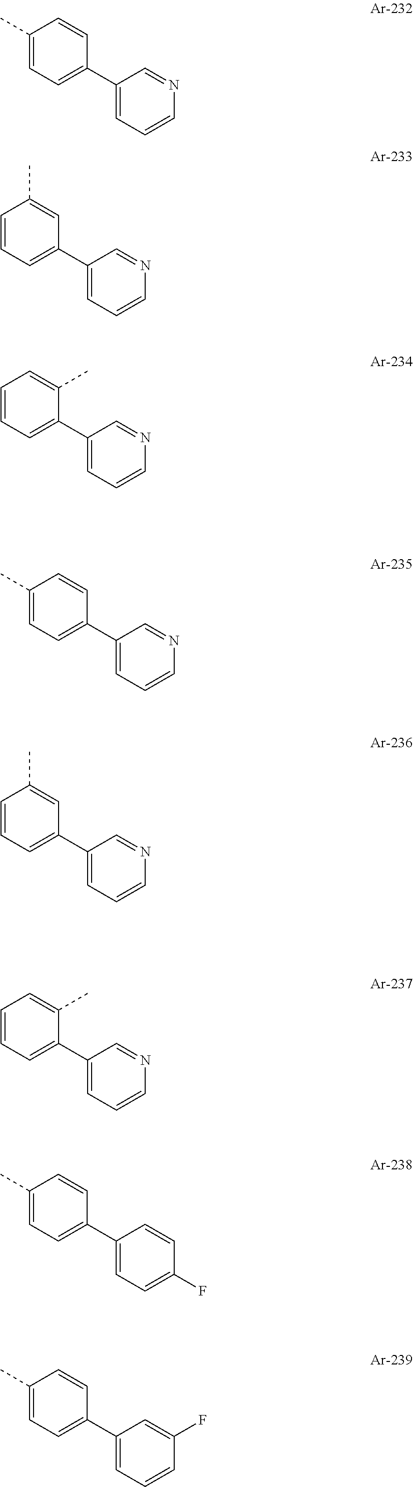

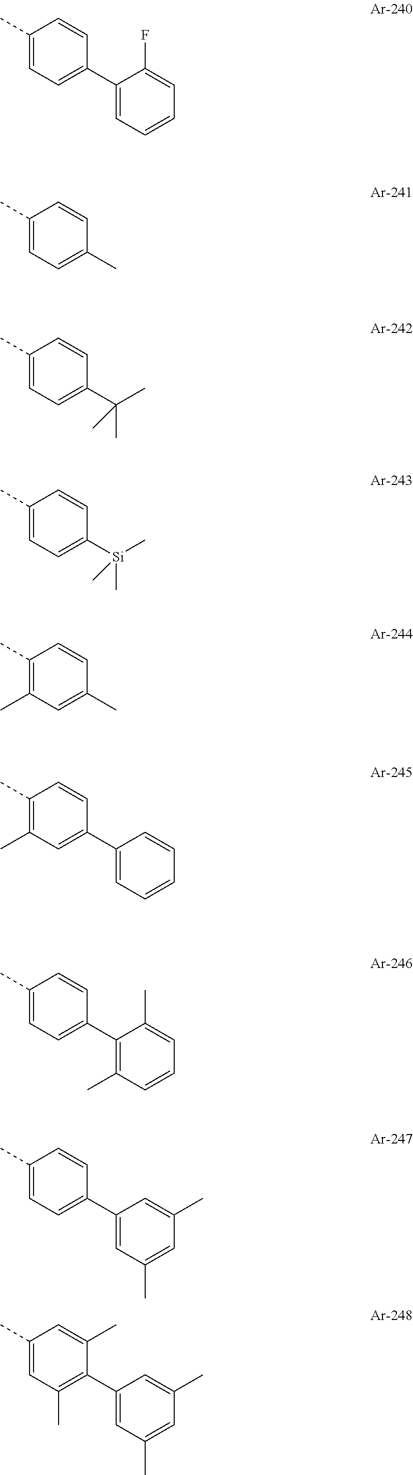

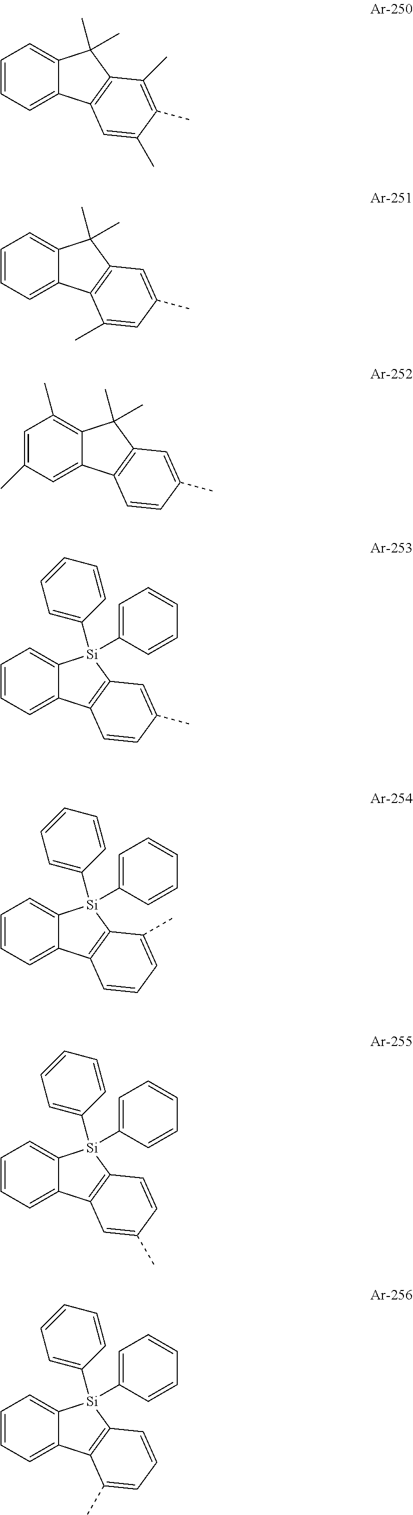

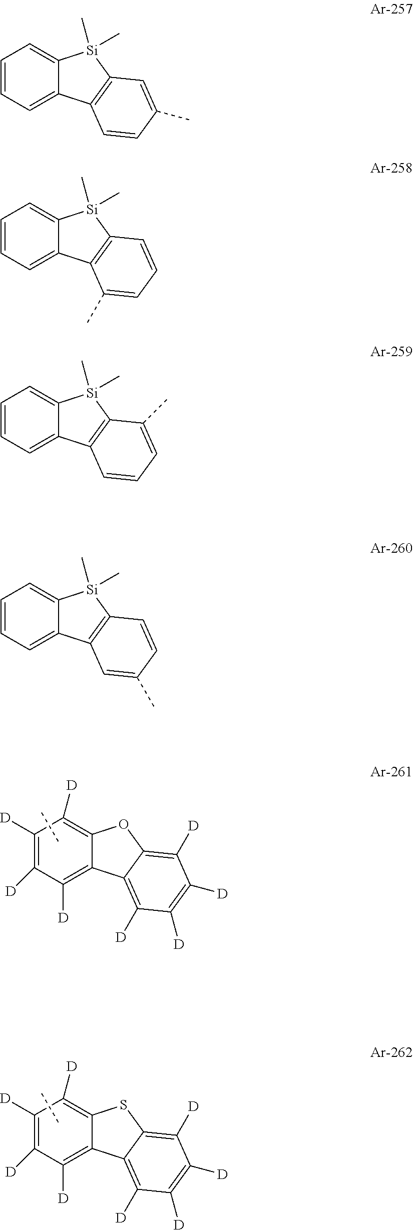

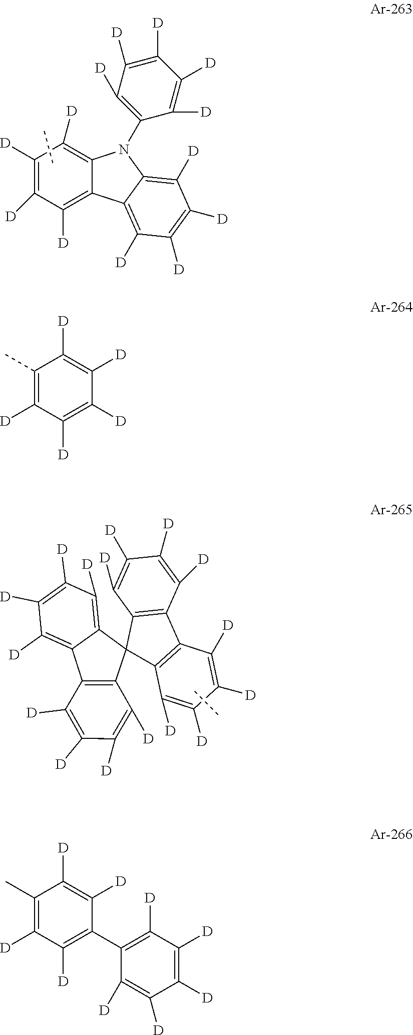

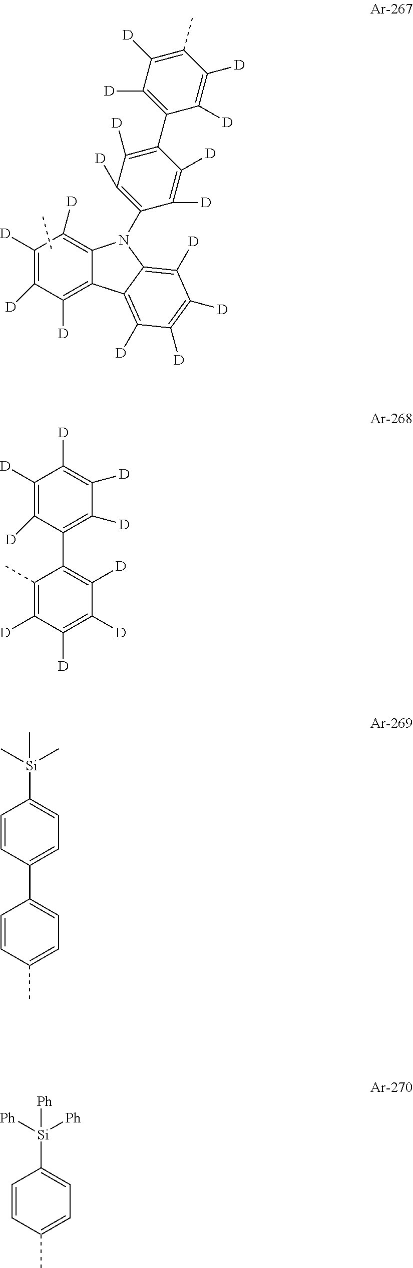

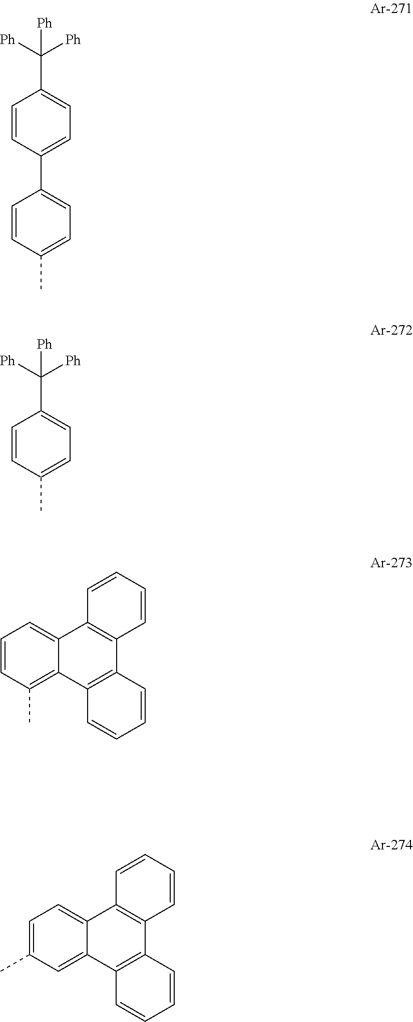

where the groups at the positions shown as unsubstituted are substituted by R³ radicals, where R³ in these positions is preferably H, and where the dotted bond is the bond to the rest of the A group.

Of the abovementioned groups, those of the formulae (Ar-1), (Ar-3), (Ar-16), (Ar-49), (Ar-63), (Ar-65), (Ar-66), (Ar-69), (Ar-78), (Ar-139) and (Ar-274) are the most preferred, and especially formula (Ar-1).

In a preferred embodiment, Ar² groups selected in formula (A) are the same. In another preferred embodiment, the Ar² groups selected are different.

Examples of very particularly preferred ET groups are the following groups, where the dotted lines represent the bonds to the rest of the formula (I):

ET-23a to ET-23s groups having the general formula

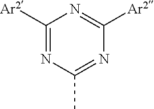

where

| ET group | Ar^{2′} | Ar^{2″} |
|---|---|---|
| Formula (ET-23a) | Formula (Ar-1) | Formula (Ar-1) |
| Formula (ET-23b) | Formula (Ar-16) | Formula (Ar-16) |
| Formula (ET-23c) | Formula (Ar-1) | Formula (Ar-16) |
| Formula (ET-23d) | Formula (Ar-63) | Formula (Ar-65) |
| Formula (ET-23e) | Formula (Ar-63) | Formula (Ar-63) |
| Formula (ET-23f) | Formula (Ar-1) | Formula (Ar-63) |
| Formula (ET-23g) | Formula (Ar-1) | Formula (Ar-65) |
| Formula (ET-23h) | Formula (Ar-65) | Formula (Ar-65) |
| Formula (ET-23i) | Formula (Ar-1) | Formula (Ar-66) |
| Formula (ET-23j) | Formula (Ar-1) | Formula (Ar-274) |
| Formula (ET-23k) | Formula (Ar-1) | Formula (Ar-69) |
| Formula (ET-23l) | Formula (Ar-69) | Formula (Ar-69) |
| Formula (ET-23m) | Formula (Ar-1) | Formula (Ar-49) |
| Formula (ET-23n) | Formula (Ar-49) | Formula (Ar-49) |
| Formula (ET-23o) | Formula (Ar-1) | Formula (Ar-3) |
| Formula (ET-23p) | Formula (Ar-3) | Formula (Ar-3) |
| Formula (ET-23q) | Formula (Ar-66) | Formula (Ar-66) |
| Formula (ET-23r) | Formula (Ar-1) | Formula (Ar-139) |
| Formula (ET-23s) | Formula (Ar-139) | Formula (Ar-139) |

ET-24a to ET-24s groups having the general formula

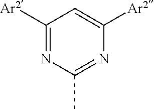

where:

| ET group | Ar^{2′} | Ar^{2″} |
|---|---|---|
| Formula (ET-24a) | Formula (Ar-1) | Formula (Ar-1) |
| Formula (ET-24b) | Formula (Ar-16) | Formula (Ar-16) |
| Formula (ET-24c) | Formula (Ar-1) | Formula (Ar-16) |
| Formula (ET-24d) | Formula (Ar-63) | Formula (Ar-65) |
| Formula (ET-24e) | Formula (Ar-63) | Formula (Ar-63) |
| Formula (ET-24f) | Formula (Ar-1) | Formula (Ar-63) |
| Formula (ET-24g) | Formula (Ar-1) | Formula (Ar-65) |
| Formula (ET-24h) | Formula (Ar-65) | Formula (Ar-65) |
| Formula (ET-24i) | Formula (Ar-1) | Formula (Ar-66) |
| Formula (ET-24j) | Formula (Ar-1) | Formula (Ar-274) |
| Formula (ET-24k) | Formula (Ar-1) | Formula (Ar-69) |
| Formula (ET-24l) | Formula (Ar-69) | Formula (Ar-69) |
| Formula (ET-24m) | Formula (Ar-1) | Formula (Ar-49) |
| Formula (ET-24n) | Formula (Ar-49) | Formula (Ar-49) |
| Formula (ET-24o) | Formula (Ar-1) | Formula (Ar-3) |
| Formula (ET-24p) | Formula (Ar-3) | Formula (Ar-3) |
| Formula (ET-24q) | Formula (Ar-66) | Formula (Ar-66) |
| Formula (ET-24r) | Formula (Ar-1) | Formula (Ar-139) |
| Formula (ET-24s) | Formula (Ar-139) | Formula (Ar-139) |

ET-25a to ET-25s groups having the general formula

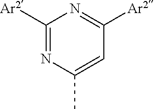

where:

| ET group | Ar^{2′} | Ar^{2″} |
|---|---|---|
| Formula (ET-25a) | Formula (Ar-1) | Formula (Ar-1) |
| Formula (ET-25b) | Formula (Ar-16) | Formula (Ar-16) |
| Formula (ET-25c) | Formula (Ar-1) | Formula (Ar-16) |
| Formula (ET-25d) | Formula (Ar-63) | Formula (Ar-65) |
| Formula (ET-25e) | Formula (Ar-63) | Formula (Ar-63) |
| Formula (ET-25f) | Formula (Ar-1) | Formula (Ar-63) |
| Formula (ET-25g) | Formula (Ar-1) | Formula (Ar-65) |
| Formula (ET-25h) | Formula (Ar-65) | Formula (Ar-65) |
| Formula (ET-25i) | Formula (Ar-1) | Formula (Ar-66) |
| Formula (ET-25j) | Formula (Ar-1) | Formula (Ar-274) |
| Formula (ET-25k) | Formula (Ar-1) | Formula (Ar-69) |
| Formula (ET-25l) | Formula (Ar-69) | Formula (Ar-69) |
| Formula (ET-25m) | Formula (Ar-1) | Formula (Ar-49) |
| Formula (ET-25n) | Formula (Ar-49) | Formula (Ar-49) |
| Formula (ET-25o) | Formula (Ar-1) | Formula (Ar-3) |
| Formula (ET-25p) | Formula (Ar-3) | Formula (Ar-3) |
| Formula (ET-25q) | Formula (Ar-66) | Formula (Ar-66) |
| Formula (ET-25r) | Formula (Ar-1) | Formula (Ar-139) |
| Formula (ET-25s) | Formula (Ar-139) | Formula (Ar-139) |

ET-26a to ET-26k groups having the general formula

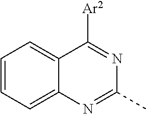

where

| ET group | Ar² |
|---|---|
| Formula (ET-26a) | Formula (Ar-1) |
| Formula (ET-26b) | Formula (Ar-16) |
| Formula (ET-26c) | Formula (Ar-3) |
| Formula (ET-26d) | Formula (Ar-49) |
| Formula (ET-26e) | Formula (Ar-63) |
| Formula (ET-26f) | Formula (Ar-65) |
| Formula (ET-26g) | Formula (Ar-66) |
| Formula (ET-26h) | Formula (Ar-69) |
| Formula (ET-26i) | Formula (Ar-78) |
| Formula (ET-26j) | Formula (Ar-139) |
| Formula (ET-26k) | Formula (Ar-274) |

ET-27a to ET-27k groups having the general formula

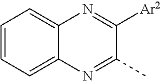

where:

| ET group | Ar² |
|---|---|
| Formula (ET-27a) | Formula (Ar-1) |
| Formula (ET-27b) | Formula (Ar-16) |
| Formula (ET-27c) | Formula (Ar-3) |
| Formula (ET-27d) | Formula (Ar-49) |
| Formula (ET-27e) | Formula (Ar-63) |
| Formula (ET-27f) | Formula (Ar-65) |
| Formula (ET-27g) | Formula (Ar-66) |
| Formula (ET-27h) | Formula (Ar-69) |
| Formula (ET-27i) | Formula (Ar-78) |
| Formula (ET-27j) | Formula (Ar-139) |
| Formula (ET-27k) | Formula (Ar-274) |

ET-28a to ET-28k groups having the general formula

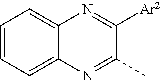

where:

| ET group | Ar² |
|---|---|
| Formula (ET-28a) | Formula (Ar-1) |
| Formula (ET-28b) | Formula (Ar-16) |
| Formula (ET-28c) | Formula (Ar-3) |
| Formula (ET-28d) | Formula (Ar-49) |
| Formula (ET-28e) | Formula (Ar-63) |
| Formula (ET-28f) | Formula (Ar-65) |
| Formula (ET-28g) | Formula (Ar-66) |
| Formula (ET-28h) | Formula (Ar-69) |
| Formula (ET-28i) | Formula (Ar-78) |
| Formula (ET-28j) | Formula (Ar-139) |
| Formula (ET-28k) | Formula (Ar-274) |

Preferably, R³ is the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, R⁵C═CR⁵—, Si(R⁵)₂, C═O, C═NR⁵, —NR⁵—, —O—, —S—, —O(═O)O— or —C(═O)NR⁵—. More preferably, R³ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H. Even more preferably, R³ is H.

Preferably, R⁵ is the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—. More preferably, R⁵ is the same or different at each instance and is selected from H, D, Si(R⁶)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁶ radicals, which are preferably H. Even more preferably, R⁵ is H.

In a preferred embodiment of the invention, the two R⁰ radicals are not joined to one another and do not form an aliphatic or heteroaromatic ring, such that there is no formation of a spiro compound, especially not of a spirobifluorene derivative, in position 8 of the fluorene derivative.

Accordingly, formula (I) in a preferred embodiment of the invention conforms to the following formula (I-A)

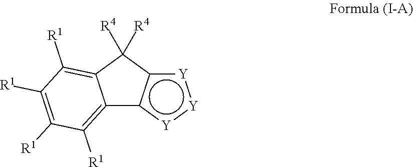

where the variables are as defined above for formula (I) and at least one A group as defined above is present per formula; and

R⁴ is the same or different at each instance and is selected from H, D, F, Cl, Br, I, C(═O)R¹, CN, Si(R¹)₃, N(R¹)₂, P(═O)(R¹)₂, OR¹, S(═O)R¹, S(═O)₂R¹, straight-chain alkyl, alkoxy or thioalkyl groups having 1 to 20 carbon atoms, branched or cyclic alkyl, alkoxy or thioalkyl groups having 3 to 20 carbon atoms, alkenyl or alkynyl groups having 2 to 20 carbon atoms, aromatic ring systems having 6 to 40 aromatic ring atoms, heteroaromatic ring systems having 5 to 40 aromatic ring atoms, aryloxy or heteroaryloxy groups having 5 to 40 aromatic ring atoms, and aralkyl groups having 5 to 40 aromatic ring atoms; with exclusion of joining of the two R⁴ radicals to one another to form an aliphatic or heteroaliphatic ring; where the alkyl, alkoxy, thioalkyl, alkenyl, alkynyl, aryloxy, heteroaryloxy and aralkyl groups mentioned and the aromatic ring systems and heteroaromatic ring systems mentioned are each substituted by R¹ radicals; and where one or more CH₂ groups in the alkyl, alkoxy, thioalkyl, alkenyl, alkynyl, aryloxy, heteroaryloxy and aralkyl groups mentioned may be replaced by —R¹C═CR¹—, —C═C—, Si(R¹)₂, C═O, C═NR¹, —C(═O)O—, —C(═O)NR¹—, NR¹, P(═O)(R¹), —O—, —S—, SO or SO₂.

Preferred embodiments of the formula (I-A) are the following formulae (II-A), (III-A) and (IV-A):

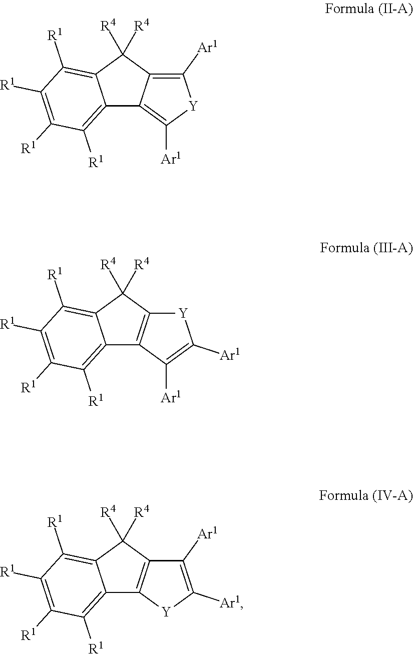

where the variables are as defined above and at least one A group as defined above is present per formula.

Preferably, R⁴ is the same or different at each instance and is selected from F, CN, Si(R¹)₃, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where the alkyl groups mentioned and the aromatic ring systems and heteroaromatic ring systems mentioned are each substituted by R¹ radicals, and with exclusion of bonding of the two R⁴ radicals to one another to form an aliphatic or heteroaliphatic ring.

In respect of the abovementioned formulae (II-A), (III-A) and (IV-A), the aforementioned preferred embodiments of the variables are preferably applicable. Preferably, in the abovementioned groups, exactly two A groups are or one A group is incorporated in each formula, more preferably exactly one (meaning that, (more) preferably, one or two R¹ radicals, or one or two Ar¹ groups, or one R¹ radical and one Ar¹ group are correspondingly replaced by one or two A groups). When two A groups are present in the compounds of the formulae (II-A), (III-A) and (IV-A), these may either both be bonded to the heteroaromatic five-membered ring; or one A group is bonded to the heteroaromatic five-membered ring and the other A group is bonded to the benzene ring (aromatic six-membered ring); or both A groups are bonded to the benzene ring. Preferably, the one or two A groups is/are bonded to the heteroaromatic five-membered ring. The preferred bonding positions on the benzene ring of the fluorene derivative base skeleton are positions 4 and 6. If no A group is bonded to the heteroaromatic five-membered ring, both Ar¹ groups may be H, or one Ar¹ group is H and the other a radical as defined above, or the two Ar¹ groups may be the same or different and selected from the above-defined radicals. If one A group is bonded to the heteroaromatic five-membered ring, the one Ar¹ group may either be H or a radical as defined above. X in the abovementioned formulae is preferably S or O, more preferably S. Among the abovementioned formulae, preference is given to the formulae (II-A) and (IV-A), especially the formula (II-A).

Further preferably, in the abovementioned formulae (II-A), (III-A) and (IV-A), all R¹ radicals bonded to the benzene ring are H. In a further preferred embodiment, in the abovementioned formulae (II-A), (III-A) and (IV-A), in addition to the A group(s) present, exactly two R¹ radicals bonded to the benzene ring, more preferably exactly one, is/are not H, especially when the A group(s) is/are bonded to the heteroaromatic five-membered ring. The preferred bonding positions of the R¹ radicals that are not H are positions 4 and 6 on the benzene ring of the fluorene derivative base skeleton, but of course only when these positions are not occupied by the A group(s). If R¹ is not H, particularly preferred R¹ groups are the (R¹-1), (R¹-8), (R¹-31), (R¹-33), (R¹-163), (R¹-189) and (R¹-190) groups defined further up.

In a further-preferred embodiment of the invention, R⁴ is selected identically at each instance. More preferably, R⁴ in the abovementioned formulae is the same and is selected from straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where the alkyl groups mentioned and the aromatic ring systems mentioned and the heteroaromatic ring systems mentioned are each substituted by R¹ radicals, and with exclusion of bonding of the two R⁴ radicals to one another to form an aliphatic or heteroaliphatic ring. Most preferably, the two R⁴ radicals are selected identically here from straight-chain alkyl groups having 1 to 4 carbon atoms and phenyl, each substituted by R¹ radicals, where R¹ in this case is preferably H.

In another further-preferred embodiment of the invention, R⁴ is selected differently at each instance. More preferably, R⁴ in the abovementioned formulae is different and is selected from straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where the alkyl groups mentioned and the aromatic ring systems mentioned and the heteroaromatic ring systems mentioned are each substituted by R¹ radicals, and with exclusion of bonding of the two R⁴ radicals to one another to form an aliphatic or heteroaliphatic ring. Most preferably, the two R⁴ radicals are selected differently here from straight-chain alkyl groups having 1 to 4 carbon atoms and phenyl, each substituted by R¹ radicals, where R¹ in this case is preferably H.

Preferred embodiments of the formulae (II-A), (III-A) and (IV-A) conform to the following formulae:

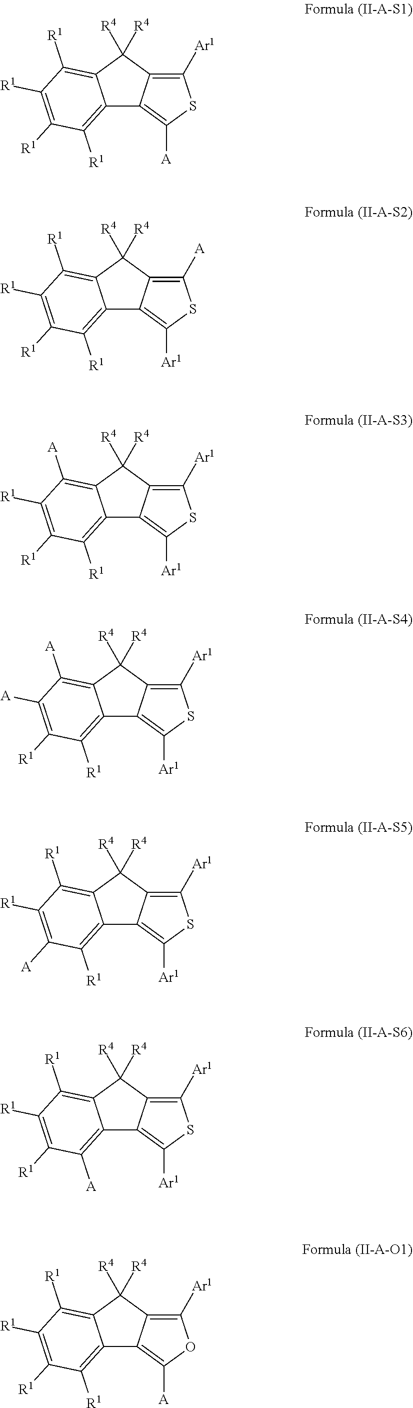

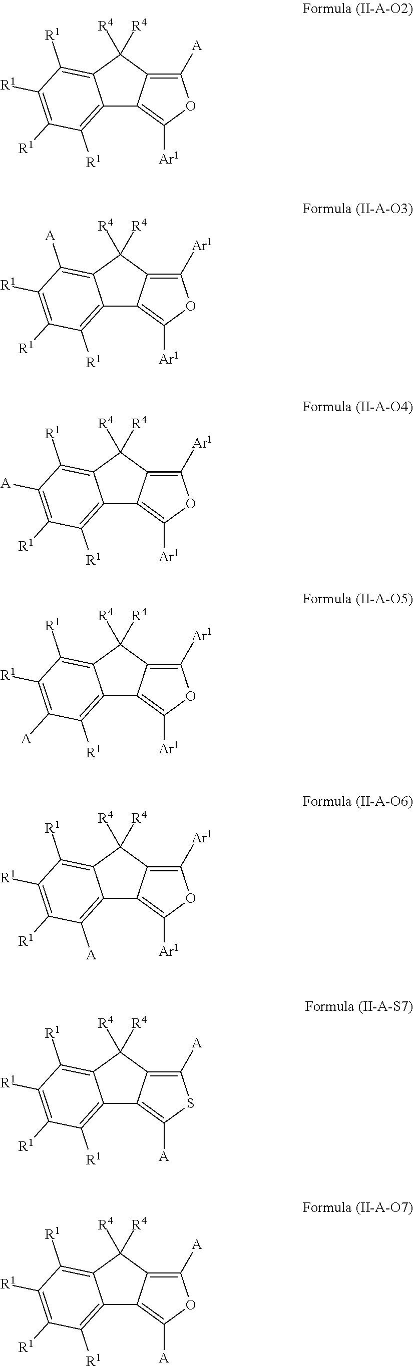

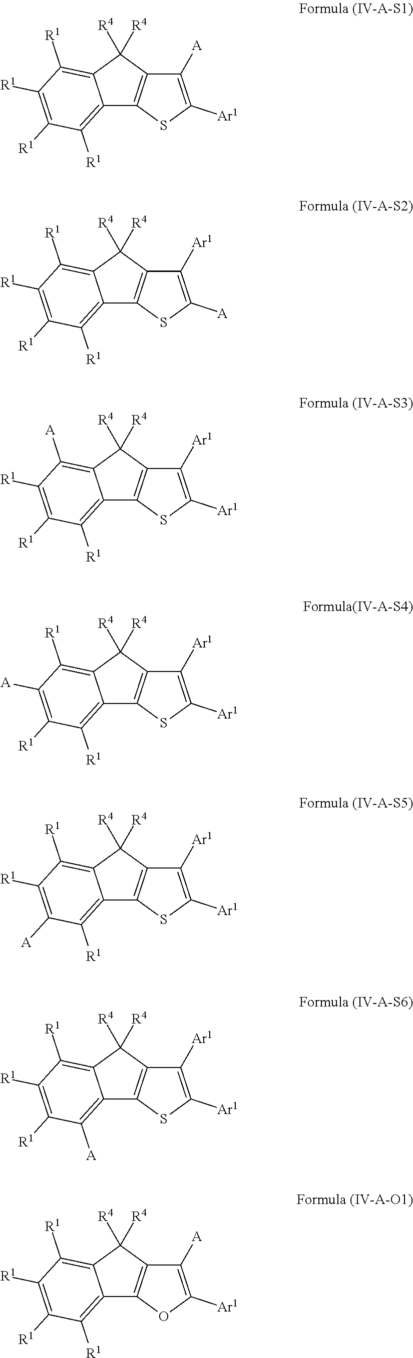

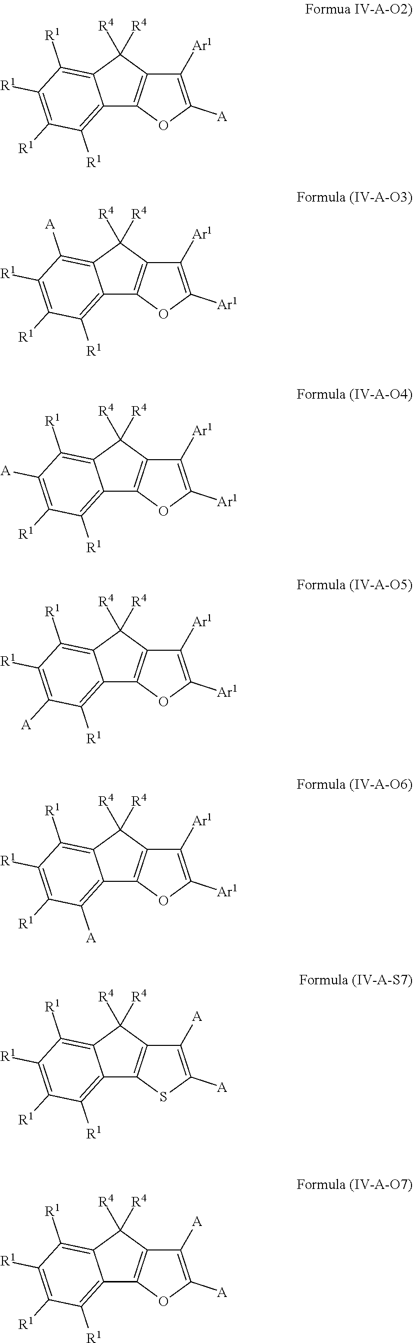

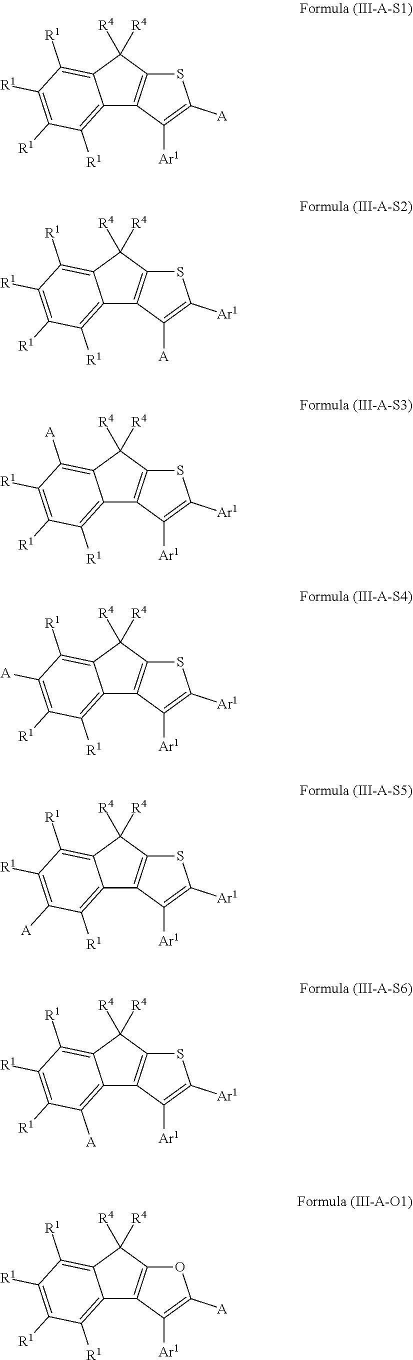

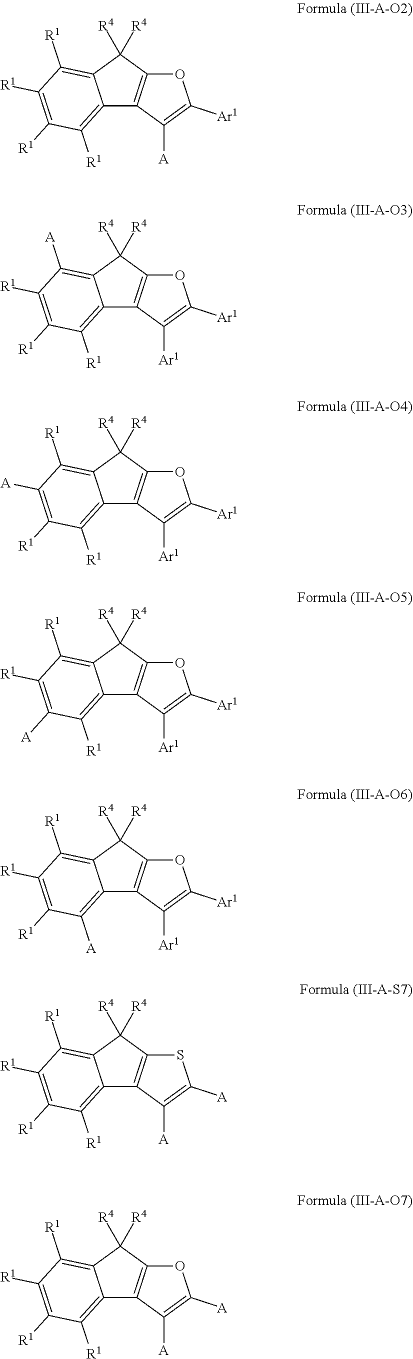

where the variables have the definitions given above and preferably conform to their abovementioned preferred embodiments.

Among the abovementioned formulae, particular preference is given to the formulae (II-A-S1) to (IV-A-S1), (II-A-S2) to (IV-A-S2), (II-A-S4) to (IV-A-S4), (II-A-S6) to (IV-A-S6), (II-A-O1) to (IV-A-O1), (II-A-O2) to (IV-A-O2), (II-A-O4) to (IV-A-O4) and (II-A-O6) to (IV-A-O6). Very particular preference is given to the formulae (II-A-S1) to (IV-A-S1), (II-A-S2) to (IV-A-S2), (II-A-S4) to (IV-A-S4), (II-A-O1) to (IV-A-O1), (II-A-O2) to (IV-A-O2) and (II-A-O4) to (IV-A-O4).

Even greater preference is given to the formulae (II-A-S1), (IV-A-S1), (II-A-S2), (IV-A-S2), (II-A-S4), (IV-A-S4), (II-A-S6), (IV-A-S6), (II-A-O1), (IV-A-O1), (II-A-O2), (IV-A-O2), (II-A-O4), (IV-A-O4), (II-A-O6) and (IV-A-O6).

Most preferred among the abovementioned formulae are the formulae (II-A-S1), (IV-A-S1), (II-A-S2), (IV-A-S2), (II-A-S4), (IV-A-S4), (II-A-O1), (IV-A-O1), (II-A-O2), (IV-A-O2), (II-A-O4) and (IV-A-O4).

More preferably, the compound according to the application therefore conforms to a formula (II-A-S1) to (IV-A-S1), (II-A-S2) to (IV-A-S2), (II-A-S4) to (IV-A-S4), (II-A-S6) to (IV-A-S6), (II-A-O1) to (IV-A-O1), (II-A-O2) to (IV-A-O2), (II-A-O4) to (IV-A-O4) or (II-A-O6) to (IV-A-O6), where the variables that occur are as follows:

Ar¹ is the same or different at each instance and is selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R² radicals, and H;

R¹ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H;

R⁴ is the same or different at each instance and is selected from straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where the alkyl groups mentioned and the aromatic ring systems mentioned and the heteroaromatic ring systems mentioned are each substituted by R¹ radicals, and with exclusion of bonding of the two R⁴ radicals to one another to form an aliphatic or heteroaliphatic ring;

A is a unit of the formula (A)

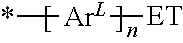

Formula (A), where the variables in formula (A) are defined as follows:

Ar^{L} is the same or different at each instance and is selected from divalent groups derived from benzene, biphenyl, terphenyl, naphthalene, fluorene, indenofluorene, indenocarbazole, spirobifluorene, dibenzofuran and dibenzothiophene, each of which are substituted by R² radicals;

ET is the same or different at each instance and is selected from groups derived from pyridine, pyrazine, pyrimidine, pyridazine, 1,2,4-triazine, 1,3,5-triazine, quinoline, isoquinoline, quinoxaline, quinazoline, pyrazole, imidazole, benzimidazole, thiazole, benzothiazole, oxazole, oxadiazole and benzoxazole, each of which are substituted by Ar² groups;

Ar² is the same or different at each instance and is selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R³ radicals, and H;

n=1 or 0;

and the further variables are as defined in their above-specified broadest embodiment, preferably the above-specified preferred embodiments.

Preferred compounds of the formula (I) in this embodiment are shown in table 1 below.

In another preferred embodiment of the invention, the two R⁰ radicals are joined to one another and form an aliphatic or heteroaromatic ring, such that there is formation of a spiro compound, especially a spirobifluorene derivative, in position 8 of the fluorene derivative.

Accordingly, formula (I) in another preferred embodiment of the invention conforms to the following formula (I-B):

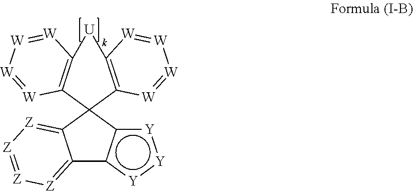

where

W is the same or different at each instance and is selected from N and CR¹;

U is selected from O and S;

k is 0 or 1, where, in the case that k=0, the relevant heteroatom U is absent and U is instead a single bond that connects the corresponding two aromatic six-membered rings; and

the variables Z, Y and R¹ are as defined above, and where at least one A group as defined above is present in the formula (I-B).

Preferably, in formula (I-B), not more than three W groups, more preferably not more than two W groups, even more preferably not more than one W group, most preferably no W group, in an aromatic six-membered ring is N. The remaining groups are correspondingly CR¹. It is additionally preferable that no adjacent W groups in an aromatic six-membered ring are N. It is still further preferred that not more than 3 W groups in any formula (I-B) are N; more preferably, not more than 2 W groups in any formula (I-B) are N; even more preferably, not more than one W group in any formula (I-B) is N; most preferably, no W group is N.

Preferred embodiments of the formula (I-B) conform to the following formulae (II-B) to (IV-B) and (II-C) to (IV-C):

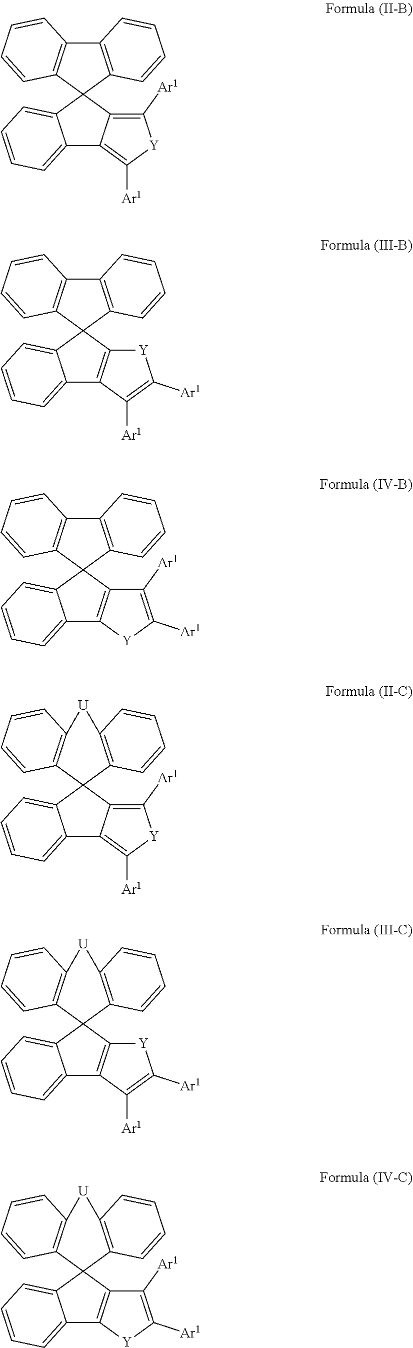

where the variables are as defined above and the unoccupied positions on the benzene rings are each substituted by R¹ radicals, and where at least one A group as defined above is present per formula, where preferably exactly one A group per formula is incorporated in the abovementioned formulae, bonded either to one of the benzene rings (i.e. one R¹ radical is correspondingly replaced by an A group) or to the heteroaromatic five-membered ring (i.e. one Ar¹ group is correspondingly replaced by an A group), it being particularly preferred when the A group binds to the heteroaromatic five-membered ring. The preferred bonding positions on the benzene rings of the spirobifluorene derivative base skeleton are positions 4, 6, 9, 11, 12 and 14. If no A group is bonded to the heteroaromatic five-membered ring, both Ar¹ groups may be H, or one Ar¹ group is H and the other a radical as defined above, or the two Ar¹ groups may be the same or different and selected from the above-defined radicals. If one A group is bonded to the heteroaromatic five-membered ring, the one Ar¹ group may either be H or a radical as defined above. X in the abovementioned formulae is preferably S or O, more preferably S.

Further preferably, in the abovementioned formulae (II-B) to (IV-B) and (II-F) to (IV-C), all R¹ radicals bonded to the benzene rings are H. In a further preferred embodiment, in the abovementioned formulae (II-B) to (IV-B) and (II-C) to (IV-C), in addition to the at least one A group present, exactly two R¹ radicals bonded to the benzene rings, more preferably exactly one R¹ radical, is/are not H, especially when the at least one A group is bonded to the heteroaromatic five-membered ring. The preferred bonding positions for R¹ radicals that are not H are positions 4, 6, 9, 11, 12 and 14 on the benzene rings of the spirobifluorene derivative base skeleton, and of course only when these positions are not occupied by the at least one A group. If R¹ is not H, particularly preferred R¹ groups are the (R¹-1), (R¹-8), (R¹-31), (R¹-33), (R¹-163), (R¹-189) and (R¹-190) groups defined further up.

Preferably, k is 0. Among the abovementioned formulae, preference is accordingly given to the formulae (II-B) to (IV-B), especially the formula (II-B).

Preferred embodiments of the formulae (II-B) to (IV-B) conform to the formulae shown below:

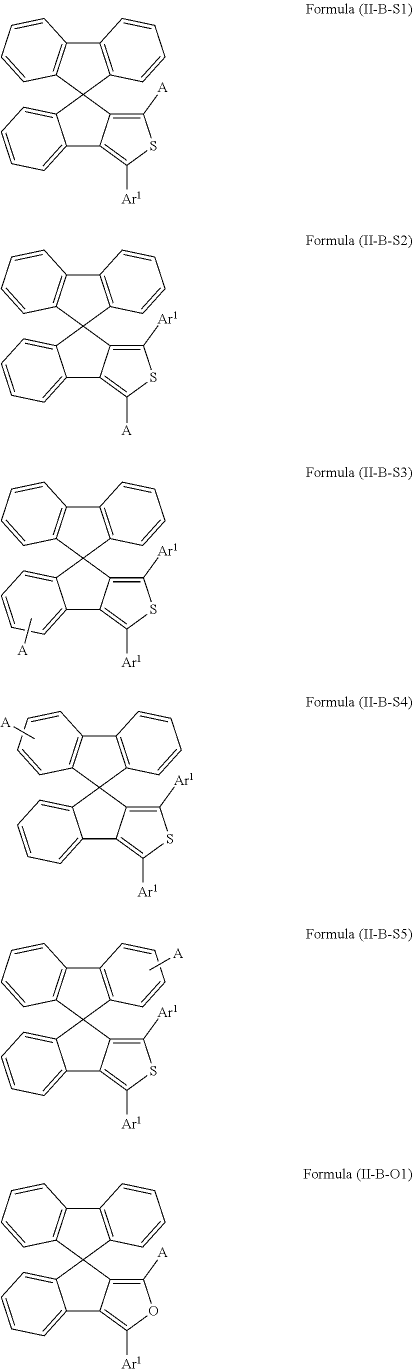

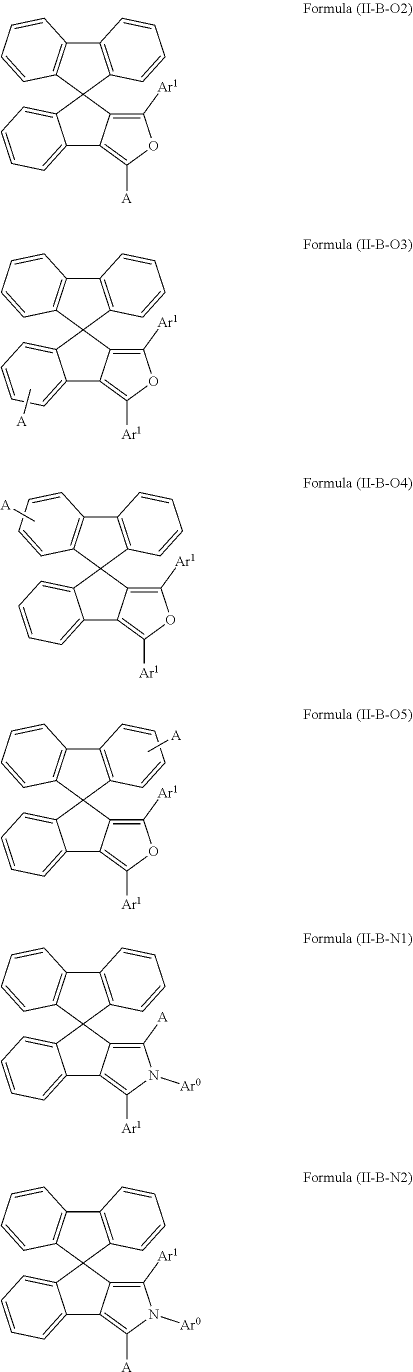

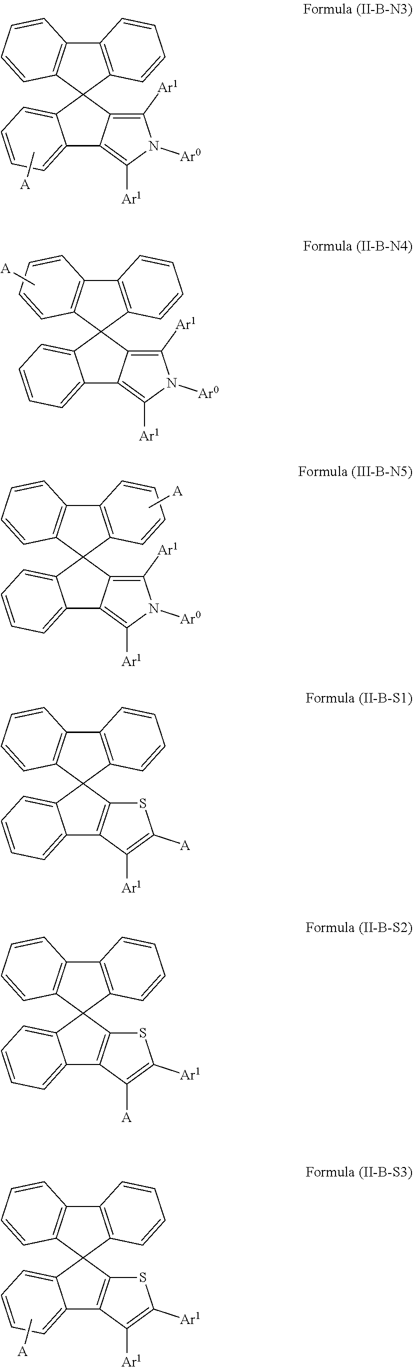

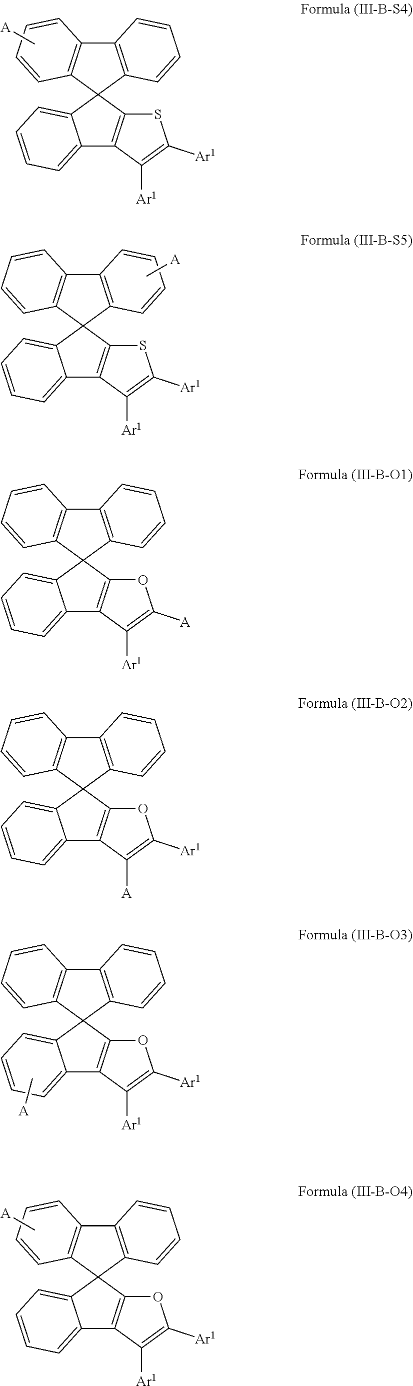

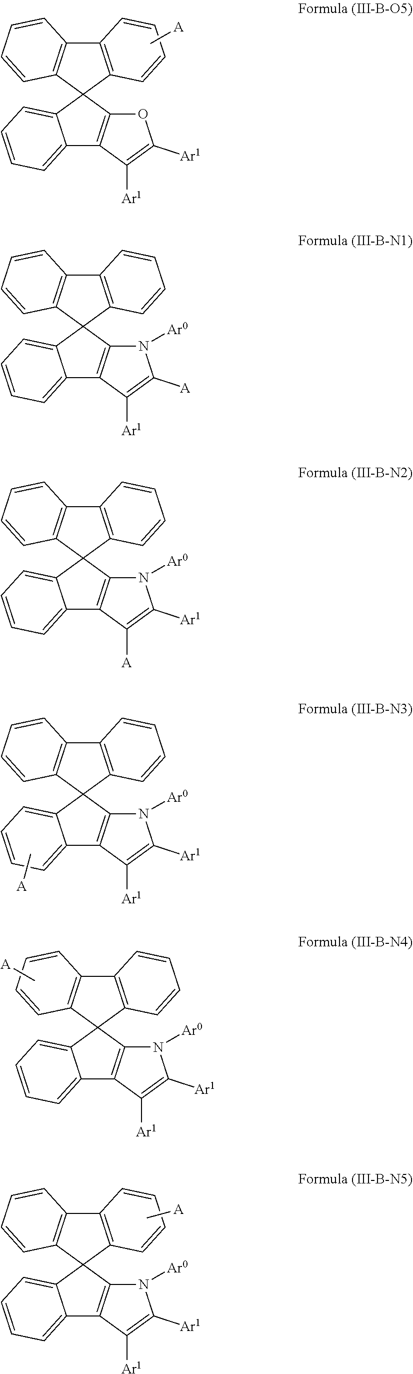

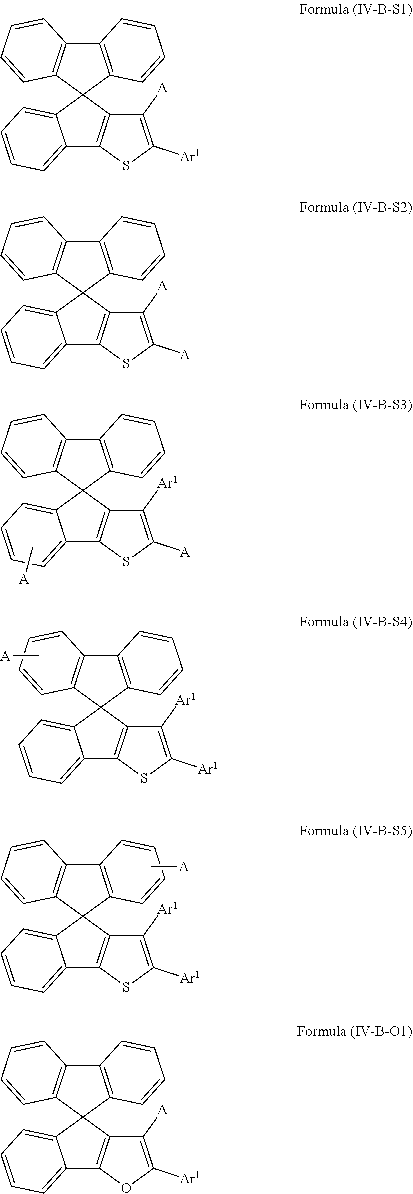

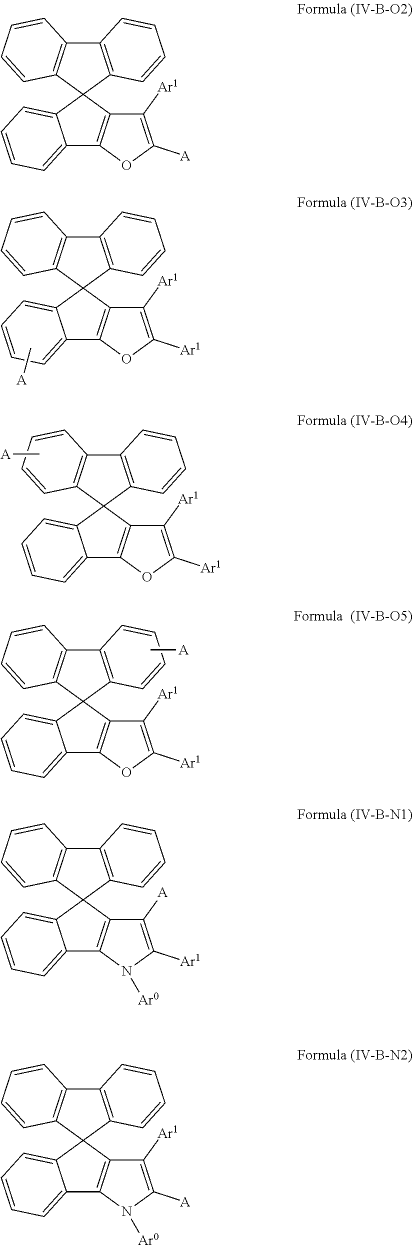

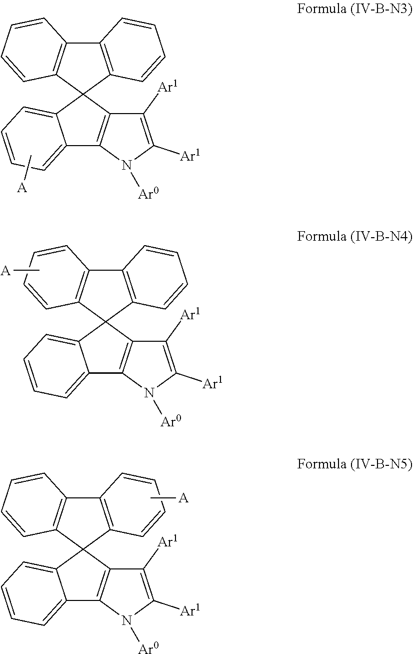

where the variables have the definitions given above and preferably conform to their abovementioned preferred embodiments, and where the unoccupied positions on the benzene rings are each substituted by R¹ radicals that are preferably H.

Ar⁰ in the abovementioned formulae is preferably phenyl substituted by R² radicals, where R² in these cases is preferably H.

Among the abovementioned formulae, particular preference is given to the formulae (II-B-S1) to (II-B-S4), (II-B-N1) to (II-B-N4) and (II-B-O1) to (II-B-O4), (III-B-S1) to (III-B-S4), (III-B-N1) to (III-B-N4) and (III-B-O1) to (III-B-O4), and (IV-B-S1) to (IV-B-S4), (IV-B-N1) to (IV-B-N4) and (IV-B-O1) to (IV-B-O4). Particular preference is given to the formulae (II-B-S1) to (II-B-S4) and (II-B-O1) to (II-B-O4), (III-B-S1) to (III-B-S4) and (III-B-O1) to (III-B-O4), and (IV-B-S1) to (IV-B-S4) and (IV-B-O1) to (IV-B-O4). Very particular preference is given to the formulae (II-B-S1) to (II-B-S4) and (II-B-O1) to (II-B-O4).

More particular preference is given to the formulae (II-B-S1) to (II-B-N1) and (II-B-S2) to (II-B-N2).

Greatest preference is given to the formulae (II-B-S1), (II-B-O1), (II-B-S2) and (II-B-O2).

More particular preference is given to the formulae (II-B-S1), (II-B-S2), (II-B-S3), (II-B-S4), (II-B-O1), (II-B-O2), (II-B-O3), (II-B-O4), (III-B-S1), (III-B-S2), (III-B-S3), (III-B-S4), (III-B-O1), (III-B-O2), (III-B-O3), (III-B-O4), (IV-B-S1), (IV-B-S2), (IV-B-S3), (IV-B-S4), (IV-B-O1), (IV-B-O2), (IV-B-O3), (IV-B-O4), (II-B-N1) and (II-B-N2).

Preferred embodiments of the formulae (II-B-S3), (II-B-S4), (III-B-S3), (III-B-S4), (IV-B-S3) and (IV-B-S4) are the following formulae:

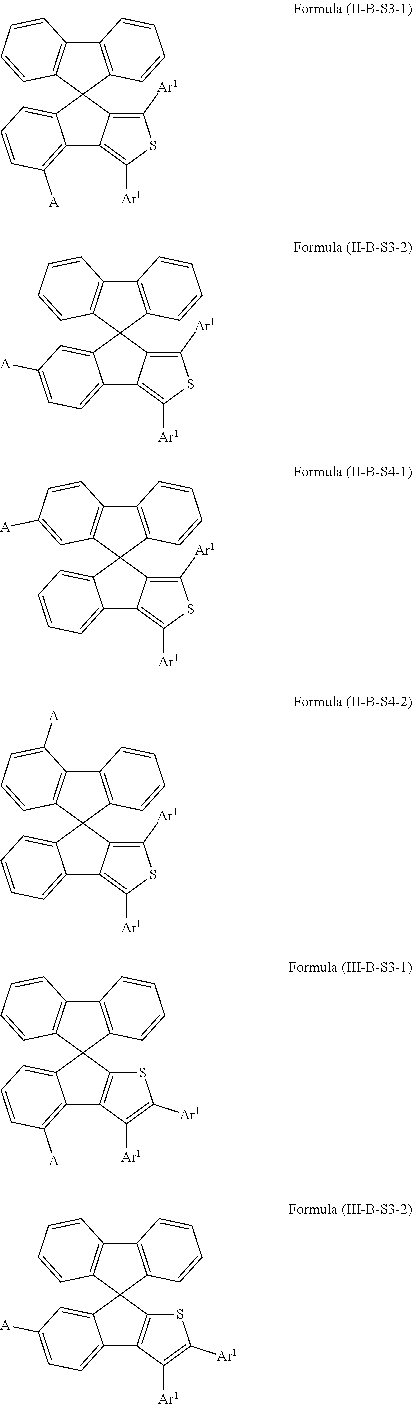

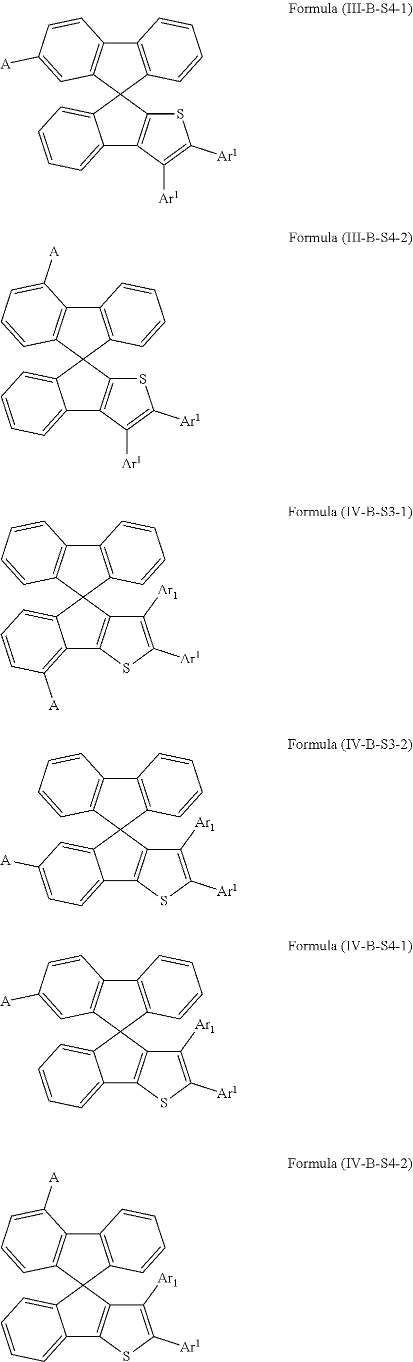

where the variables that occur are as defined above and preferably conform to the preferred embodiments, and where the unoccupied positions on the benzene rings are each substituted by R¹ radicals that are preferably H.

Preferred embodiments of the formula (II-B-O3), (II-B-O4), (III-B-O3), (III-B-O4), (IV-B-O3) and (IV-B-O4) are the following formulae:

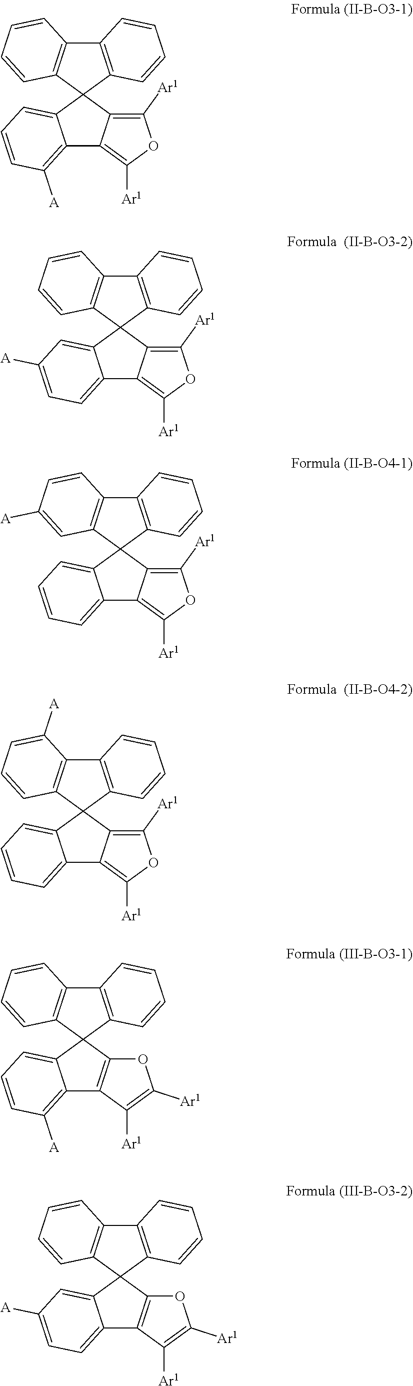

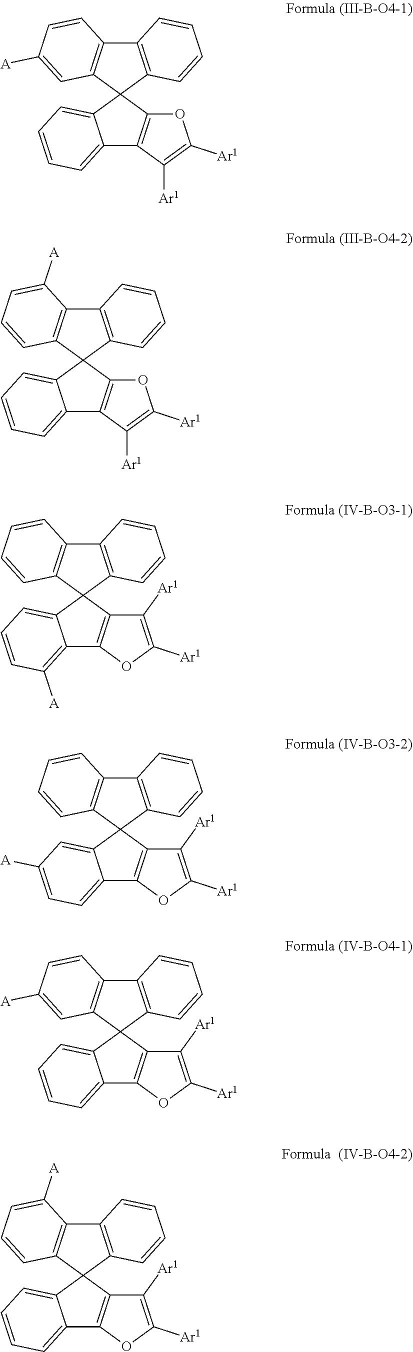

where the variables that occur are as defined above and preferably conform to the preferred embodiments, and where the unoccupied positions on the benzene rings are each substituted by R¹ radicals that are preferably H.

Among the abovementioned formulae, particular preference is given to the formulae (II-B-S3-1), (II-B-S3-2), (II-B-S4-1) (II-B-S4-2), (II-B-O3-1), (II-B-O3-2), (II-B-O4-1) (II-B-O4-2).

Most preferably, compounds of the formula (I) in this embodiment conform to one of the formulae (II-B-S1), (II-B-O1), (II-B-S2), (II-B-O2), (II-B-S3-1), (II-B-S3-2), (II-B-S4-1) (II-B-S4-2), (II-B-O3-1), (II-B-O3-2), (II-B-O4-1) and (II-B-O4-2), where the variables in these cases preferably conform to the above-specified preferred embodiments.

More preferably, the compound according to the application therefore likewise conforms to a formula (II-B-S1), (II-B-S2), (II-B-S3), (II-B-S4), (II-B-O1), (II-B-O2), (II-B-O3), (II-B-O4), (III-B-S1), (III-B-S2), (III-B-S3), (III-B-S4), (III-B-O1), (III-B-O2), (III-B-O3), (III-B-O4), (IV-B-S1), (IV-B-S2), (IV-B-S3), (IV-B-S4), (IV-B-O1), (IV-B-O2), (IV-B-O3), (IV-B-O4), (II-B-N1) and (II-B-N2), where the variables that occur are as follows:

Ar¹ is the same or different at each instance and is selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R² radicals, and H;

R¹ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H;

R⁴ is the same or different at each instance and is selected from straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where the alkyl groups mentioned and the aromatic ring systems mentioned and the heteroaromatic ring systems mentioned are each substituted by R¹ radicals, and with exclusion of bonding of the two R⁴ radicals to one another to form an aliphatic or heteroaliphatic ring;

A is a unit of the formula (A)

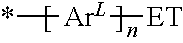

Formula (A), where the variables in formula (A) are defined as follows:

Ar^{L} is the same or different at each instance and is selected from divalent groups derived from benzene, biphenyl, terphenyl, naphthalene, fluorene, indenofluorene, indenocarbazole, spirobifluorene, dibenzofuran and dibenzothiophene, each of which are substituted by R² radicals;

ET is the same or different at each instance and is selected from groups derived from pyridine, pyrazine, pyrimidine, pyridazine, 1,2,4-triazine, 1,3,5-triazine, quinoline, isoquinoline, quinoxaline, quinazoline, pyrazole, imidazole, benzimidazole, thiazole, benzothiazole, oxazole, oxadiazole and benzoxazole, each of which are substituted by Ar² groups;

Ar² is the same or different at each instance and is selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R³ radicals, and H;

n=1 or 0;

and the further variables are as defined in their above-specified broadest embodiment, preferably the above-specified preferred embodiments.

Preferred compounds of the formula (I) in this embodiment are shown in table 1 below.

TABLE 1

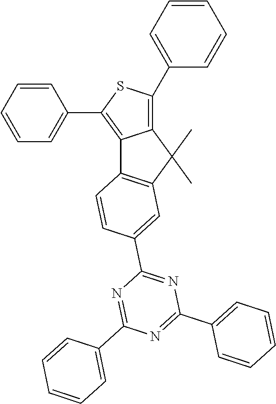 [SMILES: CC1(C)c2cc(-c3nc(-c4ccccc4)nc(-c4ccccc4)n3)ccc2-c2c(-c3ccccc3)sc(-c3ccccc3)c21]

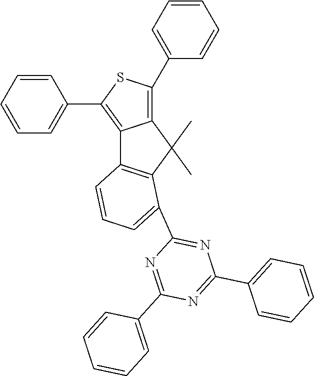 [SMILES: CC1(C)c2c(-c3nc(-c4ccccc4)nc(-c4ccccc4)n3)cccc2-c2c(-c3ccccc3)sc(-c3ccccc3)c21]

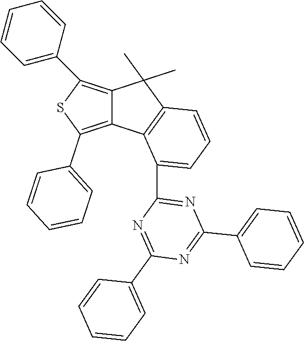 [SMILES: CC1(C)c2cccc(-c3nc(-c4ccccc4)nc(-c4ccccc4)n3)c2-c2c(-c3ccccc3)sc(-c3ccccc3)c21]

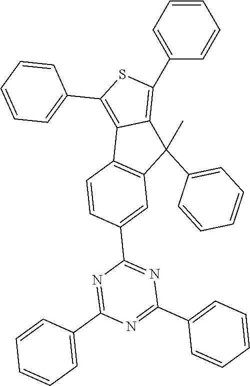 [SMILES: CC1(c2ccccc2)c2cc(-c3nc(-c4ccccc4)nc(-c4ccccc4)n3)ccc2-c2c(-c3ccccc3)sc(-c3ccccc3)c21]

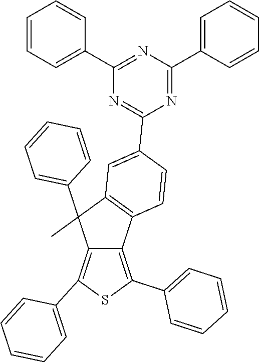 [SMILES: CC1(c2ccccc2)c2cc(-c3nc(-c4ccccc4)nc(-c4ccccc4)n3)ccc2-c2c(-c3ccccc3)sc(-c3ccccc3)c21]

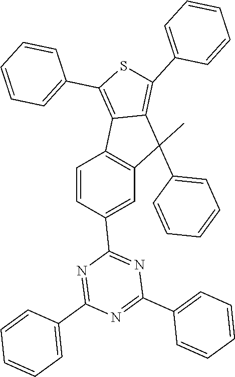 [SMILES: CC1(c2ccccc2)c2cc(-c3nc(-c4ccccc4)nc(-c4ccccc4)n3)ccc2-c2c(-c3ccccc3)sc(-c3ccccc3)c21]

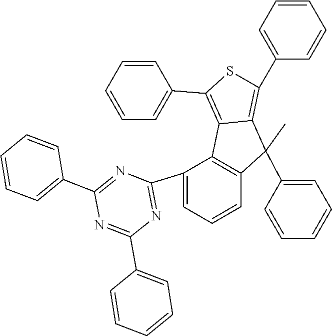 [SMILES: CC1(c2ccccc2)c2cccc(-c3nc(-c4ccccc4)nc(-c4ccccc4)n3)c2-c2c(-c3ccccc3)sc(-c3ccccc3)c21]

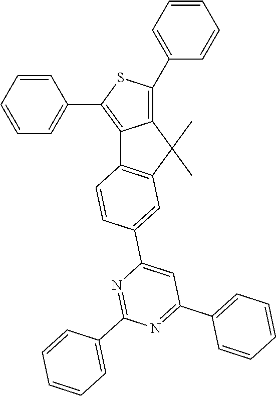 [SMILES: CC1(C)c2cc(-c3cc(-c4ccccc4)nc(-c4ccccc4)n3)ccc2-c2c(-c3ccccc3)sc(-c3ccccc3)c21]

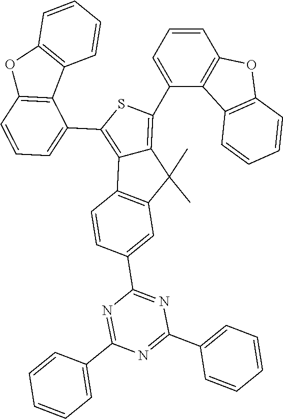 [SMILES: CC1(C)c2cc(-c3nc(-c4ccccc4)nc(-c4ccccc4)n3)ccc2-c2c(-c3cccc4oc5ccccc5c34)sc(-c3cccc4oc5ccccc5c34)c21]

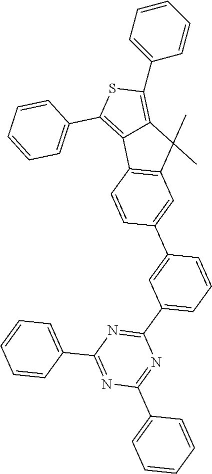 [SMILES: CC1(C)c2cc(-c3cccc(-c4nc(-c5ccccc5)nc(-c5ccccc5)n4)c3)ccc2-c2c(-c3ccccc3)sc(-c3ccccc3)c21]

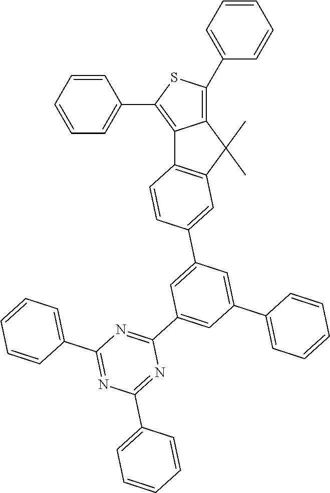 [SMILES: CC1(C)c2cc(-c3cc(-c4ccccc4)cc(-c4nc(-c5ccccc5)nc(-c5ccccc5)n4)c3)ccc2-c2c(-c3ccccc3)sc(-c3ccccc3)c21]

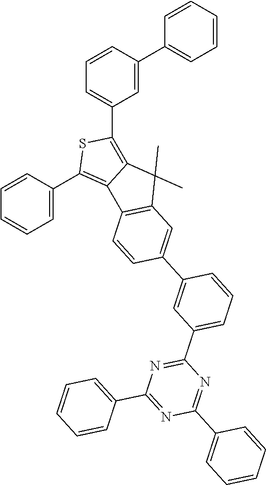 [SMILES: CC1(C)c2cc(-c3cccc(-c4nc(-c5ccccc5)nc(-c5ccccc5)n4)c3)ccc2-c2c(-c3ccccc3)sc(-c3cccc(-c4ccccc4)c3)c21]

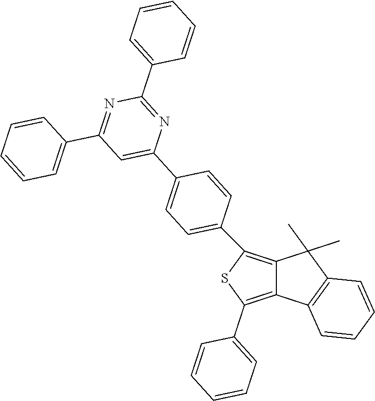 [SMILES: CC1(C)c2ccccc2-c2c(-c3ccccc3)sc(-c3ccc(-c4cc(-c5ccccc5)nc(-c5ccccc5)n4)cc3)c21]

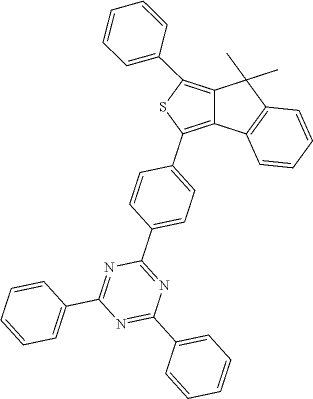 [SMILES: CC1(C)c2ccccc2-c2c(-c3ccc(-c4nc(-c5ccccc5)nc(-c5ccccc5)n4)cc3)sc(-c3ccccc3)c21]

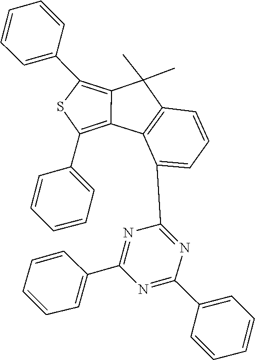 [SMILES: CC1(C)c2cccc(-c3nc(-c4ccccc4)nc(-c4ccccc4)n3)c2-c2c(-c3ccccc3)sc(-c3ccccc3)c21]

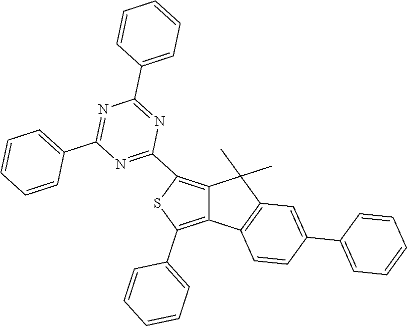 [SMILES: CC1(C)c2cc(-c3ccccc3)ccc2-c2c(-c3ccccc3)sc(-c3nc(-c4ccccc4)nc(-c4ccccc4)n3)c21]

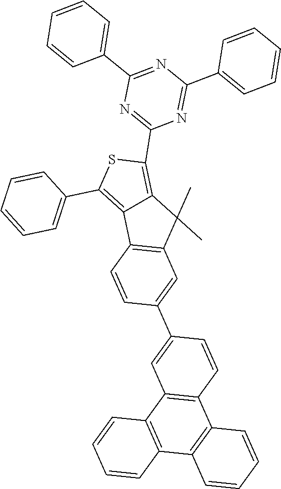 [SMILES: CC1(C)c2cc(-c3ccc4c5ccccc5c5ccccc5c4c3)ccc2-c2c(-c3ccccc3)sc(-c3nc(-c4ccccc4)nc(-c4ccccc4)n3)c21]

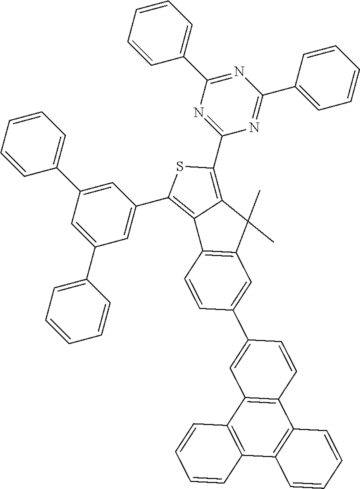 [SMILES: CC1(C)c2cc(-c3ccc4c5ccccc5c5ccccc5c4c3)ccc2-c2c(-c3cc(-c4ccccc4)cc(-c4ccccc4)c3)sc(-c3nc(-c4ccccc4)nc(-c4ccccc4)n3)c21]

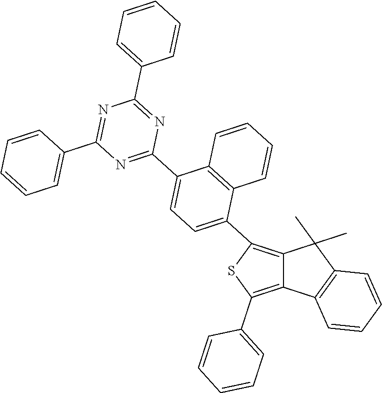 [SMILES: CC1(C)c2ccccc2-c2c(-c3ccccc3)sc(-c3ccc(-c4nc(-c5ccccc5)nc(-c5ccccc5)n4)c4ccccc34)c21]

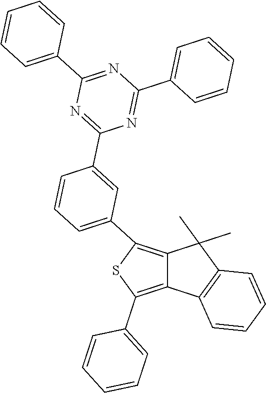 [SMILES: CC1(C)c2ccccc2-c2c(-c3ccccc3)sc(-c3cccc(-c4nc(-c5ccccc5)nc(-c5ccccc5)n4)c3)c21]

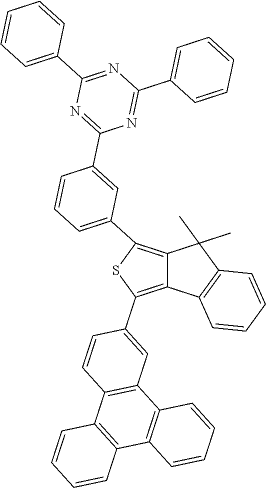 [SMILES: CC1(C)c2ccccc2-c2c(-c3ccc4c5ccccc5c5ccccc5c4c3)sc(-c3cccc(-c4nc(-c5ccccc5)nc(-c5ccccc5)n4)c3)c21]

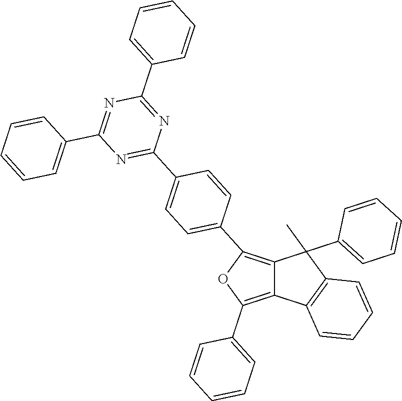 [SMILES: CC1(c2ccccc2)c2ccccc2-c2c(-c3ccccc3)oc(-c3ccc(-c4nc(-c5ccccc5)nc(-c5ccccc5)n4)cc3)c21]

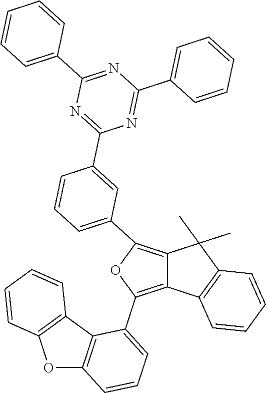 [SMILES: CC1(C)c2ccccc2-c2c(-c3cccc4oc5ccccc5c34)oc(-c3cccc(-c4nc(-c5ccccc5)nc(-c5ccccc5)n4)c3)c21]

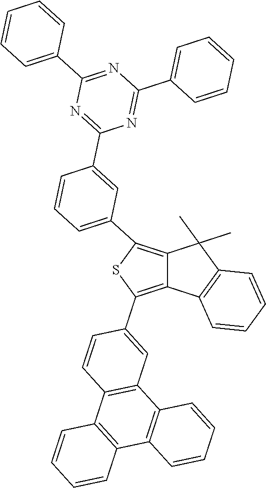 [SMILES: CC1(C)c2ccccc2-c2c(-c3ccc4c5ccccc5c5ccccc5c4c3)sc(-c3cccc(-c4nc(-c5ccccc5)nc(-c5ccccc5)n4)c3)c21]

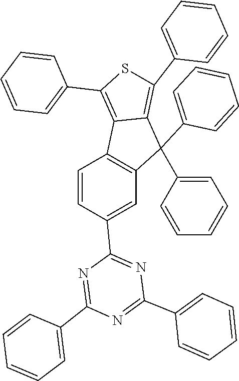 [SMILES: c1ccc(-c2nc(-c3ccccc3)nc(-c3ccc4c(c3)C(c3ccccc3)(c3ccccc3)c3c(-c5ccccc5)sc(-c5ccccc5)c3-4)n2)cc1]

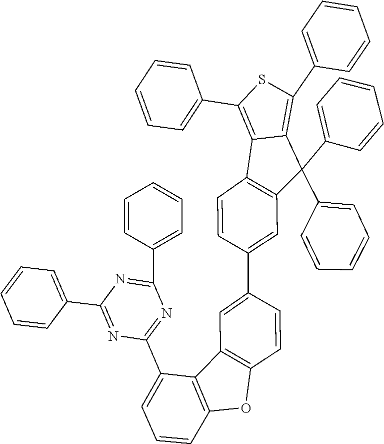 [SMILES: c1ccc(-c2nc(-c3ccccc3)nc(-c3cccc4oc5ccc(-c6ccc7c(c6)C(c6ccccc6)(c6ccccc6)c6c(-c8ccccc8)sc(-c8ccccc8)c6-7)cc5c34)n2)cc1]

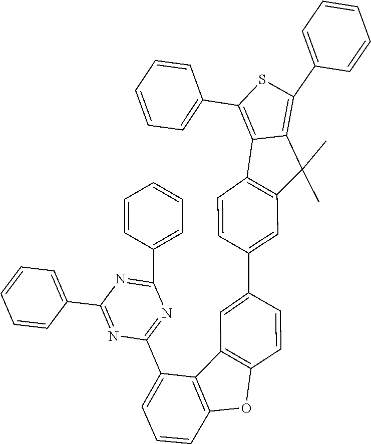 [SMILES: CC1(C)c2cc(-c3ccc4oc5cccc(-c6nc(-c7ccccc7)nc(-c7ccccc7)n6)c5c4c3)ccc2-c2c(-c3ccccc3)sc(-c3ccccc3)c21]

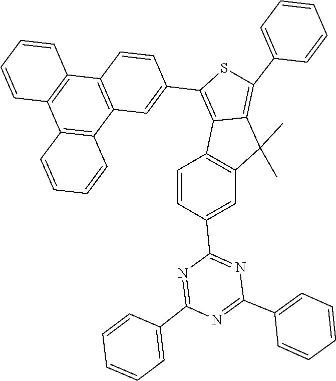 [SMILES: CC1(C)c2cc(-c3nc(-c4ccccc4)nc(-c4ccccc4)n3)ccc2-c2c(-c3ccc4c5ccccc5c5ccccc5c4c3)sc(-c3ccccc3)c21]

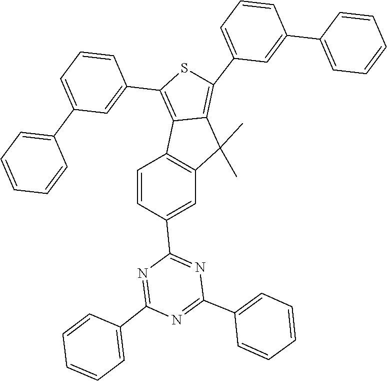 [SMILES: CC1(C)c2cc(-c3nc(-c4ccccc4)nc(-c4ccccc4)n3)ccc2-c2c(-c3cccc(-c4ccccc4)c3)sc(-c3cccc(-c4ccccc4)c3)c21]

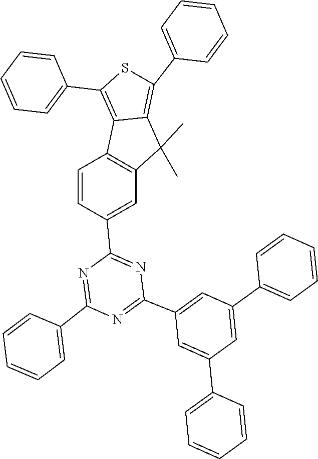 [SMILES: CC1(C)c2cc(-c3nc(-c4ccccc4)nc(-c4cc(-c5ccccc5)cc(-c5ccccc5)c4)n3)ccc2-c2c(-c3ccccc3)sc(-c3ccccc3)c21]

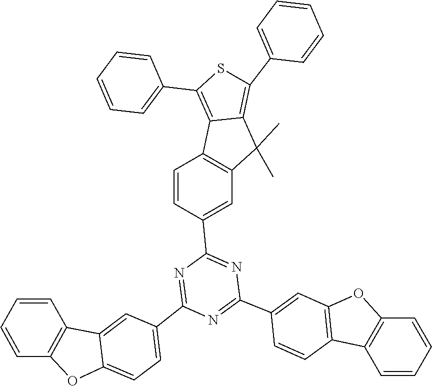 [SMILES: CC1(C)c2cc(-c3nc(-c4ccc5c(c4)oc4ccccc45)nc(-c4ccc5oc6ccccc6c5c4)n3)ccc2-c2c(-c3ccccc3)sc(-c3ccccc3)c21]

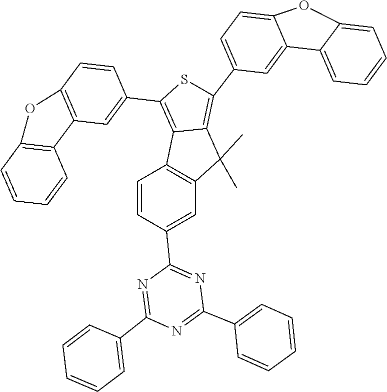 [SMILES: CC1(C)c2cc(-c3nc(-c4ccccc4)nc(-c4ccccc4)n3)ccc2-c2c(-c3ccc4oc5ccccc5c4c3)sc(-c3ccc4oc5ccccc5c4c3)c21]

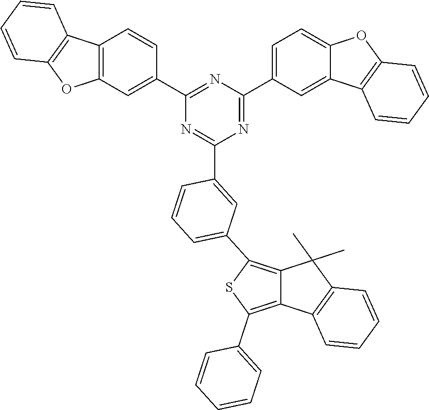 [SMILES: CC1(C)c2ccccc2-c2c(-c3ccccc3)sc(-c3cccc(-c4nc(-c5ccc6c(c5)oc5ccccc56)nc(-c5ccc6oc7ccccc7c6c5)n4)c3)c21]

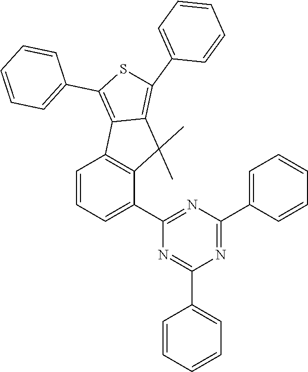 [SMILES: CC1(C)c2c(-c3nc(-c4ccccc4)nc(-c4ccccc4)n3)cccc2-c2c(-c3ccccc3)sc(-c3ccccc3)c21]

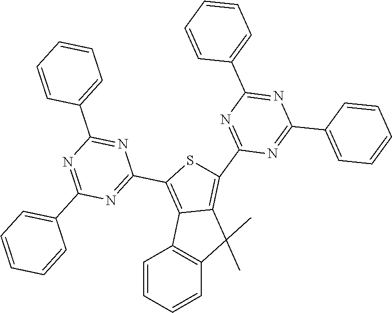 [SMILES: CC1(C)c2ccccc2-c2c(-c3nc(-c4ccccc4)nc(-c4ccccc4)n3)sc(-c3nc(-c4ccccc4)nc(-c4ccccc4)n3)c21]

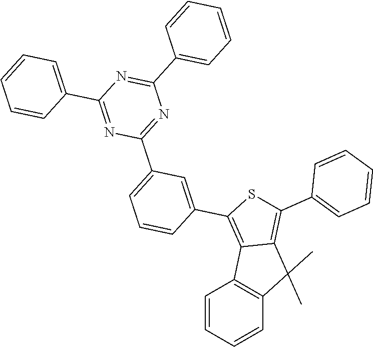 [SMILES: CC1(C)c2ccccc2-c2c(-c3cccc(-c4nc(-c5ccccc5)nc(-c5ccccc5)n4)c3)sc(-c3ccccc3)c21]

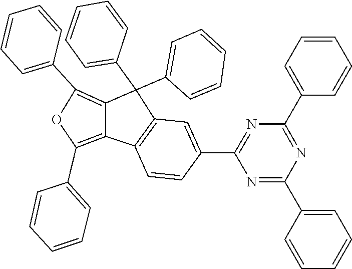 [SMILES: c1ccc(-c2nc(-c3ccccc3)nc(-c3ccc4c(c3)C(c3ccccc3)(c3ccccc3)c3c(-c5ccccc5)oc(-c5ccccc5)c3-4)n2)cc1]

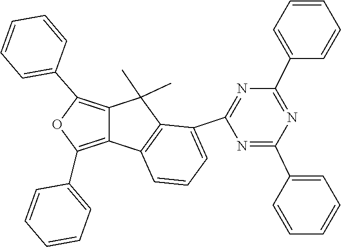 [SMILES: CC1(C)c2c(-c3nc(-c4ccccc4)nc(-c4ccccc4)n3)cccc2-c2c(-c3ccccc3)oc(-c3ccccc3)c21]

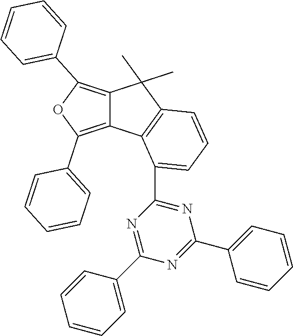 [SMILES: CC1(C)c2cccc(-c3nc(-c4ccccc4)nc(-c4ccccc4)n3)c2-c2c(-c3ccccc3)oc(-c3ccccc3)c21]

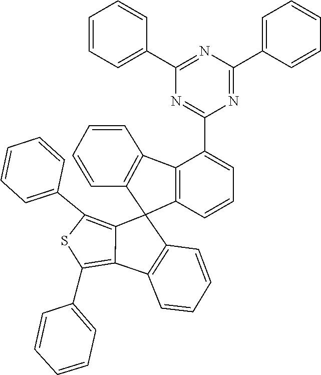 [SMILES: c1ccc(-c2nc(-c3ccccc3)nc(-c3cccc4c3-c3ccccc3C43c4ccccc4-c4c(-c5ccccc5)sc(-c5ccccc5)c43)n2)cc1]

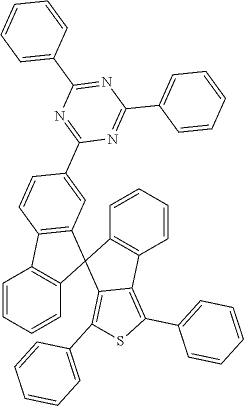 [SMILES: c1ccc(-c2nc(-c3ccccc3)nc(-c3ccc4c(c3)C3(c5ccccc5-4)c4ccccc4-c4c(-c5ccccc5)sc(-c5ccccc5)c43)n2)cc1]

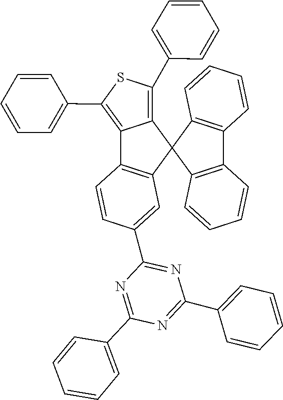 [SMILES: c1ccc(-c2nc(-c3ccccc3)nc(-c3ccc4c(c3)C3(c5ccccc5-c5ccccc53)c3c(-c5ccccc5)sc(-c5ccccc5)c3-4)n2)cc1]

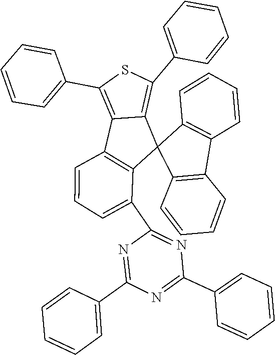 [SMILES: c1ccc(-c2nc(-c3ccccc3)nc(-c3cccc4c3C3(c5ccccc5-c5ccccc53)c3c(-c5ccccc5)sc(-c5ccccc5)c3-4)n2)cc1]

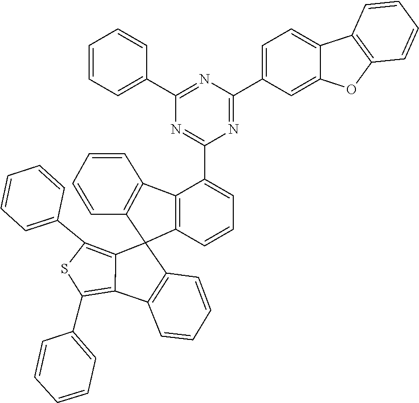 [SMILES: c1ccc(-c2nc(-c3ccc4c(c3)oc3ccccc34)nc(-c3cccc4c3-c3ccccc3C43c4ccccc4-c4c(-c5ccccc5)sc(-c5ccccc5)c43)n2)cc1]

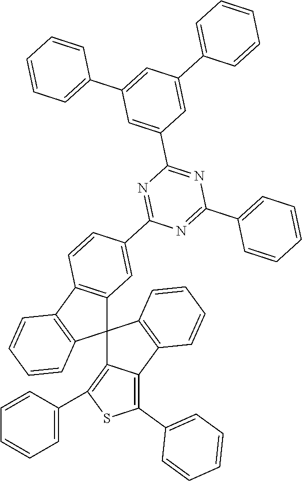 [SMILES: c1ccc(-c2cc(-c3ccccc3)cc(-c3nc(-c4ccccc4)nc(-c4ccc5c(c4)C4(c6ccccc6-5)c5ccccc5-c5c(-c6ccccc6)sc(-c6ccccc6)c54)n3)c2)cc1]

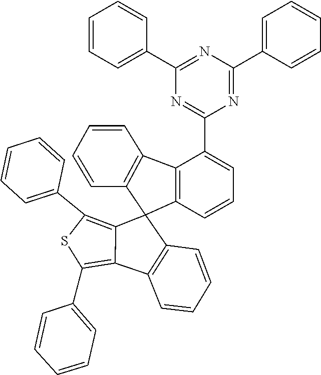 [SMILES: c1ccc(-c2nc(-c3ccccc3)nc(-c3cccc4c3-c3ccccc3C43c4ccccc4-c4c(-c5ccccc5)sc(-c5ccccc5)c43)n2)cc1]

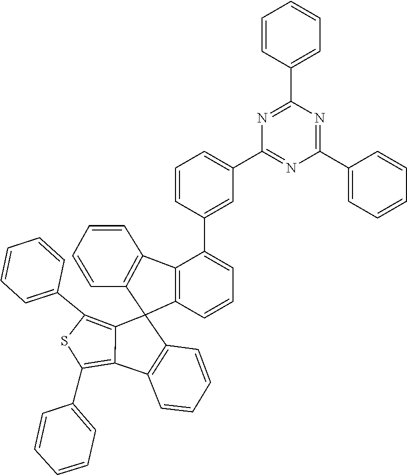 [SMILES: c1ccc(-c2nc(-c3ccccc3)nc(-c3cccc(-c4cccc5c4-c4ccccc4C54c5ccccc5-c5c(-c6ccccc6)sc(-c6ccccc6)c54)c3)n2)cc1]

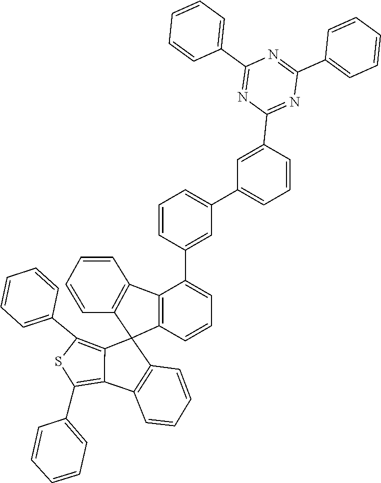 [SMILES: c1ccc(-c2nc(-c3ccccc3)nc(-c3cccc(-c4cccc(-c5cccc6c5-c5ccccc5C65c6ccccc6-c6c(-c7ccccc7)sc(-c7ccccc7)c65)c4)c3)n2)cc1]

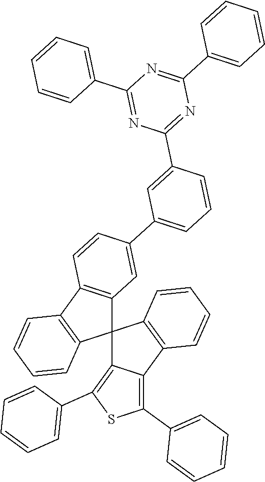 [SMILES: c1ccc(-c2nc(-c3ccccc3)nc(-c3cccc(-c4ccc5c(c4)C4(c6ccccc6-5)c5ccccc5-c5c(-c6ccccc6)sc(-c6ccccc6)c54)c3)n2)cc1]

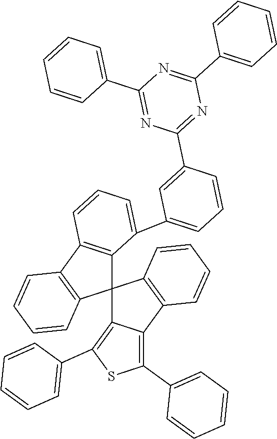 [SMILES: c1ccc(-c2nc(-c3ccccc3)nc(-c3cccc(-c4cccc5c4C4(c6ccccc6-5)c5ccccc5-c5c(-c6ccccc6)sc(-c6ccccc6)c54)c3)n2)cc1]

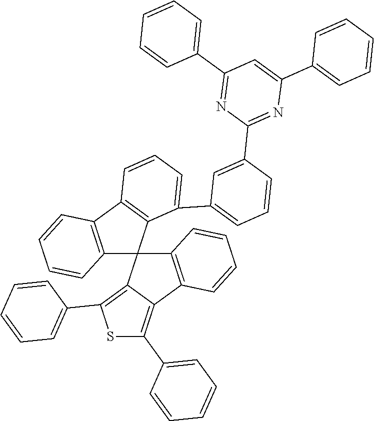 [SMILES: c1ccc(-c2cc(-c3ccccc3)nc(-c3cccc(-c4cccc5c4C4(c6ccccc6-5)c5ccccc5-c5c(-c6ccccc6)sc(-c6ccccc6)c54)c3)n2)cc1]

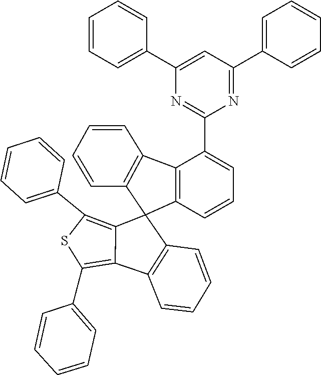 [SMILES: c1ccc(-c2cc(-c3ccccc3)nc(-c3cccc4c3-c3ccccc3C43c4ccccc4-c4c(-c5ccccc5)sc(-c5ccccc5)c43)n2)cc1]

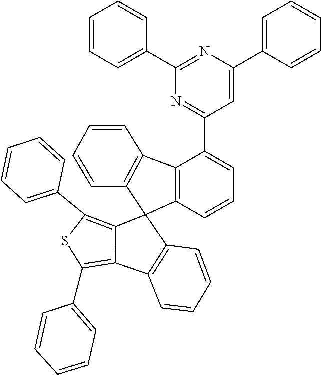 [SMILES: c1ccc(-c2cc(-c3cccc4c3-c3ccccc3C43c4ccccc4-c4c(-c5ccccc5)sc(-c5ccccc5)c43)nc(-c3ccccc3)n2)cc1]

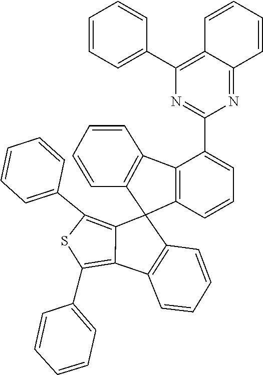 [SMILES: c1ccc(-c2sc(-c3ccccc3)c3c2-c2ccccc2C32c3ccccc3-c3c(-c4nc(-c5ccccc5)c5ccccc5n4)cccc32)cc1]

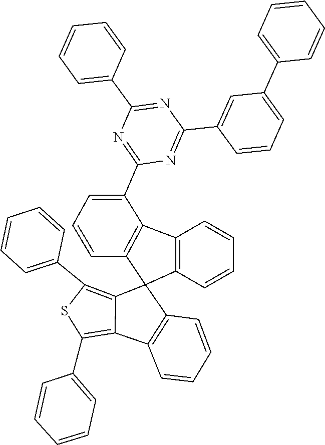 [SMILES: c1ccc(-c2cccc(-c3nc(-c4ccccc4)nc(-c4cccc5c4-c4ccccc4C54c5ccccc5-c5c(-c6ccccc6)sc(-c6ccccc6)c54)n3)c2)cc1]

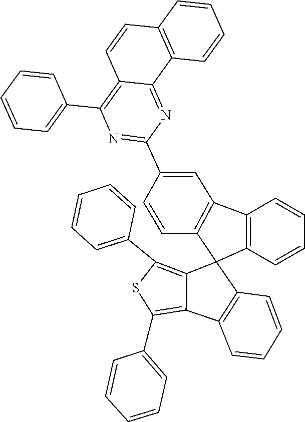 [SMILES: c1ccc(-c2sc(-c3ccccc3)c3c2-c2ccccc2C32c3ccccc3-c3cc(-c4nc(-c5ccccc5)c5ccc6ccccc6c5n4)ccc32)cc1]

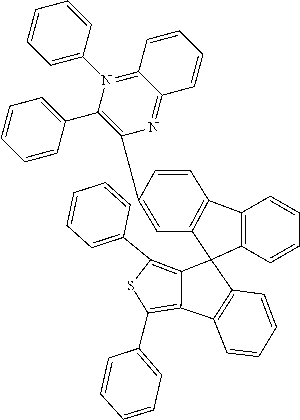

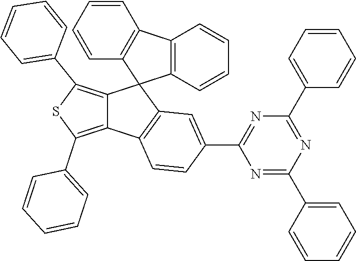 [SMILES: c1ccc(-c2nc(-c3ccccc3)nc(-c3ccc4c(c3)C3(c5ccccc5-c5ccccc53)c3c(-c5ccccc5)sc(-c5ccccc5)c3-4)n2)cc1]

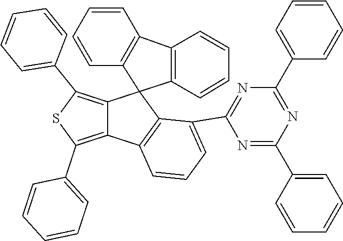 [SMILES: c1ccc(-c2nc(-c3ccccc3)nc(-c3cccc4c3C3(c5ccccc5-c5ccccc53)c3c(-c5ccccc5)sc(-c5ccccc5)c3-4)n2)cc1]

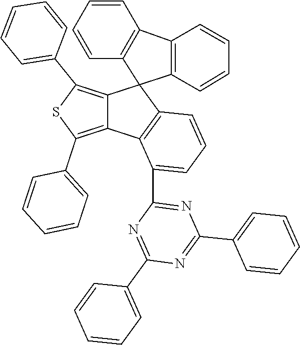 [SMILES: c1ccc(-c2nc(-c3ccccc3)nc(-c3cccc4c3-c3c(-c5ccccc5)sc(-c5ccccc5)c3C43c4ccccc4-c4ccccc43)n2)cc1]

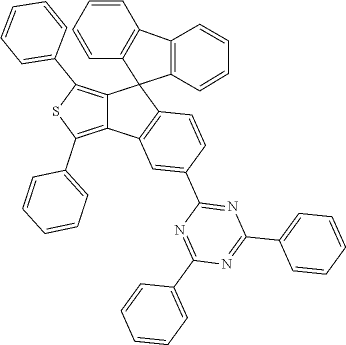 [SMILES: c1ccc(-c2nc(-c3ccccc3)nc(-c3ccc4c(c3)-c3c(-c5ccccc5)sc(-c5ccccc5)c3C43c4ccccc4-c4ccccc43)n2)cc1]

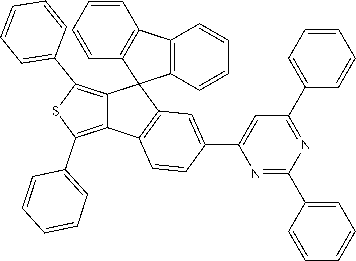 [SMILES: c1ccc(-c2cc(-c3ccc4c(c3)C3(c5ccccc5-c5ccccc53)c3c(-c5ccccc5)sc(-c5ccccc5)c3-4)nc(-c3ccccc3)n2)cc1]

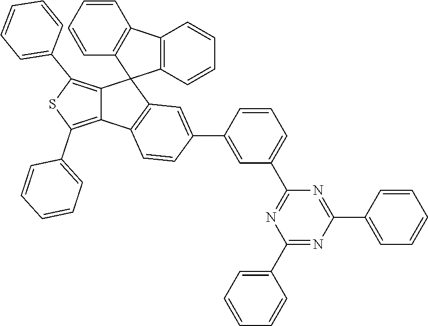 [SMILES: c1ccc(-c2nc(-c3ccccc3)nc(-c3cccc(-c4ccc5c(c4)C4(c6ccccc6-c6ccccc64)c4c(-c6ccccc6)sc(-c6ccccc6)c4-5)c3)n2)cc1]

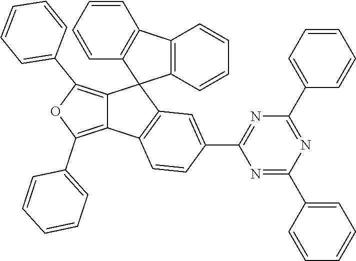 [SMILES: c1ccc(-c2nc(-c3ccccc3)nc(-c3ccc4c(c3)C3(c5ccccc5-c5ccccc53)c3c(-c5ccccc5)oc(-c5ccccc5)c3-4)n2)cc1]

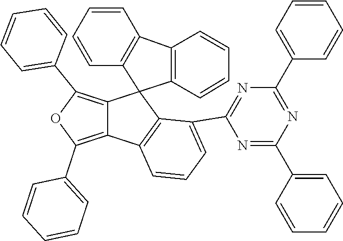 [SMILES: c1ccc(-c2nc(-c3ccccc3)nc(-c3cccc4c3C3(c5ccccc5-c5ccccc53)c3c(-c5ccccc5)oc(-c5ccccc5)c3-4)n2)cc1]

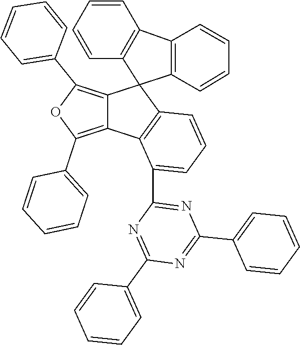 [SMILES: c1ccc(-c2nc(-c3ccccc3)nc(-c3cccc4c3-c3c(-c5ccccc5)oc(-c5ccccc5)c3C43c4ccccc4-c4ccccc43)n2)cc1]

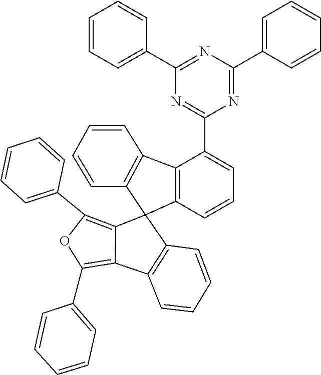 [SMILES: c1ccc(-c2nc(-c3ccccc3)nc(-c3cccc4c3-c3ccccc3C43c4ccccc4-c4c(-c5ccccc5)oc(-c5ccccc5)c43)n2)cc1]

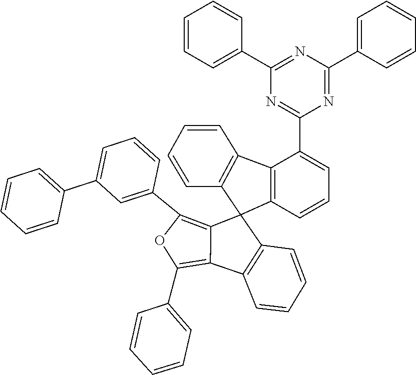 [SMILES: c1ccc(-c2cccc(-c3oc(-c4ccccc4)c4c3C3(c5ccccc5-c5c(-c6nc(-c7ccccc7)nc(-c7ccccc7)n6)cccc53)c3ccccc3-4)c2)cc1]

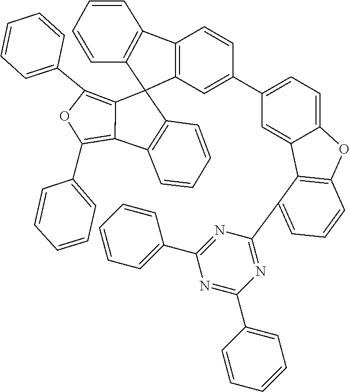 [SMILES: c1ccc(-c2nc(-c3ccccc3)nc(-c3cccc4oc5ccc(-c6ccc7c(c6)C6(c8ccccc8-7)c7ccccc7-c7c(-c8ccccc8)oc(-c8ccccc8)c76)cc5c34)n2)cc1]

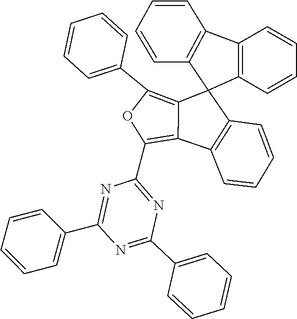 [SMILES: c1ccc(-c2nc(-c3ccccc3)nc(-c3oc(-c4ccccc4)c4c3-c3ccccc3C43c4ccccc4-c4ccccc43)n2)cc1]

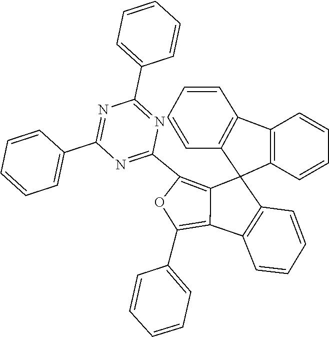 [SMILES: c1ccc(-c2nc(-c3ccccc3)nc(-c3oc(-c4ccccc4)c4c3C3(c5ccccc5-c5ccccc53)c3ccccc3-4)n2)cc1]

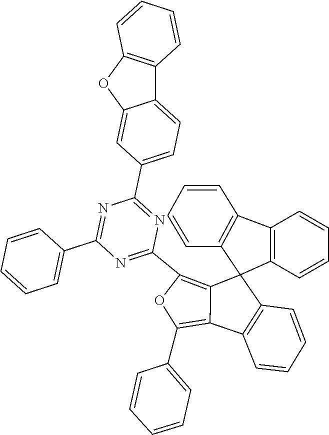 [SMILES: c1ccc(-c2nc(-c3ccc4c(c3)oc3ccccc34)nc(-c3oc(-c4ccccc4)c4c3C3(c5ccccc5-c5ccccc53)c3ccccc3-4)n2)cc1]

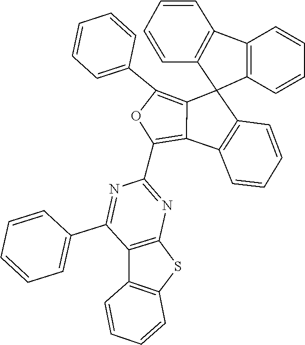 [SMILES: c1ccc(-c2oc(-c3nc(-c4ccccc4)c4c(n3)sc3ccccc34)c3c2C2(c4ccccc4-c4ccccc42)c2ccccc2-3)cc1]

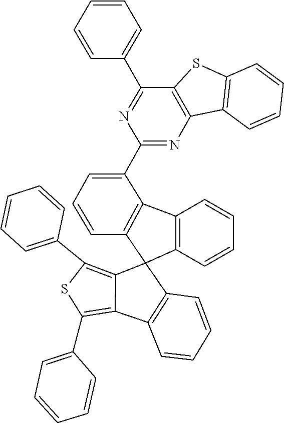 [SMILES: c1ccc(-c2sc(-c3ccccc3)c3c2-c2ccccc2C32c3ccccc3-c3c(-c4nc(-c5ccccc5)c5sc6ccccc6c5n4)cccc32)cc1]

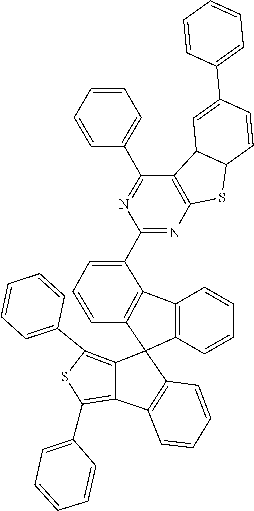 [SMILES: C1=CC2Sc3nc(-c4cccc5c4-c4ccccc4C54c5ccccc5-c5c(-c6ccccc6)sc(-c6ccccc6)c54)nc(-c4ccccc4)c3C2C=C1c1ccccc1]

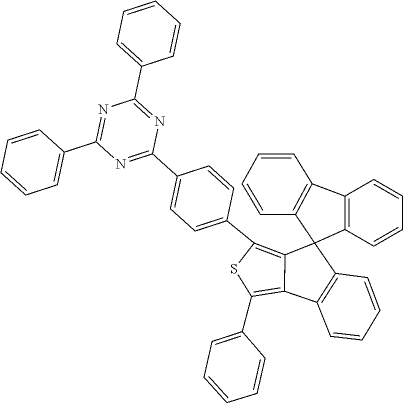 [SMILES: c1ccc(-c2nc(-c3ccccc3)nc(-c3ccc(-c4sc(-c5ccccc5)c5c4C4(c6ccccc6-c6ccccc64)c4ccccc4-5)cc3)n2)cc1]

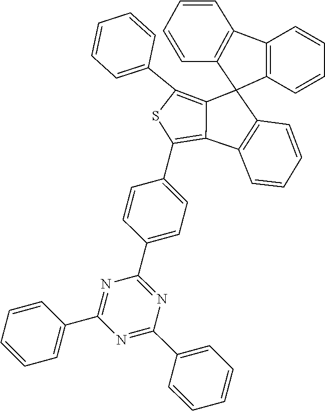 [SMILES: c1ccc(-c2nc(-c3ccccc3)nc(-c3ccc(-c4sc(-c5ccccc5)c5c4-c4ccccc4C54c5ccccc5-c5ccccc54)cc3)n2)cc1]

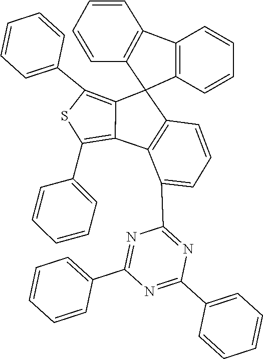 [SMILES: c1ccc(-c2nc(-c3ccccc3)nc(-c3cccc4c3-c3c(-c5ccccc5)sc(-c5ccccc5)c3C43c4ccccc4-c4ccccc43)n2)cc1]

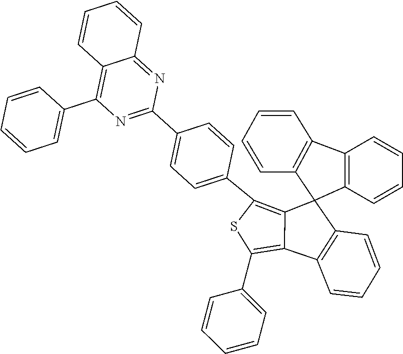 [SMILES: c1ccc(-c2sc(-c3ccc(-c4nc(-c5ccccc5)c5ccccc5n4)cc3)c3c2-c2ccccc2C32c3ccccc3-c3ccccc32)cc1]

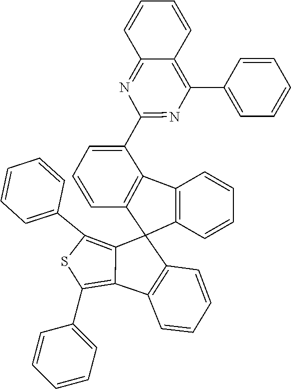 [SMILES: c1ccc(-c2sc(-c3ccccc3)c3c2-c2ccccc2C32c3ccccc3-c3c(-c4nc(-c5ccccc5)c5ccccc5n4)cccc32)cc1]

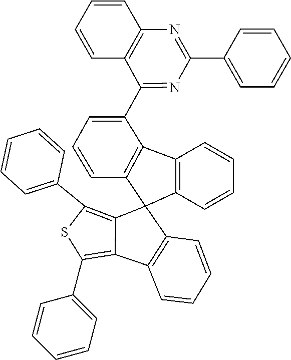 [SMILES: c1ccc(-c2nc(-c3cccc4c3-c3ccccc3C43c4ccccc4-c4c(-c5ccccc5)sc(-c5ccccc5)c43)c3ccccc3n2)cc1]

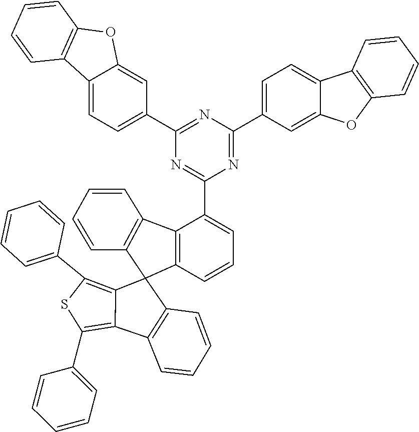 [SMILES: c1ccc(-c2sc(-c3ccccc3)c3c2-c2ccccc2C32c3ccccc3-c3c(-c4nc(-c5ccc6c(c5)oc5ccccc56)nc(-c5ccc6c(c5)oc5ccccc56)n4)cccc32)cc1]

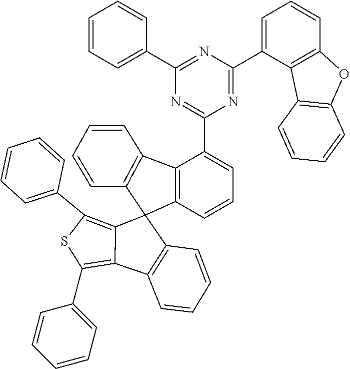 [SMILES: c1ccc(-c2nc(-c3cccc4c3-c3ccccc3C43c4ccccc4-c4c(-c5ccccc5)sc(-c5ccccc5)c43)nc(-c3cccc4oc5ccccc5c34)n2)cc1]

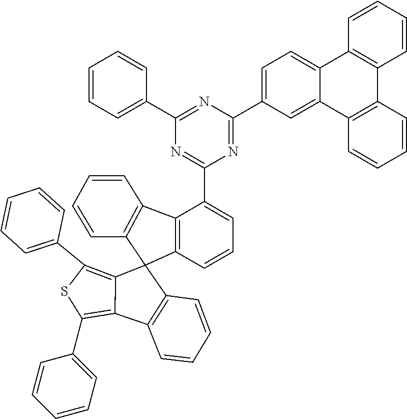 [SMILES: c1ccc(-c2nc(-c3ccc4c5ccccc5c5ccccc5c4c3)nc(-c3cccc4c3-c3ccccc3C43c4ccccc4-c4c(-c5ccccc5)sc(-c5ccccc5)c43)n2)cc1]

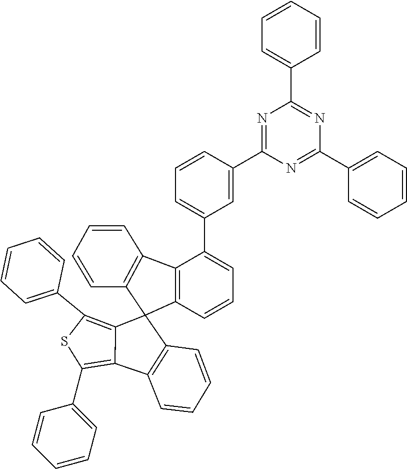 [SMILES: c1ccc(-c2nc(-c3ccccc3)nc(-c3cccc(-c4cccc5c4-c4ccccc4C54c5ccccc5-c5c(-c6ccccc6)sc(-c6ccccc6)c54)c3)n2)cc1]

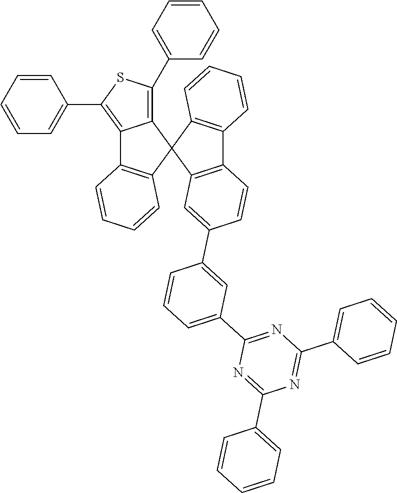 [SMILES: c1ccc(-c2nc(-c3ccccc3)nc(-c3cccc(-c4ccc5c(c4)C4(c6ccccc6-5)c5ccccc5-c5c(-c6ccccc6)sc(-c6ccccc6)c54)c3)n2)cc1]

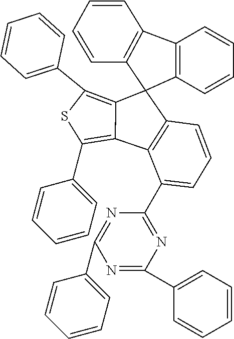 [SMILES: c1ccc(-c2nc(-c3ccccc3)nc(-c3cccc4c3-c3c(-c5ccccc5)sc(-c5ccccc5)c3C43c4ccccc4-c4ccccc43)n2)cc1]

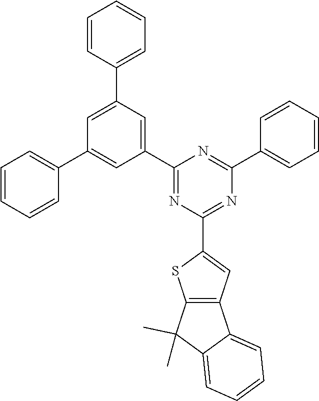 [SMILES: CC1(C)c2ccccc2-c2cc(-c3nc(-c4ccccc4)nc(-c4cc(-c5ccccc5)cc(-c5ccccc5)c4)n3)sc21]

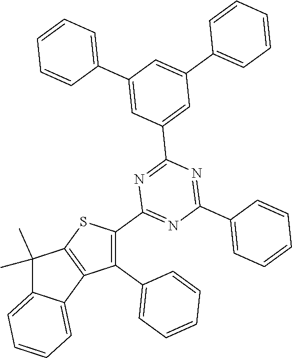 [SMILES: CC1(C)c2ccccc2-c2c1sc(-c1nc(-c3ccccc3)nc(-c3cc(-c4ccccc4)cc(-c4ccccc4)c3)n1)c2-c1ccccc1]

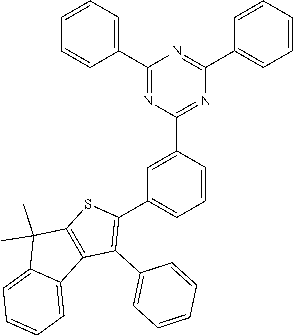 [SMILES: CC1(C)c2ccccc2-c2c1sc(-c1cccc(-c3nc(-c4ccccc4)nc(-c4ccccc4)n3)c1)c2-c1ccccc1]

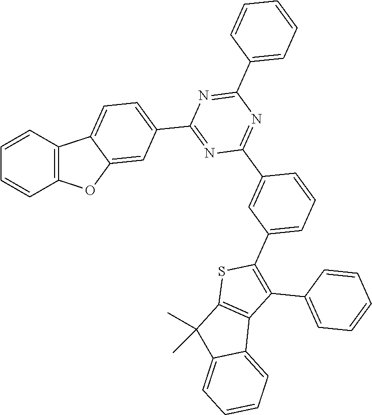 [SMILES: CC1(C)c2ccccc2-c2c1sc(-c1cccc(-c3nc(-c4ccccc4)nc(-c4ccc5c(c4)oc4ccccc45)n3)c1)c2-c1ccccc1]

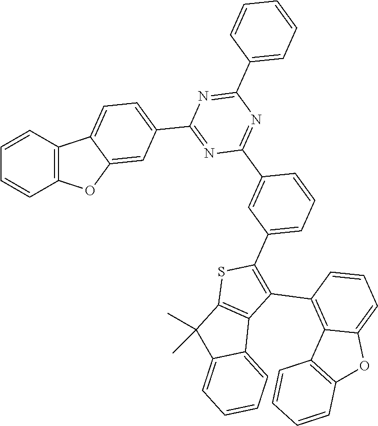 [SMILES: CC1(C)c2ccccc2-c2c1sc(-c1cccc(-c3nc(-c4ccccc4)nc(-c4ccc5c(c4)oc4ccccc45)n3)c1)c2-c1cccc2oc3ccccc3c12]

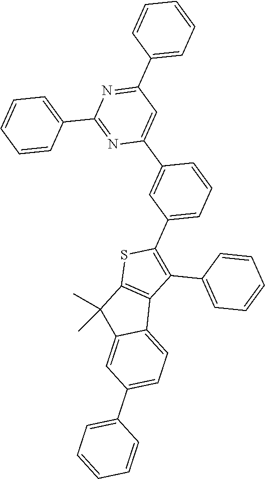 [SMILES: CC1(C)c2cc(-c3ccccc3)ccc2-c2c1sc(-c1cccc(-c3cc(-c4ccccc4)nc(-c4ccccc4)n3)c1)c2-c1ccccc1]

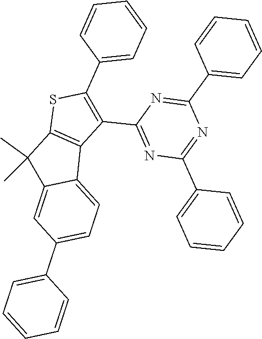 [SMILES: CC1(C)c2cc(-c3ccccc3)ccc2-c2c1sc(-c1ccccc1)c2-c1nc(-c2ccccc2)nc(-c2ccccc2)n1]

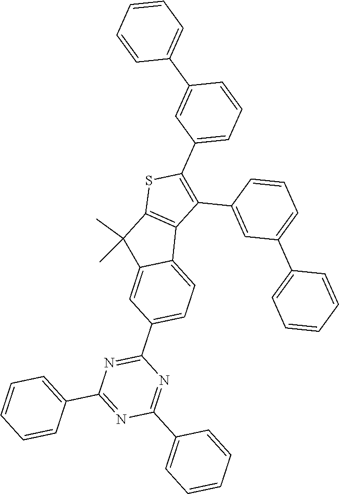 [SMILES: CC1(C)c2cc(-c3nc(-c4ccccc4)nc(-c4ccccc4)n3)ccc2-c2c1sc(-c1cccc(-c3ccccc3)c1)c2-c1cccc(-c2ccccc2)c1]

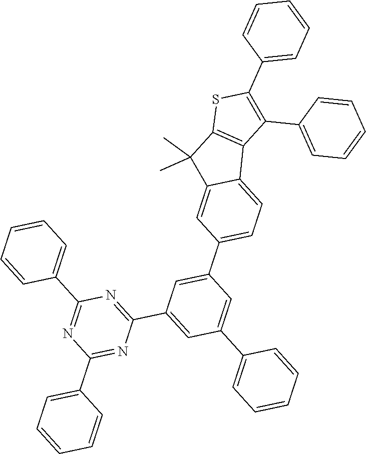 [SMILES: CC1(C)c2cc(-c3cc(-c4ccccc4)cc(-c4nc(-c5ccccc5)nc(-c5ccccc5)n4)c3)ccc2-c2c1sc(-c1ccccc1)c2-c1ccccc1]

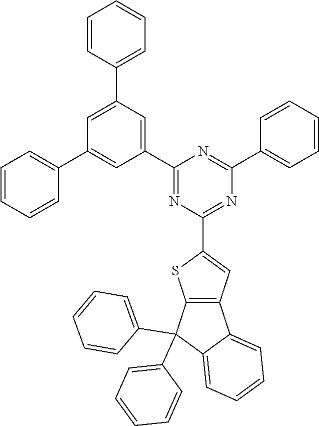 [SMILES: c1ccc(-c2cc(-c3ccccc3)cc(-c3nc(-c4ccccc4)nc(-c4cc5c(s4)C(c4ccccc4)(c4ccccc4)c4ccccc4-5)n3)c2)cc1]

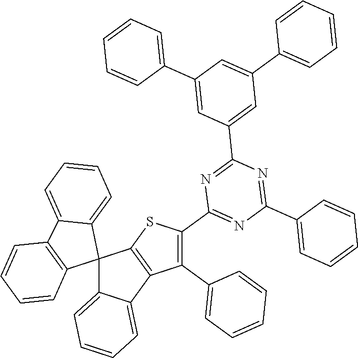 [SMILES: c1ccc(-c2cc(-c3ccccc3)cc(-c3nc(-c4ccccc4)nc(-c4sc5c(c4-c4ccccc4)-c4ccccc4C54c5ccccc5-c5ccccc54)n3)c2)cc1]

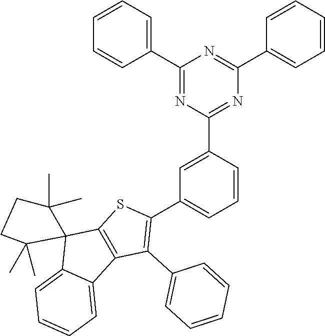 [SMILES: CC1(C)CCC(C)(C)C12c1ccccc1-c1c2sc(-c2cccc(-c3nc(-c4ccccc4)nc(-c4ccccc4)n3)c2)c1-c1ccccc1]

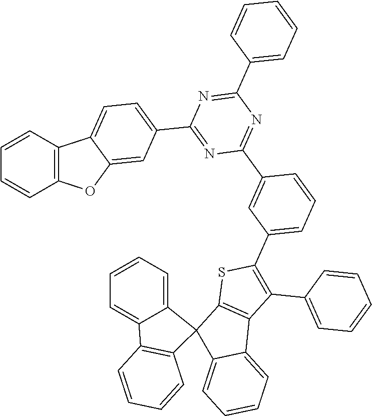 [SMILES: c1ccc(-c2nc(-c3cccc(-c4sc5c(c4-c4ccccc4)-c4ccccc4C54c5ccccc5-c5ccccc54)c3)nc(-c3ccc4c(c3)oc3ccccc34)n2)cc1]

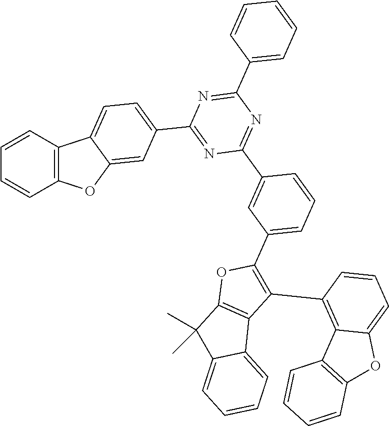 [SMILES: CC1(C)c2ccccc2-c2c1oc(-c1cccc(-c3nc(-c4ccccc4)nc(-c4ccc5c(c4)oc4ccccc45)n3)c1)c2-c1cccc2oc3ccccc3c12]

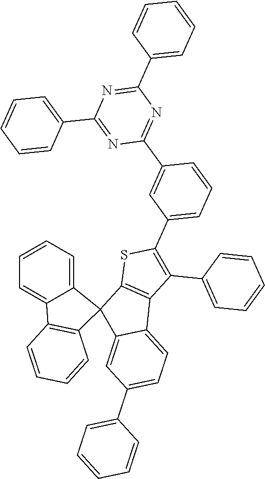 [SMILES: c1ccc(-c2ccc3c(c2)C2(c4ccccc4-c4ccccc42)c2sc(-c4cccc(-c5nc(-c6ccccc6)nc(-c6ccccc6)n5)c4)c(-c4ccccc4)c2-3)cc1]

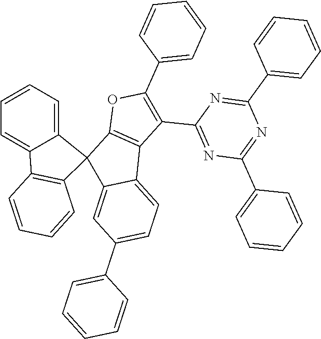 [SMILES: c1ccc(-c2ccc3c(c2)C2(c4ccccc4-c4ccccc42)c2oc(-c4ccccc4)c(-c4nc(-c5ccccc5)nc(-c5ccccc5)n4)c2-3)cc1]

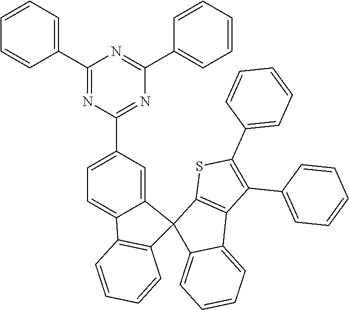 [SMILES: c1ccc(-c2nc(-c3ccccc3)nc(-c3ccc4c(c3)C3(c5ccccc5-4)c4ccccc4-c4c3sc(-c3ccccc3)c4-c3ccccc3)n2)cc1]

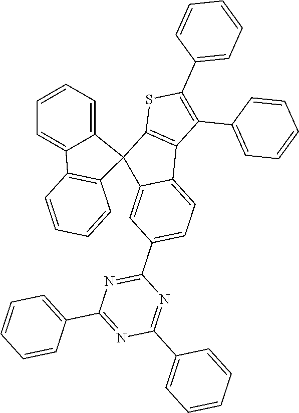 [SMILES: c1ccc(-c2nc(-c3ccccc3)nc(-c3ccc4c(c3)C3(c5ccccc5-c5ccccc53)c3sc(-c5ccccc5)c(-c5ccccc5)c3-4)n2)cc1]

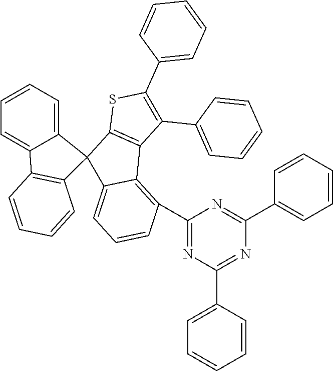 [SMILES: c1ccc(-c2nc(-c3ccccc3)nc(-c3cccc4c3-c3c(sc(-c5ccccc5)c3-c3ccccc3)C43c4ccccc4-c4ccccc43)n2)cc1]

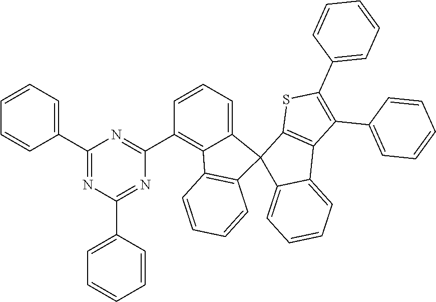 [SMILES: c1ccc(-c2nc(-c3ccccc3)nc(-c3cccc4c3-c3ccccc3C43c4ccccc4-c4c3sc(-c3ccccc3)c4-c3ccccc3)n2)cc1]

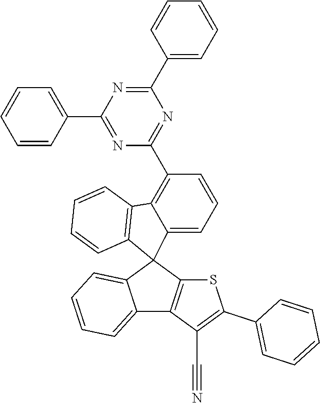 [SMILES: N#Cc1c(-c2ccccc2)sc2c1-c1ccccc1C21c2ccccc2-c2c(-c3nc(-c4ccccc4)nc(-c4ccccc4)n3)cccc21]

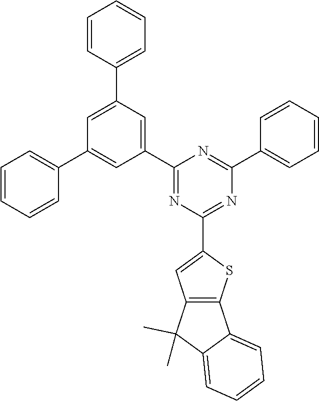 [SMILES: CC1(C)c2ccccc2-c2sc(-c3nc(-c4ccccc4)nc(-c4cc(-c5ccccc5)cc(-c5ccccc5)c4)n3)cc21]

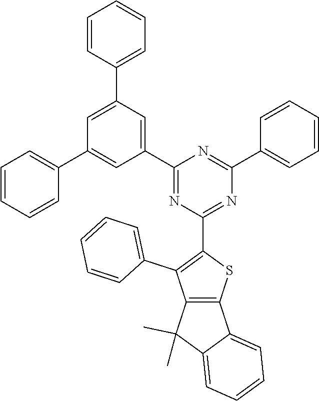 [SMILES: CC1(C)c2ccccc2-c2sc(-c3nc(-c4ccccc4)nc(-c4cc(-c5ccccc5)cc(-c5ccccc5)c4)n3)c(-c3ccccc3)c21]

The compounds according to the application can be prepared by means of organic chemistry synthesis steps known to the person skilled in the art, for example by means of metallation, addition of nucleophiles onto carbonyl groups, Suzuki reaction and Hartwig-Buchwald reaction.

Preferred processes for preparing compounds according to the application are detailed hereinafter. The processes should be understood in an illustrative and non-limiting manner. The person skilled in the art will be able to depart from the illustrative processes detailed and make changes within the scope of his common art knowledge, if this is technically advantageous, in order to arrive at the compounds according to the application.

In a preferred process, in a first step, a heteroaromatic five-membered ring (more preferably pyrrole, furan or thiophene) bearing a carboxylic acid group or a carboxylic ester group is coupled to a benzene ring in a Suzuki reaction (Scheme 1). Alternatively, the carboxylic acid or carboxylic ester group may also be bonded to the benzene ring.

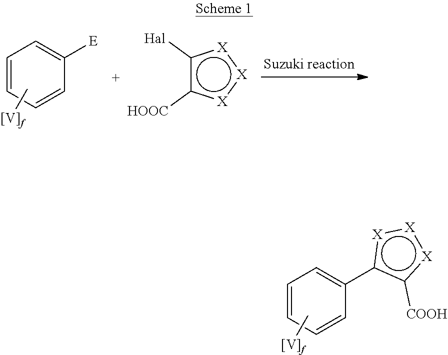

The variables here are defined as follows:

V is the same or different at each instance and is selected from reactive groups, preferably Cl, Br or I;

X is the same or different at each instance and is selected from S, O, NAr⁰, C—H and C—V, where Ar⁰ is as defined in formula (I), and preferably exactly two X, more preferably exactly one X is selected from S, O and NAr⁰;

Hal is Cl, Br or I;

E is a reactive group, preferably a boronic acid group or a boronic ester group; and

f is 0, 1 or 2;

where the compounds are each substituted at the unoccupied positions on the benzene ring by an R¹ radical, as defined above for formula (I), and where at least one V group is present.

The compounds obtained according to scheme 1 are used to prepare intermediates of a formula (Int-1) by ring closure reaction under acidic conditions (Scheme 2).

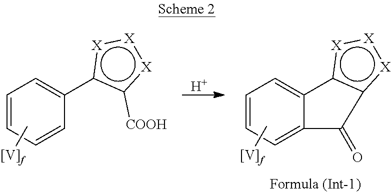

The variables here are as defined for scheme 1.

For preparation of the fluorene derivatives of formula (I) or formula (I-A) according to the application, in each of which a benzene ring has been exchanged for a five-membered heteroaryl ring (more preferably pyrrole, furan or thiophene ring), the compounds of the formula (Int-1) are reduced to compounds of the formula (Int-2), for example by means of addition of a solution of hydrazine hydrate in the presence of Raney nickel as catalyst. The compounds of the formula (Int-2) are converted in a subsequent step by addition of an organohalogen compound or an organometallic reagent, preferably Grignard reagent, to intermediate compounds of the formula (Int-3) (Scheme 3).

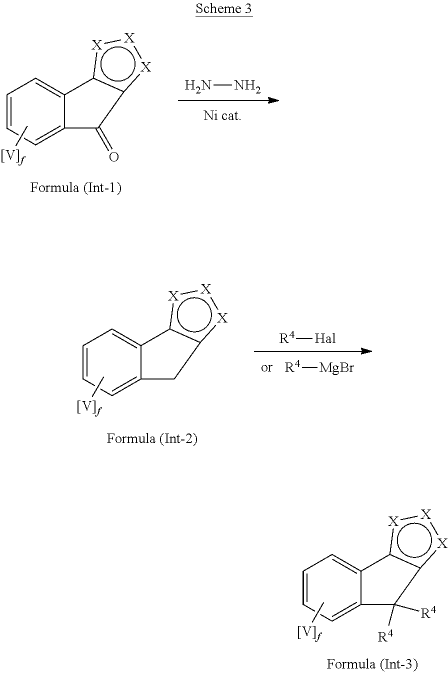

The variables here are as defined for scheme 1, where R⁴ is as defined above for formula (I-A).

For introduction of the at least one A group into the five-membered heteroaryl ring and/or benzene ring, the compound of the formula (Int-3) is coupled in a Suzuki reaction, after conversion of the corresponding V group(s) to one or more reactive groups E that are preferably a boronic acid group or a boronic ester group, to at least one A group (Hal-A). It is optionally also possible for one or two Ar¹ groups to be bonded to the five-membered heteroaryl ring (when X corresponds to C—V) beforehand, likewise preferably by Suzuki reaction. This affords compounds of formula (I-A), as shown in scheme 4:

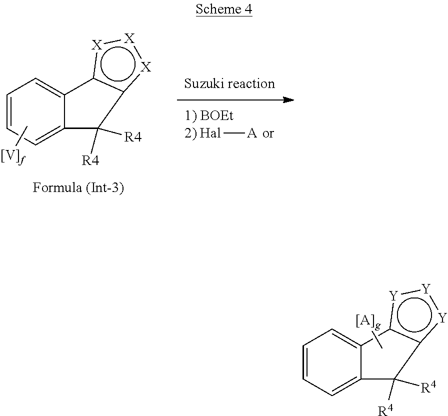

The variables are as defined above for formula (I-A), where index g is 1 or 2, preferably 1, and where the formulae are each substituted by an R¹ radical at the unoccupied positions on the benzene ring.

For preparation of the spirobifluorene derivatives of formula (I) or formula (I-B) according to the application, in each of which a benzene ring has been exchanged for a five-membered heteroaryl ring (more preferably pyrrole, furan or thiophene ring), the compounds of the formula (Int-1) are reacted in a subsequent step, for example by addition of ortho-halogenated or ortho-metallated bisaryl on to the carbonyl function of the compounds of the formula (Int-1) and subsequent acid-catalysed ring closure, giving compounds of the formula (Int-4) (scheme 5).

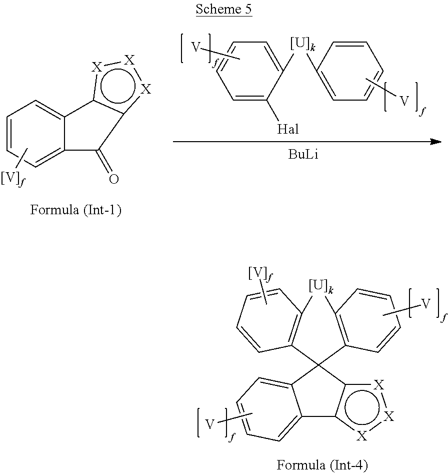

The variables in the compounds of the formula (Int-4) are as defined above, where index f is preferably 0 or 1 and index k is 0 or 1, preferably 0, and where at least one V group is present, and where the formulae are each substituted by an R¹ radical at the unoccupied positions on the benzene rings.

For introduction of the at least one A group into the five-membered heteroaryl ring and/or benzene rings, the compound of the formula (Int-4) is coupled in a Suzuki reaction, after conversion of the corresponding V group(s) to one or more reactive groups E that are preferably a boronic acid group or a boronic ester group, to at least one A group (Hal-A). It is optionally also possible for one or two Ar¹ groups to be bonded to the five-membered heteroaryl ring (when X corresponds to C—V) beforehand, likewise preferably by Suzuki reaction. This affords compounds of formula (I-B), as shown in scheme 6:

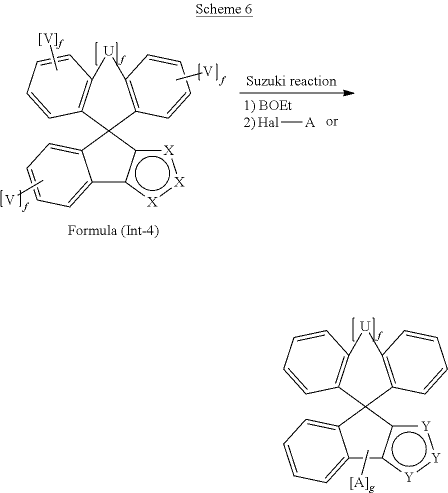

The variables are as defined above for formula (I-B), where index g is 1 or 2, preferably 1, and where the formulae are each substituted by an R¹ radical at the unoccupied positions on the benzene rings.

As shown above in scheme 4 or 6, the intermediates of the formulae (Int-3) and (Int-4) may be reacted by means of a Suzuki coupling with a heteroaryl compound corresponding to group A (Hal-A). This affords compounds of formula (I).

The present application thus provides a process for preparing a fluorene derivative of the formula (I), especially of the formula (I-A), or a spirobifluorene derivative of the formula (I), especially of the formula (I-B), characterized in that in a first step a Suzuki coupling is conducted, in which a heteroaromatic five-membered ring is coupled to a benzene ring, where the heteroaromatic five-membered ring or the benzene ring bears a carboxylic acid group; in that in a second step the carboxylic acid group is cyclized by ring closure reaction under acidic conditions to form a bridging carbonyl group between the heteroaromatic five-membered ring and the benzene ring; in that in a third step the carbonyl group is reduced and, with addition of an organohalogen compound or an organometallic reagent, a substituted methylene bridge is obtained between the heteroaromatic five-membered ring and the benzene ring; and in that in a fourth step a Suzuki coupling with a heteroaryl compound is conducted to obtain a compound of the formula (I), especially of the formula (I-A); or in that in a third step an ortho-halogenated or ortho-metallated bisaryl is added on and a further ring closure reaction is conducted; and in that in a fourth step a Suzuki coupling with a heteroaryl compound is conducted to obtain a compound of the formula (I), especially of the formula (I-B).

The above-described compounds of the invention, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C—C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more fluorene derivatives or spirobifluorene derivatives of formula (I), or formula (I-A) or (I-B), wherein the bond(s) to the polymer, oligomer or dendrimer may be localized at any desired positions substituted by R⁰, R¹, R², R³ or R⁴ in the formula (I), or (I-A) or (I-B). According to the linkage of the compound of formula (I), or formula (I-A) or (I-B), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of formula (I), or (I-A) or (I-B), may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (I), or (I-A) or (I-B), to be joined via a trivalent or higher-valency group, for example via a trivalent or higher-valency aromatic or heteroaromatic group, to give a branched or dendritic oligomer or polymer.

For the repeat units of formula (I), or (I-A) or (I-B), in oligomers, dendrimers and polymers, the same preferences apply as described above for compounds of formula (I), or (I-A) or (I-B).

For preparation of the oligomers or polymers, the monomers of the invention are homopolymerized or copolymerized with further monomers. Suitable and preferred comonomers are selected from fluorenes, spirobifluorenes, paraphenylenes, carbazoles, thiophenes, dihydrophenanthrenes, cis- and trans-indenofluorenes, ketones, phenanthrenes or else two or more of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers, oligomers and dendrimers of the invention have advantageous properties, especially high lifetimes, high efficiencies and good colour coordinates.

The polymers and oligomers of the invention are generally prepared by polymerization of one or more monomer types, of which at least one monomer leads to repeat units of the formula (I), or (I-A) or (I-B), in the polymer. Suitable polymerization reactions are known to those skilled in the art and are described in the literature. Particularly suitable and preferred polymerization reactions which lead to C—C and C—N couplings are as follows:

(A) SUZUKI polymerization;

(B) YAMAMOTO polymerization;

(C) STILLE polymerization; and

(D) HARTWIG-BUCHWALD polymerization.

How the polymerization can be conducted by these methods and how the polymers can then be separated from the reaction medium and purified is known to those skilled in the art and is described in detail in the literature.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one fluorene derivative or spirobifluorene derivative of formula (I), or (I-A) or (I-B), or at least one polymer, oligomer or dendrimer containing at least one unit of formula (I), or (I-A) or (I-B), and at least one solvent, preferably an organic solvent. The way in which such solutions can be prepared is known to those skilled in the art.

The compounds of the invention or the polymers, oligomers and dendrimers of the invention may additionally be present in a composition together with at least one further compound, especially at least one further organic or inorganic compound which is likewise used in the electronic device, for example a hole injection material, a hole transport material, a hole blocker material, wide bandgap material, fluorescent emitter, delayed fluorescent material, phosphorescent emitter, host material, matrix material, electron blocker material, electron transport material and electron injection material, n-dopant and p-dopant.

The invention therefore further provides a composition comprising at least one compound of the invention or at least one polymer, oligomer or dendrimer of the invention and a further compound selected from hole injection materials, hole transport materials, hole blocker materials, wide bandgap materials, fluorescent emitters, delayed fluorescence materials, phosphorescent emitters, host materials, matrix materials, electron blocker materials, electron transport materials and electron injection materials, n-dopants and p-dopants. Suitable examples of these further compounds are listed below.

The compound of formula (I) is suitable for use in an electronic device, especially an organic electroluminescent device (OLED). Depending on the substitution, the compound of the formula (I) can be used in different functions and layers. Preference is given to use as an electron-transporting material in an electron-transporting layer and/or as matrix material in an emitting layer, more preferably in combination with a phosphorescent emitter.

The invention therefore further provides for the use of a compound of formula (I) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound of formula (I). This electronic device is preferably selected from the abovementioned devices.

Particular preference is given to an organic electroluminescent device comprising anode, cathode and at least one emitting layer, characterized in that at least one organic layer comprising at least one compound of formula (I) is present in the device. Preference is given to an organic electroluminescent device comprising anode, cathode and at least one emitting layer, characterized in that at least one organic layer in the device, selected from electron-transporting and emitting layers, comprises at least one compound of formula (I).

An electron-transporting layer is understood here to mean all layers disposed between cathode and emitting layer, preferably electron injection layer (EIL), electron transport layer (ETL), and hole blocker layer (HBL). An electron injection layer is understood here to mean a layer that directly adjoins the cathode. An electron transport layer is understood here to mean a layer which is between cathode and emitting layer but does not directly adjoin the cathode, and preferably does not directly adjoin the emitting layer either. A hole blocker layer is understood here to mean a layer which is between cathode and emitting layer and directly adjoins the emitting layer.

A hole blocker layer preferably includes, for energy purposes, an organic material having a lower-lying HOMO than the first organic matrix material and/or the second organic matrix material. Thus, the holes (the injection and transport of which generally constitutes the smaller problem) are stopped by the electron transport layer from migrating directly to the cathode (for that reason, the electron transport layer is frequently also referred to as hole blocker layer).

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:

—anode—

—hole injection layer—

—hole transport layer—

—optionally further hole transport layers—

—optionally electron blocker layer—

—emitting layer—

—optionally hole blocker layer—

—electron transport layer—

—electron injection layer—

—cathode—.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of colour-emitting emitter compounds, an emitter compound used individually which emits over a broad wavelength range may also be suitable.

It is preferable that the compound of the formula (I) is used as electron transport material (ETM). The emitting layer here may be a fluorescent emitting layer, or it may be a phosphorescent emitting layer. The emitting layer is preferably a blue-fluorescing layer or a green-phosphorescing layer.

When the device containing the compound of the formula (I) contains a phosphorescent emitting layer, it is preferable that this layer contains two or more, preferably exactly two, different matrix materials (mixed matrix system). Preferred embodiments of mixed matrix systems are described in detail further down.

If the compound of formula (I) is used as electron transport material in an electron transport layer, an electron injection layer or a hole blocker layer, the compound can be used as pure material, i.e. in a proportion of 100%, in the electron transport, electron injection or hole blocker layer, or it can be used in combination with one or more further compounds, preferably organic compounds.

In a preferred embodiment, an electron-transporting layer comprising the compound of the formula (I) additionally comprises one or more further electron-transporting compounds. These further electron-transporting compounds are preferably selected from the preferred embodiments of electron transport materials that are specified further down. Most preferably, an electron-transporting layer comprises the compound of the formula (I) together with LiQ (lithium 8-quinolinate) or an LiQ derivative as further electron-transporting compound. In the preferred embodiment described, the compound of the formula (I) and the one or more further electron-transporting compounds are preferably each present in a proportion of at least 10%, more preferably each in a proportion of at least 20%.

In another preferred embodiment, an electron transport layer comprises the compound of the formula (I) as pure material, and an electron injection layer comprises one or more further electron-transporting compounds, more preferably LiQ or an LiQ derivative.

In a preferred embodiment, a hole-transporting layer comprising the compound of the formula (I) additionally contains one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, I₂, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site. Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (III) oxidation state, more particularly bismuth(III) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-dopants are especially the following compounds:

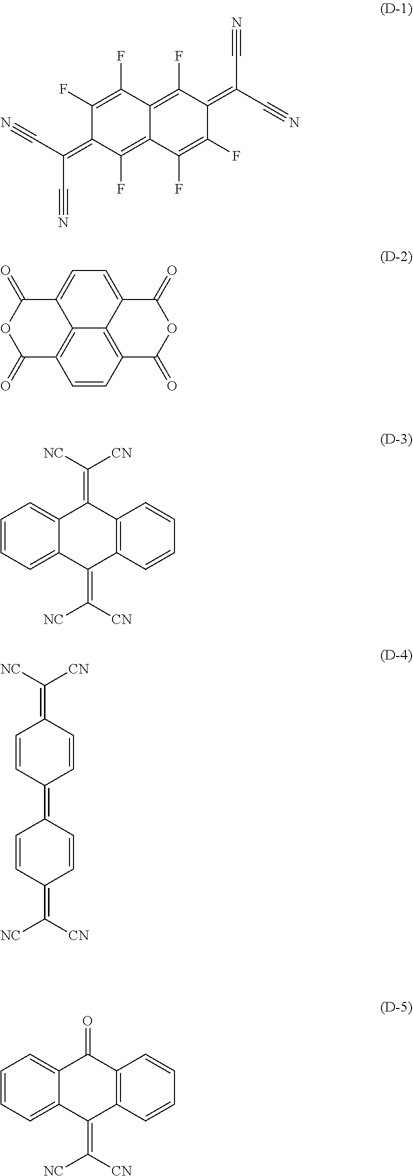 [SMILES: N#CC(C#N)=c1c(F)c(F)c2c(F)c(=C(C#N)C#N)c(F)c(F)c2c1F ; O=C1OC(=O)c2ccc3c4c(ccc1c24)C(=O)OC3=O ; N#CC(C#N)=c1c2ccccc2c(=C(C#N)C#N)c2ccccc12 ; N#CC(C#N)=c1ccc(=c2ccc(=C(C#N)C#N)cc2)cc1 ; N#CC(C#N)=C1c2ccccc2C(=O)c2ccccc21]

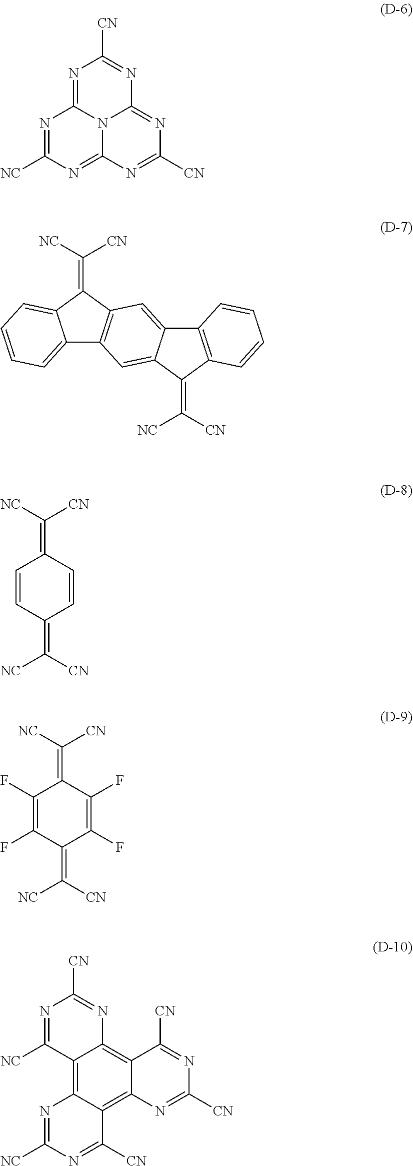 [SMILES: N#CC1=NC2=NC(C#N)=NC3=NC(C#N)=NC(=N1)N23 ; N#CC(C#N)=C1c2ccccc2-c2cc3c(cc21)-c1ccccc1C3=C(C#N)C#N ; N#CC(C#N)=c1ccc(=C(C#N)C#N)cc1 ; N#CC(C#N)=c1c(F)c(F)c(=C(C#N)C#N)c(F)c1F ; N#Cc1nc(C#N)c2c(n1)c1c(C#N)nc(C#N)nc1c1c(C#N)nc(C#N)nc21]

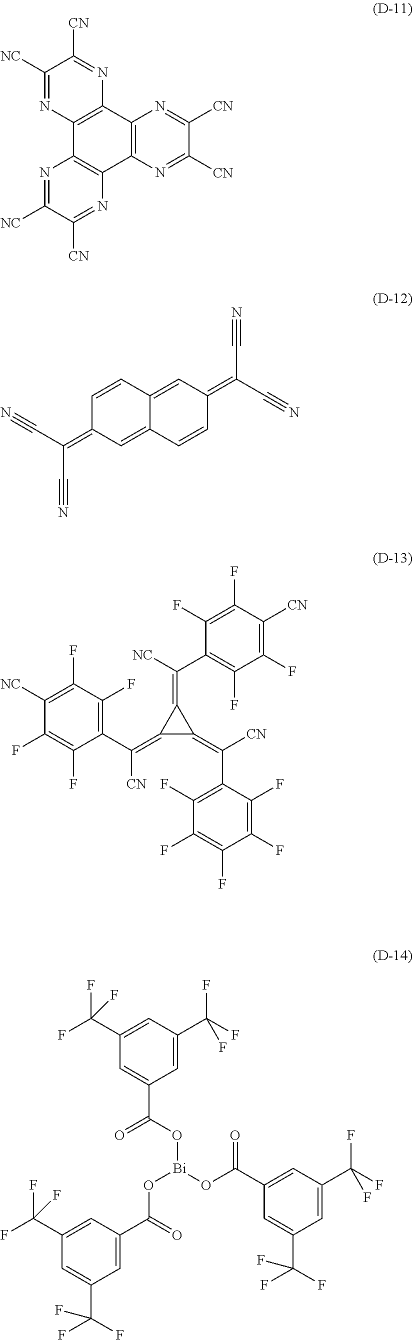

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (I) may be present in an electron injection layer, in an electron transport layer and/or in a hole blocker layer of the device. When the compound is present in an electron injection layer or in an electron transport layer, it has preferably been p-doped, meaning that it is in mixed form with a p-dopant, as described above, in the layer.

The compound of the formula (I) is preferably present in an electron transport layer and/or in a hole blocker layer. Most preferably, the compound of the formula (I) is present in an electron transport layer. In this case, it is preferably not p-doped. Further preferably, in this case, it is not present as a single compound in the electron transport layer, but is present in combination with one or more further compounds, preferably with further electron transport and/or electron injection materials, and most preferably with LiQ (lithium 8-quinolinolate) or LiQ derivatives.

In an alternative preferred embodiment, the compound of the formula (I) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) are used as a component of mixed matrix systems, preferably for phosphorescent emitters. The mixed matrix systems preferably comprise two or three different matrix materials, more preferably two different matrix materials. Preferably, in this case, one of the two materials is a material having electron-transporting properties and the other material is a material having hole-transporting properties. It is further preferable when one of the materials is selected from compounds having a large energy differential between HOMO and LUMO (wide-bandgap materials). The compound of the formula (I) in a mixed matrix system is preferably the matrix material having electron-transporting properties. Correspondingly, when the compound of the formula (I) is used as matrix material for a phosphorescent emitter in the emitting layer of an OLED, a second matrix compound having hole-transporting properties is present in the emitting layer. It is especially preferable here when the compound of the formula (I) as electron-transporting material is present in the EML as a mixture together with carbazole derivatives, especially biscarbazoles, as hole-transporting material. The two different matrix materials may be present here in a ratio of 1:50 to 1:1, preferably 1:20 to 1:1, more preferably 1:10 to 1:1 and most preferably 1:4 to 1:1.

Preferred biscarbazoles as matrix material are those of the following formula:

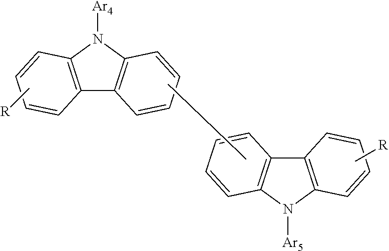

Very preferred biscarbazoles are those of the following formula:

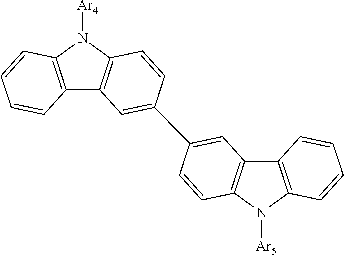

where the R group has the same definition as the R⁰ group defined above and/or in claim 1; Ar₄ and Ar₅ have the same definition as the Ar⁰ group defined above and/or in claim 1.

Particularly preferred biscarbazoles are those of the following formulae:

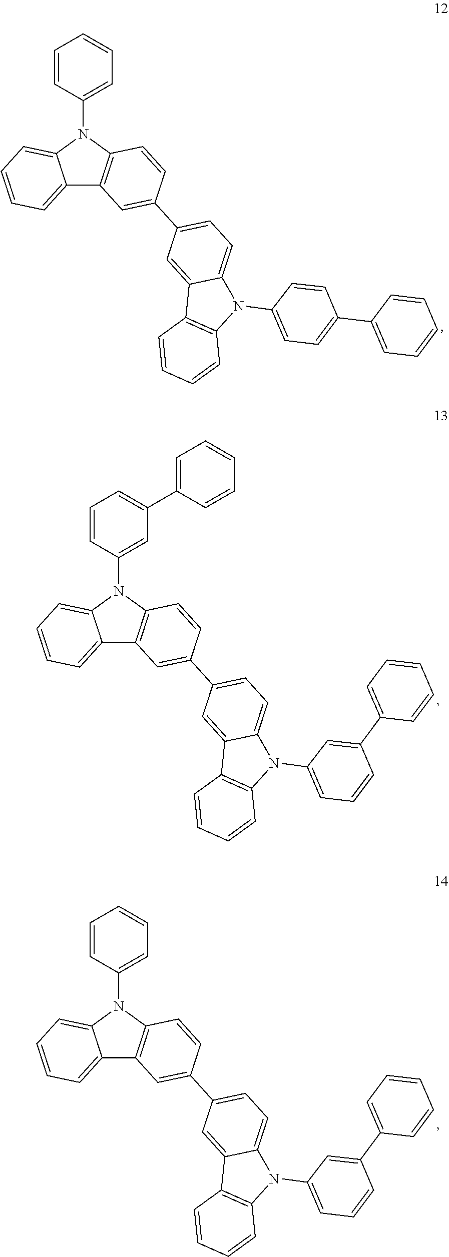 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1]

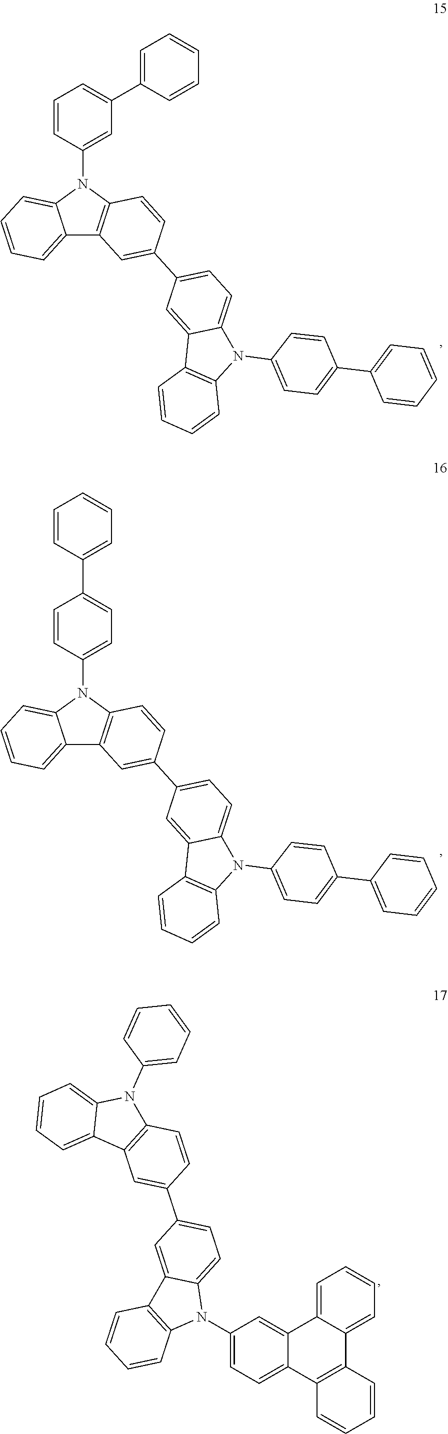 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc5c6ccccc6c6ccccc6c5c4)ccc32)cc1]

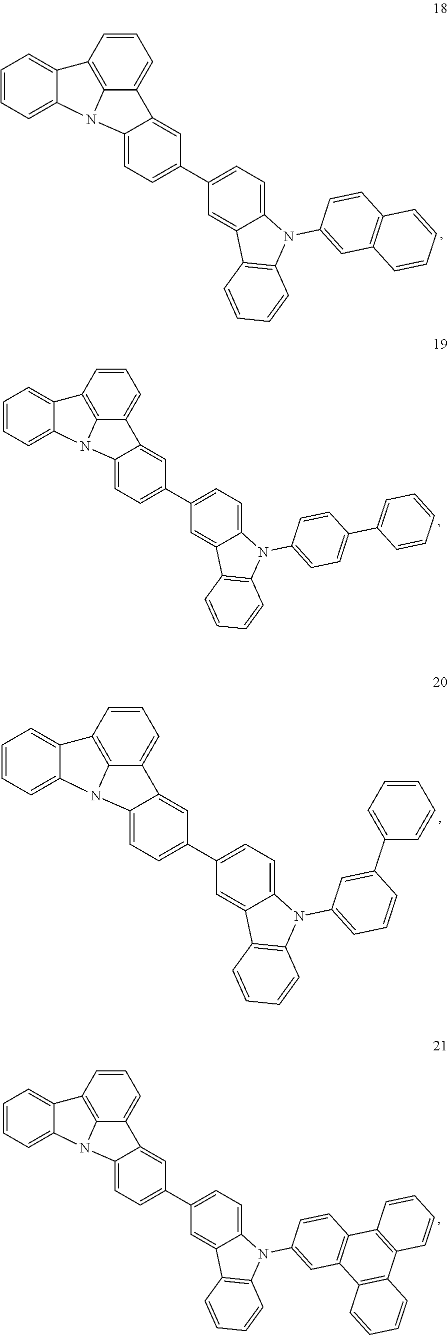 [SMILES: c1ccc2cc(-n3c4ccccc4c4cc(-c5ccc6c(c5)c5cccc7c8ccccc8n6c75)ccc43)ccc2c1 ; c1ccc(-c2ccc(-n3c4ccccc4c4cc(-c5ccc6c(c5)c5cccc7c8ccccc8n6c75)ccc43)cc2)cc1 ; c1ccc(-c2cccc(-n3c4ccccc4c4cc(-c5ccc6c(c5)c5cccc7c8ccccc8n6c75)ccc43)c2)cc1 ; c1ccc2c(c1)c1ccccc1c1cc(-n3c4ccccc4c4cc(-c5ccc6c(c5)c5cccc7c8ccccc8n6c75)ccc43)ccc21]

The desired electron-transporting and hole-transporting properties of the mixed matrix components may, however, also be combined mainly or entirely in a single mixed matrix component, in which case the further mixed matrix component(s) fulfil(s) other functions.

Preference is given to using the following material classes in the abovementioned layers of the device:

Phosphorescent Emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are shown in the following table:

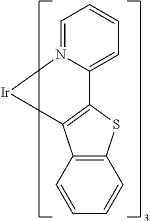

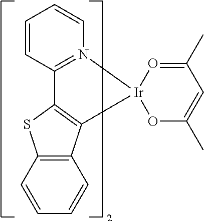

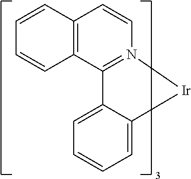

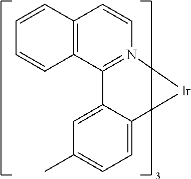

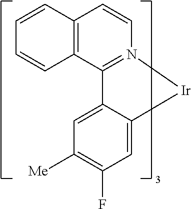

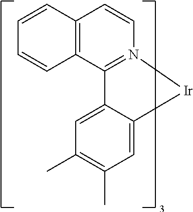

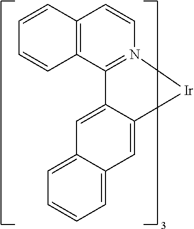

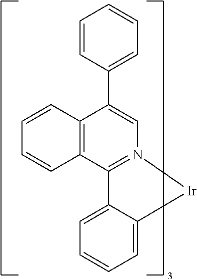

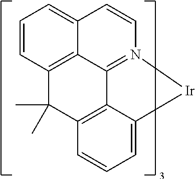

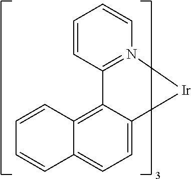

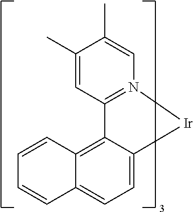

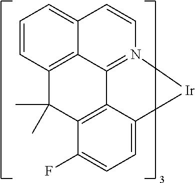

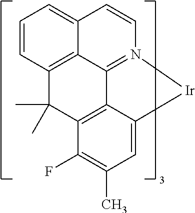

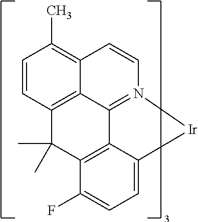

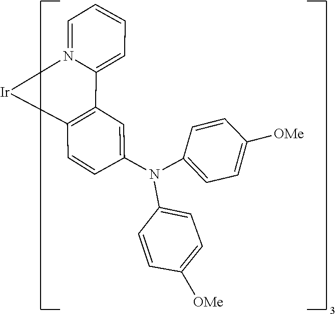

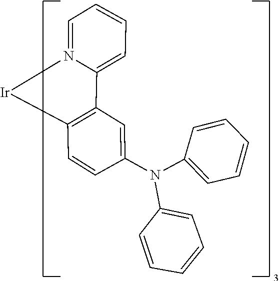

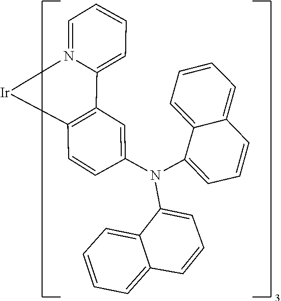

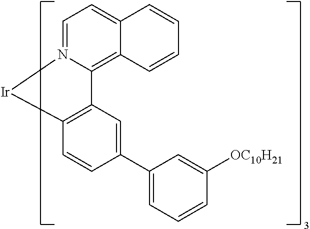

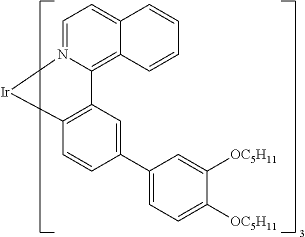

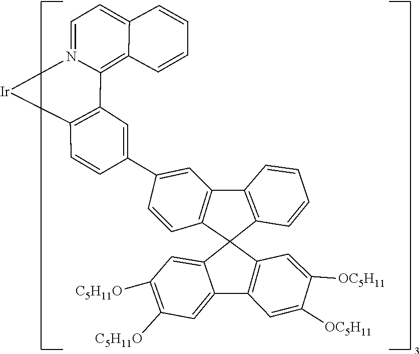

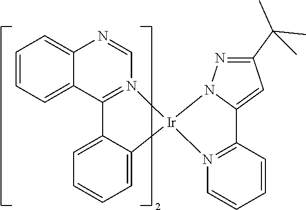

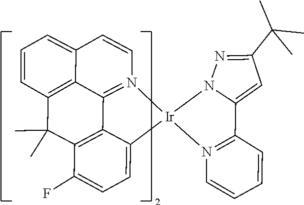

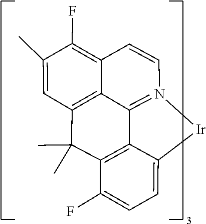

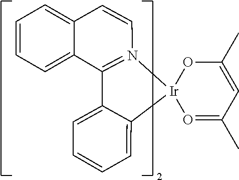

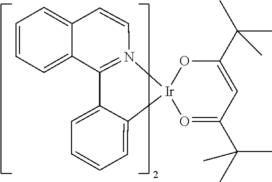

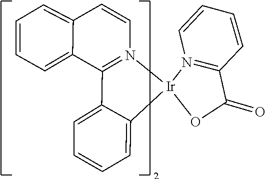

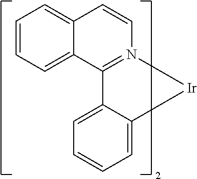

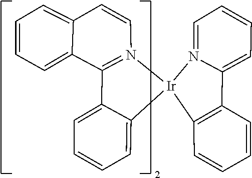

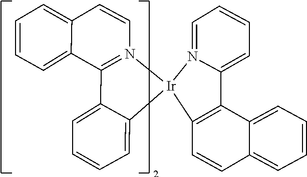

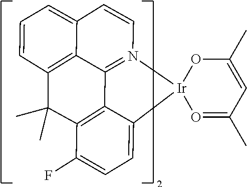

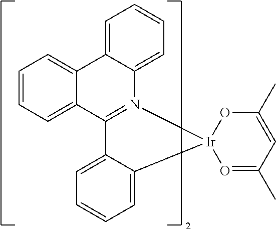

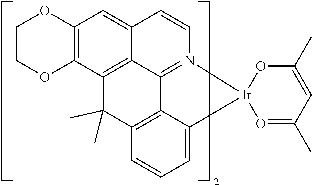

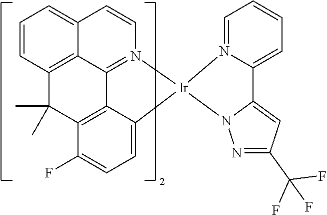

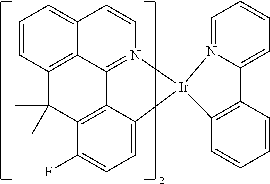

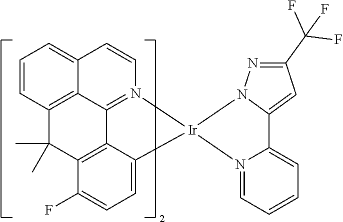

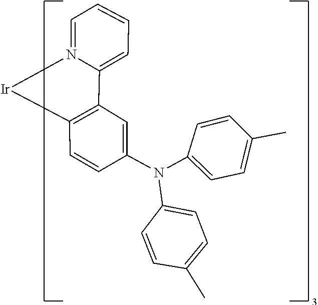

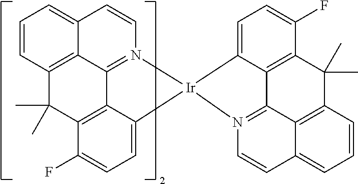

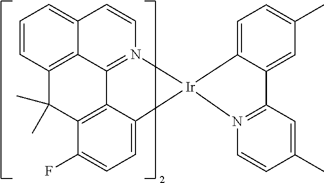

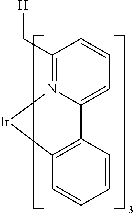

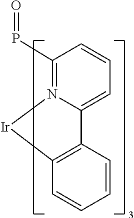

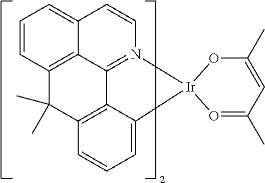

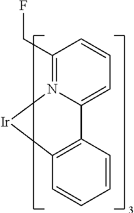

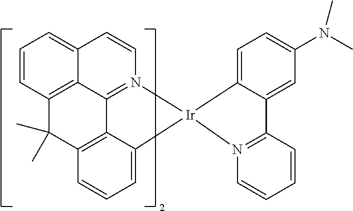

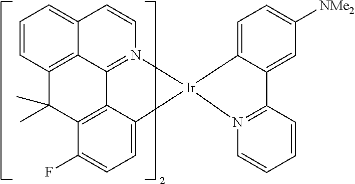

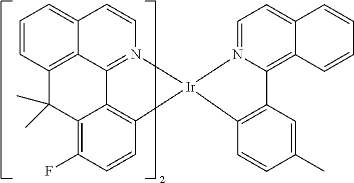

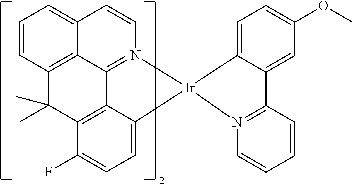

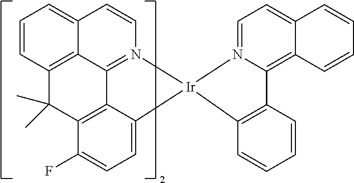

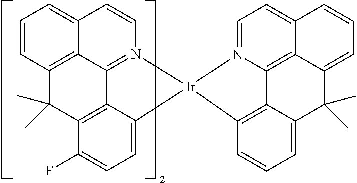

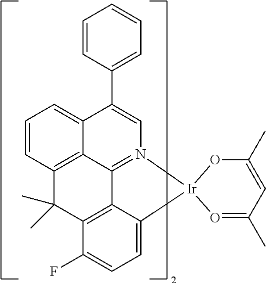

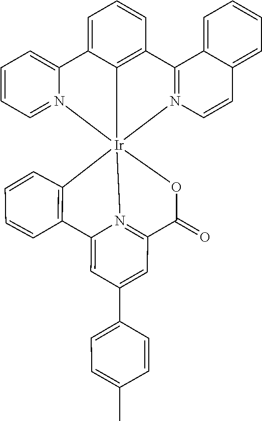

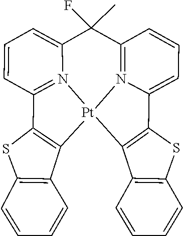

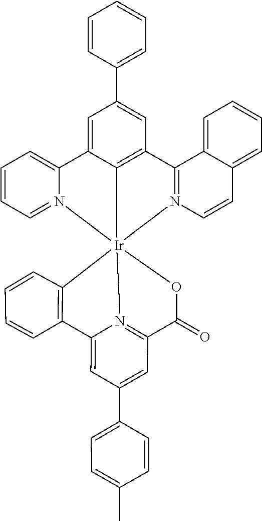

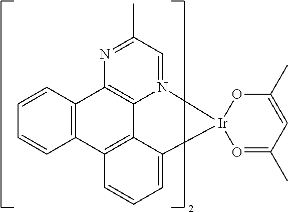

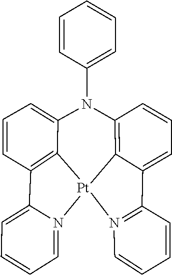

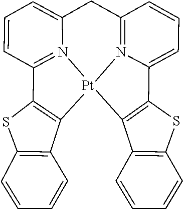

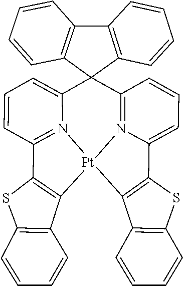

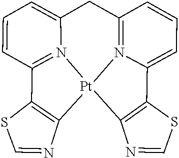

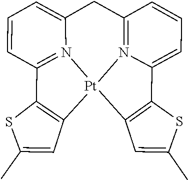

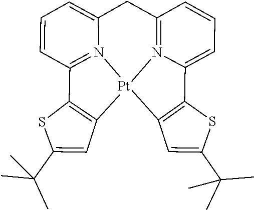

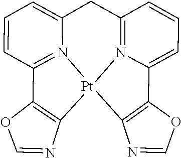

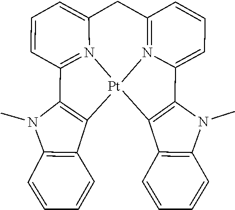

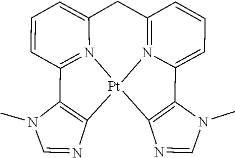

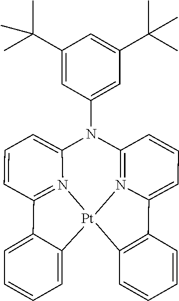

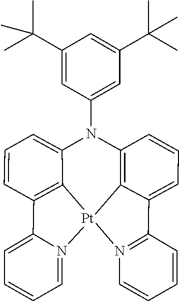

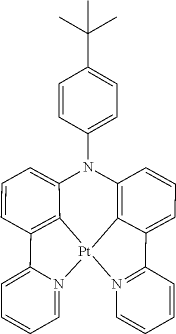

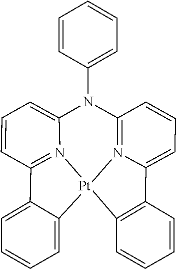

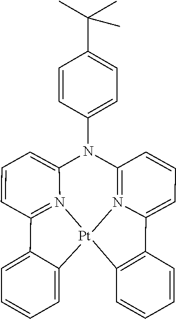

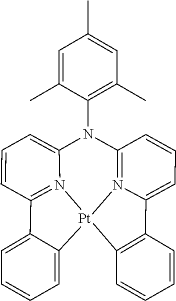

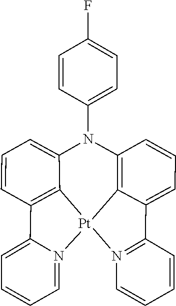

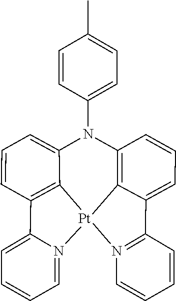

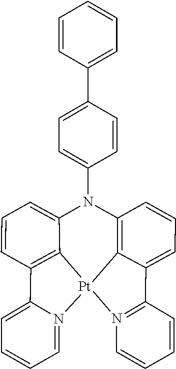

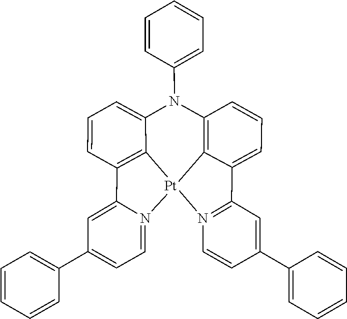

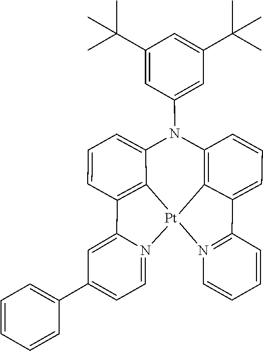

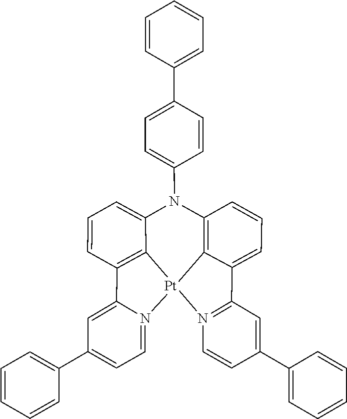

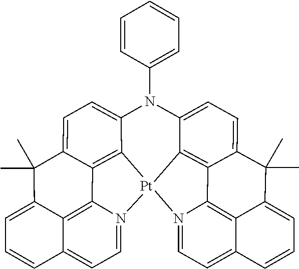

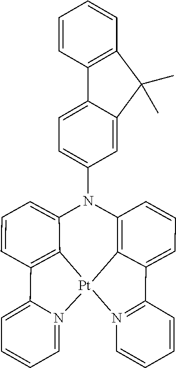

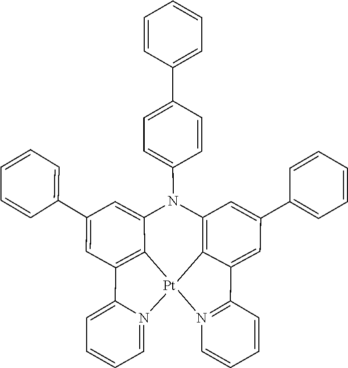

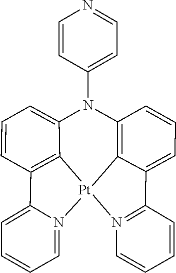

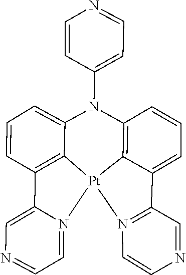

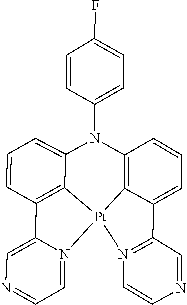

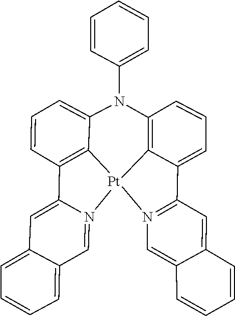

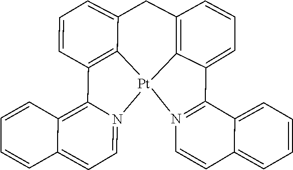

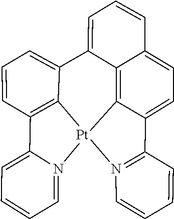

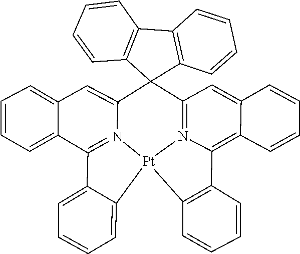

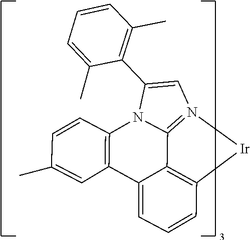

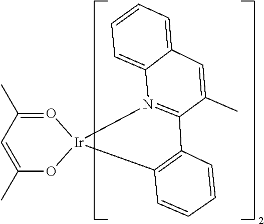

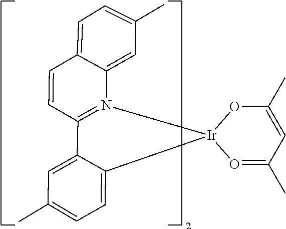

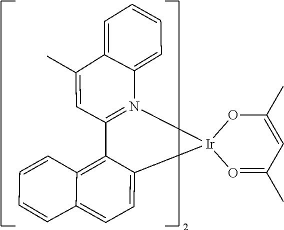

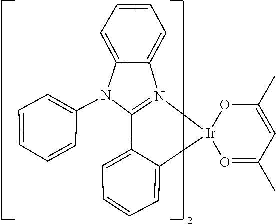

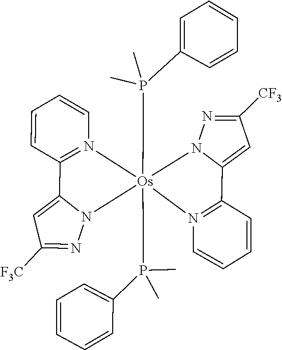

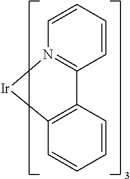

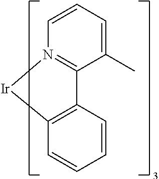

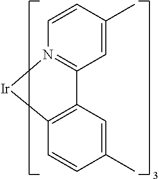

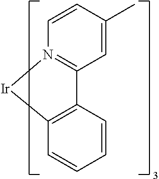

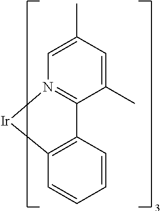

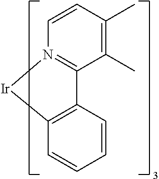

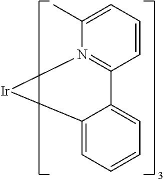

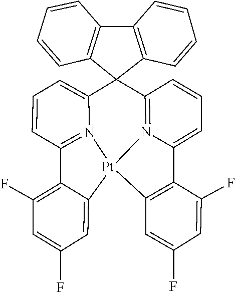

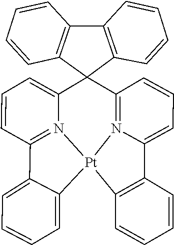

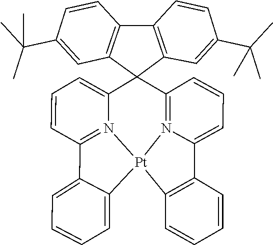

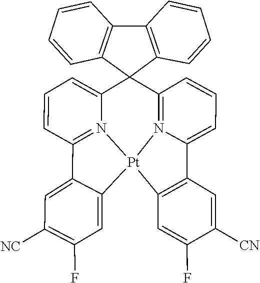

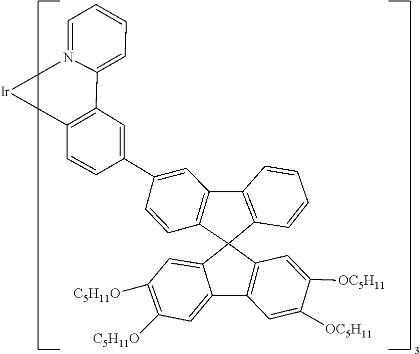

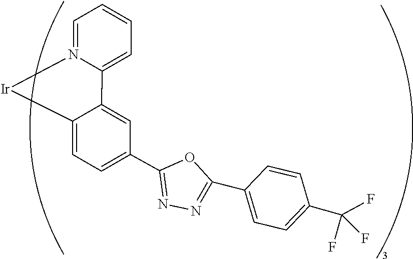

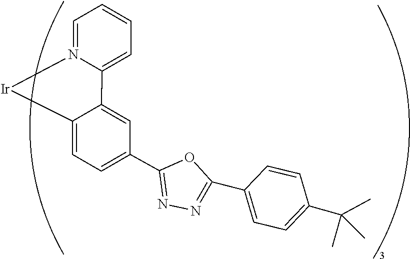

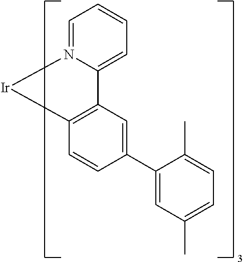

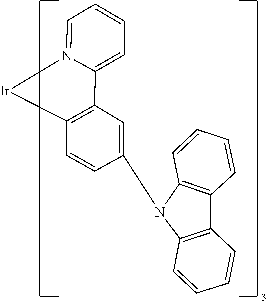

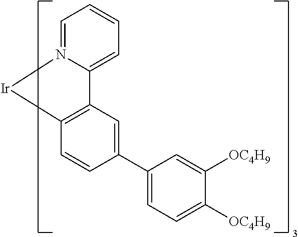

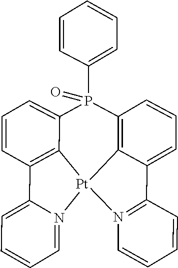

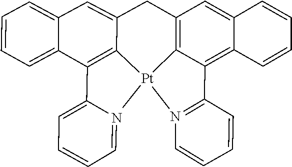

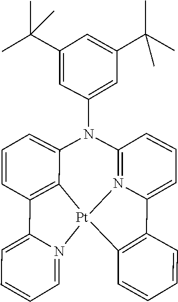

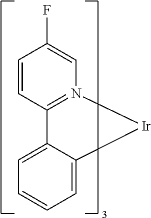

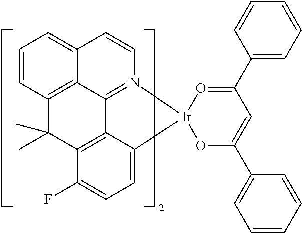

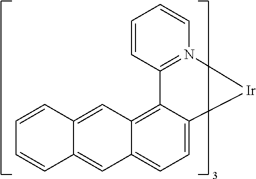

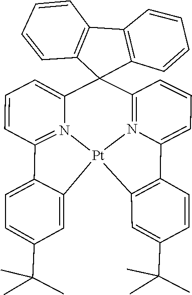

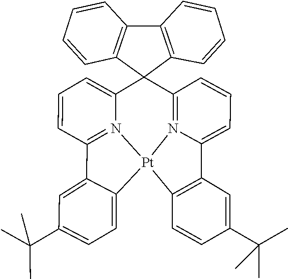

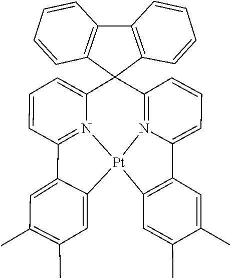

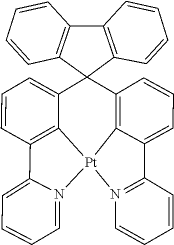

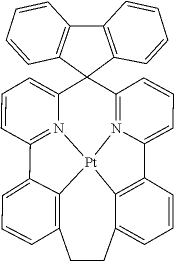

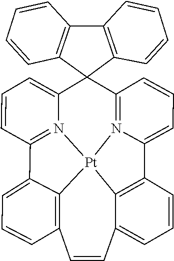

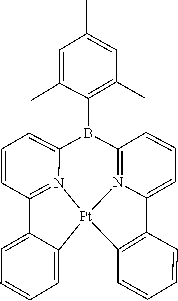

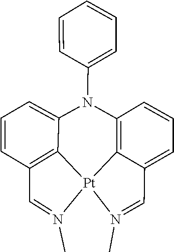

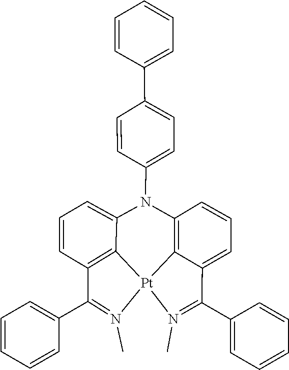

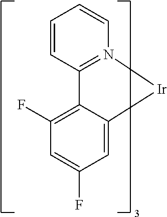

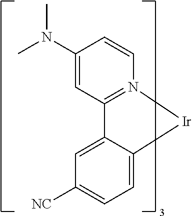

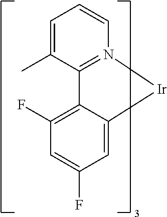

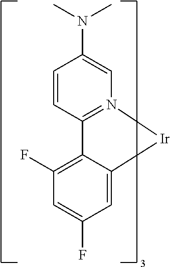

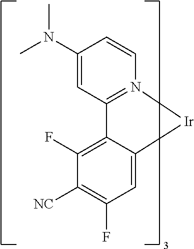

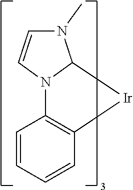

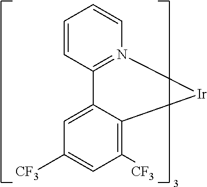

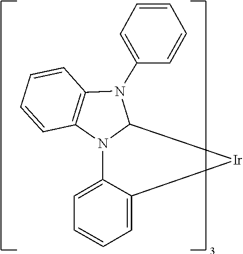

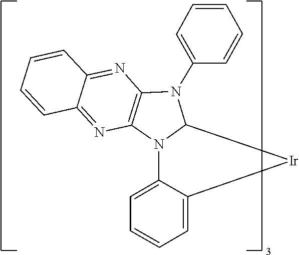

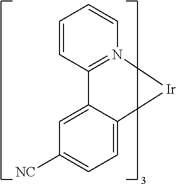

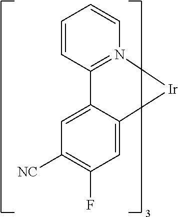

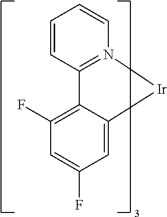

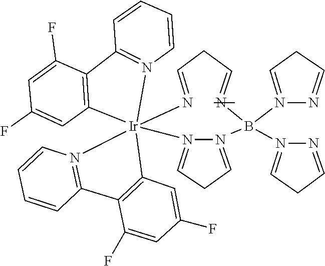

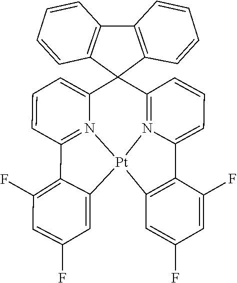

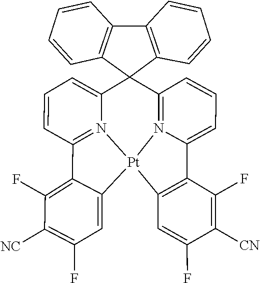

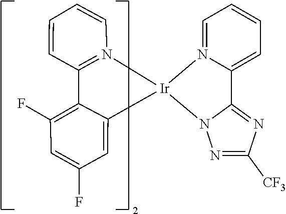

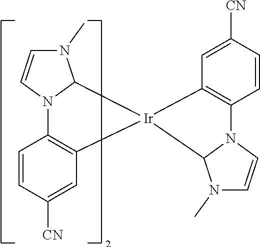

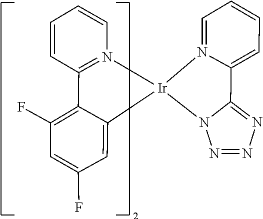

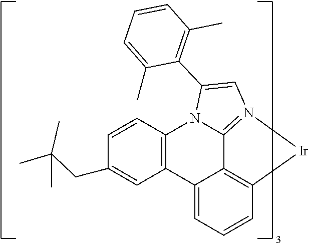

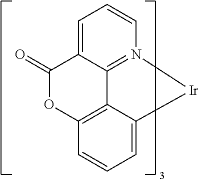

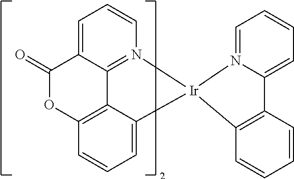

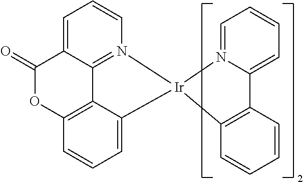

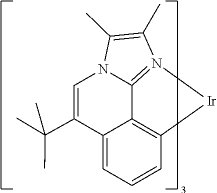

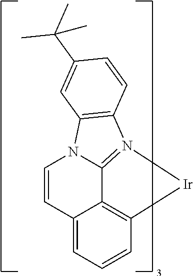

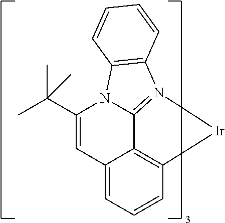

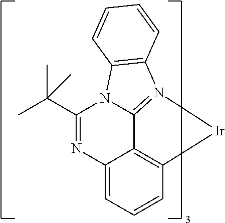

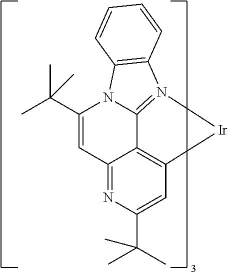

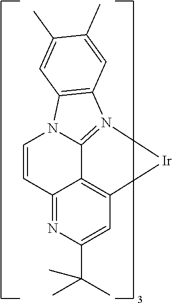

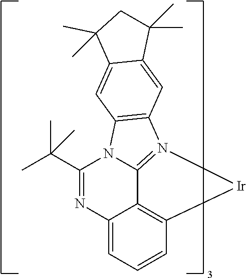

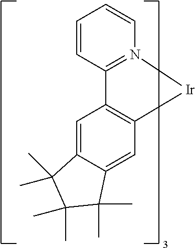

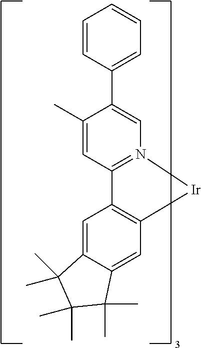

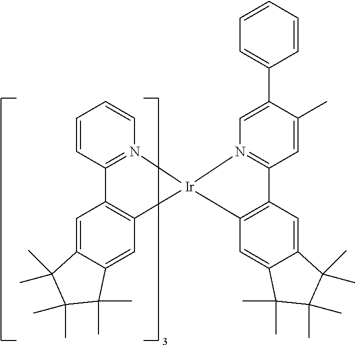

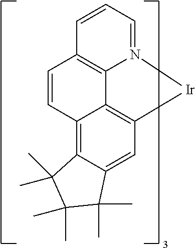

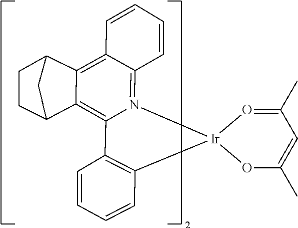

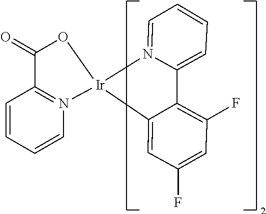

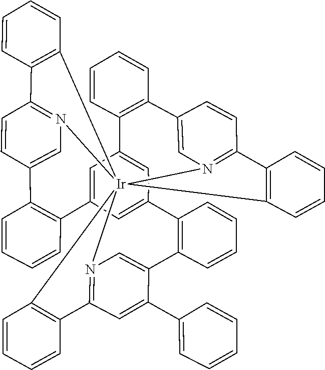

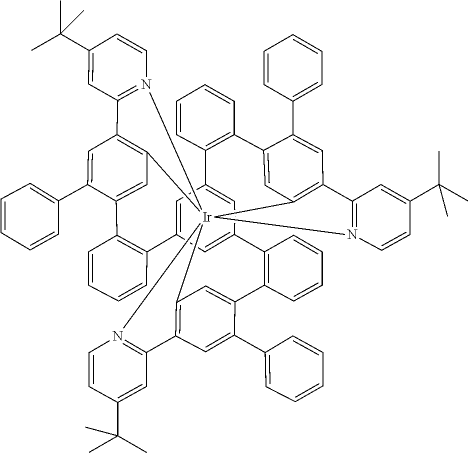

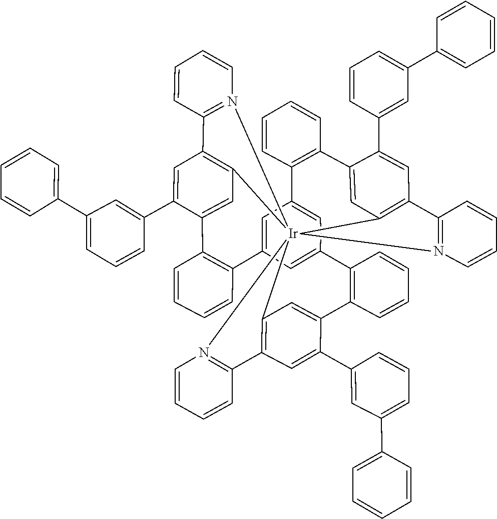

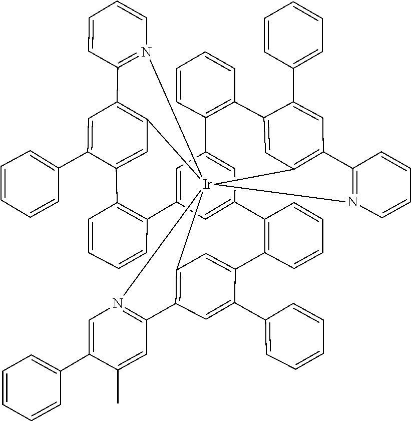

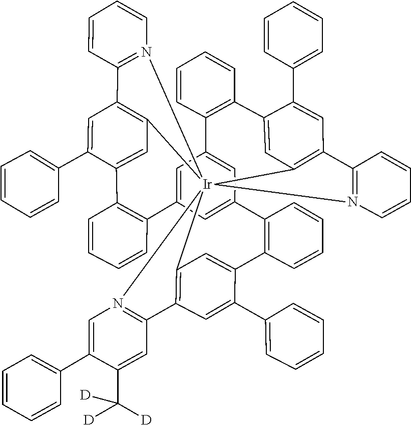

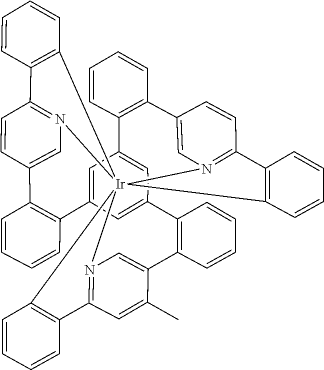

Fluorescent Emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 position. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

Delayed Fluorescent Materials:

Suitable delayed fluorescent materials, such as delayed fluorescent emitters or delayed fluorescent hosts, are well known in the prior art, for example in Ye Tao et al., Adv. Mater. 2014, 26, 7931-7958, M. Y. Wong et al., Adv. Mater. 2017, 29, 1605444, WO 2011/070963, WO 2012/133188, WO 2015/022974 and WO 2015/098975. Typically, delayed fluorescent materials are characterized in that they have quite a small energy gap between the singlet energy level (Si) and the triplet energy level (Ti). Preferably, ΔE_{ST} is less than 0.5 eV, more preferably less than 0.3 eV and most preferably less than 0.2 eV, where ΔE_{ST} represents the energy differential between the singlet energy level (S₁) and the triplet energy level (T₁).

Matrix Materials for Fluorescent Emitters:

Preferred matrix materials for fluorescent emitters are selected from the classes of the oligoarylenes (e.g. 2,2′,7,7-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

Matrix Materials for Phosphorescent Emitters:

Preferred matrix materials for phosphorescent emitters are, as well as the compounds of the formula (I), aromatic ketones, aromatic phosphine oxides or aromatic sulfoxides or sulfones, triarylamines, carbazole derivatives, e.g. CBP (N,N-biscarbazolylbiphenyl), indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

Electron-Transporting Materials:

Further compounds that are used with preference alongside the compounds of the formula (I) in electron-transporting layers of the OLEDs of the invention are, for example, the compounds disclosed in Y. Shirota et al., Chem. Rev. 2007, 107(4), 953-1010, or other materials as used in these layers according to the prior art.

Further compounds that are used alongside the compounds of the formula (I) in the electron transport layer are any materials that can be used as electron transport materials in the electron transport layer according to the prior art. Especially suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Preferred electron-transporting compounds are shown in the following table:

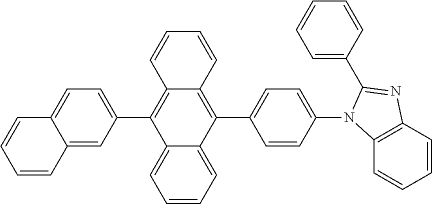 [SMILES: c1ccc(-c2nc3ccccc3n2-c2ccc(-c3c4ccccc4c(-c4ccc5ccccc5c4)c4ccccc34)cc2)cc1]

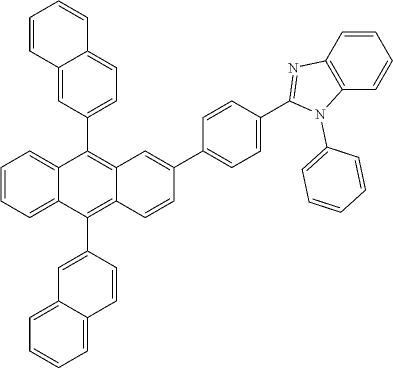 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

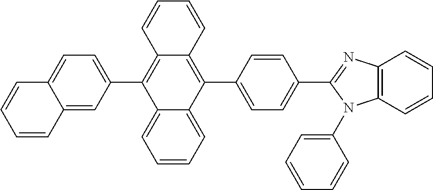 [SMILES: c1ccc(-n2c(-c3ccc(-c4c5ccccc5c(-c5ccc6ccccc6c5)c5ccccc45)cc3)nc3ccccc32)cc1]

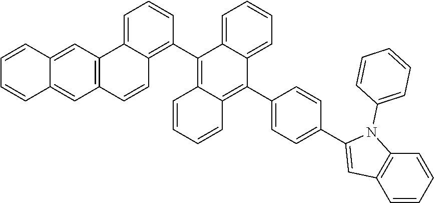 [SMILES: c1ccc(-n2c(-c3ccc(-c4c5ccccc5c(-c5cccc6c5ccc5cc7ccccc7cc56)c5ccccc45)cc3)cc3ccccc32)cc1]

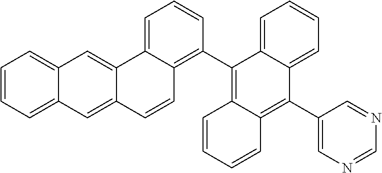 [SMILES: c1ccc2cc3c(ccc4c(-c5c6ccccc6c(-c6cncnc6)c6ccccc56)cccc43)cc2c1]

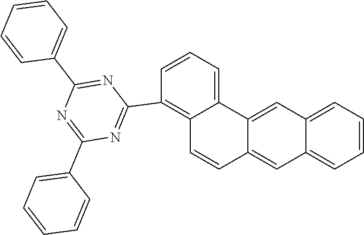 [SMILES: c1ccc(-c2nc(-c3ccccc3)nc(-c3cccc4c3ccc3cc5ccccc5cc34)n2)cc1]

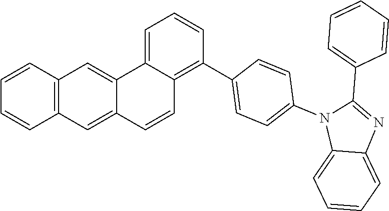 [SMILES: c1ccc(-c2nc3ccccc3n2-c2ccc(-c3cccc4c3ccc3cc5ccccc5cc34)cc2)cc1]

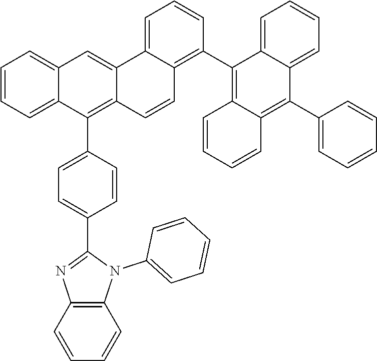 [SMILES: c1ccc(-c2c3ccccc3c(-c3cccc4c3ccc3c(-c5ccc(-c6nc7ccccc7n6-c6ccccc6)cc5)c5ccccc5cc34)c3ccccc23)cc1]

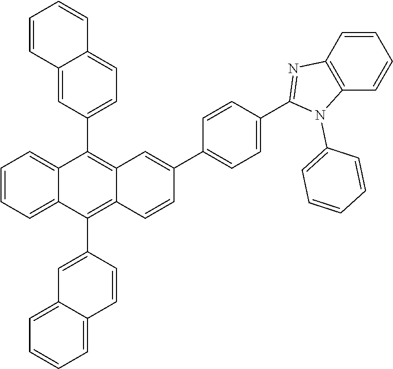 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

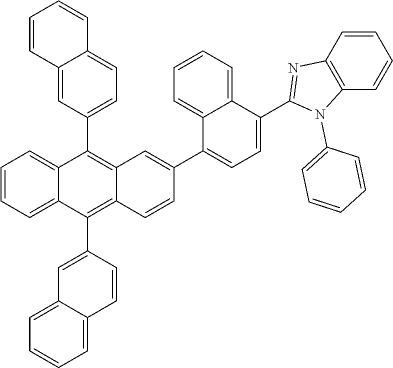 [SMILES: c1ccc(-n2c(-c3ccc(-c4ccc5c(-c6ccc7ccccc7c6)c6ccccc6c(-c6ccc7ccccc7c6)c5c4)c4ccccc34)nc3ccccc32)cc1]

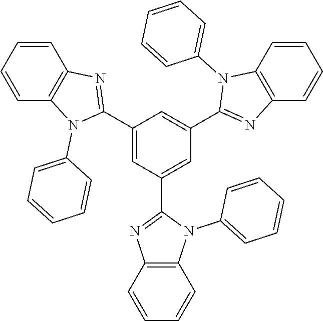 [SMILES: c1ccc(-n2c(-c3cc(-c4nc5ccccc5n4-c4ccccc4)cc(-c4nc5ccccc5n4-c4ccccc4)c3)nc3ccccc32)cc1]

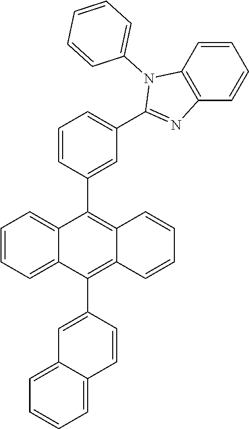 [SMILES: c1ccc(-n2c(-c3cccc(-c4c5ccccc5c(-c5ccc6ccccc6c5)c5ccccc45)c3)nc3ccccc32)cc1]

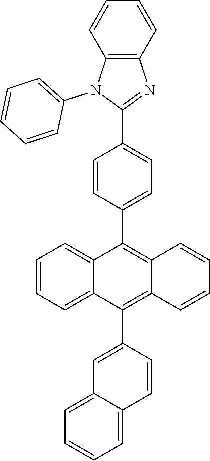 [SMILES: c1ccc(-n2c(-c3ccc(-c4c5ccccc5c(-c5ccc6ccccc6c5)c5ccccc45)cc3)nc3ccccc32)cc1]

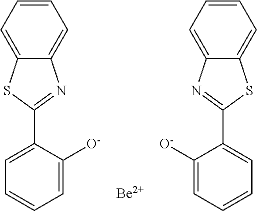

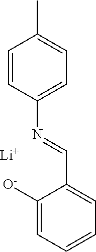 [SMILES: [Li+] ; Cc1ccc(/N=C/c2ccccc2[O-])cc1]

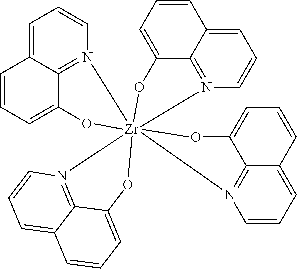

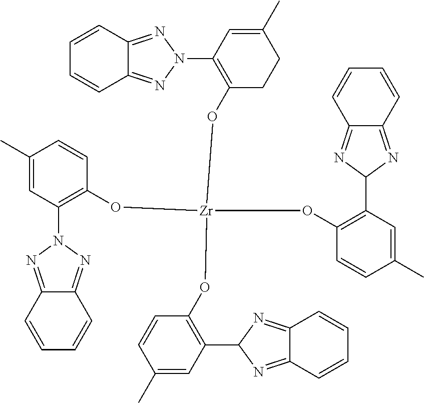

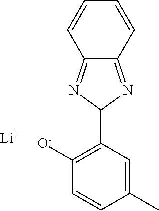 [SMILES: [Li+] ; Cc1ccc([O-])c(C2N=c3ccccc3=N2)c1]

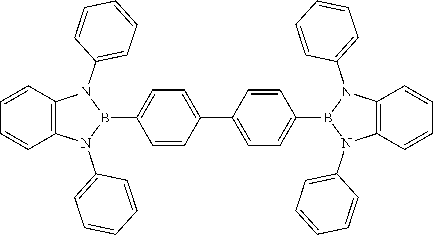 [SMILES: c1ccc(N2B(c3ccc(-c4ccc(B5N(c6ccccc6)c6ccccc6N5c5ccccc5)cc4)cc3)N(c3ccccc3)c3ccccc32)cc1]

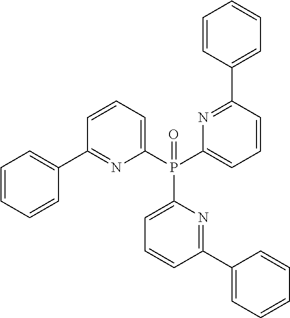 [SMILES: O=P(c1cccc(-c2ccccc2)n1)(c1cccc(-c2ccccc2)n1)c1cccc(-c2ccccc2)n1]

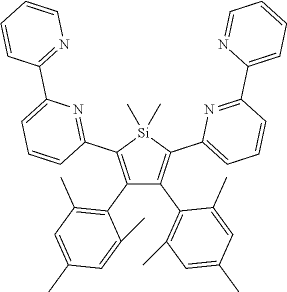 [SMILES: Cc1cc(C)c(C2=C(c3cccc(-c4ccccn4)n3)[Si](C)(C)C(c3cccc(-c4ccccn4)n3)=C2c2c(C)cc(C)cc2C)c(C)c1]

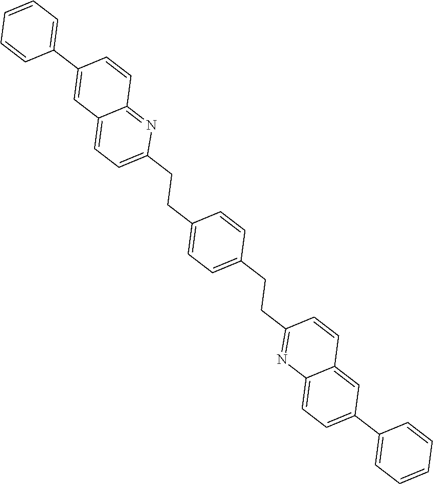 [SMILES: c1ccc(-c2ccc3nc(CCc4ccc(CCc5ccc6cc(-c7ccccc7)ccc6n5)cc4)ccc3c2)cc1]

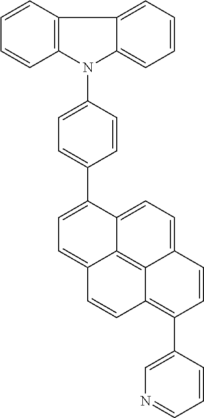 [SMILES: c1cncc(-c2ccc3ccc4c(-c5ccc(-n6c7ccccc7c7ccccc76)cc5)ccc5ccc2c3c54)c1]

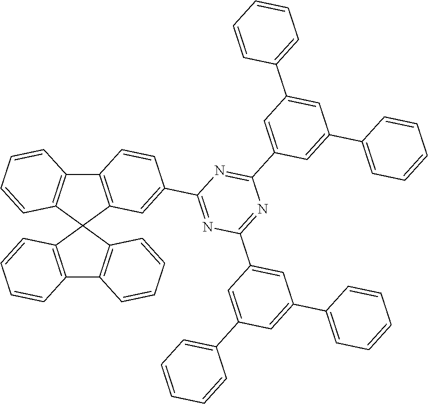 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

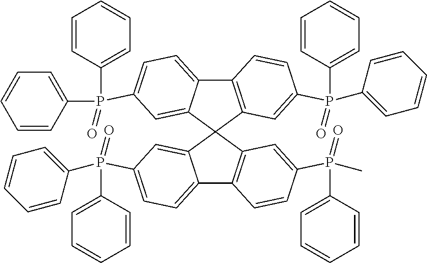 [SMILES: CP(=O)(c1ccccc1)c1ccc2c(c1)C1(c3cc(P(=O)(c4ccccc4)c4ccccc4)ccc3-2)c2cc(P(=O)(c3ccccc3)c3ccccc3)ccc2-c2ccc(P(=O)(c3ccccc3)c3ccccc3)cc21]

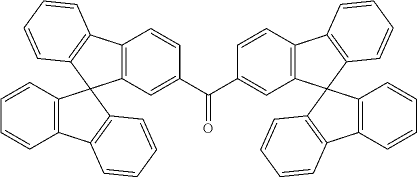 [SMILES: O=C(c1ccc2c(c1)C1(c3ccccc3-c3ccccc31)c1ccccc1-2)c1ccc2c(c1)C1(c3ccccc3-c3ccccc31)c1ccccc1-2]

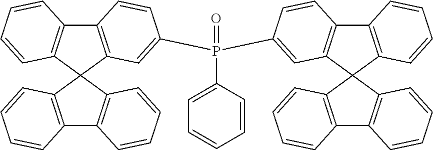 [SMILES: O=P(c1ccccc1)(c1ccc2c(c1)C1(c3ccccc3-c3ccccc31)c1ccccc1-2)c1ccc2c(c1)C1(c3ccccc3-c3ccccc31)c1ccccc1-2]

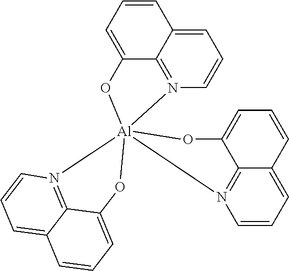

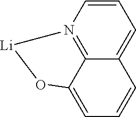

Hole-Transporting Materials:

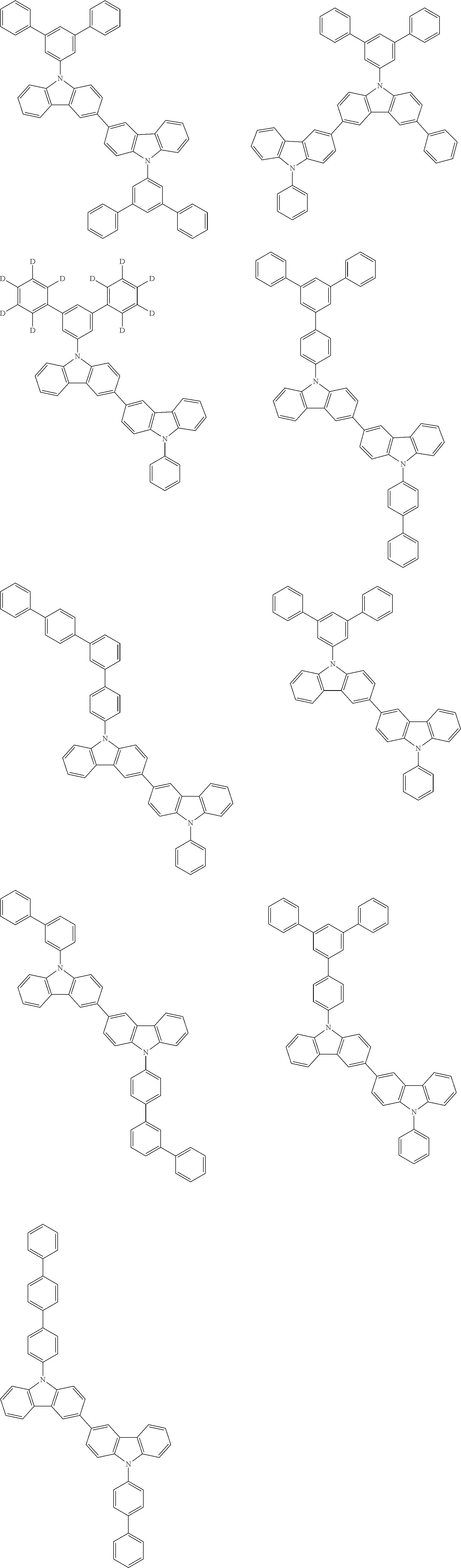

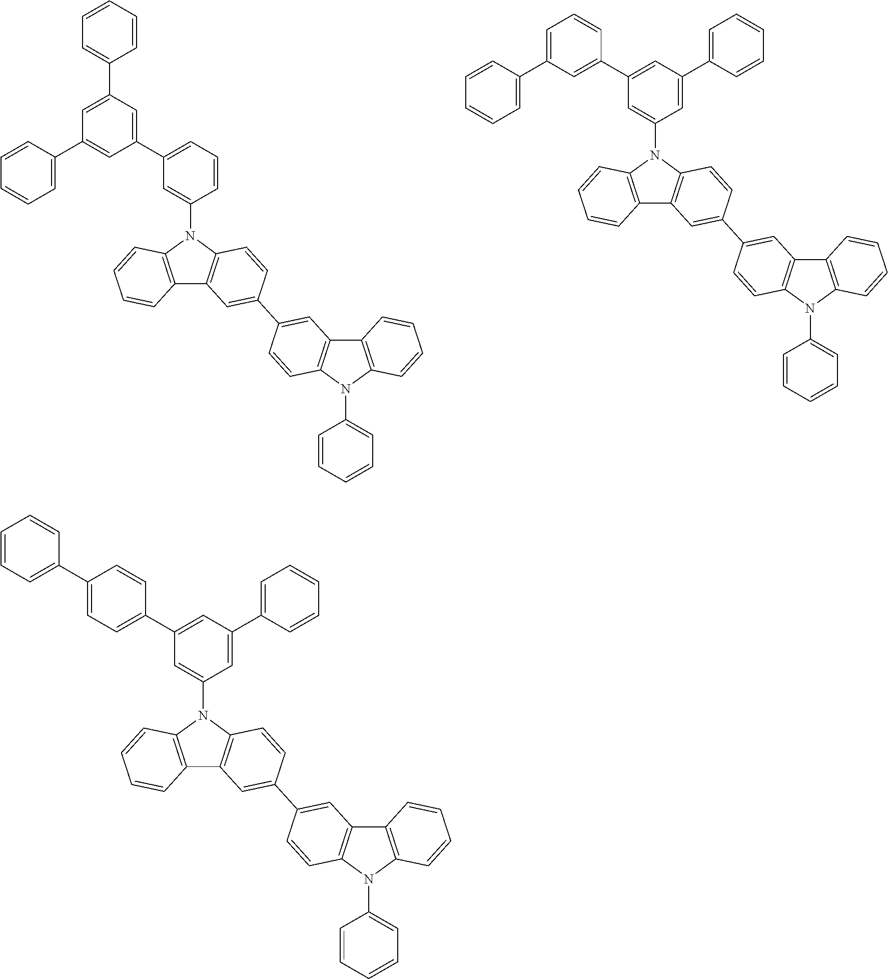 [SMILES: c1ccc(-c2cc(-c3ccccc3)cc(-c3cccc(-n4c5ccccc5c5cc(-c6ccc7c(c6)c6ccccc6n7-c6ccccc6)ccc54)c3)c2)cc1 ; c1ccc(-c2cccc(-c3cc(-c4ccccc4)cc(-n4c5ccccc5c5cc(-c6ccc7c(c6)c6ccccc6n7-c6ccccc6)ccc54)c3)c2)cc1 ; c1ccc(-c2ccc(-c3cc(-c4ccccc4)cc(-n4c5ccccc5c5cc(-c6ccc7c(c6)c6ccccc6n7-c6ccccc6)ccc54)c3)cc2)cc1]

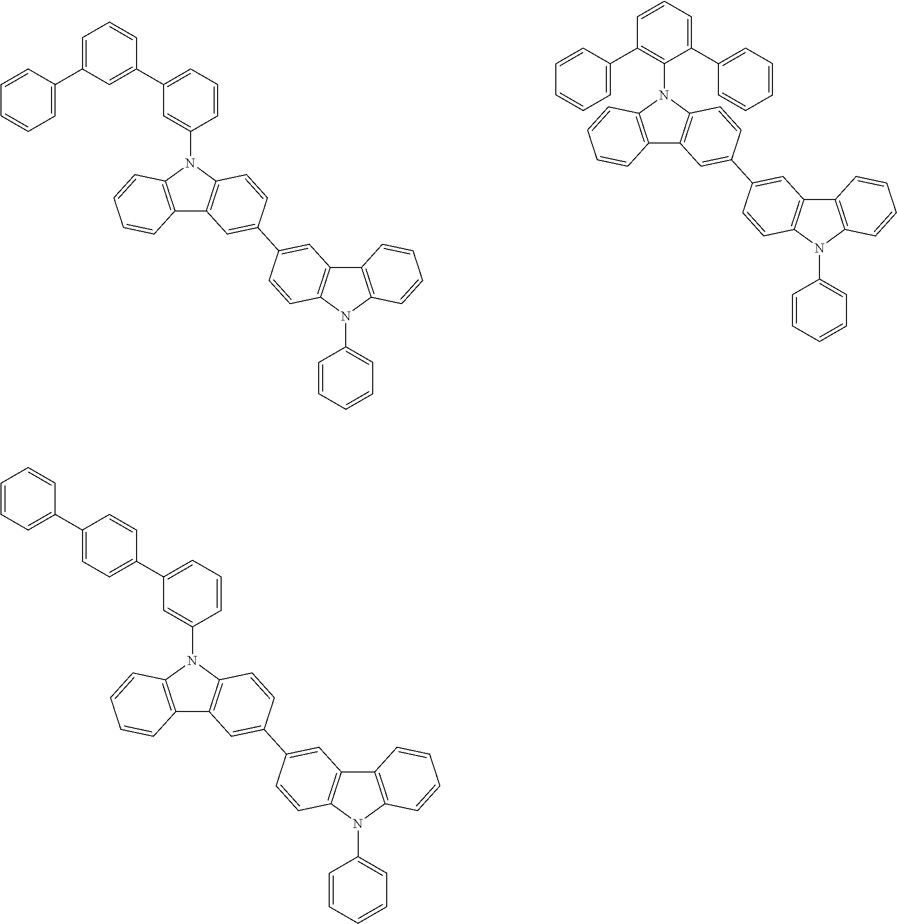 [SMILES: c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2cccc(-c3ccccc3)c2-n2c3ccccc3c3cc(-c4ccc5c(c4)c4ccccc4n5-c4ccccc4)ccc32)cc1 ; c1ccc(-c2ccc(-c3cccc(-n4c5ccccc5c5cc(-c6ccc7c(c6)c6ccccc6n7-c6ccccc6)ccc54)c3)cc2)cc1]

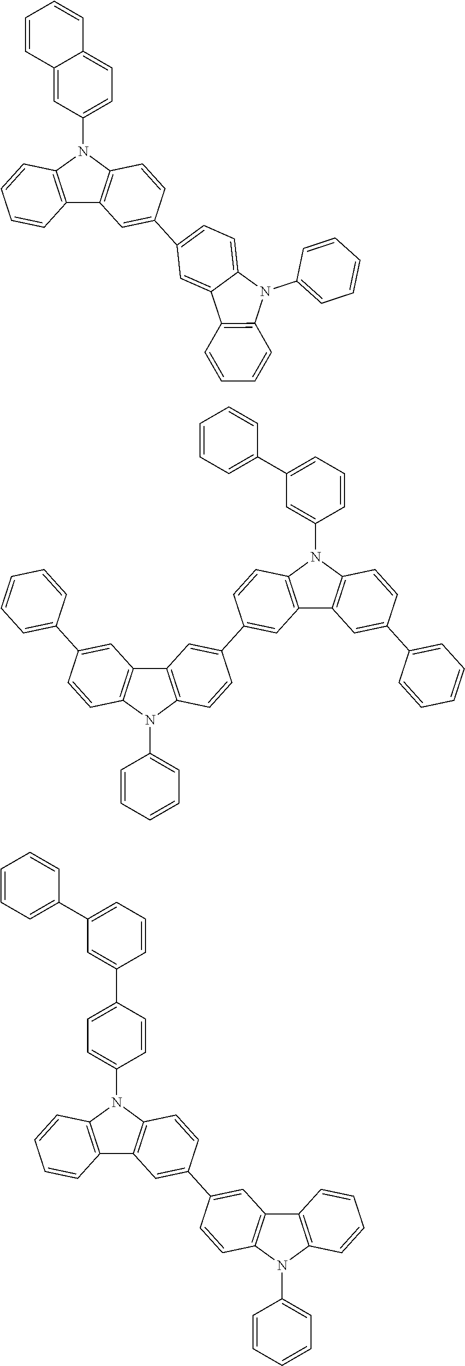

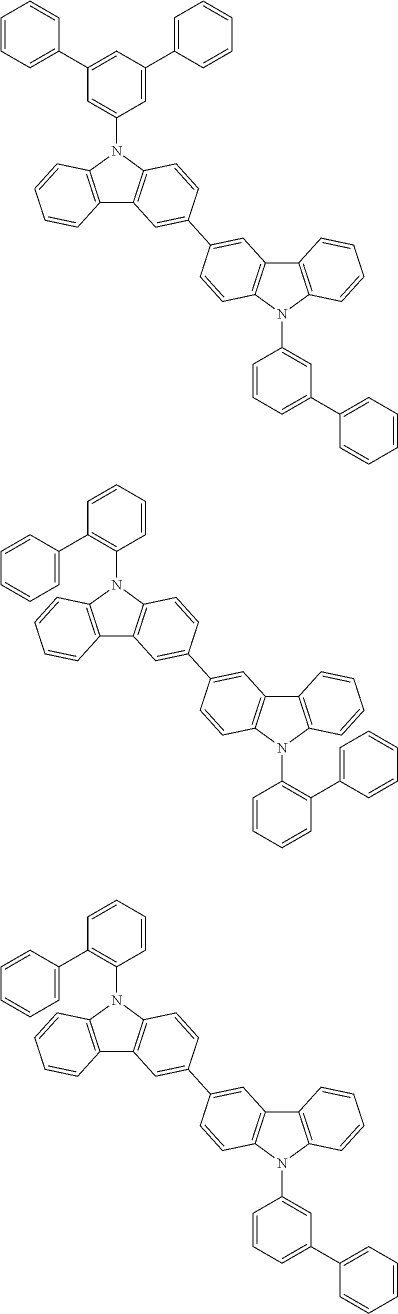 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1]

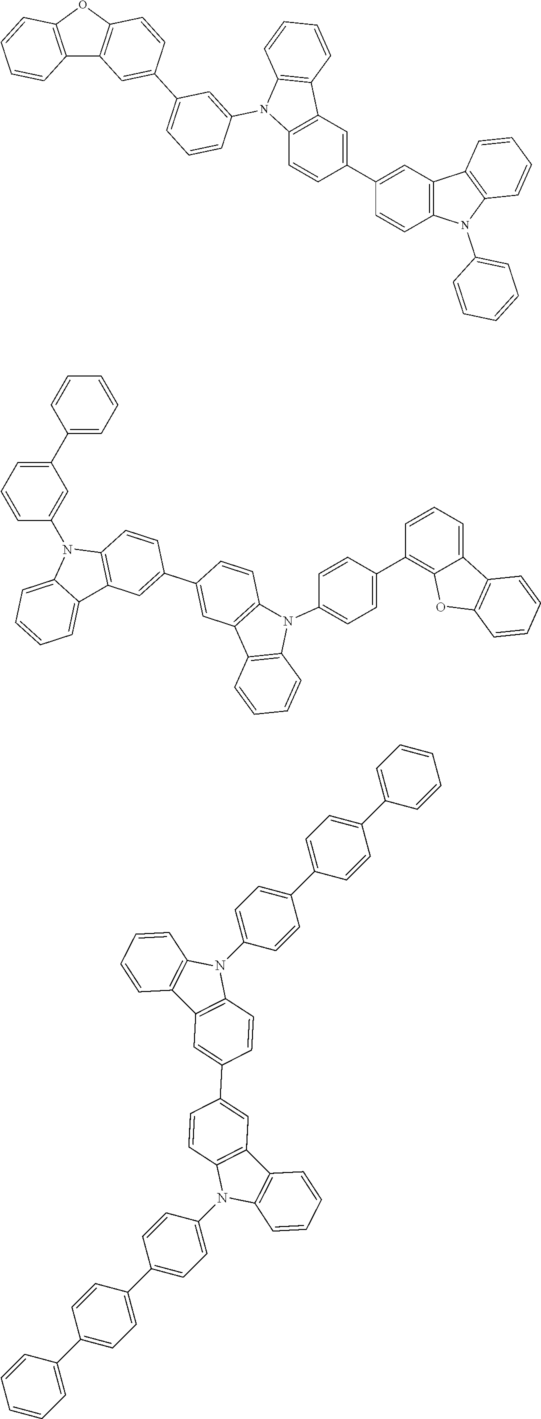 [SMILES: c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1]

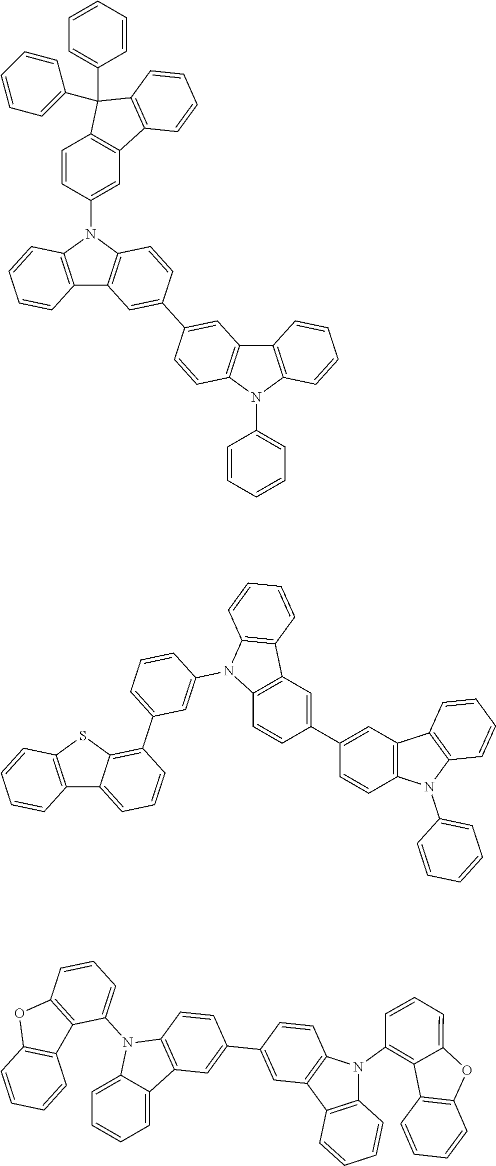 [SMILES: c1ccc(-n2c3ccccc3c3cc(-c4ccc5c(c4)c4ccccc4n5-c4ccc5c(c4)-c4ccccc4C5(c4ccccc4)c4ccccc4)ccc32)cc1 ; c1ccc(-n2c3ccccc3c3cc(-c4ccc5c(c4)c4ccccc4n5-c4cccc(-c5cccc6c5sc5ccccc56)c4)ccc32)cc1 ; c1ccc2c(c1)oc1cccc(-n3c4ccccc4c4cc(-c5ccc6c(c5)c5ccccc5n6-c5cccc6oc7ccccc7c56)ccc43)c12]

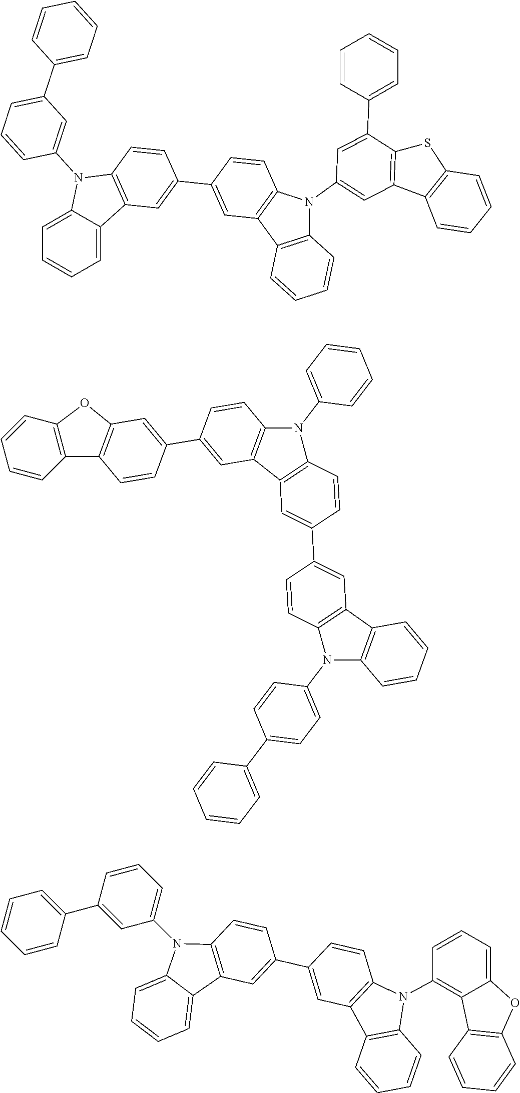 [SMILES: c1ccc(-c2cccc(-n3c4ccccc4c4cc(-c5ccc6c(c5)c5ccccc5n6-c5cc(-c6ccccc6)c6sc7ccccc7c6c5)ccc43)c2)cc1 ; c1ccc(-c2ccc(-n3c4ccccc4c4cc(-c5ccc6c(c5)c5cc(-c7ccc8c(c7)oc7ccccc78)ccc5n6-c5ccccc5)ccc43)cc2)cc1 ; c1ccc(-c2cccc(-n3c4ccccc4c4cc(-c5ccc6c(c5)c5ccccc5n6-c5cccc6oc7ccccc7c56)ccc43)c2)cc1]

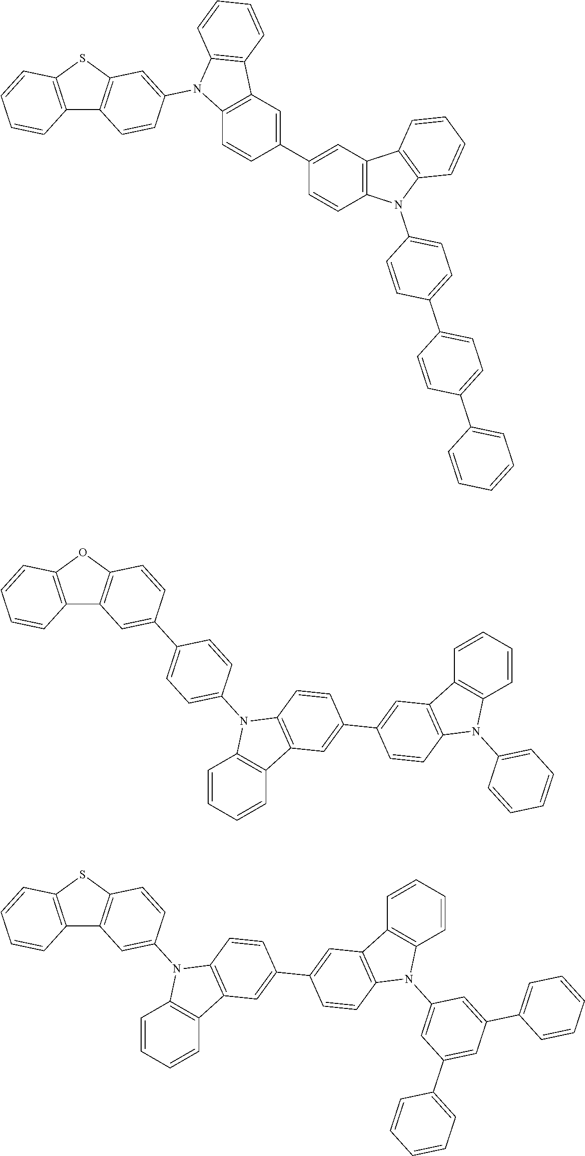 [SMILES: c1ccc(-c2ccc(-c3ccc(-n4c5ccccc5c5cc(-c6ccc7c(c6)c6ccccc6n7-c6ccc7c(c6)sc6ccccc67)ccc54)cc3)cc2)cc1 ; c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1]

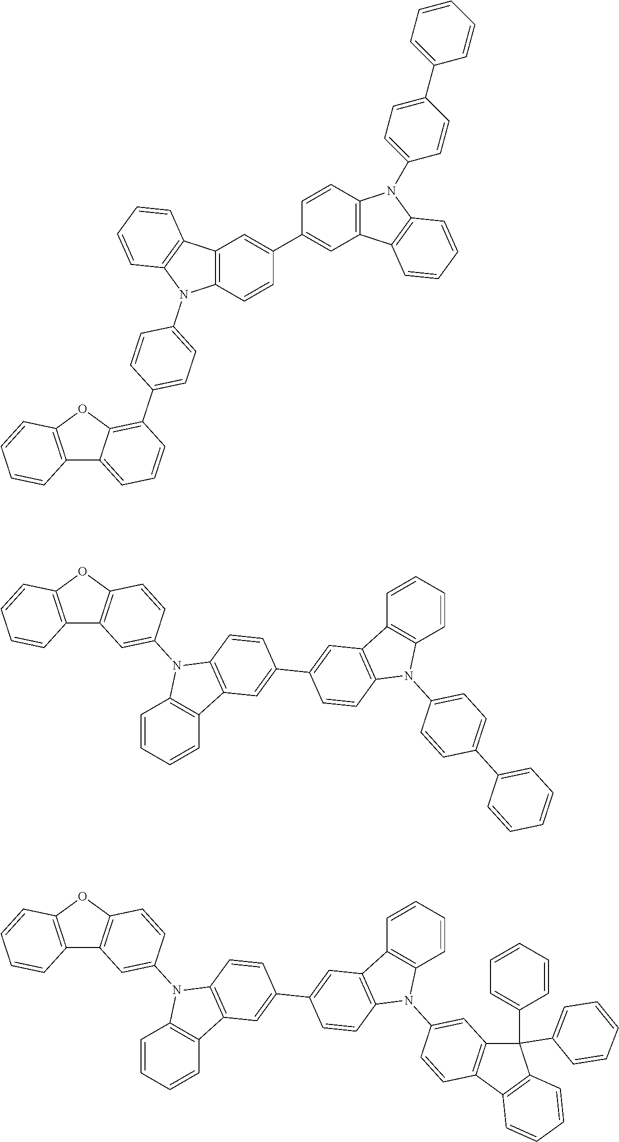 [SMILES: c1ccc(-c2ccc(-n3c4ccccc4c4cc(-c5ccc6c(c5)c5ccccc5n6-c5ccc(-c6cccc7c6oc6ccccc67)cc5)ccc43)cc2)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(C2(c3ccccc3)c3ccccc3-c3ccc(-n4c5ccccc5c5cc(-c6ccc7c(c6)c6ccccc6n7-c6ccc7oc8ccccc8c7c6)ccc54)cc32)cc1]

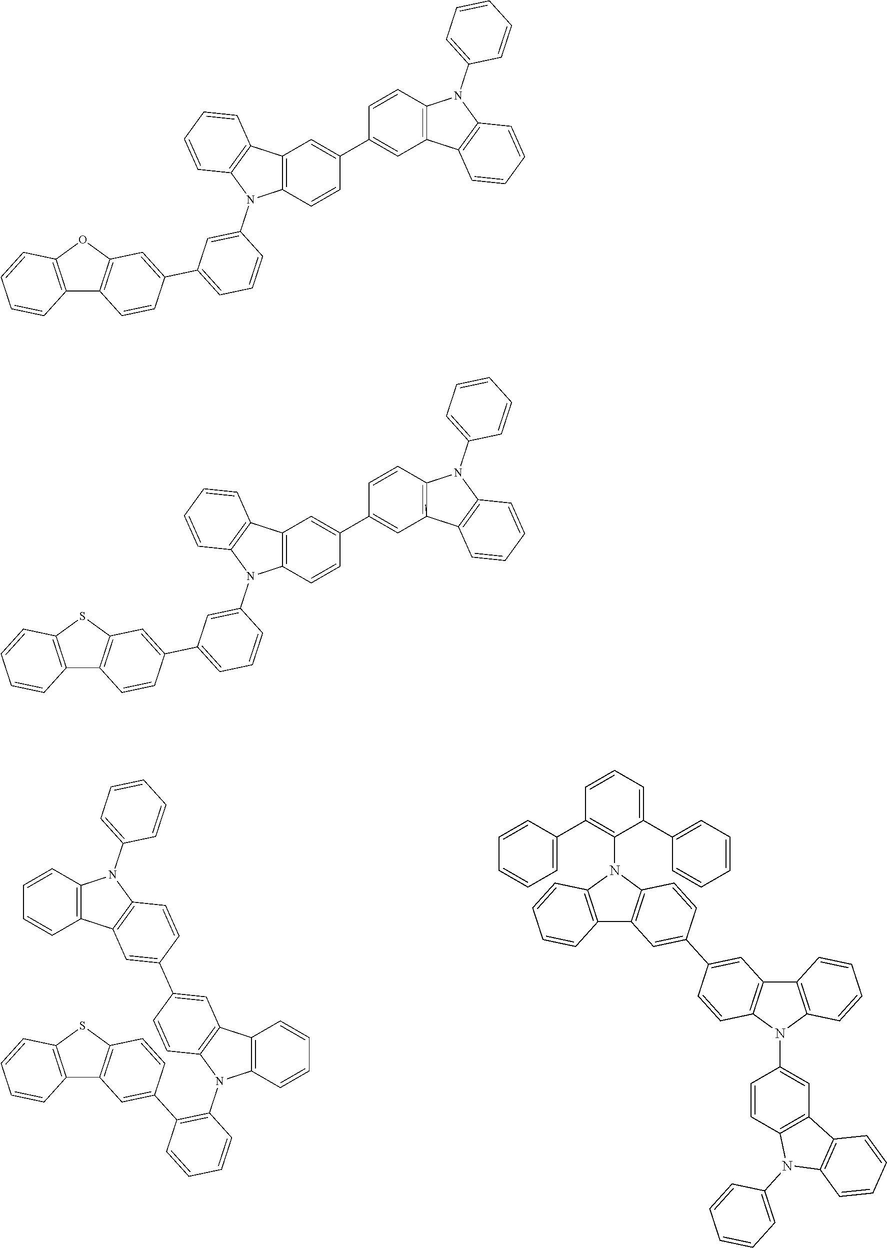

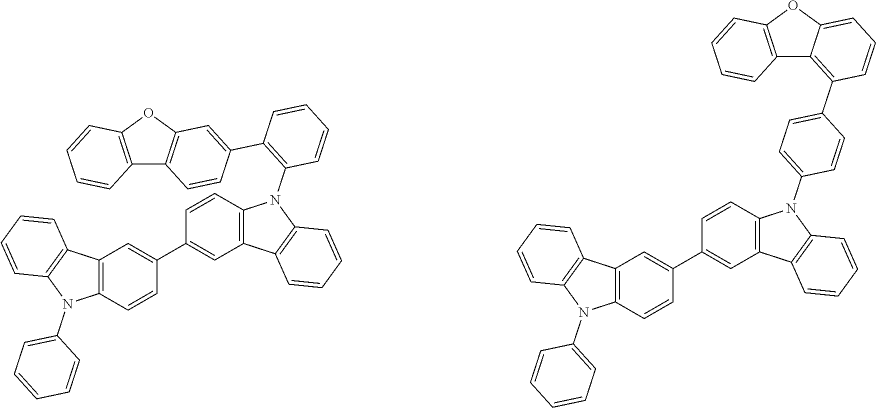 [SMILES: c1ccc(-n2c3ccccc3c3cc(-c4ccc5c(c4)c4ccccc4n5-c4ccccc4-c4ccc5c(c4)oc4ccccc45)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1]

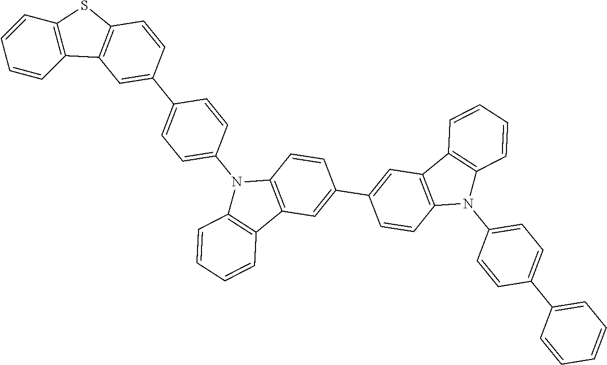 [SMILES: c1ccc(-c2ccc(-n3c4ccccc4c4cc(-c5ccc6c(c5)c5ccccc5n6-c5ccc(-c6ccc7sc8ccccc8c7c6)cc5)ccc43)cc2)cc1]

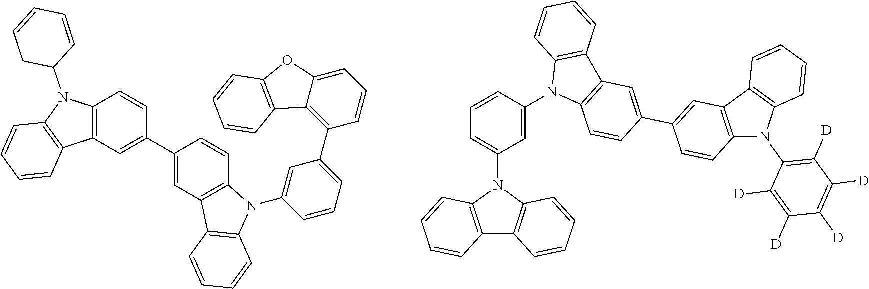 [SMILES: C1=CCC(n2c3ccccc3c3cc(-c4ccc5c(c4)c4ccccc4n5-c4cccc(-c5cccc6oc7ccccc7c56)c4)ccc32)C=C1 ; [2H]c1c([2H])c([2H])c(-n2c3ccccc3c3cc(-c4ccc5c(c4)c4ccccc4n5-c4cccc(-n5c6ccccc6c6ccccc65)c4)ccc32)c([2H])c1[2H]]

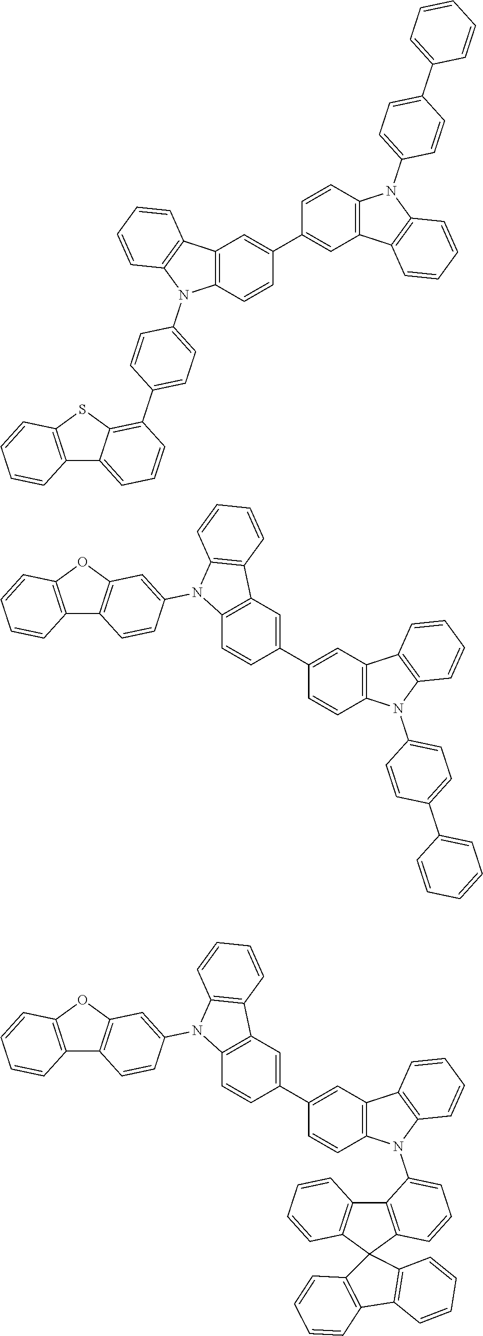 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc2c(c1)-c1ccccc1C21c2ccccc2-c2c(-n3c4ccccc4c4cc(-c5ccc6c(c5)c5ccccc5n6-c5ccc6c(c5)oc5ccccc56)ccc43)cccc21]

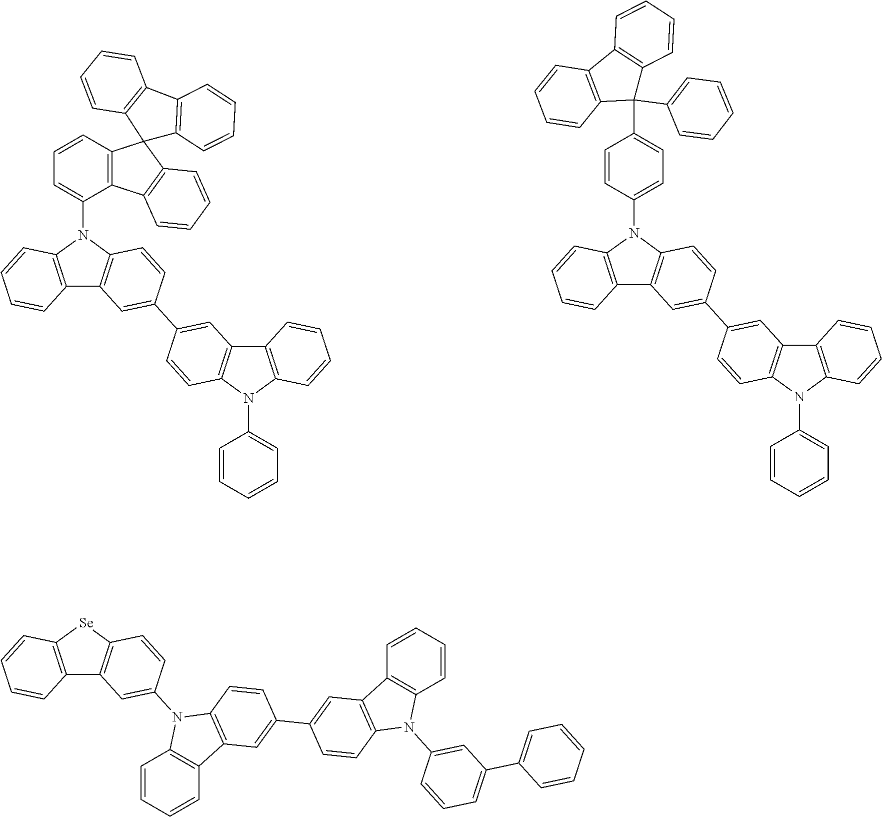 [SMILES: c1ccc(-n2c3ccccc3c3cc(-c4ccc5c(c4)c4ccccc4n5-c4cccc5c4-c4ccccc4C54c5ccccc5-c5ccccc54)ccc32)cc1 ; c1ccc(-n2c3ccccc3c3cc(-c4ccc5c(c4)c4ccccc4n5-c4ccc(C5(c6ccccc6)c6ccccc6-c6ccccc65)cc4)ccc32)cc1 ; c1ccc(-c2cccc(-n3c4ccccc4c4cc(-c5ccc6c(c5)c5ccccc5n6-c5ccc6[se]c7ccccc7c6c5)ccc43)c2)cc1]

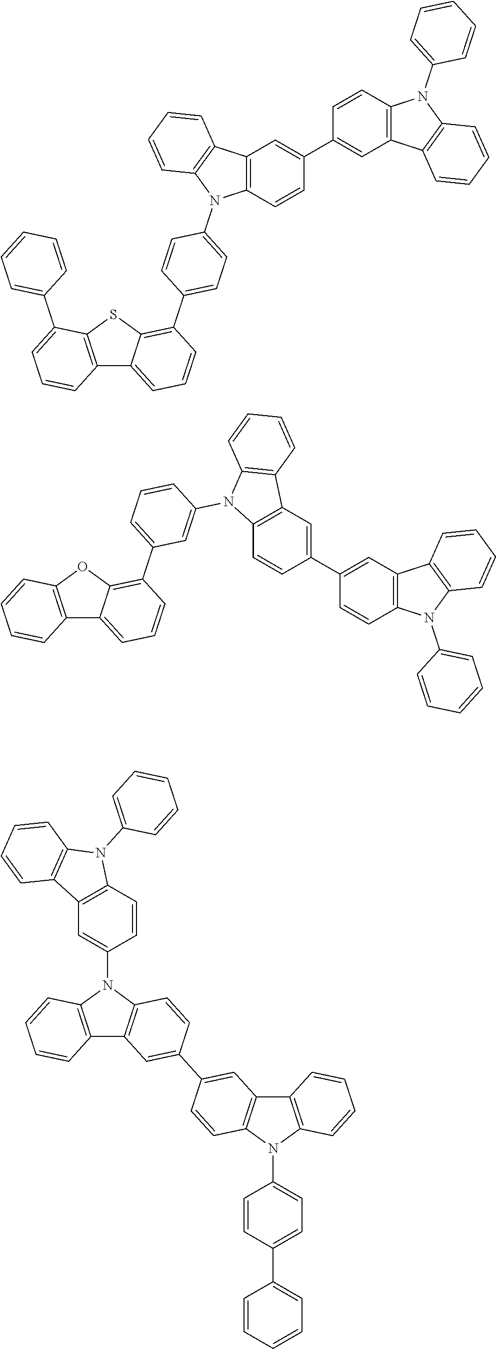 [SMILES: c1ccc(-c2cccc3c2sc2c(-c4ccc(-n5c6ccccc6c6cc(-c7ccc8c(c7)c7ccccc7n8-c7ccccc7)ccc65)cc4)cccc23)cc1 ; c1ccc(-n2c3ccccc3c3cc(-c4ccc5c(c4)c4ccccc4n5-c4cccc(-c5cccc6c5oc5ccccc56)c4)ccc32)cc1 ; c1ccc(-c2ccc(-n3c4ccccc4c4cc(-c5ccc6c(c5)c5ccccc5n6-c5ccc6c(c5)c5ccccc5n6-c5ccccc5)ccc43)cc2)cc1]

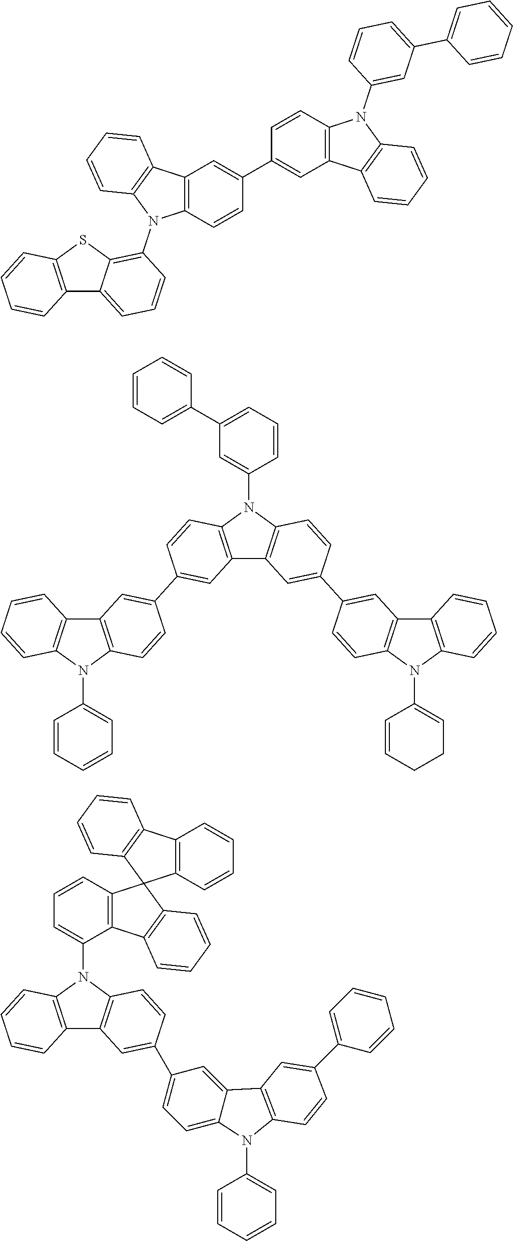

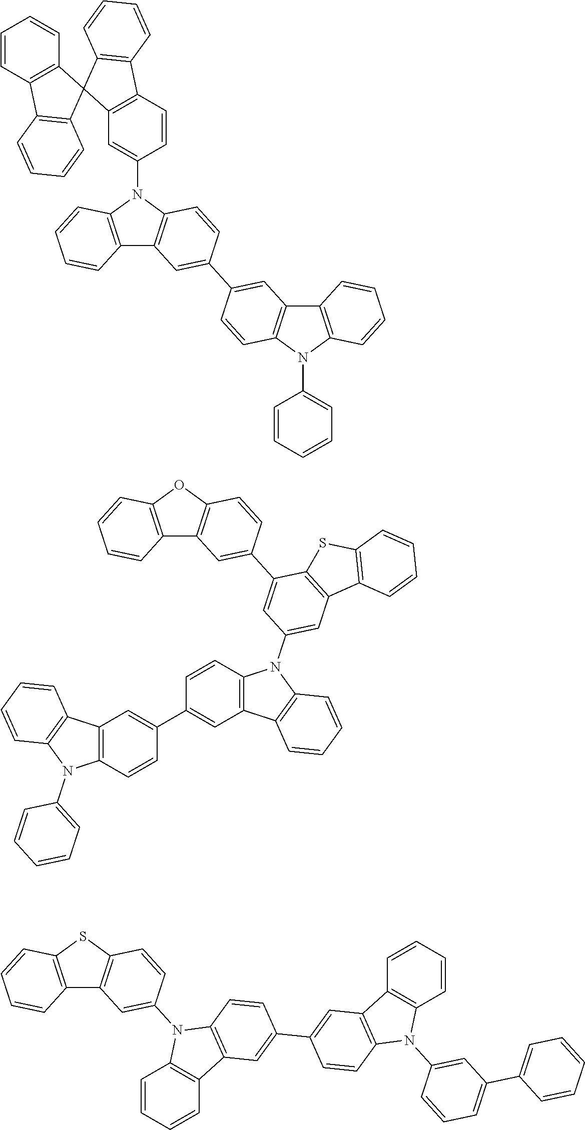 [SMILES: c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4cc(-c5ccc6oc7ccccc7c6c5)c5sc6ccccc6c5c4)ccc32)cc1 ; c1ccc(-c2cccc(-n3c4ccccc4c4cc(-c5ccc6c(c5)c5ccccc5n6-c5ccc6sc7ccccc7c6c5)ccc43)c2)cc1]

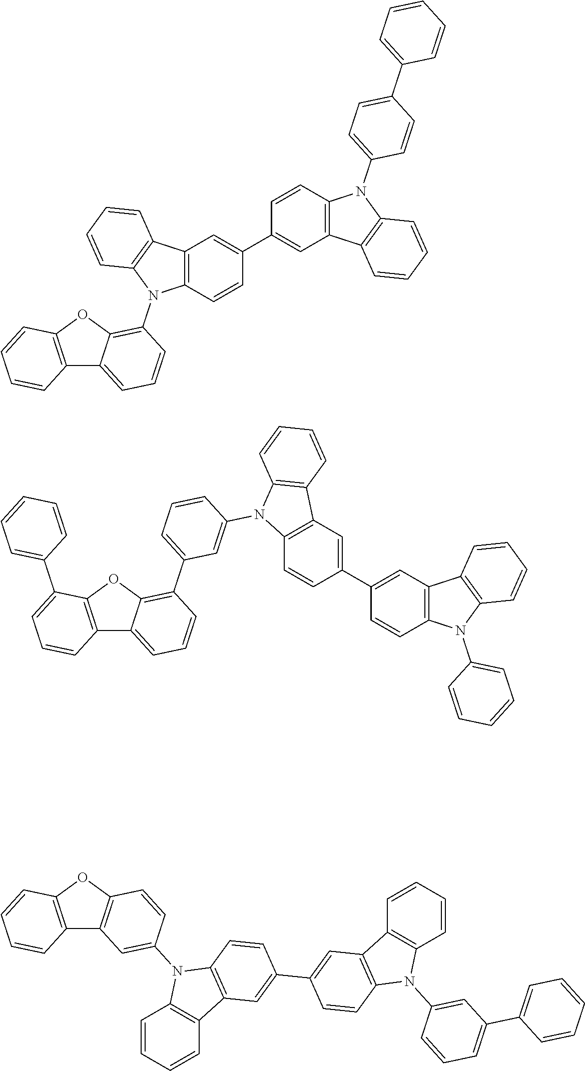 [SMILES: c1ccc(-c2ccc(-n3c4ccccc4c4cc(-c5ccc6c(c5)c5ccccc5n6-c5cccc6c5oc5ccccc56)ccc43)cc2)cc1 ; c1ccc(-c2cccc3c2oc2c(-c4cccc(-n5c6ccccc6c6cc(-c7ccc8c(c7)c7ccccc7n8-c7ccccc7)ccc65)c4)cccc23)cc1 ; c1ccc(-c2cccc(-n3c4ccccc4c4cc(-c5ccc6c(c5)c5ccccc5n6-c5ccc6oc7ccccc7c6c5)ccc43)c2)cc1]

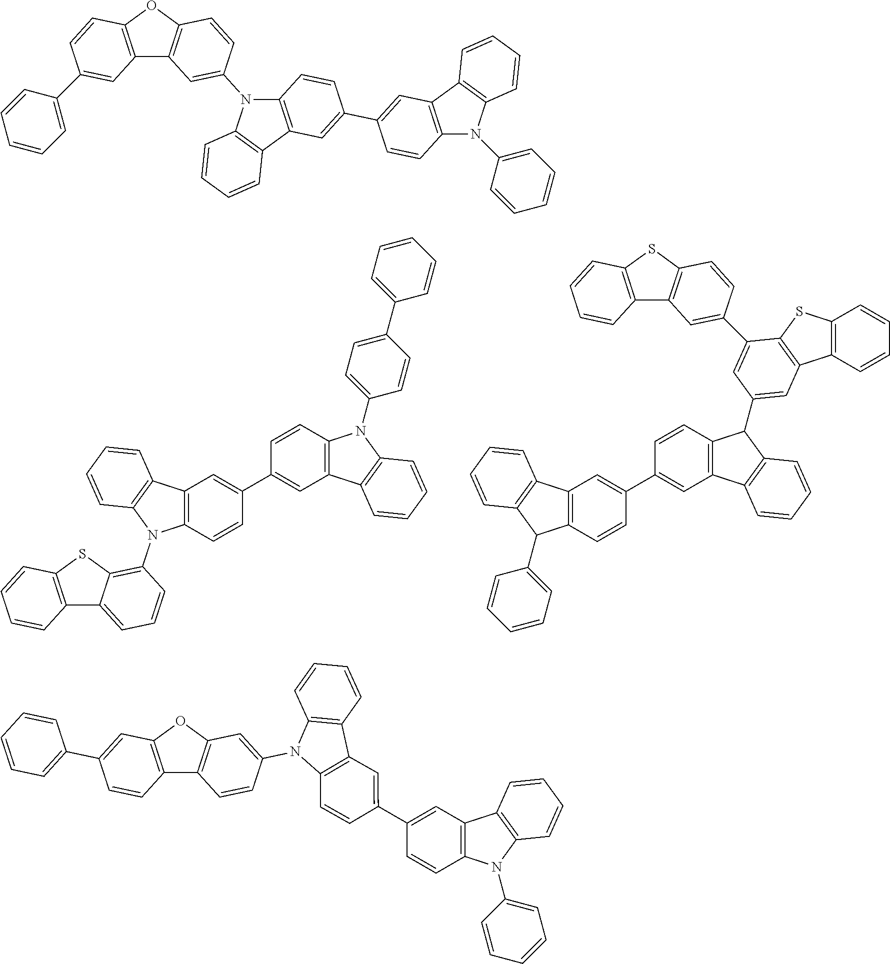 [SMILES: c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-c2ccc(-n3c4ccccc4c4cc(-c5ccc6c(c5)c5ccccc5n6-c5cccc6c5sc5ccccc56)ccc43)cc2)cc1 ; c1ccc(C2c3ccccc3-c3cc(-c4ccc5c(c4)-c4ccccc4C5c4cc(-c5ccc6sc7ccccc7c6c5)c5sc6ccccc6c5c4)ccc32)cc1 ; c1ccc(-c2ccc3c(c2)oc2cc(-n4c5ccccc5c5cc(-c6ccc7c(c6)c6ccccc6n7-c6ccccc6)ccc54)ccc23)cc1]

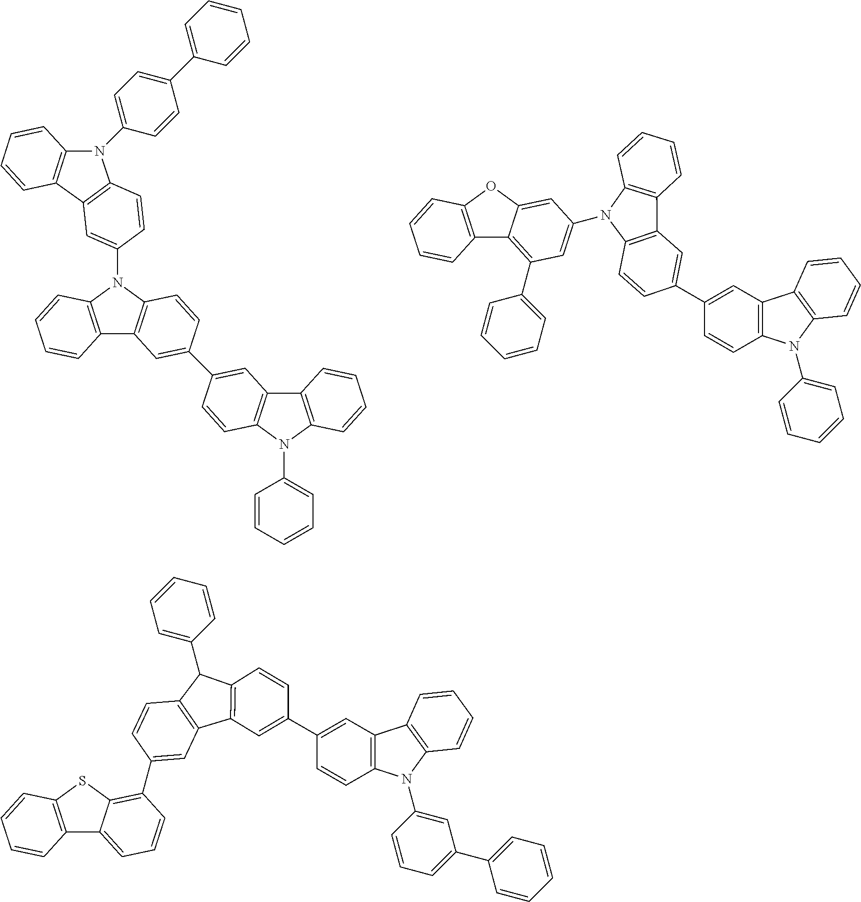 [SMILES: c1ccc(-c2ccc(-n3c4ccccc4c4cc(-n5c6ccccc6c6cc(-c7ccc8c(c7)c7ccccc7n8-c7ccccc7)ccc65)ccc43)cc2)cc1 ; c1ccc(-c2cc(-n3c4ccccc4c4cc(-c5ccc6c(c5)c5ccccc5n6-c5ccccc5)ccc43)cc3oc4ccccc4c23)cc1 ; c1ccc(-c2cccc(-n3c4ccccc4c4cc(-c5ccc6c(c5)-c5cc(-c7cccc8c7sc7ccccc78)ccc5C6c5ccccc5)ccc43)c2)cc1]

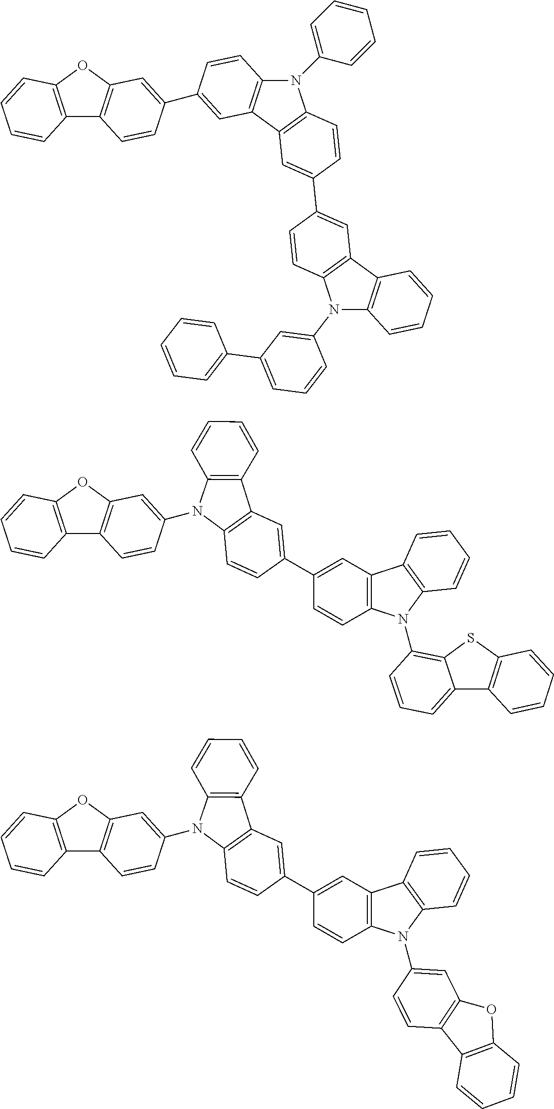

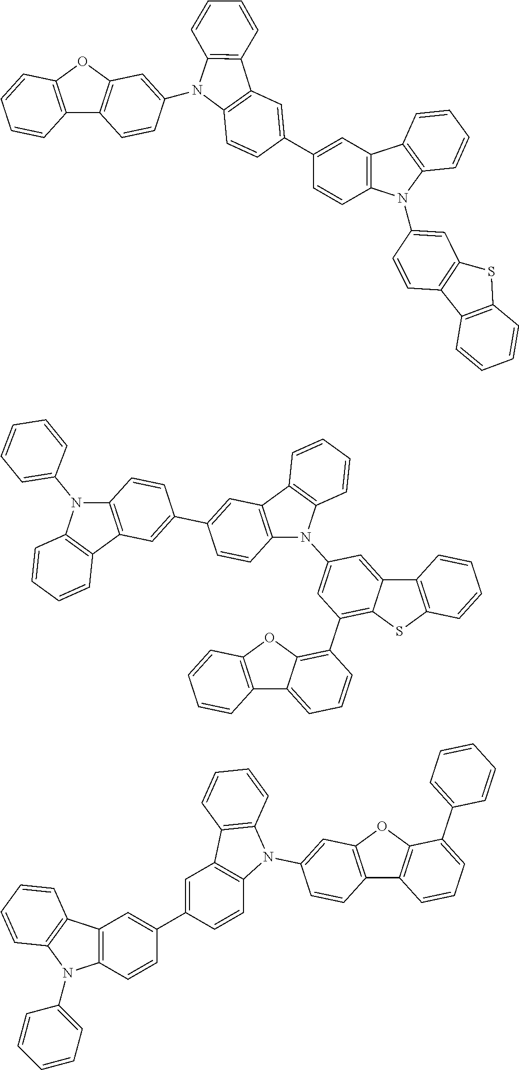 [SMILES: c1ccc2c(c1)oc1cc(-n3c4ccccc4c4cc(-c5ccc6c(c5)c5ccccc5n6-c5ccc6c(c5)sc5ccccc56)ccc43)ccc12 ; c1ccc(-n2c3ccccc3c3cc(-c4ccc5c(c4)c4ccccc4n5-c4cc(-c5cccc6c5oc5ccccc56)c5sc6ccccc6c5c4)ccc32)cc1 ; c1ccc(-c2cccc3c2oc2cc(-n4c5ccccc5c5cc(-c6ccc7c(c6)c6ccccc6n7-c6ccccc6)ccc54)ccc23)cc1]

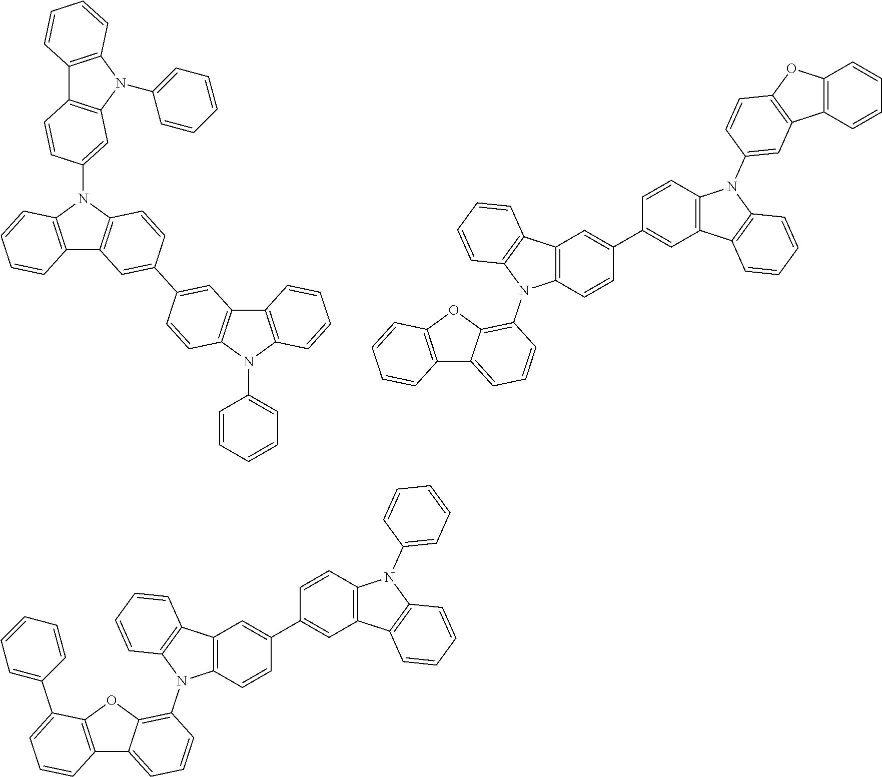 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc2c(c1)oc1ccc(-n3c4ccccc4c4cc(-c5ccc6c(c5)c5ccccc5n6-c5cccc6c5oc5ccccc56)ccc43)cc12 ; c1ccc(-c2cccc3c2oc2c(-n4c5ccccc5c5cc(-c6ccc7c(c6)c6ccccc6n7-c6ccccc6)ccc54)cccc23)cc1]

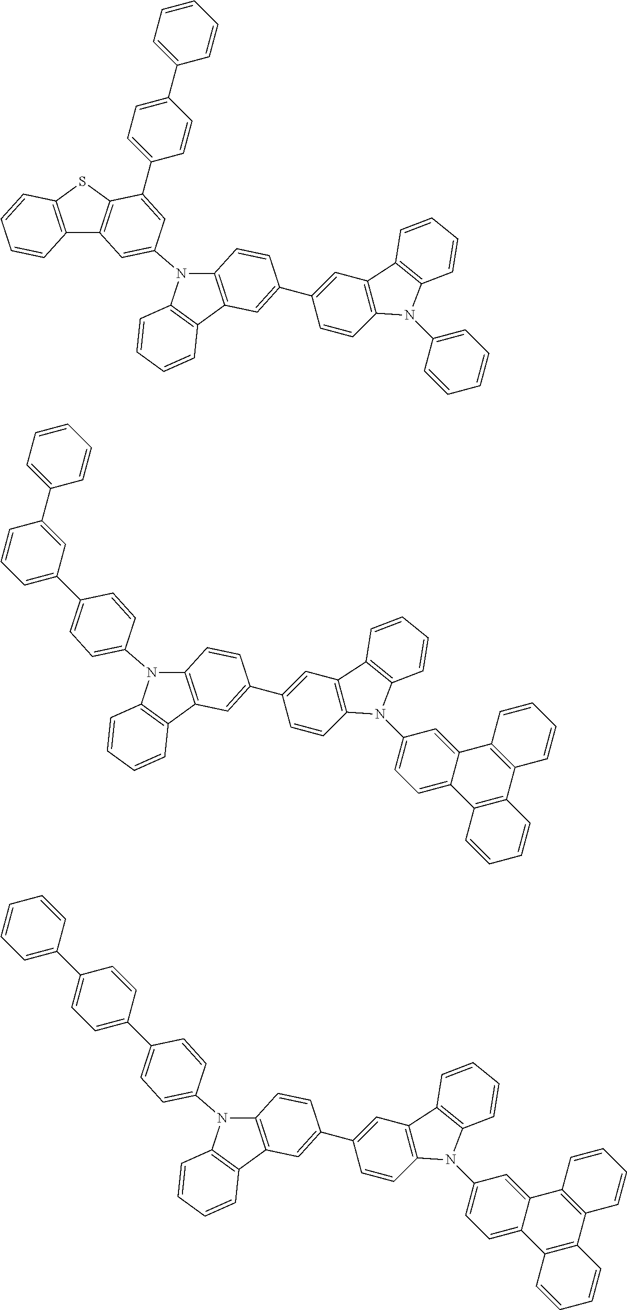 [SMILES: c1ccc(-c2ccc(-c3cc(-n4c5ccccc5c5cc(-c6ccc7c(c6)c6ccccc6n7-c6ccccc6)ccc54)cc4c3sc3ccccc34)cc2)cc1 ; c1ccc(-c2cccc(-c3ccc(-n4c5ccccc5c5cc(-c6ccc7c(c6)c6ccccc6n7-c6ccc7c8ccccc8c8ccccc8c7c6)ccc54)cc3)c2)cc1 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1]

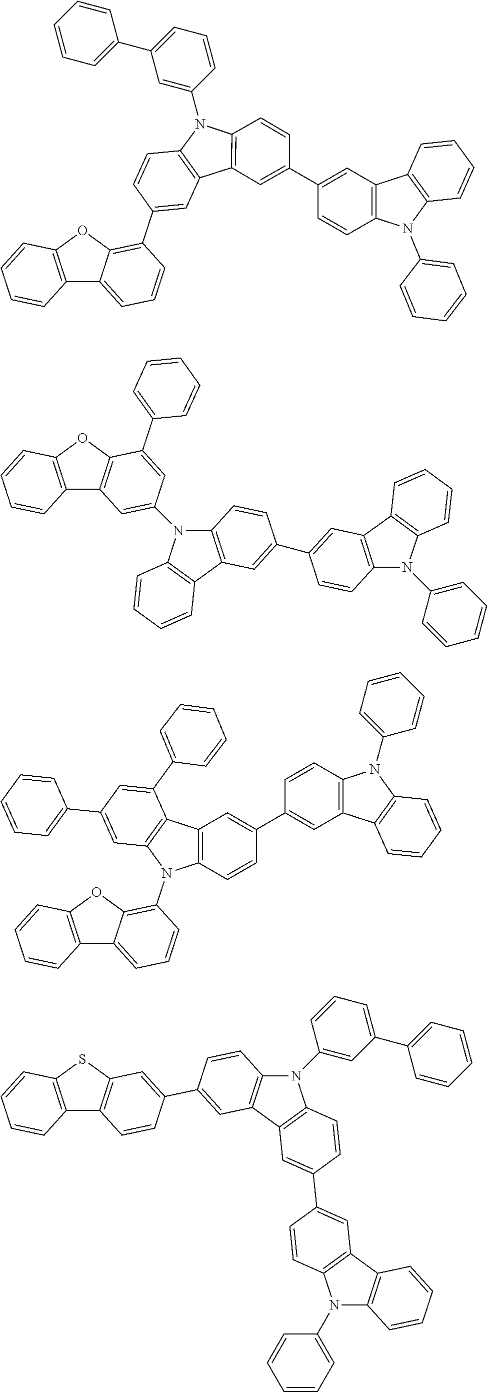 [SMILES: c1ccc(-c2cccc(-n3c4ccc(-c5ccc6c(c5)c5ccccc5n6-c5ccccc5)cc4c4cc(-c5cccc6c5oc5ccccc56)ccc43)c2)cc1 ; c1ccc(-c2cc(-n3c4ccccc4c4cc(-c5ccc6c(c5)c5ccccc5n6-c5ccccc5)ccc43)cc3c2oc2ccccc23)cc1 ; c1ccc(-c2cc(-c3ccccc3)c3c4cc(-c5ccc6c(c5)c5ccccc5n6-c5ccccc5)ccc4n(-c4cccc5c4oc4ccccc45)c3c2)cc1 ; c1ccc(-c2cccc(-n3c4ccc(-c5ccc6c(c5)sc5ccccc56)cc4c4cc(-c5ccc6c(c5)c5ccccc5n6-c5ccccc5)ccc43)c2)cc1]

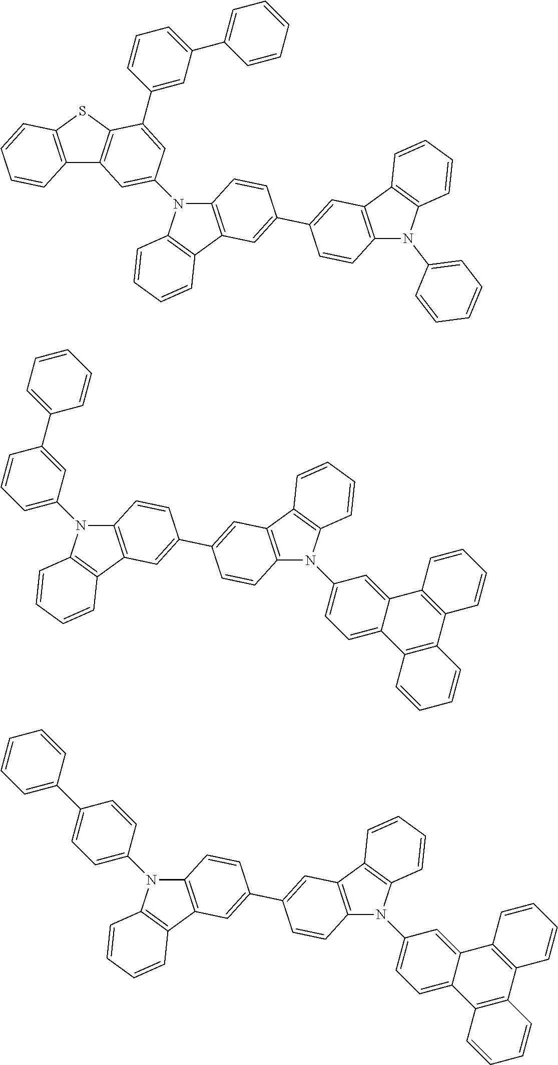 [SMILES: c1ccc(-c2cccc(-c3cc(-n4c5ccccc5c5cc(-c6ccc7c(c6)c6ccccc6n7-c6ccccc6)ccc54)cc4c3sc3ccccc34)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1]

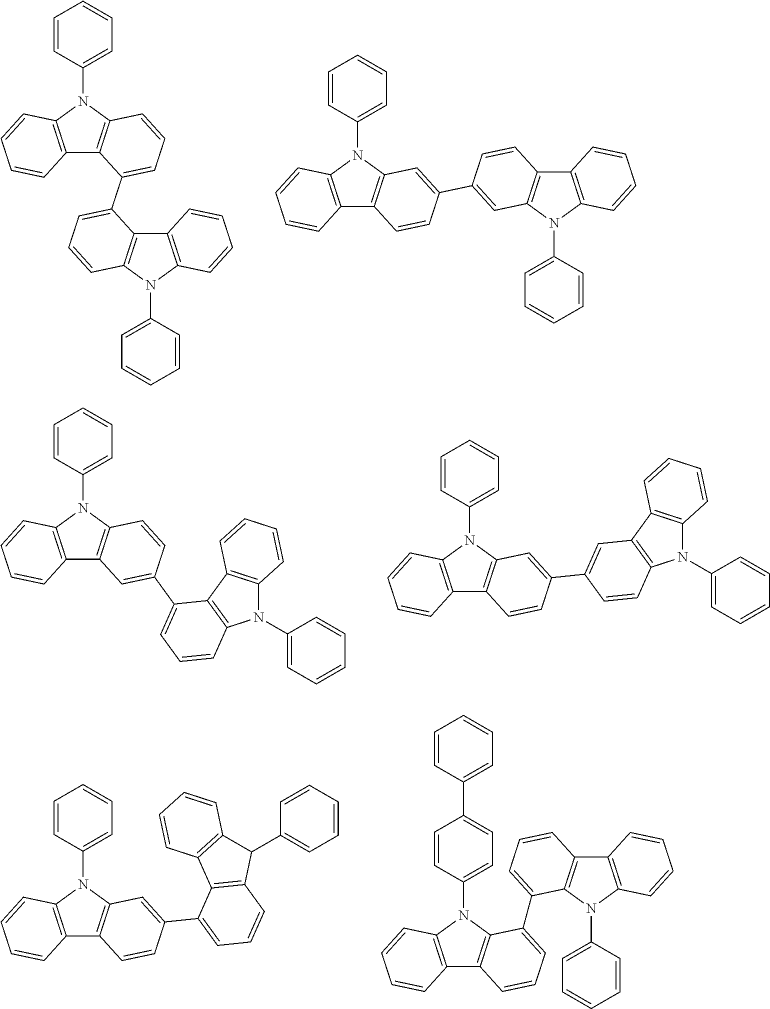 [SMILES: c1ccc(-n2c3ccccc3c3c(-c4cccc5c4c4ccccc4n5-c4ccccc4)cccc32)cc1 ; c1ccc(-n2c3ccccc3c3ccc(-c4ccc5c6ccccc6n(-c6ccccc6)c5c4)cc32)cc1 ; c1ccc(-n2c3ccccc3c3cc(-c4cccc5c4c4ccccc4n5-c4ccccc4)ccc32)cc1 ; c1ccc(-n2c3ccccc3c3cc(-c4ccc5c6ccccc6n(-c6ccccc6)c5c4)ccc32)cc1 ; c1ccc(C2c3ccccc3-c3c(-c4ccc5c6ccccc6n(-c6ccccc6)c5c4)cccc32)cc1 ; c1ccc(-c2ccc(-n3c4ccccc4c4cccc(-c5cccc6c7ccccc7n(-c7ccccc7)c56)c43)cc2)cc1]

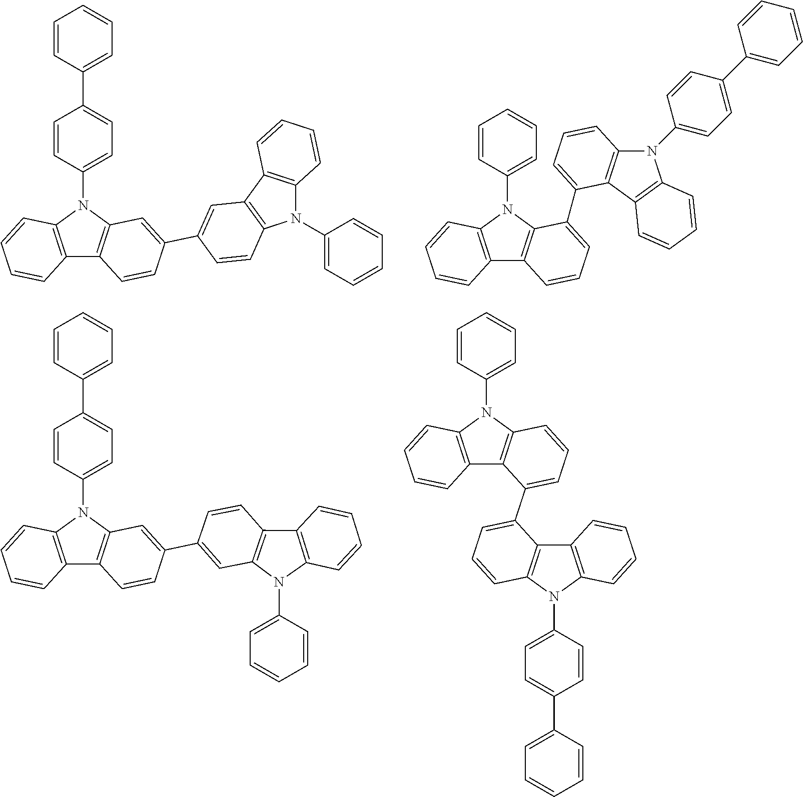

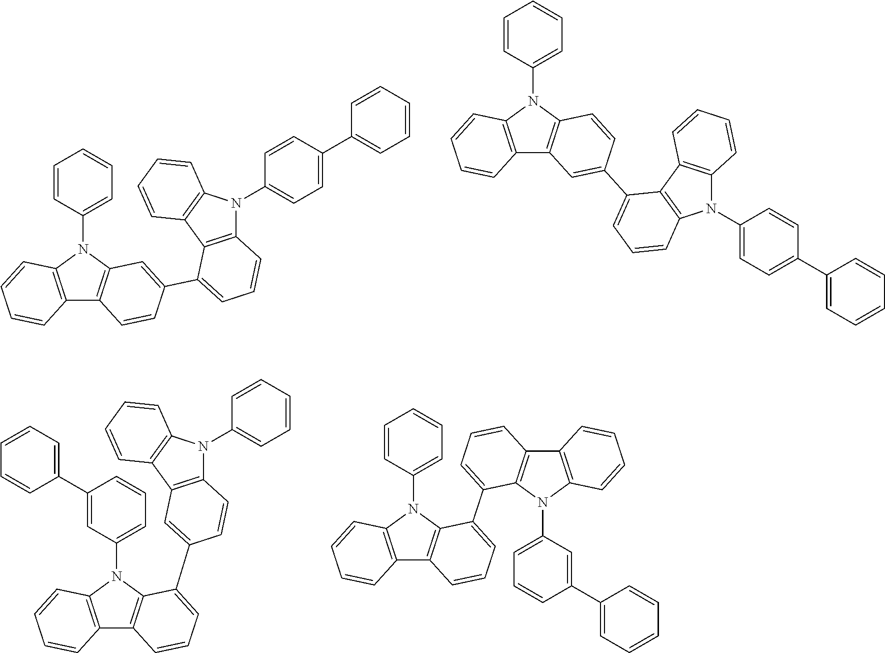

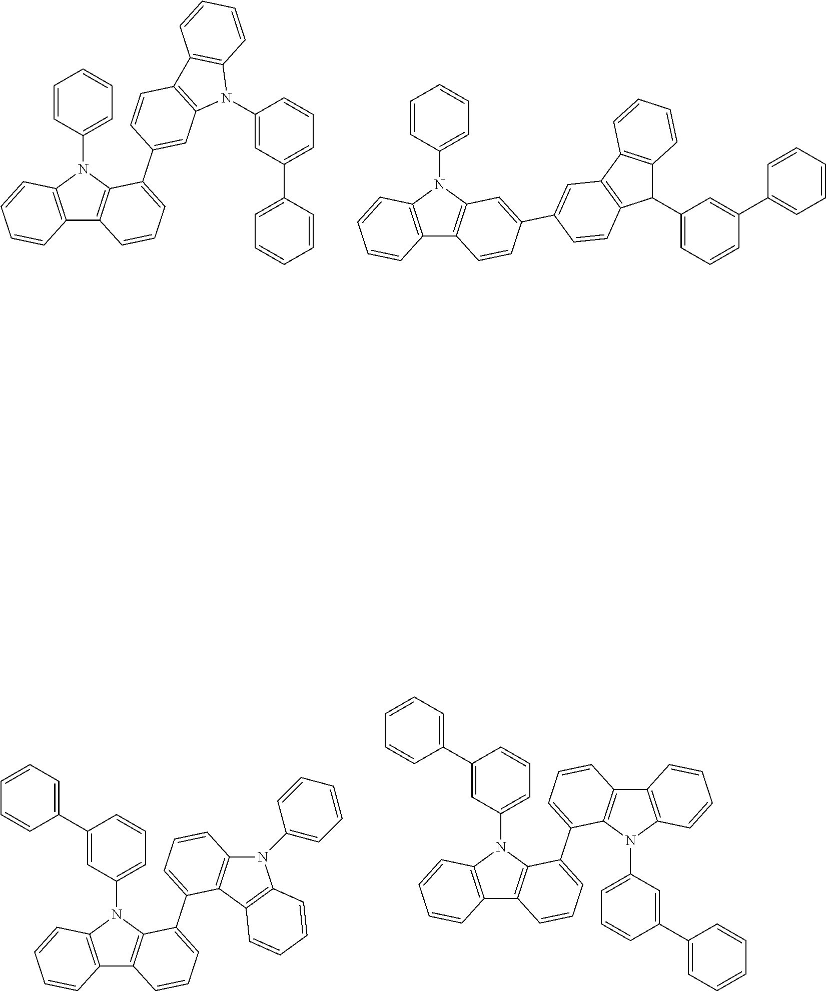

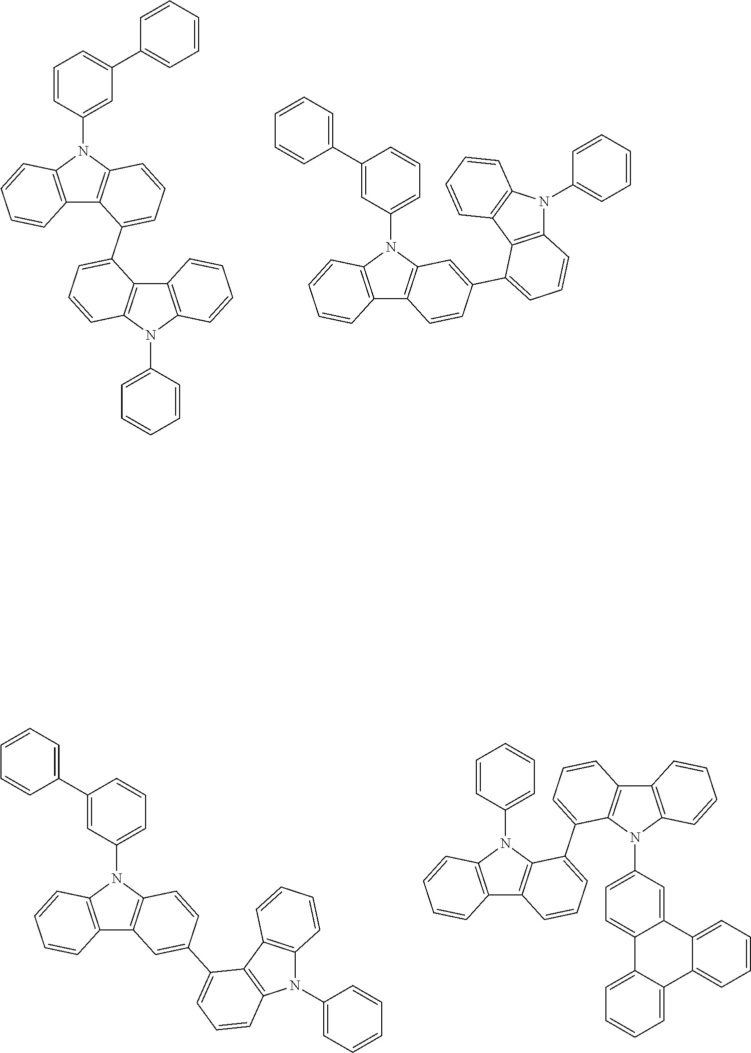 [SMILES: c1ccc(-c2cccc(-n3c4ccccc4c4c(-c5cccc6c5c5ccccc5n6-c5ccccc5)cccc43)c2)cc1 ; c1ccc(-c2cccc(-n3c4ccccc4c4ccc(-c5cccc6c5c5ccccc5n6-c5ccccc5)cc43)c2)cc1 ; c1ccc(-c2cccc(-n3c4ccccc4c4cc(-c5cccc6c5c5ccccc5n6-c5ccccc5)ccc43)c2)cc1 ; c1ccc(-n2c3ccccc3c3cccc(-c4cccc5c6ccccc6n(-c6ccc7c8ccccc8c8ccccc8c7c6)c45)c32)cc1]

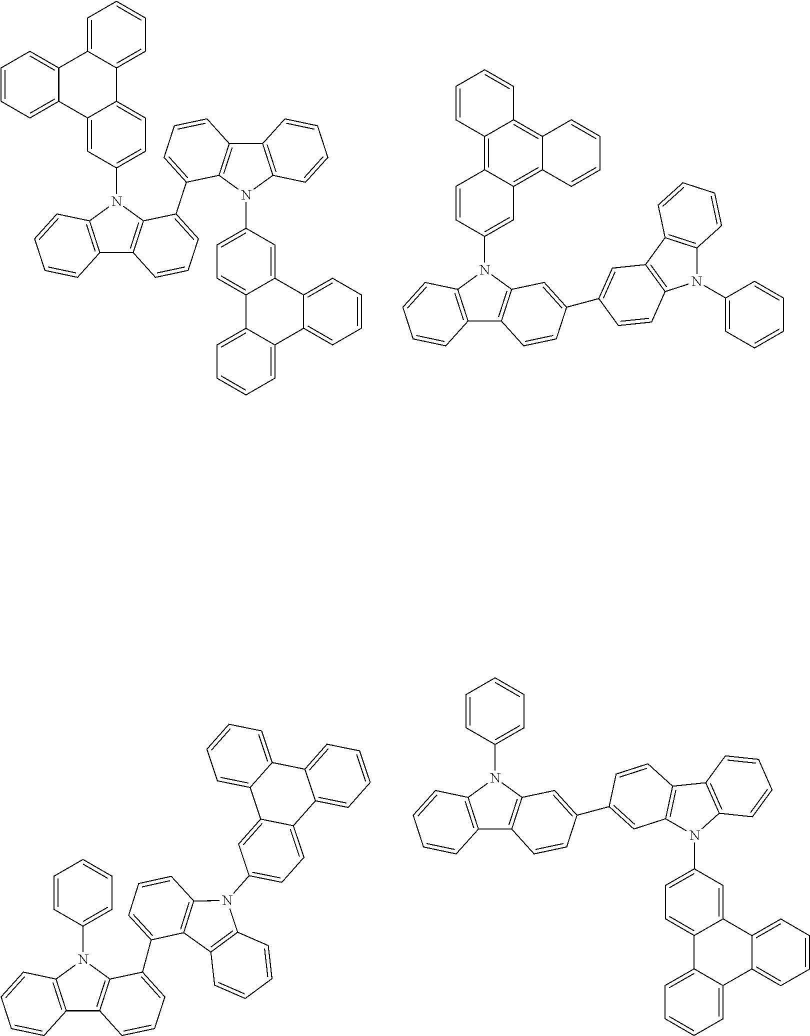

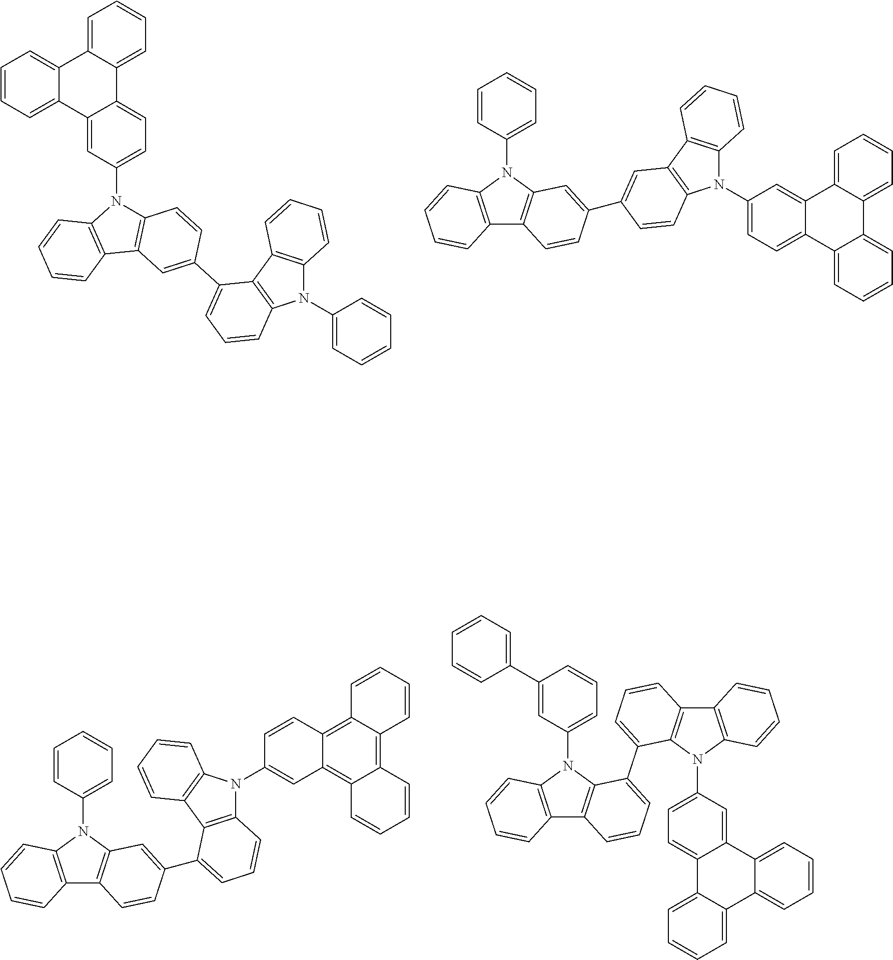 [SMILES: c1ccc(-n2c3ccccc3c3c(-c4ccc5c(c4)c4ccccc4n5-c4ccc5c6ccccc6c6ccccc6c5c4)cccc32)cc1 ; c1ccc(-n2c3ccccc3c3ccc(-c4ccc5c(c4)c4ccccc4n5-c4ccc5c6ccccc6c6ccccc6c5c4)cc32)cc1 ; c1ccc(-n2c3ccccc3c3ccc(-c4cccc5c4c4ccccc4n5-c4ccc5c6ccccc6c6ccccc6c5c4)cc32)cc1 ; c1ccc(-c2cccc(-n3c4ccccc4c4cccc(-c5cccc6c7ccccc7n(-c7ccc8c9ccccc9c9ccccc9c8c7)c56)c43)c2)cc1]

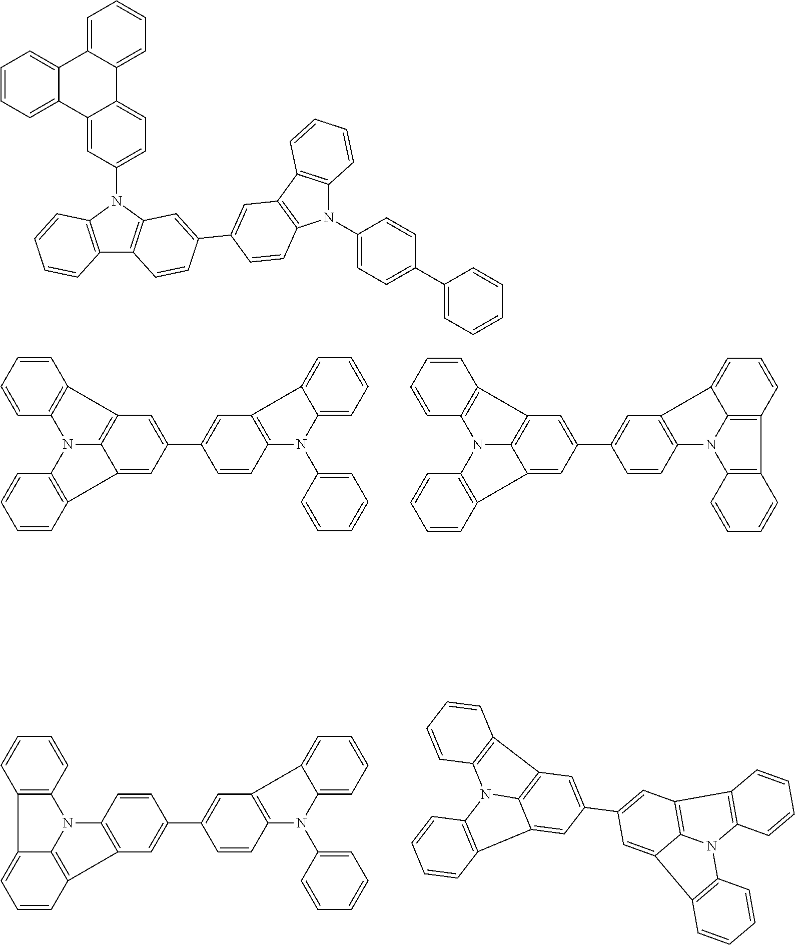

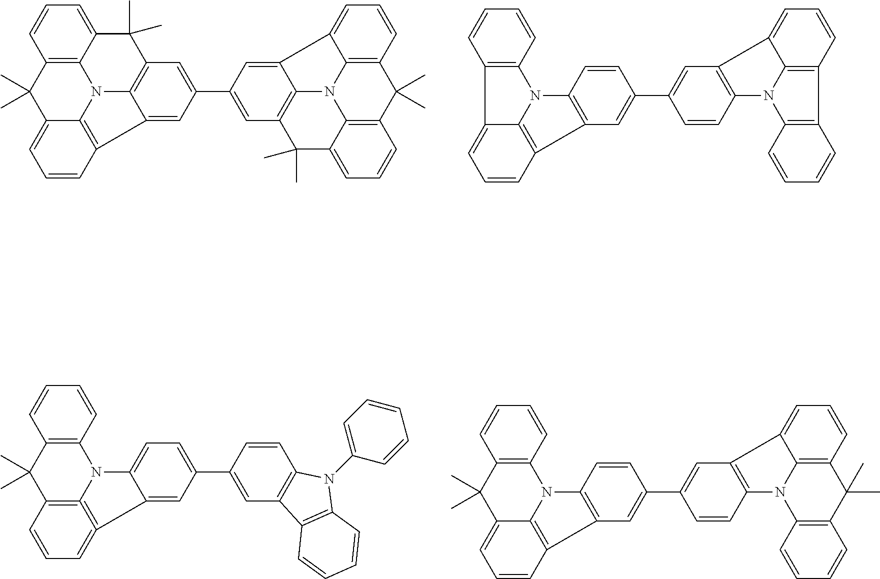

Materials used for hole-transporting layers may be any materials that are used in these layers according to the prior art. The following are especially suitable: indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. Preferred hole-transporting compounds are shown in the following table:

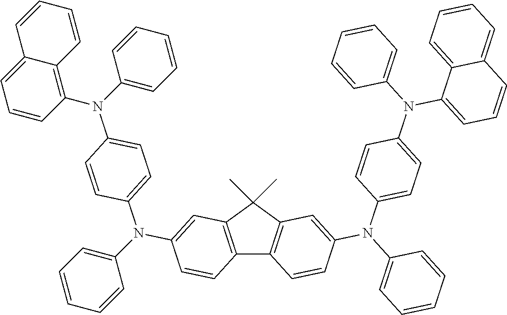 [SMILES: CC1(C)c2cc(N(c3ccccc3)c3ccc(N(c4ccccc4)c4cccc5ccccc45)cc3)ccc2-c2ccc(N(c3ccccc3)c3ccc(N(c4ccccc4)c4cccc5ccccc45)cc3)cc21]

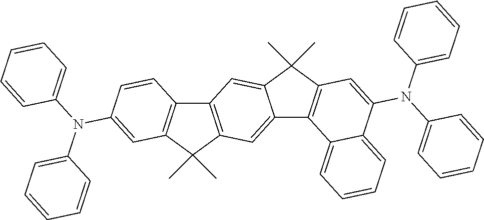 [SMILES: CC1(C)c2cc(N(c3ccccc3)c3ccccc3)ccc2-c2cc3c(cc21)-c1c(cc(N(c2ccccc2)c2ccccc2)c2ccccc12)C3(C)C]

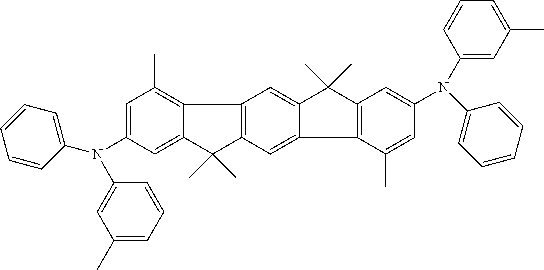 [SMILES: Cc1cccc(N(c2ccccc2)c2cc(C)c3c(c2)C(C)(C)c2cc4c(cc2-3)C(C)(C)c2cc(N(c3ccccc3)c3cccc(C)c3)cc(C)c2-4)c1]

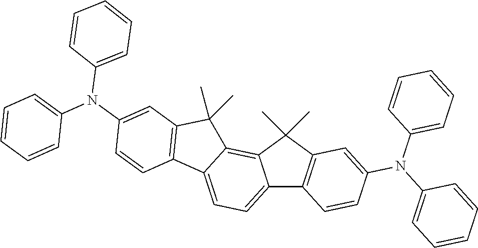 [SMILES: CC1(C)c2cc(N(c3ccccc3)c3ccccc3)ccc2-c2ccc3c(c21)C(C)(C)c1cc(N(c2ccccc2)c2ccccc2)ccc1-3]

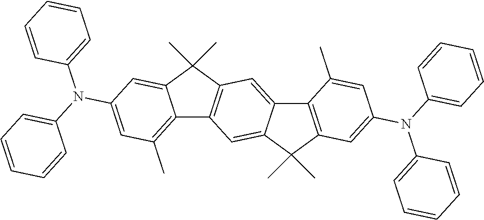 [SMILES: Cc1cc(N(c2ccccc2)c2ccccc2)cc2c1-c1cc3c(cc1C2(C)C)-c1c(C)cc(N(c2ccccc2)c2ccccc2)cc1C3(C)C]

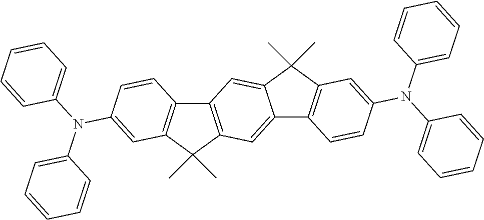 [SMILES: CC1(C)c2cc(N(c3ccccc3)c3ccccc3)ccc2-c2cc3c(cc21)-c1ccc(N(c2ccccc2)c2ccccc2)cc1C3(C)C]

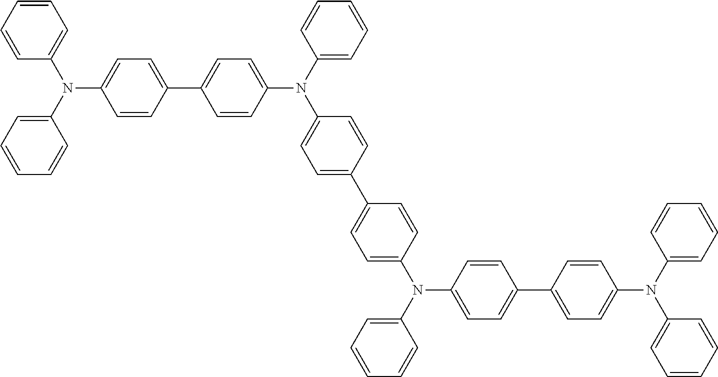 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

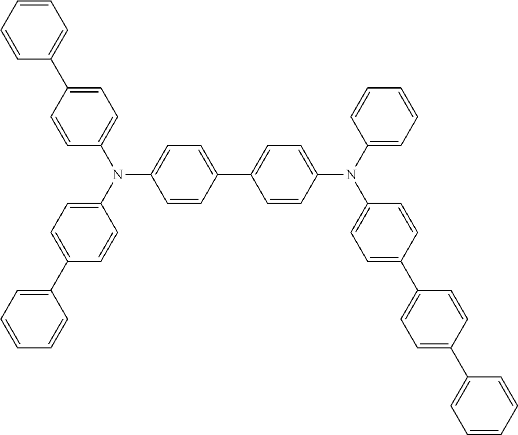 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

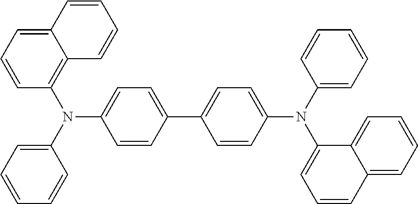 [SMILES: c1ccc(N(c2ccc(-c3ccc(N(c4ccccc4)c4cccc5ccccc45)cc3)cc2)c2cccc3ccccc23)cc1]

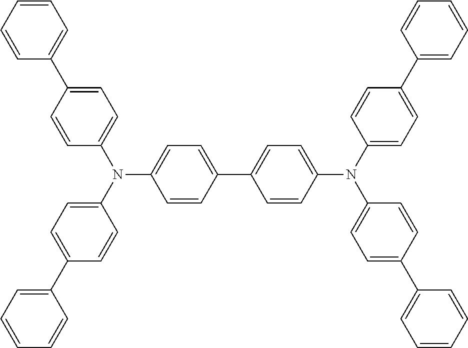 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

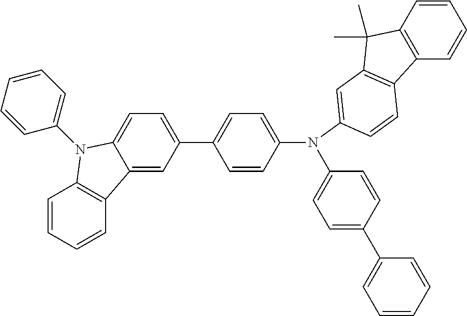 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

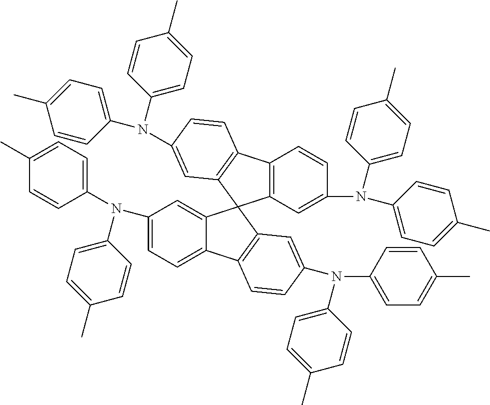

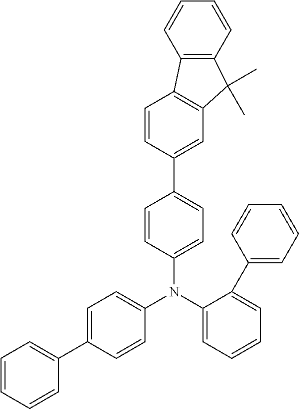 [SMILES: CC1(C)c2ccccc2-c2ccc(-c3ccc(N(c4ccc(-c5ccccc5)cc4)c4ccccc4-c4ccccc4)cc3)cc21]

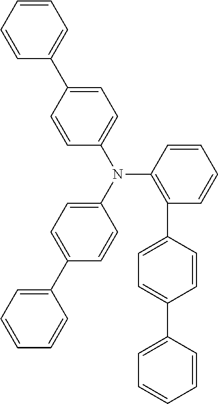 [SMILES: c1ccc(-c2ccc(-c3ccccc3N(c3ccc(-c4ccccc4)cc3)c3ccc(-c4ccccc4)cc3)cc2)cc1]

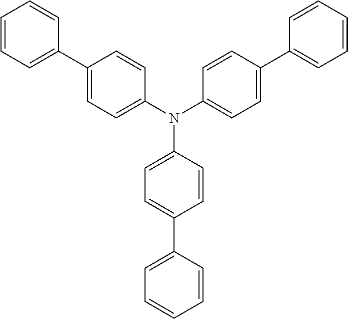 [SMILES: c1ccc(-c2ccc(N(c3ccc(-c4ccccc4)cc3)c3ccc(-c4ccccc4)cc3)cc2)cc1]

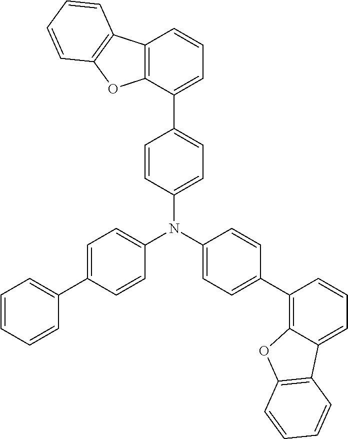 [SMILES: c1ccc(-c2ccc(N(c3ccc(-c4cccc5c4oc4ccccc45)cc3)c3ccc(-c4cccc5c4oc4ccccc45)cc3)cc2)cc1]

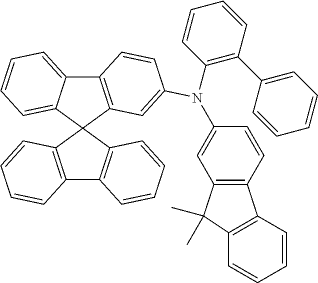 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

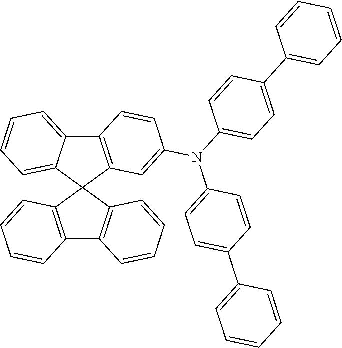 [SMILES: c1ccc(-c2ccc(N(c3ccc(-c4ccccc4)cc3)c3ccc4c(c3)C3(c5ccccc5-c5ccccc53)c3ccccc3-4)cc2)cc1]

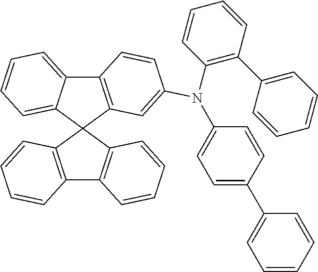 [SMILES: c1ccc(-c2ccc(N(c3ccc4c(c3)C3(c5ccccc5-c5ccccc53)c3ccccc3-4)c3ccccc3-c3ccccc3)cc2)cc1]

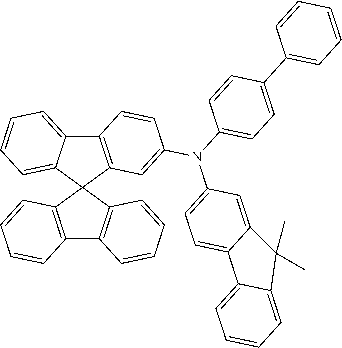 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21]

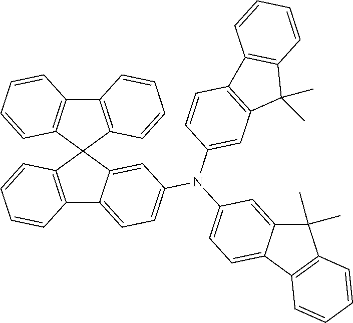 [SMILES: CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21]

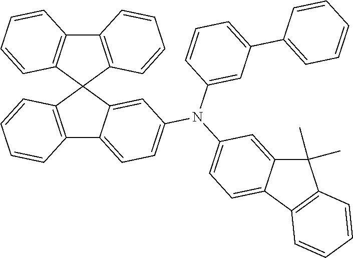 [SMILES: CC1(C)c2ccccc2-c2ccc(N(c3cccc(-c4ccccc4)c3)c3ccc4c(c3)C3(c5ccccc5-c5ccccc53)c3ccccc3-4)cc21]

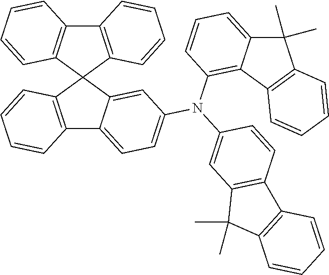 [SMILES: CC1(C)c2ccccc2-c2ccc(N(c3ccc4c(c3)C3(c5ccccc5-c5ccccc53)c3ccccc3-4)c3cccc4c3-c3ccccc3C4(C)C)cc21]

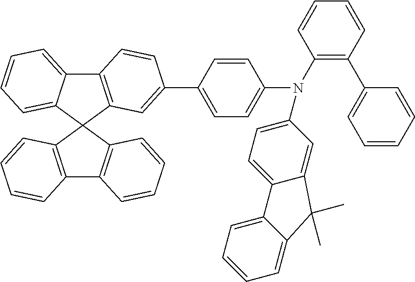 [SMILES: CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21]

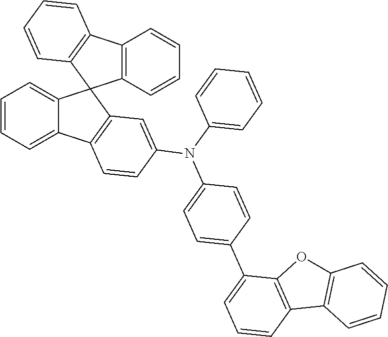 [SMILES: c1ccc(N(c2ccc(-c3cccc4c3oc3ccccc34)cc2)c2ccc3c(c2)C2(c4ccccc4-c4ccccc42)c2ccccc2-3)cc1]

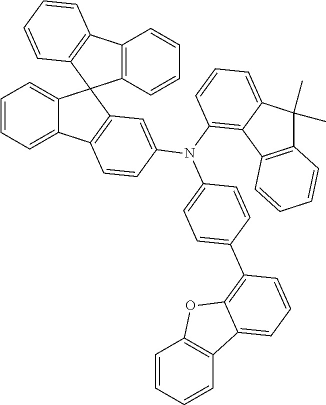 [SMILES: CC1(C)c2ccccc2-c2c(N(c3ccc(-c4cccc5c4oc4ccccc45)cc3)c3ccc4c(c3)C3(c5ccccc5-c5ccccc53)c3ccccc3-4)cccc21]

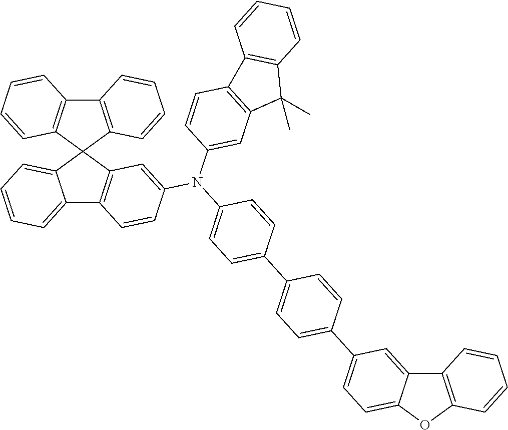 [SMILES: CC1(C)c2ccccc2-c2ccc(N(c3ccc(-c4ccc(-c5ccc6oc7ccccc7c6c5)cc4)cc3)c3ccc4c(c3)C3(c5ccccc5-c5ccccc53)c3ccccc3-4)cc21]

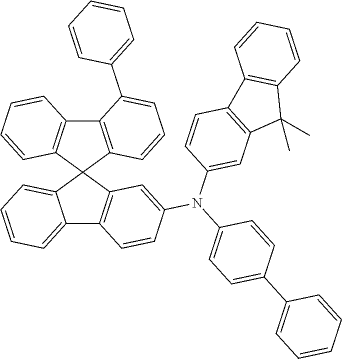 [SMILES: CC1(C)c2ccccc2-c2ccc(N(c3ccc(-c4ccccc4)cc3)c3ccc4c(c3)C3(c5ccccc5-4)c4ccccc4-c4c(-c5ccccc5)cccc43)cc21]

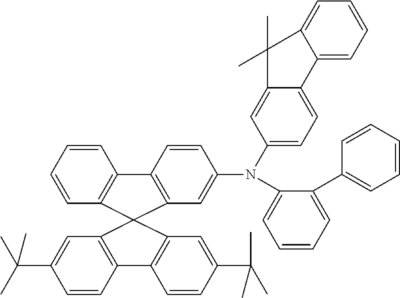 [SMILES: CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

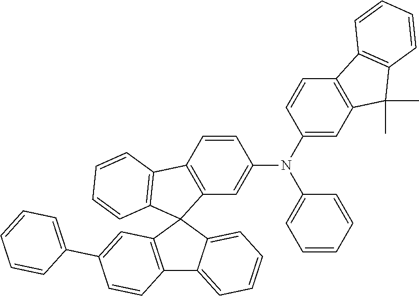 [SMILES: CC1(C)c2ccccc2-c2ccc(N(c3ccccc3)c3ccc4c(c3)C3(c5ccccc5-c5ccc(-c6ccccc6)cc53)c3ccccc3-4)cc21]

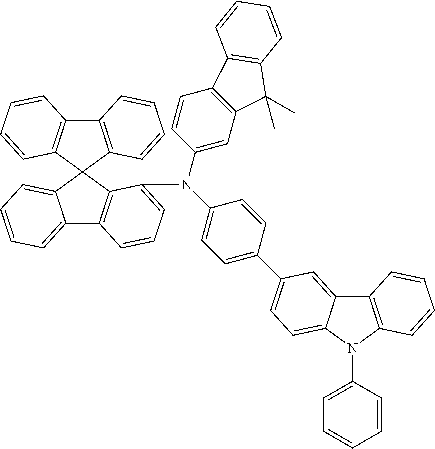 [SMILES: CC1(C)c2ccccc2-c2ccc(N(c3ccc(-c4ccc5c(c4)c4ccccc4n5-c4ccccc4)cc3)c3cccc4c3C3(c5ccccc5-c5ccccc53)c3ccccc3-4)cc21]

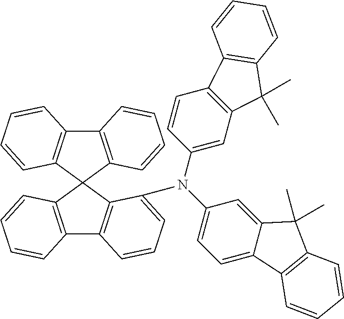 [SMILES: CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]

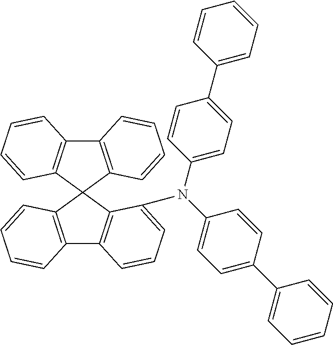 [SMILES: c1ccc(-c2ccc(N(c3ccc(-c4ccccc4)cc3)c3cccc4c3C3(c5ccccc5-c5ccccc53)c3ccccc3-4)cc2)cc1]

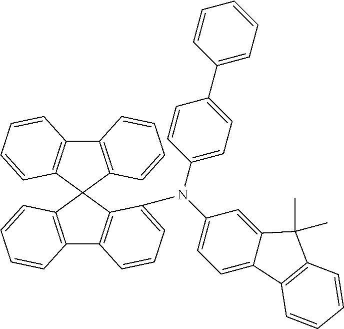 [SMILES: CC1(C)c2ccccc2-c2ccc(N(c3ccc(-c4ccccc4)cc3)c3cccc4c3C3(c5ccccc5-c5ccccc53)c3ccccc3-4)cc21]

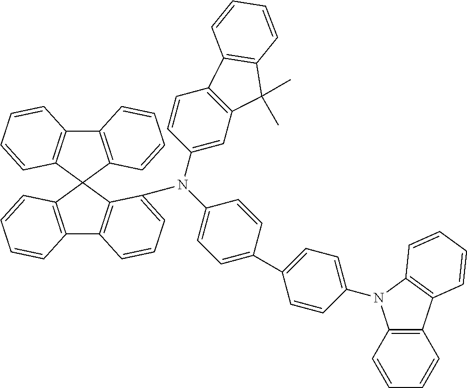 [SMILES: CC1(C)c2ccccc2-c2ccc(N(c3ccc(-c4ccc(-n5c6ccccc6c6ccccc65)cc4)cc3)c3cccc4c3C3(c5ccccc5-c5ccccc53)c3ccccc3-4)cc21]

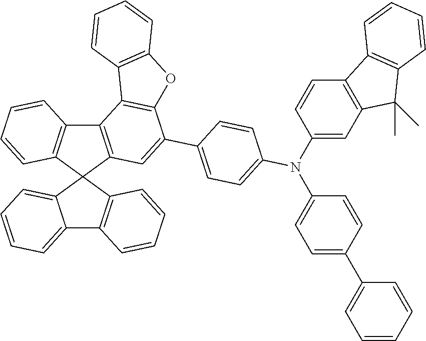 [SMILES: CC1(C)c2ccccc2-c2ccc(N(c3ccc(-c4ccccc4)cc3)c3ccc(-c4cc5c(c6c4oc4ccccc46)-c4ccccc4C54c5ccccc5-c5ccccc54)cc3)cc21]

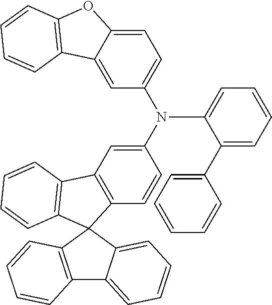 [SMILES: c1ccc(-c2ccccc2N(c2ccc3c(c2)-c2ccccc2C32c3ccccc3-c3ccccc32)c2ccc3oc4ccccc4c3c2)cc1]

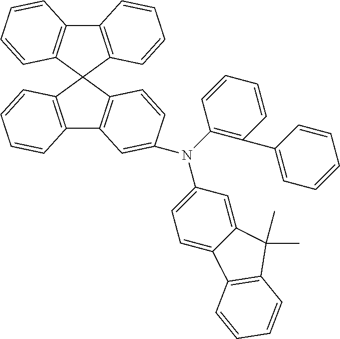 [SMILES: CC1(C)c2ccccc2-c2ccc(N(c3ccc4c(c3)-c3ccccc3C43c4ccccc4-c4ccccc43)c3ccccc3-c3ccccc3)cc21]

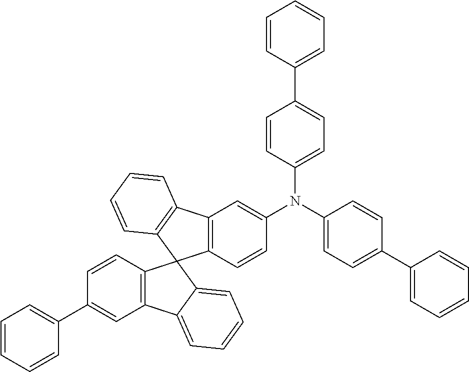 [SMILES: c1ccc(-c2ccc(N(c3ccc(-c4ccccc4)cc3)c3ccc4c(c3)-c3ccccc3C43c4ccccc4-c4cc(-c5ccccc5)ccc43)cc2)cc1]

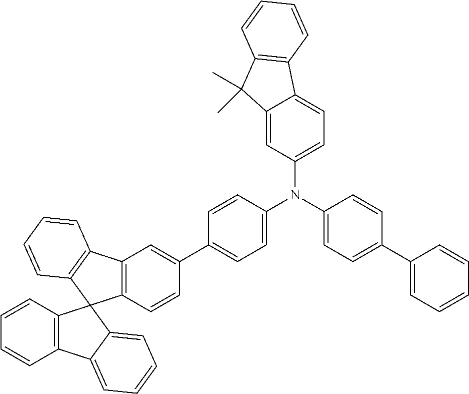 [SMILES: CC1(C)c2ccccc2-c2ccc(N(c3ccc(-c4ccccc4)cc3)c3ccc(-c4ccc5c(c4)-c4ccccc4C54c5ccccc5-c5ccccc54)cc3)cc21]

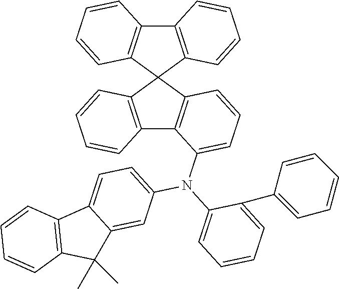 [SMILES: CC1(C)c2ccccc2-c2ccc(N(c3ccccc3-c3ccccc3)c3cccc4c3-c3ccccc3C43c4ccccc4-c4ccccc43)cc21]

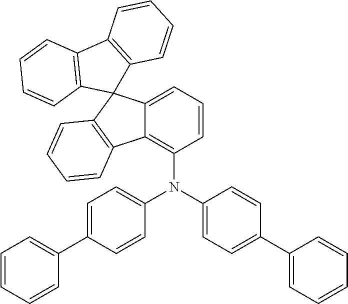 [SMILES: c1ccc(-c2ccc(N(c3ccc(-c4ccccc4)cc3)c3cccc4c3-c3ccccc3C43c4ccccc4-c4ccccc43)cc2)cc1]

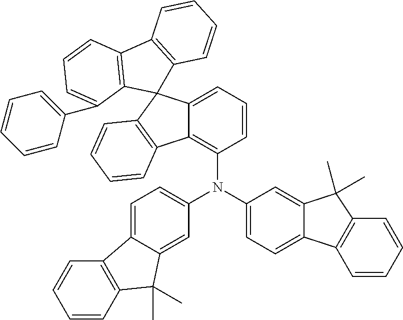 [SMILES: CC1(C)c2ccccc2-c2ccc(N(c3ccc4c(c3)C(C)(C)c3ccccc3-4)c3cccc4c3-c3ccccc3C43c4ccccc4-c4cccc(-c5ccccc5)c43)cc21]

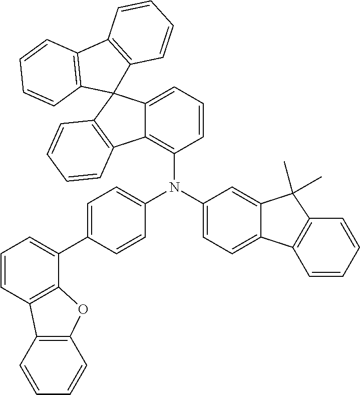 [SMILES: CC1(C)c2ccccc2-c2ccc(N(c3ccc(-c4cccc5c4oc4ccccc45)cc3)c3cccc4c3-c3ccccc3C43c4ccccc4-c4ccccc43)cc21]

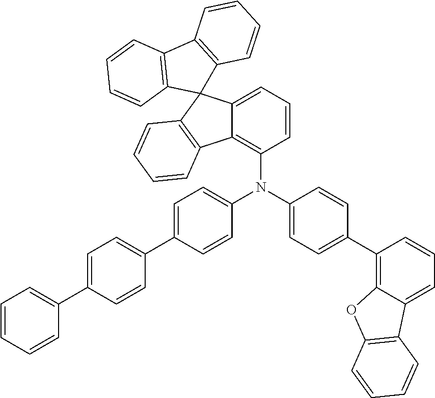 [SMILES: c1ccc(-c2ccc(-c3ccc(N(c4ccc(-c5cccc6c5oc5ccccc56)cc4)c4cccc5c4-c4ccccc4C54c5ccccc5-c5ccccc54)cc3)cc2)cc1]

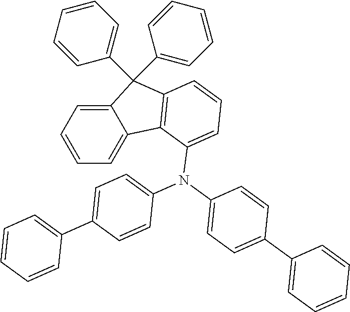 [SMILES: c1ccc(-c2ccc(N(c3ccc(-c4ccccc4)cc3)c3cccc4c3-c3ccccc3C4(c3ccccc3)c3ccccc3)cc2)cc1]

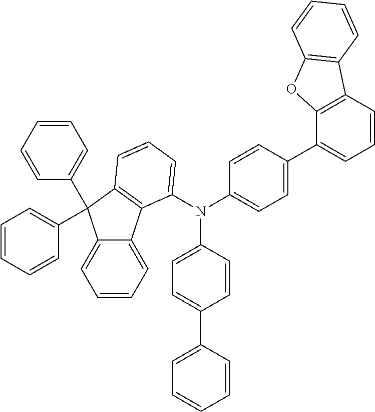 [SMILES: c1ccc(-c2ccc(N(c3ccc(-c4cccc5c4oc4ccccc45)cc3)c3cccc4c3-c3ccccc3C4(c3ccccc3)c3ccccc3)cc2)cc1]

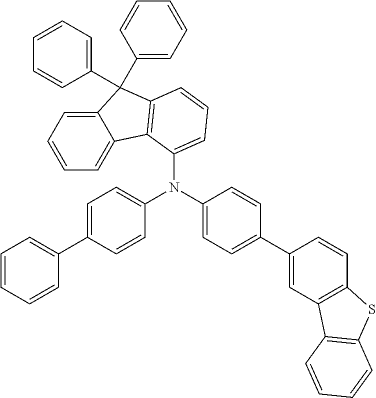 [SMILES: c1ccc(-c2ccc(N(c3ccc(-c4ccc5sc6ccccc6c5c4)cc3)c3cccc4c3-c3ccccc3C4(c3ccccc3)c3ccccc3)cc2)cc1]

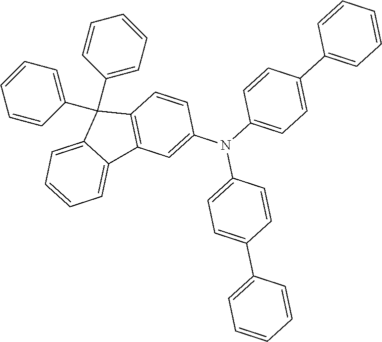 [SMILES: c1ccc(-c2ccc(N(c3ccc(-c4ccccc4)cc3)c3ccc4c(c3)-c3ccccc3C4(c3ccccc3)c3ccccc3)cc2)cc1]

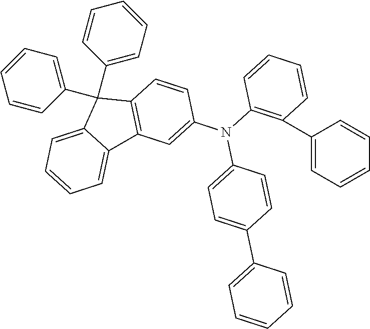 [SMILES: c1ccc(-c2ccc(N(c3ccc4c(c3)-c3ccccc3C4(c3ccccc3)c3ccccc3)c3ccccc3-c3ccccc3)cc2)cc1]

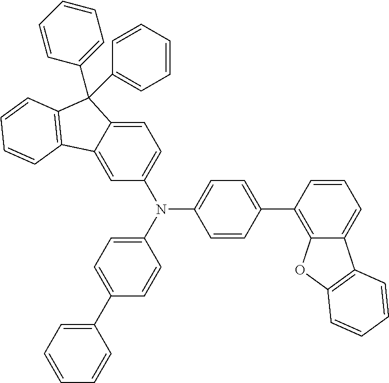 [SMILES: c1ccc(-c2ccc(N(c3ccc(-c4cccc5c4oc4ccccc45)cc3)c3ccc4c(c3)-c3ccccc3C4(c3ccccc3)c3ccccc3)cc2)cc1]

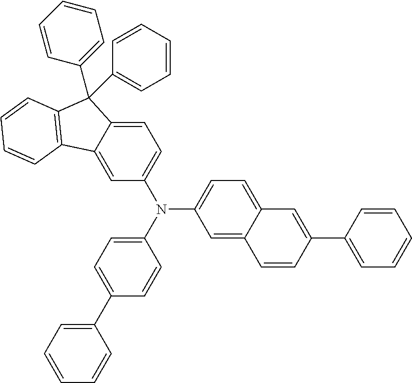 [SMILES: c1ccc(-c2ccc(N(c3ccc4c(c3)-c3ccccc3C4(c3ccccc3)c3ccccc3)c3ccc4cc(-c5ccccc5)ccc4c3)cc2)cc1]

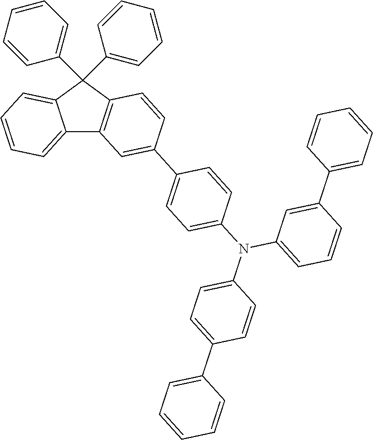 [SMILES: c1ccc(-c2ccc(N(c3ccc(-c4ccc5c(c4)-c4ccccc4C5(c4ccccc4)c4ccccc4)cc3)c3cccc(-c4ccccc4)c3)cc2)cc1]

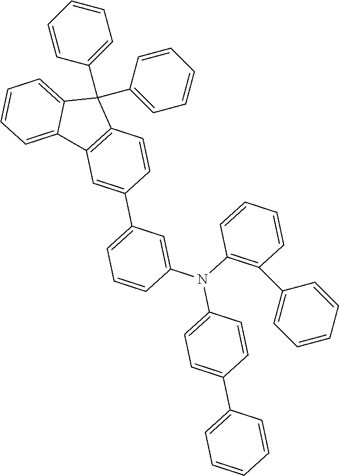 [SMILES: c1ccc(-c2ccc(N(c3cccc(-c4ccc5c(c4)-c4ccccc4C5(c4ccccc4)c4ccccc4)c3)c3ccccc3-c3ccccc3)cc2)cc1]

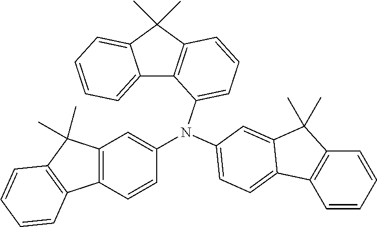 [SMILES: CC1(C)c2ccccc2-c2ccc(N(c3ccc4c(c3)C(C)(C)c3ccccc3-4)c3cccc4c3-c3ccccc3C4(C)C)cc21]

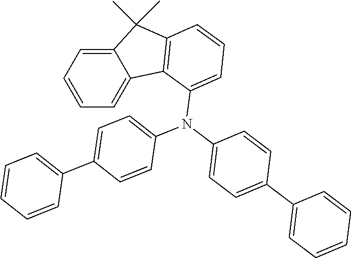 [SMILES: CC1(C)c2ccccc2-c2c(N(c3ccc(-c4ccccc4)cc3)c3ccc(-c4ccccc4)cc3)cccc21]

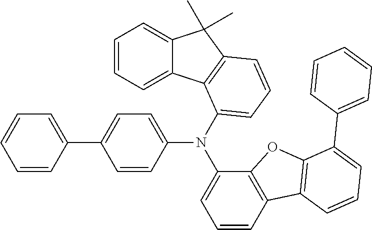 [SMILES: CC1(C)c2ccccc2-c2c(N(c3ccc(-c4ccccc4)cc3)c3cccc4c3oc3c(-c5ccccc5)cccc34)cccc21]

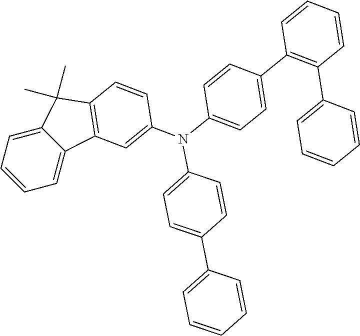 [SMILES: CC1(C)c2ccccc2-c2cc(N(c3ccc(-c4ccccc4)cc3)c3ccc(-c4ccccc4-c4ccccc4)cc3)ccc21]

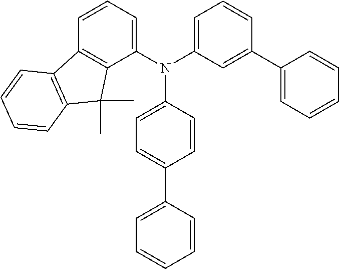 [SMILES: CC1(C)c2ccccc2-c2cccc(N(c3ccc(-c4ccccc4)cc3)c3cccc(-c4ccccc4)c3)c21]

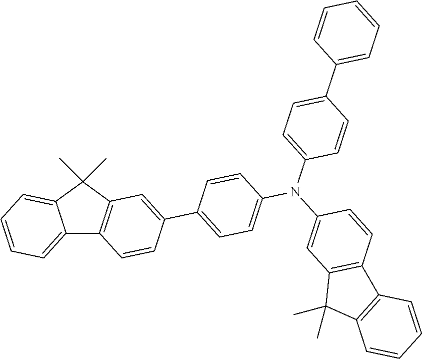 [SMILES: CC1(C)c2ccccc2-c2ccc(-c3ccc(N(c4ccc(-c5ccccc5)cc4)c4ccc5c(c4)C(C)(C)c4ccccc4-5)cc3)cc21]

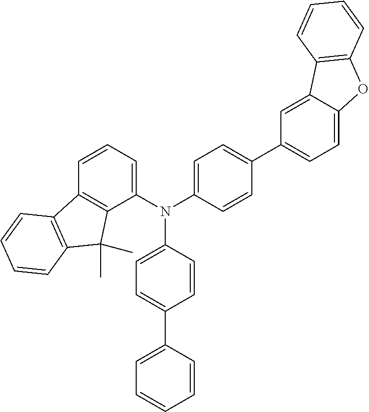 [SMILES: CC1(C)c2ccccc2-c2cccc(N(c3ccc(-c4ccccc4)cc3)c3ccc(-c4ccc5oc6ccccc6c5c4)cc3)c21]

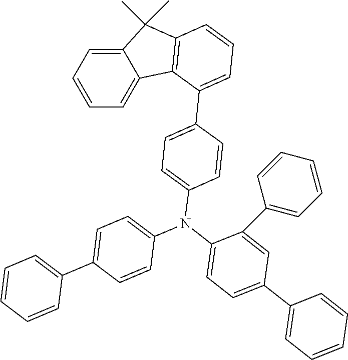 [SMILES: CC1(C)c2ccccc2-c2c(-c3ccc(N(c4ccc(-c5ccccc5)cc4)c4ccc(-c5ccccc5)cc4-c4ccccc4)cc3)cccc21]

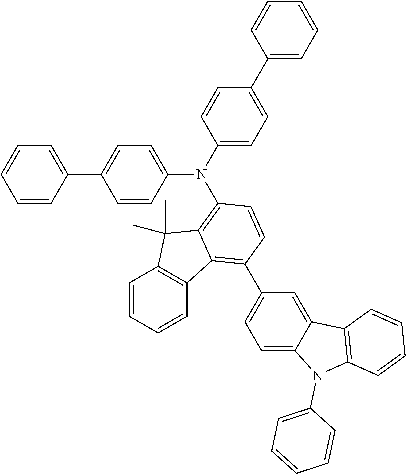 [SMILES: CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21]

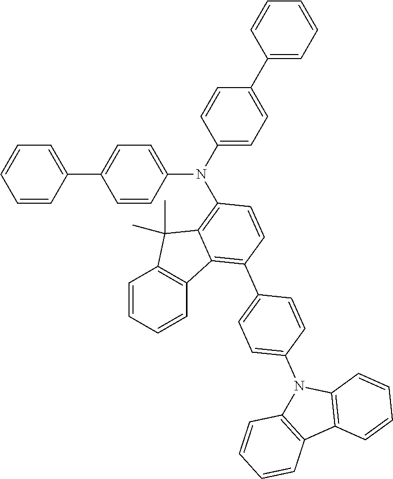 [SMILES: CC1(C)c2ccccc2-c2c(-c3ccc(-n4c5ccccc5c5ccccc54)cc3)ccc(N(c3ccc(-c4ccccc4)cc3)c3ccc(-c4ccccc4)cc3)c21]

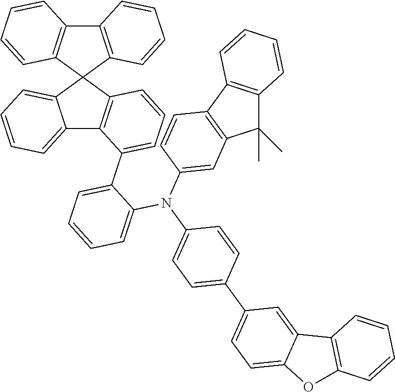 [SMILES: CC1(C)c2ccccc2-c2ccc(N(c3ccc(-c4ccc5oc6ccccc6c5c4)cc3)c3ccccc3-c3cccc4c3-c3ccccc3C43c4ccccc4-c4ccccc43)cc21]

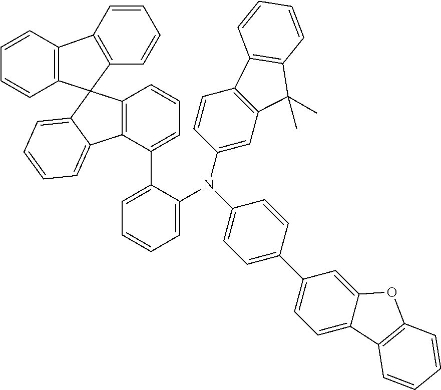 [SMILES: CC1(C)c2ccccc2-c2ccc(N(c3ccc(-c4ccc5c(c4)oc4ccccc45)cc3)c3ccccc3-c3cccc4c3-c3ccccc3C43c4ccccc4-c4ccccc43)cc21]

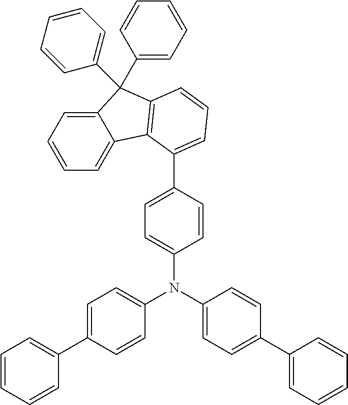 [SMILES: c1ccc(-c2ccc(N(c3ccc(-c4ccccc4)cc3)c3ccc(-c4cccc5c4-c4ccccc4C5(c4ccccc4)c4ccccc4)cc3)cc2)cc1]

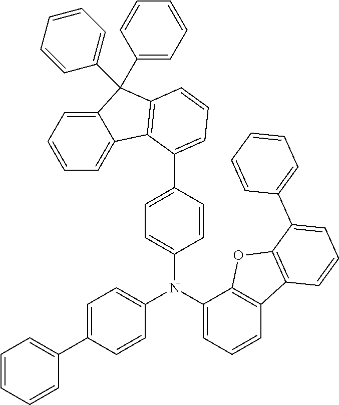 [SMILES: c1ccc(-c2ccc(N(c3ccc(-c4cccc5c4-c4ccccc4C5(c4ccccc4)c4ccccc4)cc3)c3cccc4c3oc3c(-c5ccccc5)cccc34)cc2)cc1]

Wide Bandgap Materials:

Wide bandgap materials in the context of the present application shall be understood to mean those materials as disclosed in U.S. Pat. No. 7,294,849, which are characterized in that they have a bandgap of at least 3.5 eV, the term “bandgap” referring to the energy gap between the highest occupied molecular orbital (HOMO) and the lowest unoccupied molecular orbital (LUMO). Such systems are well known in the art and have particularly advantageous performance properties in electroluminescent devices.

n-Dopants:

In the case of n-type ETLs (n-ETL), the ionization potential of the n-dopant should be above the LUMO level of electron transport matrices. The LUMO level of the n-ETLs is normally about 3.0 eV, and a higher HOMO energy (<3.0 eV) for an n-dopant is required. Possible candidates for n-dopants are alkali metals such as Li and Cs or high-lying HOMO molecules such as Ru (terpy) 2.

The introduction of an LiF layer between the electron transport layer and the contact metal has been shown to lower the operating voltage of OLEDs. This is explained by a kind of n-doping effect by released Li in Alq3.

p-Dopants:

It is further preferable when a p-doped hole transport material is applied to the anode as hole injection layer, in which case suitable p-dopants are metal oxides, for example MoO₃ or WO₃, or (per)fluorinated electron-deficient aromatic systems. Further suitable p-dopants are HAT-CN (hexacyanohexaazatriphenylene) or the compound NPD9 from Novaled. Such a layer simplifies hole injection into materials having a low HOMO, i.e. a large HOMO in terms of magnitude.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, 0-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured (for example, M. S. Arnold et al., Appl. Phys. Lett. 2008, 92,

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (I) can be used in displays, as light sources in lighting applications and as light sources in medical and/or cosmetic applications.

### EXAMPLES

### A) Synthesis Examples

The syntheses which follow, unless stated otherwise, are conducted under a protective gas atmosphere in dried solvents. The reactants can be purchased from ALDRICH (potassium fluoride (spray-dried), tri-tert-butylphosphine, palladium(II) acetate). 3-Chloro-5,6-diphenyl-1,2,4-triazine can be prepared analogously to EP 577559. 2′,7′-Di-tert-butyl-spiro-9,9″-bifluorene-2,7-bisboronic acid glycol ester can be prepared according to WO 02/077060, and 2-chloro-4,6-diphenyl-1,3,5-triazine according to U.S. Pat. No. 5,438,138. Spiro-9,9′-bifluorene-2,7-bis(boronic acid glycol ester) can be prepared analogously to WO 02/077060.

### a) 6-chloroindeno[1,2-c]thiophen-4-one 1a

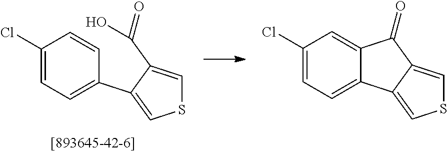 [SMILES: O=C(O)c1cscc1-c1ccc(Cl)cc1 ; O=C1c2cc(Cl)ccc2-c2cscc21]

10 g (41.9 mmol) of 4-(4-chlorophenyl)thiophene-3-carboxylic acid is suspended in 73 ml of trifluoromethanesulfonic acid and heated at 90° C. for 2 hours. After cooling, the residue is poured onto ice and extracted with ethyl acetate. After a phase separation, the solvent is removed under reduced pressure and the residue is separated by chromatography (heptane/ethyl acetate, 1:1).

The yield is 2.9 g (13.4 mmol), corresponding to 32% of theory.

### b) 6-chloro-4H-indeno[1,2-c]thiophene 1b

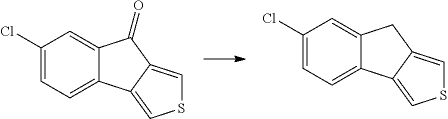 [SMILES: O=C1c2cc(Cl)ccc2-c2cscc21 ; Clc1ccc2c(c1)Cc1cscc1-2]

To a well-stirred suspension, boiling under reflux, of 6-chloroindeno[1,2-c]thiophen-4-one in a mixture of 1000 ml of toluene and 2000 ml of ethanol are added 49 ml (1000 mmol) of hydrazine hydrate and then 3 g of freshly prepared Raney nickel. After 2 h under reflux, the mixture is allowed to cool, the solvent is removed under reduced pressure, the residue is taken up in 1000 ml of warm chloroform, the solution is filtered through silica gel, the clear solution is concentrated to 100 ml, and 300 ml of ethanol is added. After the mixture had been left to stand for 12 h, the colourless crystals were filtered off with suction and then recrystallized twice from chloroform/ethanol.

Yield: 49 g (238 mmol), 96% of theory; purity: 94% by ¹H NMR.

In an analogous manner, it is possible to obtain the following compounds:

| | Reactant 1 | Product | Yield |
|---|---|---|---|
| 2b | 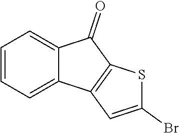 [SMILES: O=C1c2ccccc2-c2cc(Br)sc21] [1407534-82-0] | 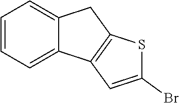 [SMILES: Brc1cc2c(s1)Cc1ccccc1-2] | 89% |
| 3b | 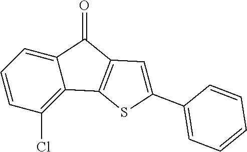 [SMILES: O=C1c2cc(-c3ccccc3)sc2-c2c(Cl)cccc21] 2d | 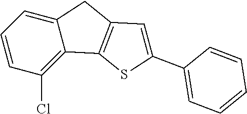 [SMILES: Clc1cccc2c1-c1sc(-c3ccccc3)cc1C2] | 875 |
| 4b | 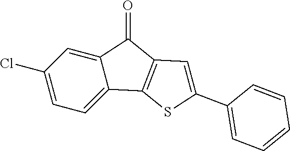 [SMILES: O=C1c2cc(Cl)ccc2-c2sc(-c3ccccc3)cc21] 3d | 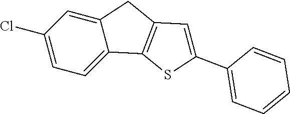 [SMILES: Clc1ccc2c(c1)Cc1cc(-c3ccccc3)sc1-2] | 90% |
| 5b | 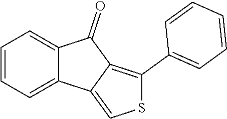 [SMILES: O=C1c2ccccc2-c2csc(-c3ccccc3)c21] [6263-90-7] | 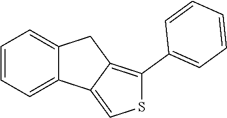 [SMILES: c1ccc(-c2scc3c2Cc2ccccc2-3)cc1] | 89% |

### c) 6-chloro-4,4-dimethylindeno[1,2-c]thiophene 1c

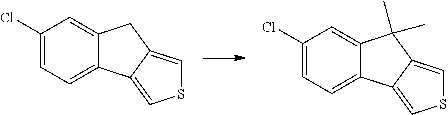

31 g (152 mmol) of 6-chloro-4H-indeno[1,2-c]thiophene is dissolved in 600 ml of dried DMSO in a baked-out flask. 43.9 g (457 mmol) of NaOtBu is added at room temperature. The now blue suspension is brought to an internal temperature of 80° C. At this temperature, 64.8 g (457 mmol) of iodomethane is added dropwise to the now violet solution at such a rate that the internal temperature does not exceed 90° C. (duration: 30 min). The mixture is stirred at internal temperature 80-90° C. for a further 30 min.

Yield: 30.5 g (132 mmol), 88% of theory; purity: 96% by ¹H NMR

In an analogous manner, it is possible to obtain the following compounds:

| | Reactant 1 | Product | Yield |
|---|---|---|---|
| 2c | 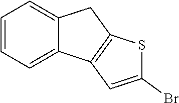 [SMILES: Brc1cc2c(s1)Cc1ccccc1-2] | 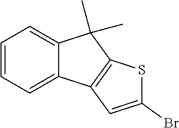 | 66% |
| 3c | 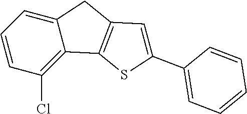 [SMILES: Clc1cccc2c1-c1sc(-c3ccccc3)cc1C2] | 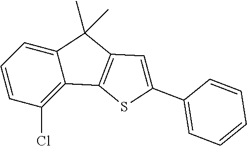 [SMILES: CC1(C)c2cc(-c3ccccc3)sc2-c2c(Cl)cccc21] | 67% |
| 4c | 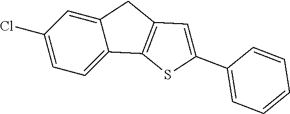 [SMILES: Clc1ccc2c(c1)Cc1cc(-c3ccccc3)sc1-2] | 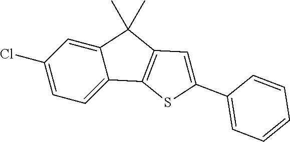 [SMILES: CC1(C)c2cc(Cl)ccc2-c2sc(-c3ccccc3)cc21] | 58% |
| 5c | 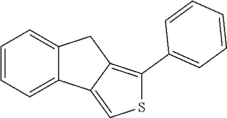 [SMILES: c1ccc(-c2scc3c2Cc2ccccc2-3)cc1] | 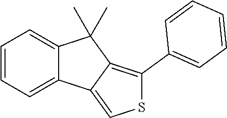 [SMILES: CC1(C)c2ccccc2-c2csc(-c3ccccc3)c21] | 60% |

### d) 6-chloro-1,3-diphenylindeno[1,2-c]thiophen-4-one 1d

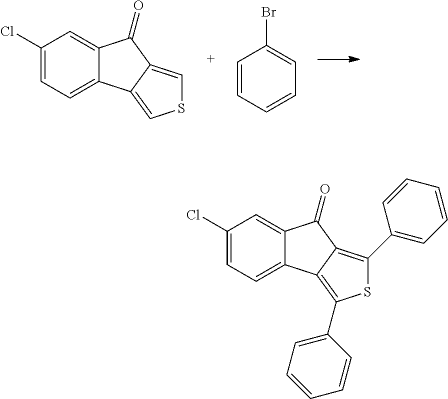 [SMILES: O=C1c2cc(Cl)ccc2-c2cscc21 ; Brc1ccccc1 ; O=C1c2cc(Cl)ccc2-c2c(-c3ccccc3)sc(-c3ccccc3)c21]

An initial charge of 4 A molecular sieve is baked out, then initially charged under protective gas with 30 g (136 mmol) of 6-chloroindeno[1,2-c]thiophen-4-one, 72 g (220 mmol) of caesium carbonate, 46 g (294 mmol) of bromobenzene, 2.2 g (7.3 mmol) of {[1,1′-biphenyl]-2-yl}di-tert-butyl)phosphine and 0.83 g (3.6 mmol) of palladium(II) acetate in 325 ml of dry DMF, and the mixture is stirred at 150° C. overnight. Thereafter, the mixture is hot-filtered through Celite, washed through with ethyl acetate and concentrated, and recrystallized in toluene/heptane.

The yield is 30.4 g (82 mmol), corresponding to 60% of theory.

In an analogous manner, it is possible to obtain the following compounds:

| | | Reactant | | |
|---|---|---|---|---|
| | Reactant 1 | 2 | Product | Yield |
| 2d | 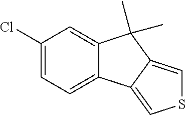 | 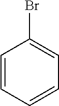 | 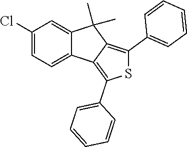 [SMILES: CC1(C)c2cc(Cl)ccc2-c2c(-c3ccccc3)sc(-c3ccccc3)c21] | 67% |
| 3d | 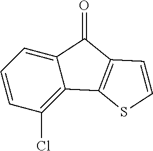 [SMILES: O=C1c2ccsc2-c2c(Cl)cccc21] | 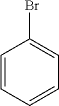 | 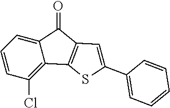 [SMILES: O=C1c2cc(-c3ccccc3)sc2-c2c(Cl)cccc21] | 57% |
| | [1367747-08-7] | | | |
| 4d | 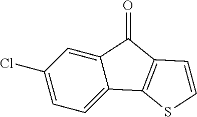 [SMILES: O=C1c2cc(Cl)ccc2-c2sccc21] | 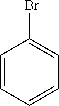 | 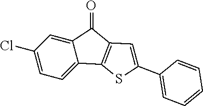 [SMILES: O=C1c2cc(Cl)ccc2-c2sc(-c3ccccc3)cc21] | 61% |
| | [1511966-22-5] | | | |
| 5d | 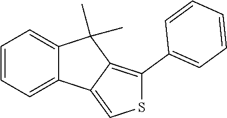 [SMILES: CC1(C)c2ccccc2-c2csc(-c3ccccc3)c21] | 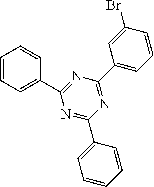 [SMILES: Brc1cccc(-c2nc(-c3ccccc3)nc(-c3ccccc3)n2)c1] [864377-31-1] | 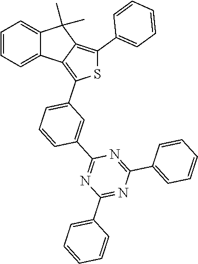 [SMILES: CC1(C)c2ccccc2-c2c(-c3cccc(-c4nc(-c5ccccc5)nc(-c5ccccc5)n4)c3)sc(-c3ccccc3)c21] | 60% |

### e) 4-bromo-1,3′-diphenyl-spiro[fluorene-9,4′-indeno[1,2-c]thiophene] 1e

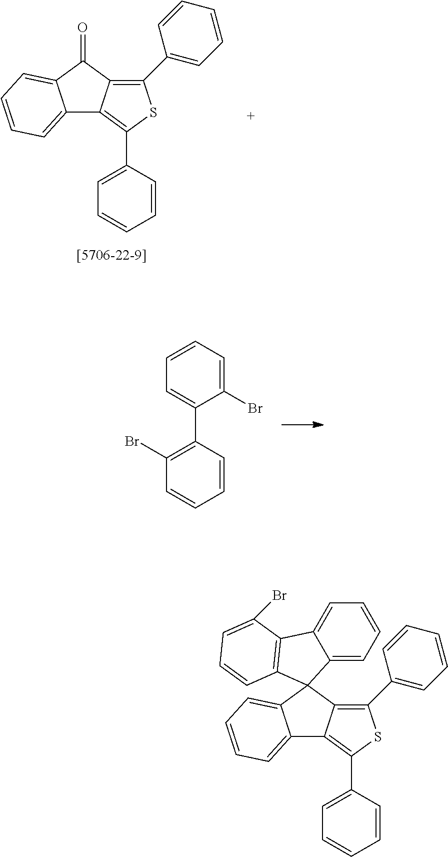 [SMILES: O=C1c2ccccc2-c2c(-c3ccccc3)sc(-c3ccccc3)c21 ; Brc1ccccc1-c1ccccc1Br ; Brc1cccc2c1-c1ccccc1C21c2ccccc2-c2c(-c3ccccc3)sc(-c3ccccc3)c21]

A 1 l four-neck flask is initially charged with 40 g (112.2 mmol) of 2,2′-dibromobiphenyl in 350 ml of THF and cooled to −78° C. By means of a dropping funnel, 49 ml (123.5 mmol) of n-butyllithium (2.5 M in n-hexane) is added dropwise at this temperature and the mixture is stirred for 1 h. Subsequently, 38 g (112 mmol) of 1,3-diphenyl-8H-indeno[1,2-c]thiophen-8-one, dissolved in 300 ml of THF, is added via a dropping funnel, and the mixture is allowed to come to room temperature overnight. Thereafter, THF is removed by rotary evaporation, then hydrolysis is effected with 500 ml of water/200 ml of ethyl acetate, followed by extractive shaking and drying. The solids are dissolved in 440 ml of toluene and boiled under reflux with 2.1 g (11.198 mmol) of toluene-4-sulfonic acid monohydrate overnight. This is followed by addition of water and ethyl acetate, separation of the phases and removal of the organic solvent on a rotary evaporator. The solids are subjected to hot extraction with n-heptane/toluene (1:1) over alumina and recrystallization in heptane/toluene.

The yield is 58.8 g (106 mmol), corresponding to 90% of theory.

In an analogous manner, it is possible to obtain the following compounds:

| | Reactant 1 | Reactant 2 | Product | Yield |
|---|---|---|---|---|
| 2e | 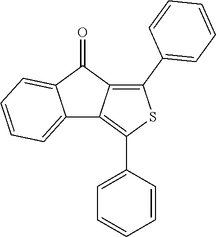 [SMILES: O=C1c2ccccc2-c2c(-c3ccccc3)sc(-c3ccccc3)c21] | 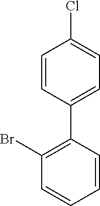 [SMILES: Clc1ccc(-c2ccccc2Br)cc1] | 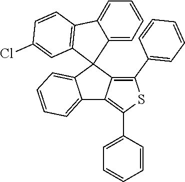 [SMILES: Clc1ccc2c(c1)C1(c3ccccc3-2)c2ccccc2-c2c(-c3ccccc3)sc(-c3ccccc3)c21] | 93% |
| | [5706-22-9] | | | |
| 3e | 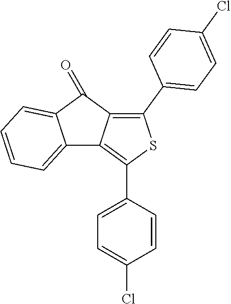 [SMILES: O=C1c2ccccc2-c2c(-c3ccc(Cl)cc3)sc(-c3ccc(Cl)cc3)c21] | 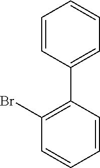 [SMILES: Brc1ccccc1-c1ccccc1] | 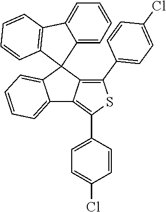 [SMILES: Clc1ccc(-c2sc(-c3ccc(Cl)cc3)c3c2-c2ccccc2C32c3ccccc3-c3ccccc32)cc1] | 89% |
| | [6072-53-3] | | | |
| 4e | 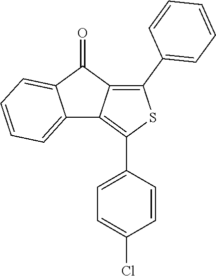 [SMILES: O=C1c2ccccc2-c2c(-c3ccc(Cl)cc3)sc(-c3ccccc3)c21] | 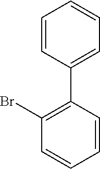 [SMILES: Brc1ccccc1-c1ccccc1] | 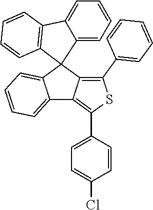 [SMILES: Clc1ccc(-c2sc(-c3ccccc3)c3c2-c2ccccc2C32c3ccccc3-c3ccccc32)cc1] | 90% |
| | [1924664-17-4] | | | |
| 5e | 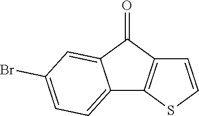 [SMILES: O=C1c2cc(Br)ccc2-c2sccc21] [1346266-91-8] | 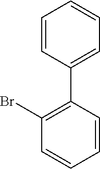 [SMILES: Brc1ccccc1-c1ccccc1] | 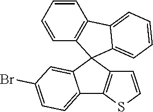 [SMILES: Brc1ccc2c(c1)C1(c3ccccc3-c3ccccc31)c1ccsc1-2] | 87% |
| 6e | 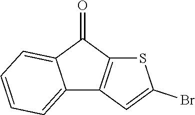 [SMILES: O=C1c2ccccc2-c2cc(Br)sc21] [1407534-82-0] | 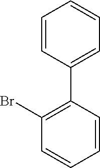 [SMILES: Brc1ccccc1-c1ccccc1] | 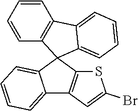 [SMILES: Brc1cc2c(s1)C1(c3ccccc3-c3ccccc31)c1ccccc1-2] | 84% |
| 7e | 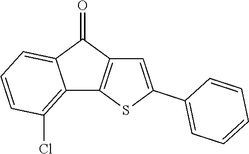 [SMILES: O=C1c2cc(-c3ccccc3)sc2-c2c(Cl)cccc21] | 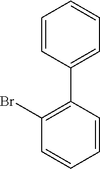 [SMILES: Brc1ccccc1-c1ccccc1] | 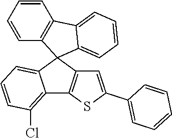 [SMILES: Clc1cccc2c1-c1sc(-c3ccccc3)cc1C21c2ccccc2-c2ccccc21] | 91% |
| 8e | 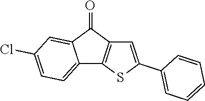 [SMILES: O=C1c2cc(Cl)ccc2-c2sc(-c3ccccc3)cc21] | 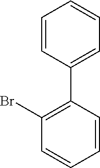 [SMILES: Brc1ccccc1-c1ccccc1] | 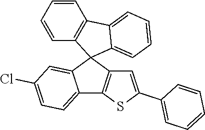 [SMILES: Clc1ccc2c(c1)C1(c3ccccc3-c3ccccc31)c1cc(-c3ccccc3)sc1-2] | 92% |

### f) 2-bromospiro[4a,8a-dihydroindeno[1,2-b]furan-4,9′-fluorene] 1f

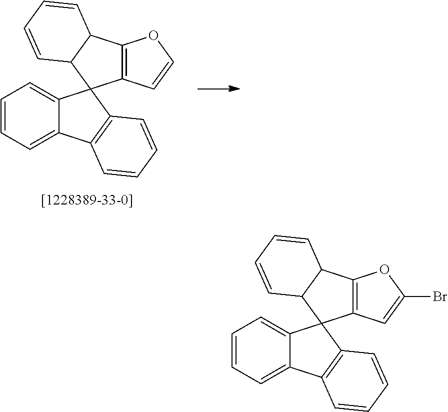 [SMILES: C1=CC2c3occc3C3(c4ccccc4-c4ccccc43)C2C=C1 ; Brc1cc2c(o1)C1C=CC=CC1C21c2ccccc2-c2ccccc21]

To a solution of 46.2 g (150 mmol) of spiro[4a,8a-dihydroindeno[1,2-b]furan-4,9′-fluorene]

in chloroform (900 ml) is added in portions, at 0° C. in the dark, N-bromosuccinimide (26.6 g, 150 mmol), and the mixture is stirred at this temperature for 2 h. The reaction is ended by addition of sodium sulfite solution and the mixture is stirred at room temperature for a further 30 min. After phase separation, the organic phase is washed with water and the aqueous phase is extracted with dichloromethane. The combined organic phases are dried over sodium sulfate and concentrated under reduced pressure. The residue is dissolved in toluene and filtered through silica gel. Subsequently, the crude product is recrystallized from toluene/heptane.

Yield: 44 g (115 mmol), 77% of theory, colourless solid.

### g) (1′,3′-diphenylspiro[fluorene-9,4′-indeno[1,2-c]thiophene]-4-yl)boronic acid 1g

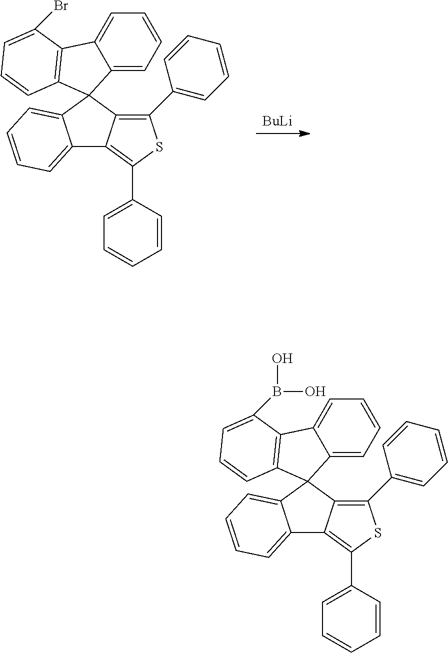

To a solution, cooled to −78° C., of 144 g (270 mmol) of 4-bromo-1′,3′-diphenyl-spiro[fluorene-9,4′-indeno[1,2-c]thiophene] in 1500 ml of diethyl ether are added dropwise 110 ml (276 mmol) of n-butyllithium (2.5 M in hexane). The reaction mixture is stirred at −78° C. for 30 min. The mixture is allowed to come to room temperature and cooled again to −78° C., and then a mixture of 40 ml (351 mmol) of trimethyl borate in 50 ml of diethyl ether is added rapidly. After warming to −10° C., hydrolysis is effected with 135 ml of 2 N hydrochloric acid. The organic phase is removed, washed with water, dried over sodium sulfate and concentrated to dryness. The residue is taken up in 300 ml of n-heptane, and the colourless solid is filtered off with suction, washed with n-heptane and dried under reduced pressure.

Yield: 135 g (262 mmol), 97% of theory; purity: 96% by HPLC.

In an analogous manner, it is possible to obtain the following compounds:

| | Reactant 1 | Product | Yield |
|---|---|---|---|
| 2g | 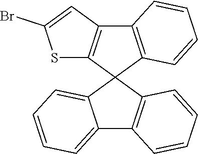 [SMILES: Brc1cc2c(s1)C1(c3ccccc3-c3ccccc31)c1ccccc1-2] [91155-62-7] | 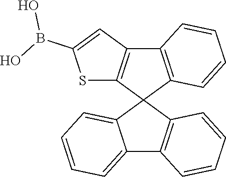 [SMILES: OB(O)c1cc2c(s1)C1(c3ccccc3-c3ccccc31)c1ccccc1-2] | 87% |
| 3g | 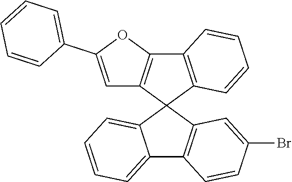 [SMILES: Brc1ccc2c(c1)C1(c3ccccc3-2)c2ccccc2-c2oc(-c3ccccc3)cc21] | 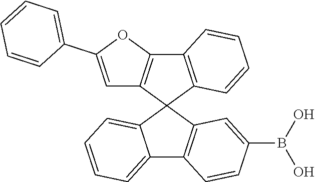 [SMILES: OB(O)c1ccc2c(c1)C1(c3ccccc3-2)c2ccccc2-c2oc(-c3ccccc3)cc21] | 84% |
| | [2291155-93-4] | | |
| 4g | 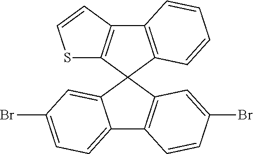 [SMILES: Brc1ccc2c(c1)C1(c3cc(Br)ccc3-2)c2ccccc2-c2ccsc21] | 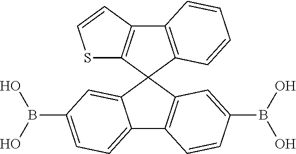 [SMILES: OB(O)c1ccc2c(c1)C1(c3cc(B(O)O)ccc3-2)c2ccccc2-c2ccsc21] | 71% |
| | [899793-65-8] | | |
| 5g | 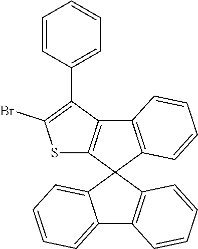 [SMILES: Brc1sc2c(c1-c1ccccc1)-c1ccccc1C21c2ccccc2-c2ccccc21] | 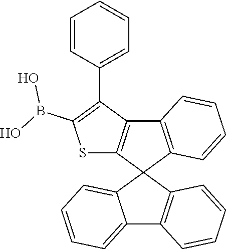 [SMILES: OB(O)c1sc2c(c1-c1ccccc1)-c1ccccc1C21c2ccccc2-c2ccccc21] | 87% |
| | [885484-69-5 | | |
| 6g | 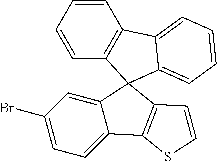 [SMILES: Brc1ccc2c(c1)C1(c3ccccc3-c3ccccc31)c1ccsc1-2] | 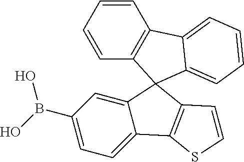 [SMILES: OB(O)c1ccc2c(c1)C1(c3ccccc3-c3ccccc31)c1ccsc1-2] | 76% |
| 7g | 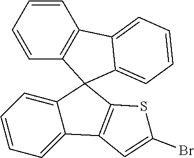 [SMILES: Brc1cc2c(s1)C1(c3ccccc3-c3ccccc31)c1ccccc1-2] | 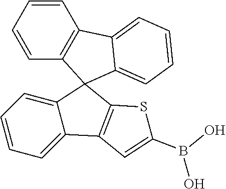 [SMILES: OB(O)c1cc2c(s1)C1(c3ccccc3-c3ccccc31)c1ccccc1-2] | 91% |
| 8g | 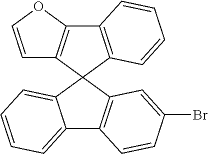 [SMILES: Brc1ccc2c(c1)C1(c3ccccc3-2)c2ccccc2-c2occc21] | 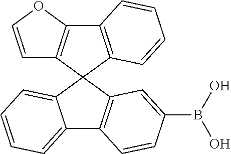 [SMILES: OB(O)c1ccc2c(c1)C1(c3ccccc3-2)c2ccccc2-c2occc21] | 77% |
| | [2291155-80-9 | | |
| 9g | 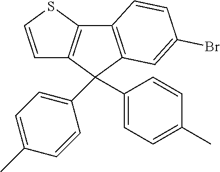 | 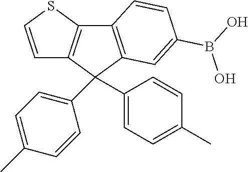 [SMILES: Cc1ccc(C2(c3ccc(C)cc3)c3cc(B(O)O)ccc3-c3sccc32)cc1] | 67% |
| | [1644610-91-2 | | |
| 10g | 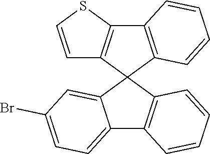 [SMILES: Brc1ccc2c(c1)C1(c3ccccc3-2)c2ccccc2-c2sccc21] | 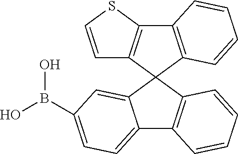 [SMILES: OB(O)c1ccc2c(c1)C1(c3ccccc3-2)c2ccccc2-c2sccc21] | 65% |
| | [2291155-62-7] | | |
| 11g | 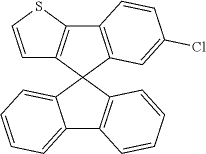 [SMILES: Clc1ccc2c(c1)C1(c3ccccc3-c3ccccc31)c1ccsc1-2] | 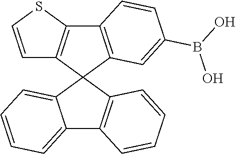 [SMILES: OB(O)c1ccc2c(c1)C1(c3ccccc3-c3ccccc31)c1ccsc1-2] | 52% |
| | [2165343-88-2 | | |
| 12g | 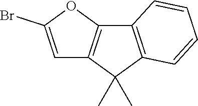 [2104697-12-10] | 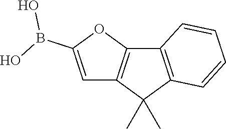 [SMILES: CC1(C)c2ccccc2-c2oc(B(O)O)cc21] | 86% |
| 13g | 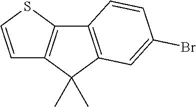 | 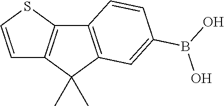 [SMILES: CC1(C)c2cc(B(O)O)ccc2-c2sccc21] | 66% |
| | [1346266-93-0] | | |
| 14g | 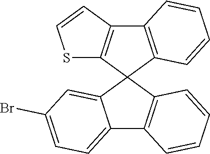 [SMILES: Brc1ccc2c(c1)C1(c3ccccc3-2)c2ccccc2-c2ccsc21] | 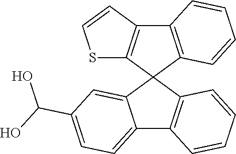 [SMILES: OC(O)c1ccc2c(c1)C1(c3ccccc3-2)c2ccccc2-c2ccsc21] | 62% |
| | [911649-11-1] | | |
| 15g | 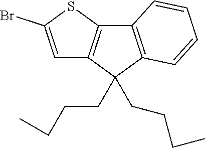 [SMILES: CCCCC1(CCCC)c2ccccc2-c2sc(Br)cc21] 1643688-07-6] | 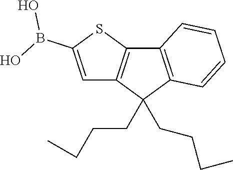 [SMILES: CCCCC1(CCCC)c2ccccc2-c2sc(B(O)O)cc21] | 87% |
| 16g | 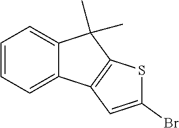 | 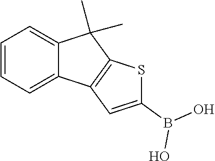 [SMILES: CC1(C)c2ccccc2-c2cc(B(O)O)sc21] | 67% |
| 17g | 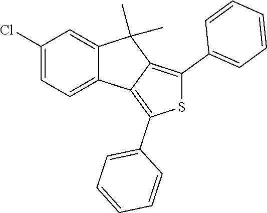 [SMILES: CC1(C)c2cc(Cl)ccc2-c2c(-c3ccccc3)sc(-c3ccccc3)c21] | 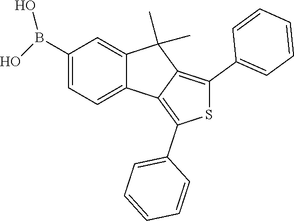 [SMILES: CC1(C)c2cc(B(O)O)ccc2-c2c(-c3ccccc3)sc(-c3ccccc3)c21] | 59% |

### h) 2-(1′,3′-diphenylspiro[fluorene-9,4′-indeno[1,2-c]thiophen]-4-yl)-4,6-diphenyl-1,3,5-triazine 1h

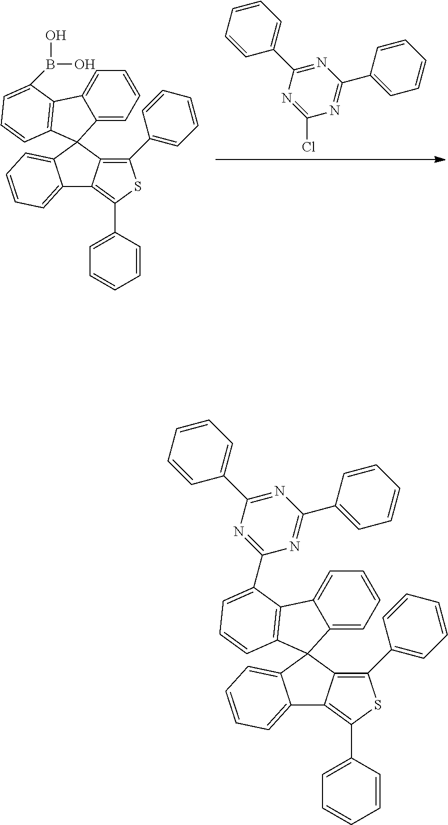 [SMILES: OB(O)c1cccc2c1-c1ccccc1C21c2ccccc2-c2c(-c3ccccc3)sc(-c3ccccc3)c21 ; Clc1nc(-c2ccccc2)nc(-c2ccccc2)n1 ; c1ccc(-c2nc(-c3ccccc3)nc(-c3cccc4c3-c3ccccc3C43c4ccccc4-c4c(-c5ccccc5)sc(-c5ccccc5)c43)n2)cc1]

57 g (110 mmol) of (1′,3′-diphenylspiro[fluorene-9,4′-indeno[1,2-c]thiophen]-4-yl)boronic acid, 30 g (110.0 mmol) of 2-chloro-4,6-diphenyl-1,3,5-triazine and 45 g (210.0 mmol) of tripotassium phosphate are suspended in 500 ml of toluene, 500 ml of dioxane and 500 ml of water. Added to this suspension are 913 mg (3.0 mmol) of tri-o-tolylphosphine and then 112 mg (0.5 mmol) of palladium(II) acetate, and the reaction mixture is heated under reflux for 16 h. After cooling, the organic phase is removed, filtered through silica gel, washed three times with 200 ml of water and then concentrated to dryness. The residue is recrystallized from toluene and from dichloromethane/iso-propanol and finally sublimed under high vacuum (p=5×10⁻⁵ mbar, T=377° C.). The yield is 63.6 g (90 mmol), corresponding to 82% of theory.

In an analogous manner, it is possible to obtain the following compounds:

| | Reactant 1 | Reactant 2 | Product | Yield |
|---|---|---|---|---|
| 2h | 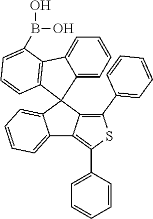 [SMILES: OB(O)c1cccc2c1-c1ccccc1C21c2ccccc2-c2c(-c3ccccc3)sc(-c3ccccc3)c21] | 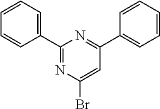 [SMILES: Brc1cc(-c2ccccc2)nc(-c2ccccc2)n1] 40734-4-5 | 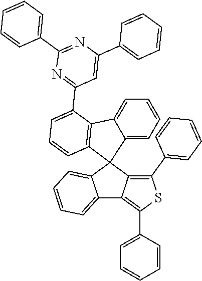 [SMILES: c1ccc(-c2cc(-c3cccc4c3-c3ccccc3C43c4ccccc4-c4c(-c5ccccc5)sc(-c5ccccc5)c43)nc(-c3ccccc3)n2)cc1] | 84% |
| 3h | 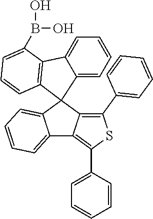 [SMILES: OB(O)c1cccc2c1-c1ccccc1C21c2ccccc2-c2c(-c3ccccc3)sc(-c3ccccc3)c21] | 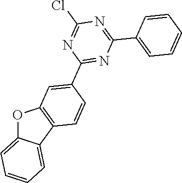 [SMILES: Clc1nc(-c2ccccc2)nc(-c2ccc3c(c2)oc2ccccc23)n1] [2142681-84-1] | 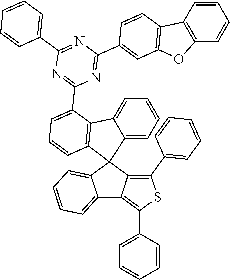 [SMILES: c1ccc(-c2nc(-c3ccc4c(c3)oc3ccccc34)nc(-c3cccc4c3-c3ccccc3C43c4ccccc4-c4c(-c5ccccc5)sc(-c5ccccc5)c43)n2)cc1] | 88% |
| 4h | 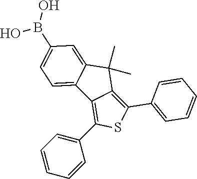 [SMILES: CC1(C)c2cc(B(O)O)ccc2-c2c(-c3ccccc3)sc(-c3ccccc3)c21] | 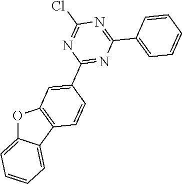 [SMILES: Clc1nc(-c2ccccc2)nc(-c2ccc3c(c2)oc2ccccc23)n1] [2142681-84-1] | 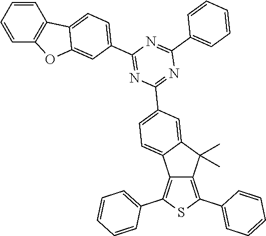 [SMILES: CC1(C)c2cc(-c3nc(-c4ccccc4)nc(-c4ccc5c(c4)oc4ccccc45)n3)ccc2-c2c(-c3ccccc3)sc(-c3ccccc3)c21] | 67% |
| 5h | 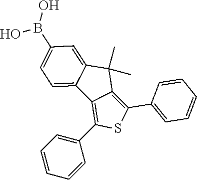 [SMILES: CC1(C)c2cc(B(O)O)ccc2-c2c(-c3ccccc3)sc(-c3ccccc3)c21] | 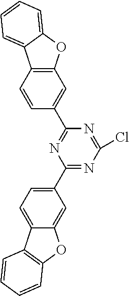 [SMILES: Clc1nc(-c2ccc3c(c2)oc2ccccc23)nc(-c2ccc3c(c2)oc2ccccc23)n1] [2251105-15-2] | 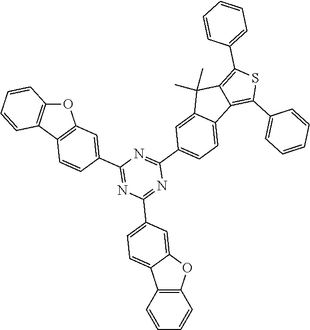 [SMILES: CC1(C)c2cc(-c3nc(-c4ccc5c(c4)oc4ccccc45)nc(-c4ccc5c(c4)oc4ccccc45)n3)ccc2-c2c(-c3ccccc3)sc(-c3ccccc3)c21] | 71% |
| 6h | 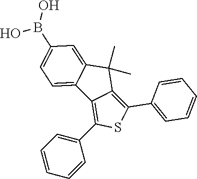 [SMILES: CC1(C)c2cc(B(O)O)ccc2-c2c(-c3ccccc3)sc(-c3ccccc3)c21] | 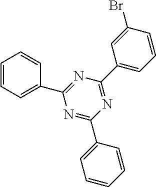 [SMILES: Brc1cccc(-c2nc(-c3ccccc3)nc(-c3ccccc3)n2)c1] [864377-31-1] | 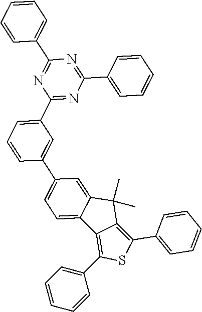 [SMILES: CC1(C)c2cc(-c3cccc(-c4nc(-c5ccccc5)nc(-c5ccccc5)n4)c3)ccc2-c2c(-c3ccccc3)sc(-c3ccccc3)c21] | 75% |
| 7h | 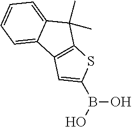 [SMILES: CC1(C)c2ccccc2-c2cc(B(O)O)sc21] | 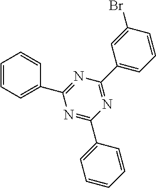 [SMILES: Brc1cccc(-c2nc(-c3ccccc3)nc(-c3ccccc3)n2)c1] [864377-31-1] | 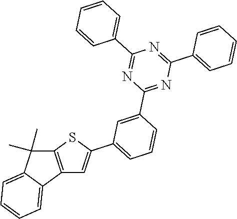 [SMILES: CC1(C)c2ccccc2-c2cc(-c3cccc(-c4nc(-c5ccccc5)nc(-c5ccccc5)n4)c3)sc21] | 79% |
| 8h | 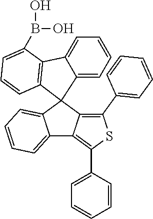 [SMILES: OB(O)c1cccc2c1-c1ccccc1C21c2ccccc2-c2c(-c3ccccc3)sc(-c3ccccc3)c21] | 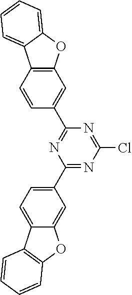 [SMILES: Clc1nc(-c2ccc3c(c2)oc2ccccc23)nc(-c2ccc3c(c2)oc2ccccc23)n1] [2251105-15-2] | 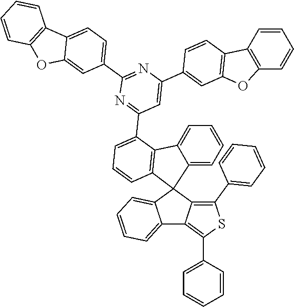 [SMILES: c1ccc(-c2sc(-c3ccccc3)c3c2-c2ccccc2C32c3ccccc3-c3c(-c4cc(-c5ccc6c(c5)oc5ccccc56)nc(-c5ccc6c(c5)oc5ccccc56)n4)cccc32)cc1] | 85% |
| 9h | 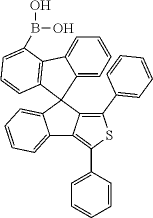 [SMILES: OB(O)c1cccc2c1-c1ccccc1C21c2ccccc2-c2c(-c3ccccc3)sc(-c3ccccc3)c21] | 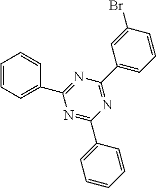 [SMILES: Brc1cccc(-c2nc(-c3ccccc3)nc(-c3ccccc3)n2)c1] [864377-31-1] | 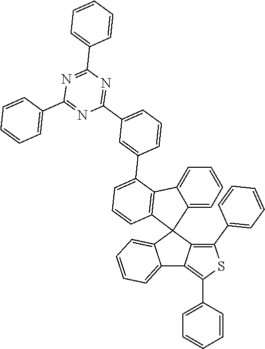 [SMILES: c1ccc(-c2nc(-c3ccccc3)nc(-c3cccc(-c4cccc5c4-c4ccccc4C54c5ccccc5-c5c(-c6ccccc6)sc(-c6ccccc6)c54)c3)n2)cc1] | 78% |
| 10h | 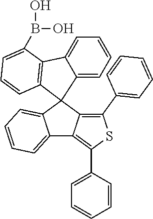 [SMILES: OB(O)c1cccc2c1-c1ccccc1C21c2ccccc2-c2c(-c3ccccc3)sc(-c3ccccc3)c21] | 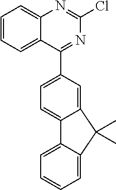 [SMILES: CC1(C)c2ccccc2-c2ccc(-c3nc(Cl)nc4ccccc34)cc21] [1373265-66-7] | 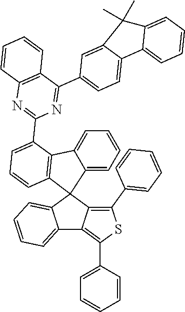 [SMILES: CC1(C)c2ccccc2-c2ccc(-c3nc(-c4cccc5c4-c4ccccc4C54c5ccccc5-c5c(-c6ccccc6)sc(-c6ccccc6)c54)nc4ccccc34)cc21] | 80% |
| 11h | 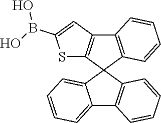 [SMILES: OB(O)c1cc2c(s1)C1(c3ccccc3-c3ccccc31)c1ccccc1-2] | 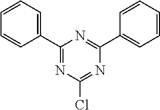 [SMILES: Clc1nc(-c2ccccc2)nc(-c2ccccc2)n1] [3842-55-5] | 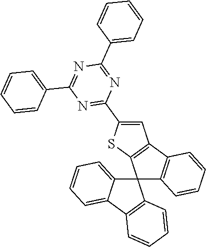 [SMILES: c1ccc(-c2nc(-c3ccccc3)nc(-c3cc4c(s3)C3(c5ccccc5-c5ccccc53)c3ccccc3-4)n2)cc1] | 81% |
| 12h | 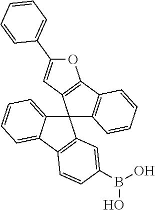 [SMILES: OB(O)c1ccc2c(c1)C1(c3ccccc3-2)c2ccccc2-c2oc(-c3ccccc3)cc21] | 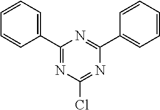 [SMILES: Clc1nc(-c2ccccc2)nc(-c2ccccc2)n1] [3842-55-5] | 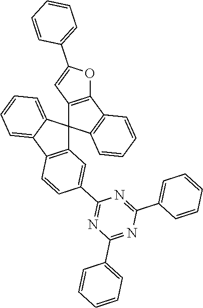 [SMILES: c1ccc(-c2nc(-c3ccccc3)nc(-c3ccc4c(c3)C3(c5ccccc5-4)c4ccccc4-c4oc(-c5ccccc5)cc43)n2)cc1] | 82% |
| 13h | 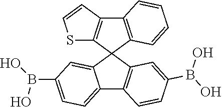 [SMILES: OB(O)c1ccc2c(c1)C1(c3cc(B(O)O)ccc3-2)c2ccccc2-c2ccsc21] | 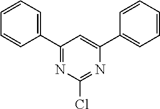 [SMILES: Clc1nc(-c2ccccc2)cc(-c2ccccc2)n1] [2915-16-4] | 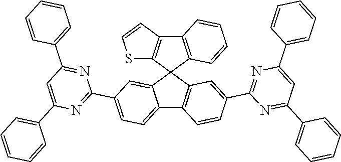 [SMILES: c1ccc(-c2cc(-c3ccccc3)nc(-c3ccc4c(c3)C3(c5cc(-c6nc(-c7ccccc7)cc(-c7ccccc7)n6)ccc5-4)c4ccccc4-c4ccsc43)n2)cc1] | 79% |
| 14h | 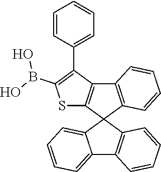 [SMILES: OB(O)c1sc2c(c1-c1ccccc1)-c1ccccc1C21c2ccccc2-c2ccccc21] | 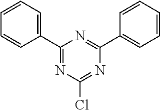 [SMILES: Clc1nc(-c2ccccc2)nc(-c2ccccc2)n1] [3842-55-5] | 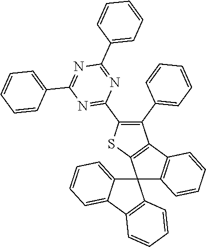 [SMILES: c1ccc(-c2nc(-c3ccccc3)nc(-c3sc4c(c3-c3ccccc3)-c3ccccc3C43c4ccccc4-c4ccccc43)n2)cc1] | 78% |
| 15h | 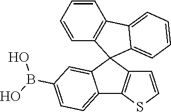 [SMILES: OB(O)c1ccc2c(c1)C1(c3ccccc3-c3ccccc31)c1ccsc1-2] | 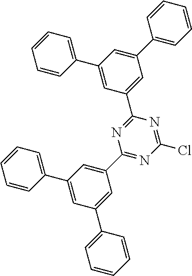 [SMILES: Clc1nc(-c2cc(-c3ccccc3)cc(-c3ccccc3)c2)nc(-c2cc(-c3ccccc3)cc(-c3ccccc3)c2)n1] [1205748-51-1] | 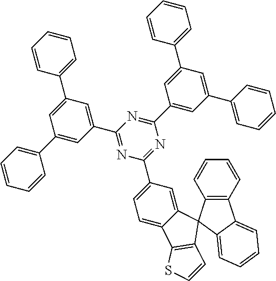 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccsc4-5)n3)c2)cc1] | 87% |
| 16h | 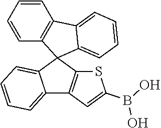 [SMILES: OB(O)c1cc2c(s1)C1(c3ccccc3-c3ccccc31)c1ccccc1-2] | 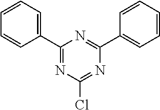 [SMILES: Clc1nc(-c2ccccc2)nc(-c2ccccc2)n1] [3842-55-5] | 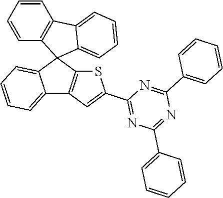 [SMILES: c1ccc(-c2nc(-c3ccccc3)nc(-c3cc4c(s3)C3(c5ccccc5-c5ccccc53)c3ccccc3-4)n2)cc1] | 72% |
| 17h | 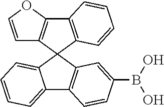 [SMILES: OB(O)c1ccc2c(c1)C1(c3ccccc3-2)c2ccccc2-c2occc21] | 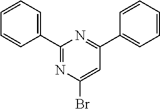 [SMILES: Brc1cc(-c2ccccc2)nc(-c2ccccc2)n1] [40734-4-5] | 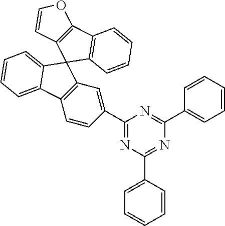 [SMILES: c1ccc(-c2nc(-c3ccccc3)nc(-c3ccc4c(c3)C3(c5ccccc5-4)c4ccccc4-c4occc43)n2)cc1] | 61% |
| 18h | 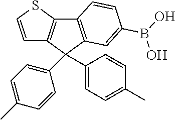 [SMILES: Cc1ccc(C2(c3ccc(C)cc3)c3cc(B(O)O)ccc3-c3sccc32)cc1] | 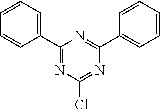 [SMILES: Clc1nc(-c2ccccc2)nc(-c2ccccc2)n1] [3842-55-5] | 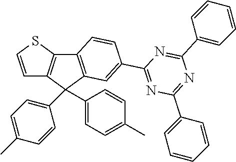 [SMILES: Cc1ccc(C2(c3ccc(C)cc3)c3cc(-c4nc(-c5ccccc5)nc(-c5ccccc5)n4)ccc3-c3sccc32)cc1] | 79% |
| 19h | 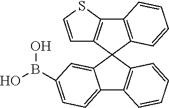 [SMILES: OB(O)c1ccc2c(c1)C1(c3ccccc3-2)c2ccccc2-c2sccc21] | 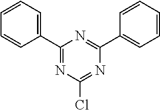 [SMILES: Clc1nc(-c2ccccc2)nc(-c2ccccc2)n1] [3842-55-5] | 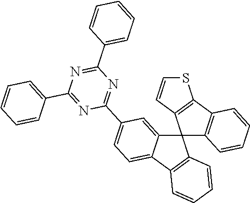 [SMILES: c1ccc(-c2nc(-c3ccccc3)nc(-c3ccc4c(c3)C3(c5ccccc5-4)c4ccccc4-c4sccc43)n2)cc1] | 75% |
| 20h | 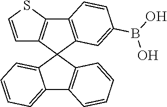 [SMILES: OB(O)c1ccc2c(c1)C1(c3ccccc3-c3ccccc31)c1ccsc1-2] | 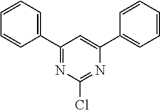 [SMILES: Clc1nc(-c2ccccc2)cc(-c2ccccc2)n1] [2915-16-4] | 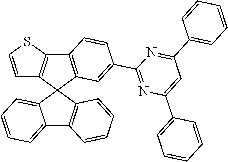 [SMILES: c1ccc(-c2cc(-c3ccccc3)nc(-c3ccc4c(c3)C3(c5ccccc5-c5ccccc53)c3ccsc3-4)n2)cc1] | 67% |
| 21h | 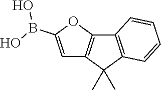 [SMILES: CC1(C)c2ccccc2-c2oc(B(O)O)cc21] | 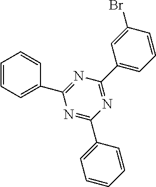 [SMILES: Brc1cccc(-c2nc(-c3ccccc3)nc(-c3ccccc3)n2)c1] [864377-31-1] | 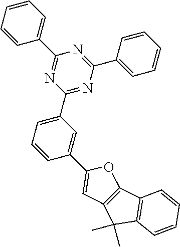 [SMILES: CC1(C)c2ccccc2-c2oc(-c3cccc(-c4nc(-c5ccccc5)nc(-c5ccccc5)n4)c3)cc21] | 82% |
| 22h | 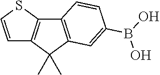 [SMILES: CC1(C)c2cc(B(O)O)ccc2-c2sccc21] | 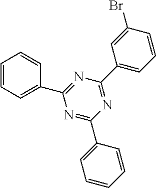 [SMILES: Brc1cccc(-c2nc(-c3ccccc3)nc(-c3ccccc3)n2)c1] [864377-31-1] | 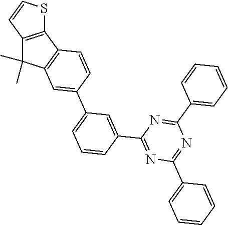 [SMILES: CC1(C)c2cc(-c3cccc(-c4nc(-c5ccccc5)nc(-c5ccccc5)n4)c3)ccc2-c2sccc21] | 76% |
| 23h | 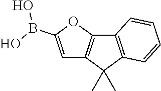 [SMILES: CC1(C)c2ccccc2-c2oc(B(O)O)cc21] | 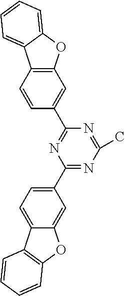 [SMILES: Cc1nc(-c2ccc3c(c2)oc2ccccc23)nc(-c2ccc3c(c2)oc2ccccc23)n1] [2251105-15-2] | 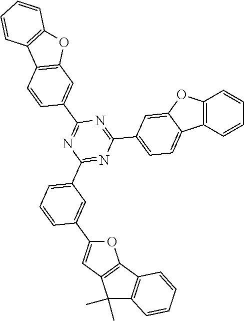 [SMILES: CC1(C)c2ccccc2-c2oc(-c3cccc(-c4nc(-c5ccc6c(c5)oc5ccccc56)nc(-c5ccc6c(c5)oc5ccccc56)n4)c3)cc21] | 71% |
| 24h | 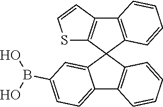 [SMILES: OB(O)c1ccc2c(c1)C1(c3ccccc3-2)c2ccccc2-c2ccsc21] | 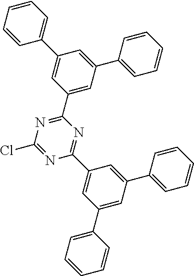 [SMILES: Clc1nc(-c2cc(-c3ccccc3)cc(-c3ccccc3)c2)nc(-c2cc(-c3ccccc3)cc(-c3ccccc3)c2)n1] [1205748-51-1] | 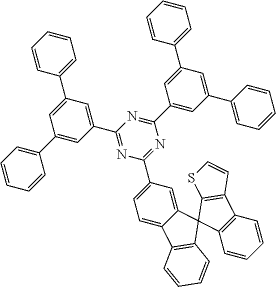 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-5)c5ccccc5-c5ccsc54)n3)c2)cc1] | 73% |
| 25h | 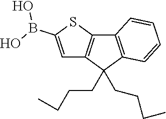 [SMILES: CCCCC1(CCCC)c2ccccc2-c2sc(B(O)O)cc21] | 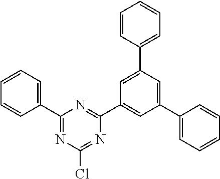 [SMILES: Clc1nc(-c2ccccc2)nc(-c2cc(-c3ccccc3)cc(-c3ccccc3)c2)n1] [1616231-59-4] | 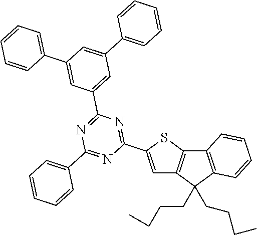 [SMILES: CCCCC1(CCCC)c2ccccc2-c2sc(-c3nc(-c4ccccc4)nc(-c4cc(-c5ccccc5)cc(-c5ccccc5)c4)n3)cc21] | 70% |
| 26h | 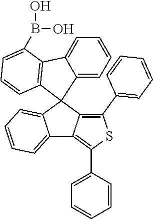 [SMILES: OB(O)c1cccc2c1-c1ccccc1C21c2ccccc2-c2c(-c3ccccc3)sc(-c3ccccc3)c21] | 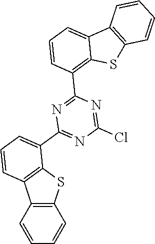 [SMILES: Clc1nc(-c2cccc3c2sc2ccccc23)nc(-c2cccc3c2sc2ccccc23)n1] [1387596-01-1] | 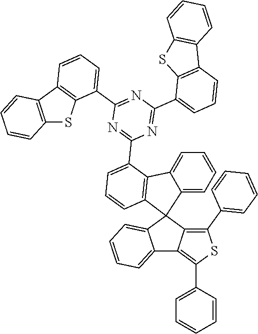 [SMILES: c1ccc(-c2sc(-c3ccccc3)c3c2-c2ccccc2C32c3ccccc3-c3c(-c4nc(-c5cccc6c5sc5ccccc56)nc(-c5cccc6c5sc5ccccc56)n4)cccc32)cc1] | 67% |
| 27h | 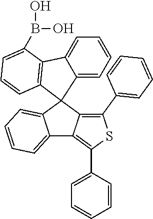 [SMILES: OB(O)c1cccc2c1-c1ccccc1C21c2ccccc2-c2c(-c3ccccc3)sc(-c3ccccc3)c21] | 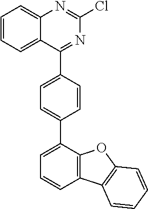 [SMILES: Clc1nc(-c2ccc(-c3cccc4c3oc3ccccc34)cc2)c2ccccc2n1] [1403252-58-3] | 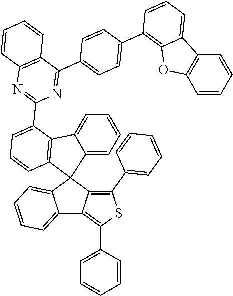 [SMILES: c1ccc(-c2sc(-c3ccccc3)c3c2-c2ccccc2C32c3ccccc3-c3c(-c4nc(-c5ccc(-c6cccc7c6oc6ccccc67)cc5)c5ccccc5n4)cccc32)cc1] | 81% |
| 28h | 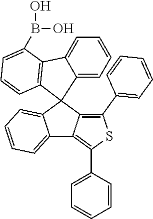 [SMILES: OB(O)c1cccc2c1-c1ccccc1C21c2ccccc2-c2c(-c3ccccc3)sc(-c3ccccc3)c21] | 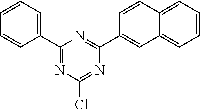 [SMILES: Clc1nc(-c2ccccc2)nc(-c2ccc3ccccc3c2)n1] [1342819-12-8] | 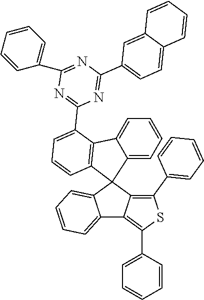 [SMILES: c1ccc(-c2nc(-c3ccc4ccccc4c3)nc(-c3cccc4c3-c3ccccc3C43c4ccccc4-c4c(-c5ccccc5)sc(-c5ccccc5)c43)n2)cc1] | 76% |
| 29h | 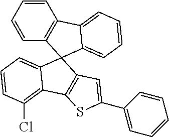 [SMILES: Clc1cccc2c1-c1sc(-c3ccccc3)cc1C21c2ccccc2-c2ccccc21] | 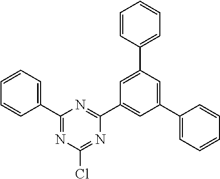 [SMILES: Clc1nc(-c2ccccc2)nc(-c2cc(-c3ccccc3)cc(-c3ccccc3)c2)n1] [1616231-59-4] | 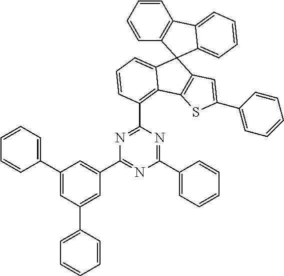 [SMILES: c1ccc(-c2cc(-c3ccccc3)cc(-c3nc(-c4ccccc4)nc(-c4cccc5c4-c4sc(-c6ccccc6)cc4C54c5ccccc5-c5ccccc54)n3)c2)cc1] | 74% |
| 30h | 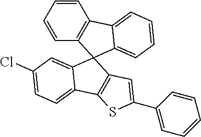 [SMILES: Clc1ccc2c(c1)C1(c3ccccc3-c3ccccc31)c1cc(-c3ccccc3)sc1-2] | 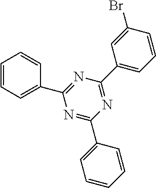 [SMILES: Brc1cccc(-c2nc(-c3ccccc3)nc(-c3ccccc3)n2)c1] [864377-31-1] | 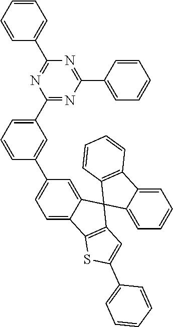 [SMILES: c1ccc(-c2nc(-c3ccccc3)nc(-c3cccc(-c4ccc5c(c4)C4(c6ccccc6-c6ccccc64)c4cc(-c6ccccc6)sc4-5)c3)n2)cc1] | 75% |
| 31h | 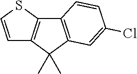 [2165343-79-1] | 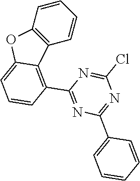 [SMILES: Clc1nc(-c2ccccc2)nc(-c2cccc3oc4ccccc4c23)n1] [1612243-82-9] | 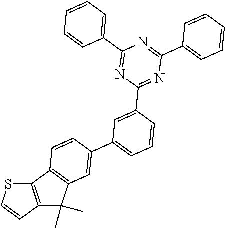 [SMILES: CC1(C)c2cc(-c3cccc(-c4nc(-c5ccccc5)nc(-c5ccccc5)n4)c3)ccc2-c2sccc21] | 72% |
| 32h | 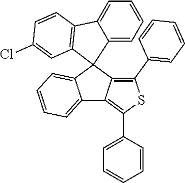 [SMILES: Clc1ccc2c(c1)C1(c3ccccc3-2)c2ccccc2-c2c(-c3ccccc3)sc(-c3ccccc3)c21] | 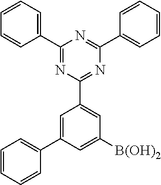 [SMILES: OB(O)c1cc(-c2ccccc2)cc(-c2nc(-c3ccccc3)nc(-c3ccccc3)n2)c1] [1518823-39-6] | 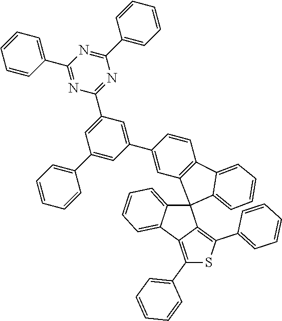 [SMILES: c1ccc(-c2cc(-c3ccc4c(c3)C3(c5ccccc5-4)c4ccccc4-c4c(-c5ccccc5)sc(-c5ccccc5)c43)cc(-c3nc(-c4ccccc4)nc(-c4ccccc4)n3)c2)cc1] | 70% |
| 33h | 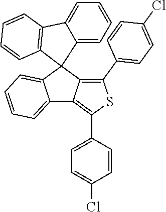 [SMILES: Clc1ccc(-c2sc(-c3ccc(Cl)cc3)c3c2-c2ccccc2C32c3ccccc3-c3ccccc32)cc1] | 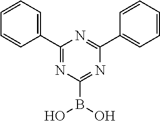 [SMILES: OB(O)c1nc(-c2ccccc2)nc(-c2ccccc2)n1] [1252825-65-6] | 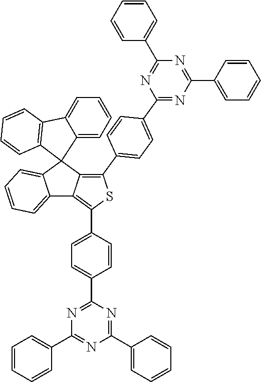 [SMILES: c1ccc(-c2nc(-c3ccccc3)nc(-c3ccc(-c4sc(-c5ccc(-c6nc(-c7ccccc7)nc(-c7ccccc7)n6)cc5)c5c4-c4ccccc4C54c5ccccc5-c5ccccc54)cc3)n2)cc1] | 69% |
| 34h | 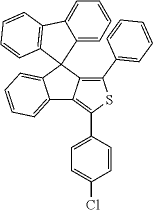 [SMILES: Clc1ccc(-c2sc(-c3ccccc3)c3c2-c2ccccc2C32c3ccccc3-c3ccccc32)cc1] | 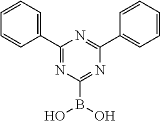 [SMILES: OB(O)c1nc(-c2ccccc2)nc(-c2ccccc2)n1] [1252825-65-6] | 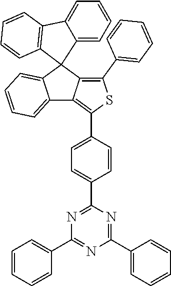 [SMILES: c1ccc(-c2nc(-c3ccccc3)nc(-c3ccc(-c4sc(-c5ccccc5)c5c4-c4ccccc4C54c5ccccc5-c5ccccc54)cc3)n2)cc1] | 81% |
| 35h | 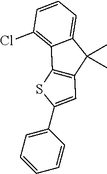 [SMILES: CC1(C)c2cc(-c3ccccc3)sc2-c2c(Cl)cccc21] | 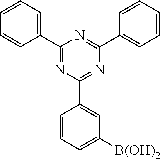 [SMILES: OB(O)c1cccc(-c2nc(-c3ccccc3)nc(-c3ccccc3)n2)c1] [1612243-82-9] | 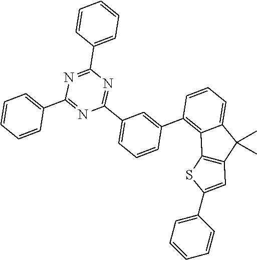 [SMILES: CC1(C)c2cc(-c3ccccc3)sc2-c2c(-c3cccc(-c4nc(-c5ccccc5)nc(-c5ccccc5)n4)c3)cccc21] | 76% |
| 36h | 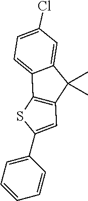 [SMILES: CC1(C)c2cc(Cl)ccc2-c2sc(-c3ccccc3)cc21] | 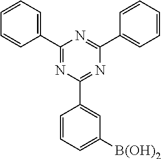 [SMILES: OB(O)c1cccc(-c2nc(-c3ccccc3)nc(-c3ccccc3)n2)c1] [1612243-82-9] | 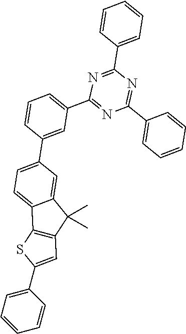 [SMILES: CC1(C)c2cc(-c3cccc(-c4nc(-c5ccccc5)nc(-c5ccccc5)n4)c3)ccc2-c2sc(-c3ccccc3)cc21] | 66% |
| 37h | 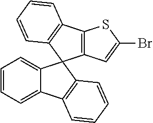 [SMILES: Brc1cc2c(s1)-c1ccccc1C21c2ccccc2-c2ccccc21] [1072830-32-0] | 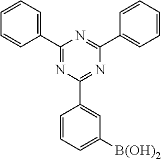 [SMILES: OB(O)c1cccc(-c2nc(-c3ccccc3)nc(-c3ccccc3)n2)c1] [1612243-82-9] | 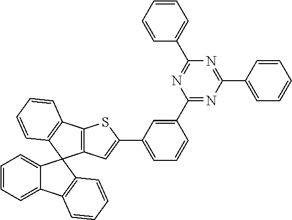 [SMILES: c1ccc(-c2nc(-c3ccccc3)nc(-c3cccc(-c4cc5c(s4)-c4ccccc4C54c5ccccc5-c5ccccc54)c3)n2)cc1] | 64% |
| 36h | 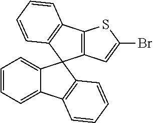 [SMILES: Brc1cc2c(s1)-c1ccccc1C21c2ccccc2-c2ccccc21] [1072830-32-0] | 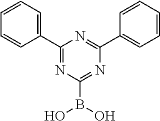 [SMILES: OB(O)c1nc(-c2ccccc2)nc(-c2ccccc2)n1] [1252825-65-6] | 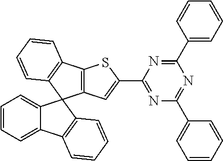 [SMILES: c1ccc(-c2nc(-c3ccccc3)nc(-c3cc4c(s3)-c3ccccc3C43c4ccccc4-c4ccccc43)n2)cc1] | 67% |
| 37h | 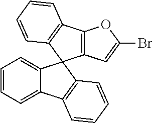 [SMILES: Brc1cc2c(o1)-c1ccccc1C21c2ccccc2-c2ccccc21] | 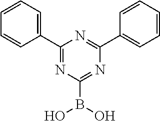 [SMILES: OB(O)c1nc(-c2ccccc2)nc(-c2ccccc2)n1] [1252825-65-6] | 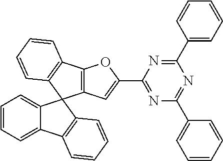 [SMILES: c1ccc(-c2nc(-c3ccccc3)nc(-c3cc4c(o3)-c3ccccc3C43c4ccccc4-c4ccccc43)n2)cc1] | 65% |
| 38h | 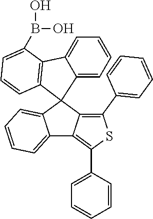 [SMILES: OB(O)c1cccc2c1-c1ccccc1C21c2ccccc2-c2c(-c3ccccc3)sc(-c3ccccc3)c21] | 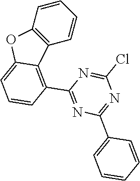 [SMILES: Clc1nc(-c2ccccc2)nc(-c2cccc3oc4ccccc4c23)n1] [1883265-32-4] | 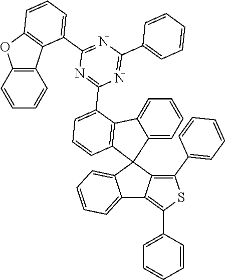 [SMILES: c1ccc(-c2nc(-c3cccc4c3-c3ccccc3C43c4ccccc4-c4c(-c5ccccc5)sc(-c5ccccc5)c43)nc(-c3cccc4oc5ccccc5c34)n2)cc1] | 83% |

### B) Device Examples

### 1) General Production Process for the OLEDs and Characterization of the OLEDs

Glass plaques which have been coated with structured ITO (indium tin oxide) in a thickness of 50 nm form the substrates to which the OLEDs are applied.

The OLEDs basically have the following layer structure: substrate/optional interlayer (IL)/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminium layer of thickness 100 nm. The exact structure of the OLEDs can be found in the tables which follow. The structure of the OLEDs produced and the materials used for production of the OLEDs are shown in tables 2 and 3 below.

All materials are applied by thermal vapour deposition in a vacuum chamber. In this case, the emission layer consists of at least one matrix material (host material) and an emitting dopant which is added to the matrix material(s) in a particular proportion by volume by co-evaporation. Details given in such a form as EG1:TER5

(97%:3%) mean here that the material EG1 is present in the layer in a proportion by volume of 97% and TER5 in a proportion of 3%. Analogously, the electron transport layer may also consist of a mixture of two materials.

The OLEDs are characterized in a standard manner. For this purpose, the electroluminescence spectra, the current efficiency (CE, measured in cd/A) and the external quantum efficiency (EQE, measured in %) are determined as a function of luminance, calculated from current-voltage-luminance characteristics assuming Lambertian emission characteristics, as is the lifetime. The electroluminescence spectra are determined at a luminance of 1000 cd/m², and the CIE 1931 x and y colour coordinates are calculated therefrom. The parameter U1000 in table 4 refers to the voltage which is required fora luminance of 1000 cd/m². CE1000 and EQE1000 respectively denote the current efficiency and external quantum efficiency that are attained at 1000 cd/m².

The lifetime LT is defined as the time after which the luminance drops from the starting luminance to a certain proportion L1 in the course of operation with constant current density j₀. A figure of L1=95% in table 4 means that the lifetime reported in the LT column corresponds to the time after which the luminance falls to 95% of its starting value.

### 2) Use and Benefit of the Inventive Compounds of the Formula (I) in OLEDs

A mixture of two host materials is typically used in the emission layer of OLEDs in order to achieve optimal charge balance and hence very good performance data of the OLED. With regard to simplified production of OLEDs, a reduction in the materials to be used is desirable. The use of just one host material in the emission layer is thus advantageous.

### 2a) Use of Compounds of the Invention in the Emission Layer of Phosphorescent Red OLEDs

By the use of the inventive compounds EG1 to EG2 in examples E1 and E2, and E5 and E6, as matrix material in the emission layer of phosphorescent red OLEDs, it is possible to show that use as a single material (E1 and E5) and particularly in a mixture with a second host material IC2 (E2 and E6) gives improved performance data of the OLEDs compared to the prior art, particularly with regard to lifetime and efficiency.

### 2b) Use of Compounds of the Invention in the Emission Layer of Phosphorescent Green OLEDs

By the use of the inventive compounds EG3 to EG6 having higher triplet energy in examples E9 to E12 as matrix material in the emission layer of phosphorescent green OLEDs, it is possible to show that use in a mixture with a second host material IC2 gives a good lifetime.

Table 4 collates the results for the performance data of the OLEDs from examples E1 to E12.

### 2c) Use of Compounds of the Invention as Electron Transport Material in the Electron Transport Layer of OLEDs

When the inventive compounds EG3 to EG6 are used as electron transport material, significantly lower voltage and better efficiency and lifetime (examples E15 to E18) are achieved than with the substance SdT1 and SdT2 according to the prior art (examples E13 to E14).

Table 5 collates the results for the performance data of the OLEDs from examples E13 to E18.

TABLE 2
Structure of the OLEDs

| | | HIL | HTL | EBL | EML | HBL | ETL | EIL |
|---|---|---|---|---|---|---|---|---|
| Ex. | IL | thickness | thickness | thickness | thickness | thickness | thickness | thickness |
| E1 | | HATCN | SpMA1 | SpMA3 | EG1:TER5 | ST2 | ST2:LiQ | LiQ 1 nm |
| | | 5 nm | 125 nm | 10 nm | (97%:3%) 35 nm | 10 nm | (50%:50%) | |
| | | | | | | | 30 nm | |
| E2 | | HATCN | SpMA1 | SpMA3 | IV1:IC2:TER5 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E3 | | HATCN | SpMA1 | SpMA3 | SdT1:TER5 | ST2 | ST2:LiQ | LiQ 1 nm |
| | | 5 nm | 125 nm | 10 nm | (97%:3%) 35 nm | 10 nm | (50%:50%) | |
| | | | | | | | 30 nm | |
| E4 | | HATCN | SpMA1 | SpMA3 | SdT1:IC2:TER5 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E5 | | HATCN | SpMA1 | SpMA3 | EG2:TER5 | ST2 | ST2:LiQ | LiQ 1 nm |
| | | 5 nm | 125 nm | 10 nm | (97%:3%) 35 nm | 10 nm | (50%:50%) | |
| | | | | | | | 30 nm | |
| E6 | | HATCN | SpMA1 | SpMA3 | EG2:IC2:TER5 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E7 | | HATCN | SpMA1 | SpMA3 | SdT2:TER5 | ST2 | ST2:LiQ | LiQ 1 nm |
| | | 5 nm | 125 nm | 10 nm | (97%:3%) 35 nm | 10 nm | (50%:50%) | |
| | | | | | | | 30 nm | |
| E8 | | HATCN | SpMA1 | SpMA3 | SdT2:IC2:TER5 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E9 | | HATCN | SpMA1 | SpMA3 | EG3:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E10 | | HATCN | SpMA1 | SpMA3 | EG4:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E11 | | HATCN | SpMA1 | SpMA3 | EG5:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E12 | | HATCN | SpMA1 | SpMA3 | EG6:IC2:TEG1 | ST2 | ST2:LiQ | LiQ 1 nm |
| | | 5 nm | 125 nm | 10 nm | (44%:44%:12%) | 10 nm | (50%:50%) | |
| | | | | | 30 nm | | 30 nm | |
| E13 | SpA1 | | HATCN | SpMA1 | M2:SEB | — | SdT1:LiQ | — |
| | 140 nm | | 5 nm | 20 nm | (95%:5%) 20 nm | | (50%:50%) | |
| | | | | | | | 30 nm | |
| E14 | SpA1 | | HATCN | SpMA1 | IC1:TEG1 | IC1 | SdT2:LiQ | — |
| | 70 nm | | 5 nm | 90 nm | (90%:10%) 30 nm | 10 nm | (50%:50%) | |
| | | | | | | | 30 nm | |
| E15 | SpA1 | | HATCN | SpMA1 | M2:SEB | — | EG4:LiQ | — |
| | 140 nm | | 5 nm | 20 nm | (95%:5%) 20 nm | | (50%:50%) | |
| | | | | | | | 30 nm | |
| E16 | SpA1 | | HATCN | SpMA1 | IC1:TEG1 | IC1 | EG4:LiQ | — |
| | 70 nm | | 5 nm | 90 nm | (90%:10%) 30 nm | 10 nm | (50%:50%) | |
| | | | | | | | 30 nm | |
| E17 | SpA1 | | HATCN | SpMA1 | M2:SEB | — | EG5:LiQ | — |
| | 140 nm | | 5 nm | 20 nm | (95%:5%) 20 nm | | (50%:50%) | |
| | | | | | | | 30 nm | |
| E18 | SpA1 | | HATCN | SpMA1 | IC1:TEG1 | — | EG6:LiQ | — |
| | 70 nm | | 5 nm | 90 nm | (90%:10%) 30 nm | | (50%:50%) | |
| | | | | | | | 40 nm | |

TABLE 3
Structural formulae of the materials for the OLEDs

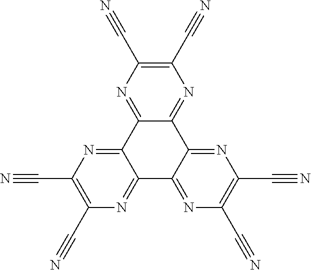 [SMILES: N#Cc1nc2c3nc(C#N)c(C#N)nc3c3nc(C#N)c(C#N)nc3c2nc1C#N]

HATCN

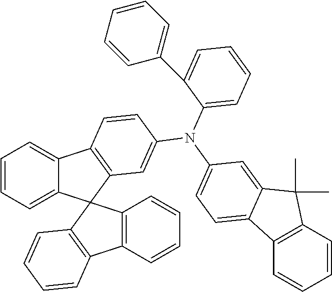 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

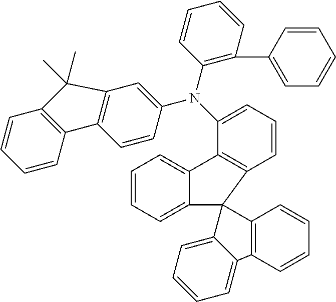 [SMILES: CC1(C)c2ccccc2-c2ccc(N(c3ccccc3-c3ccccc3)c3cccc4c3-c3ccccc3C43c4ccccc4-c4ccccc43)cc21]

SpMA3

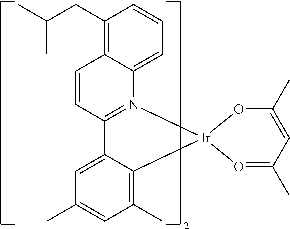

TER5

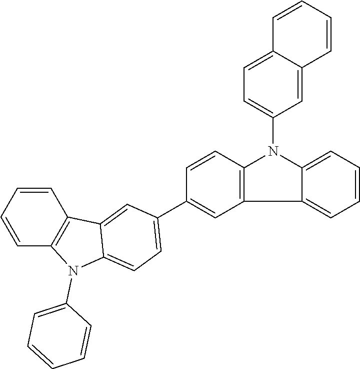 [SMILES: c1ccc(-n2c3ccccc3c3cc(-c4ccc5c(c4)c4ccccc4n5-c4ccc5ccccc5c4)ccc32)cc1]

IC2

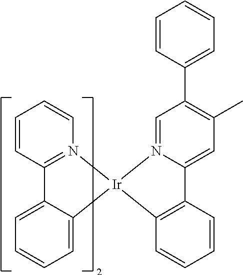

TEG1

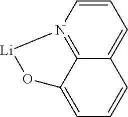

LiQ

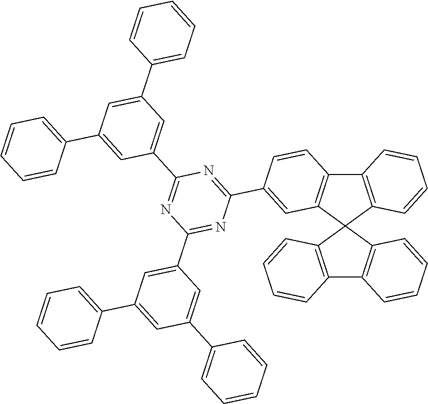 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

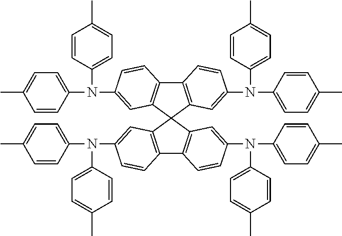

SpA1

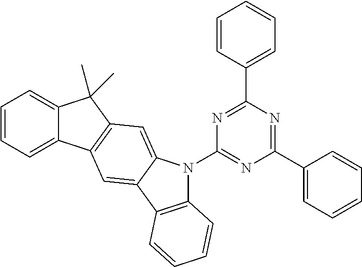 [SMILES: CC1(C)c2ccccc2-c2cc3c4ccccc4n(-c4nc(-c5ccccc5)nc(-c5ccccc5)n4)c3cc21]

IC1

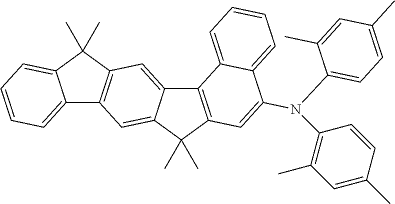 [SMILES: Cc1ccc(N(c2ccc(C)cc2C)c2cc3c(c4ccccc24)-c2cc4c(cc2C3(C)C)-c2ccccc2C4(C)C)c(C)c1]

SEB

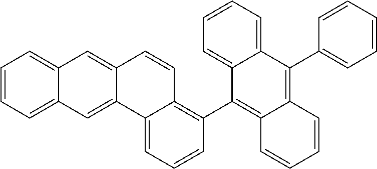 [SMILES: c1ccc(-c2c3ccccc3c(-c3cccc4c3ccc3cc5ccccc5cc34)c3ccccc23)cc1]

M2

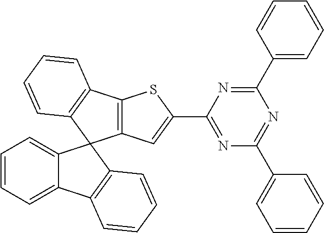 [SMILES: c1ccc(-c2nc(-c3ccccc3)nc(-c3cc4c(s3)-c3ccccc3C43c4ccccc4-c4ccccc43)n2)cc1]

EG1 (36 h)

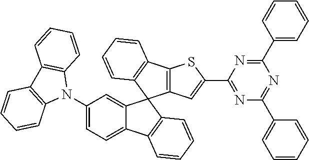 [SMILES: c1ccc(-c2nc(-c3ccccc3)nc(-c3cc4c(s3)-c3ccccc3C43c4ccccc4-c4ccc(-n5c6ccccc6c6ccccc65)cc43)n2)cc1]

SdT1

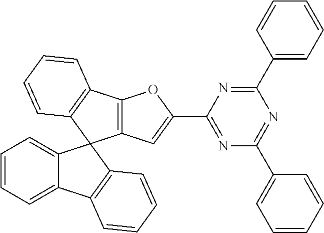 [SMILES: c1ccc(-c2nc(-c3ccccc3)nc(-c3cc4c(o3)-c3ccccc3C43c4ccccc4-c4ccccc43)n2)cc1]

EG2 (37 h)

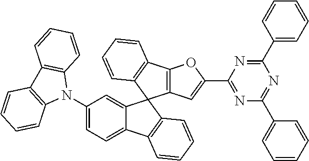 [SMILES: c1ccc(-c2nc(-c3ccccc3)nc(-c3cc4c(o3)-c3ccccc3C43c4ccccc4-c4ccc(-n5c6ccccc6c6ccccc65)cc43)n2)cc1]

SdT2

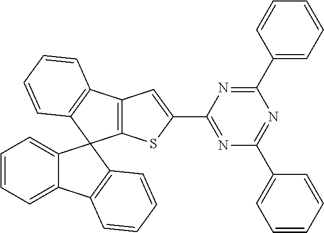 [SMILES: c1ccc(-c2nc(-c3ccccc3)nc(-c3cc4c(s3)C3(c5ccccc5-c5ccccc53)c3ccccc3-4)n2)cc1]

EG3 (11 h)

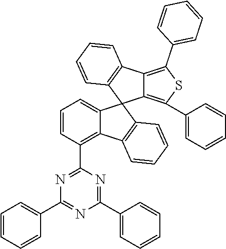 [SMILES: c1ccc(-c2nc(-c3ccccc3)nc(-c3cccc4c3-c3ccccc3C43c4ccccc4-c4c(-c5ccccc5)sc(-c5ccccc5)c43)n2)cc1]

EG4 (1 h)

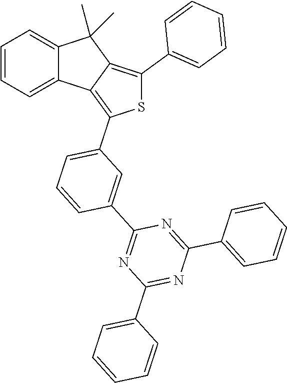 [SMILES: CC1(C)c2ccccc2-c2c(-c3cccc(-c4nc(-c5ccccc5)nc(-c5ccccc5)n4)c3)sc(-c3ccccc3)c21]

EG5 (5 d)

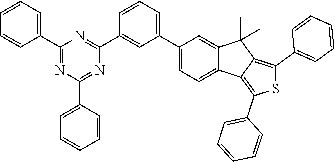 [SMILES: CC1(C)c2cc(-c3cccc(-c4nc(-c5ccccc5)nc(-c5ccccc5)n4)c3)ccc2-c2c(-c3ccccc3)sc(-c3ccccc3)c21]

EG6 (6 h)

TABLE 4
Data of the OLEDs

| | U1000 | SE1000 | EQE 1000 | CIE x/y at | j₀ | L1 | LT |
|---|---|---|---|---|---|---|---|
| Ex. | (V) | (cd/A) | (%) | 1000 cd/m² | (mA/cm²) | (%) | (h) |
| E1 | 4.3 | 23 | 15 | 0.67/0.33 | 20 | 95 | 500 |
| E2 | 3.3 | 23 | 20 | 0.66/0.34 | 20 | 95 | 1110 |
| E3 | 3.9 | 23 | 14 | 0.66/0.33 | 20 | 95 | 410 |
| E4 | 3.4 | 24 | 15 | 0.67/0.34 | 20 | 95 | 840 |
| E5 | 3.9 | 23 | 20 | 0.66/0.33 | 20 | 95 | 830 |
| E6 | 3.4 | 23 | 21 | 0.66/0.34 | 20 | 95 | 1000 |
| E7 | 3.8 | 23 | 15 | 0.67/0.33 | 20 | 95 | 540 |
| E8 | 3.8 | 24 | 18 | 0.67/0.33 | 20 | 95 | 760 |
| E9 | 3.5 | 70 | 17.5 | 0.32/0.64 | 20 | 80 | 700 |
| E10 | 3.2 | 67 | 19.1 | 0.33/0.63 | 20 | 80 | 820 |
| E11 | 3.1 | 69 | 18.8 | 0.32/0.64 | 20 | 80 | 790 |
| E12 | 3.2 | 74 | 18.5 | 0.32/0.63 | 20 | 80 | 766 |

TABLE 5
Data of the OLEDs

| | | | | | CIE x/y | | | |
|---|---|---|---|---|---|---|---|---|
| | U1000 | CE1000 | LE1000 | EQE | at 1000 | | L₁ | LT |
| Ex. | (V) | (cd/A) | (lm/W) | 1000 | cd/m² | L₀; j₀ | % | (h) |
| E13 | 6 | 8 | 5 | 7.0% | 0.13/ | 6000 | 80 | 30 |
| | | | | | 0.14 | cd/m² | | |
| E14 | 5.0 | 62 | 53 | 13% | 0.31/ | 20 mA/ | 80 | 50 |
| | | | | | 0.64 | cm² | | |
| E15 | 4.1 | 8 | 5 | 7.0% | 0.13/ | 6000 | 80 | 45 |
| | | | | | 0.14 | cd/m² | | |
| E16 | 3.6 | 62 | 53 | 17.4% | 0.31/ | 20 mA/ | 80 | 142 |
| | | | | | 0.64 | cm² | | |
| E17 | 3.5 | 8 | 6 | 7.3% | 0.14/ | 6000 | 80 | 47 |
| | | | | | 0.13 | cd/m² | | |
| E18 | 3.2 | 62 | 51 | 16.1% | 0.34/ | 20 mA/ | 80 | 124 |
| | | | | | 0.62 | cm² | | |

1.-29. (canceled)

30. Compound of formula (I) 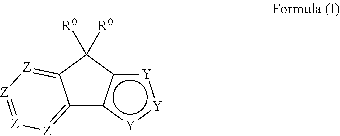
where:
Y is the same or different at each instance and is selected from O, S, NAr⁰ and CAr¹, where there must be at least one Y present which is selected from O, S and NAr⁰;
Z is the same or different at each instance and is selected from N and CR¹;
Ar⁰ is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
Ar¹ is the same or different at each instance and is selected from H, D, F, Cl, Br, I, C(═O)R², CN, Si(R²)₃, P(═O)(R²)₂, OR², S(═O)R², S(═O)₂R², straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R² radicals; and where one or more CH₂ groups in the alkyl, alkoxy, alkenyl and alkynyl groups mentioned may be replaced by —R²C═CR²—, —C≡C—, Si(R²)₂, C═O, C═NR², —C(═O)O—, —C(═O)NR²—, NR², P(═P)(R²), —O—, —S—, SO or SO₂, with exclusion of joining of two AO groups to one another to form a mono- or polycyclic, aliphatic or aromatic ring system;
R⁰ is the same or different at each instance and is selected from H, D, F, Cl, Br, I, C(═O)R¹, CN, Si(R¹)₃, N(R¹)₂, P(═O)(R¹)₂, OR¹, S(═O)R¹, S(═O)₂R¹, straight-chain alkyl, alkoxy or thioalkyl groups having 1 to 20 carbon atoms, branched or cyclic alkyl, alkoxy or thioalkyl groups having 3 to 20 carbon atoms, alkenyl or alkynyl groups having 2 to 20 carbon atoms, aromatic ring systems having 6 to 40 aromatic ring atoms, heteroaromatic ring systems having 5 to 40 aromatic ring atoms, aryloxy or heteroaryloxy groups having 5 to 40 aromatic ring atoms, and aralkyl groups having 5 to 40 aromatic ring atoms; where the two R⁰ radicals may be joined to one another and may form an aliphatic or heteroaliphatic ring, so as to form a spiro compound in position 8 of the fluorene derivative, especially a spirobifluorene derivative; where the alkyl, alkoxy, thioalkyl, alkenyl, alkynyl, aryloxy, heteroaryloxy and aralkyl groups mentioned and the aromatic ring systems and heteroaromatic ring systems mentioned are each substituted by R¹ radicals; and where one or more CH₂ groups in the alkyl, alkoxy, thioalkyl, alkenyl, alkynyl, aryloxy, heteroaryloxy and aralkyl groups mentioned may be replaced by —R¹C═CR¹—, —C≡C—, Si(R¹)₂, C═O, C═NR¹, —C(═O)O—, —C(═O)NR¹—, P(═O)(R¹), —O—, —S—, SO or SO₂;
R¹ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form an aliphatic or heteroaliphatic ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and CN; and
in the formula (I), at least one R¹ radical, one Ar⁰ group or one Ar¹ group is substituted by an A group conforming to a formula (A): 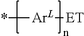
where:
* denotes the bond to the structure of the formula (I);
Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
ET is the same or different at each instance and is an electron-transporting group selected from electron-deficient heteroaromatic groups to which one or more Ar² groups are bonded;
Ar² is the same or different at each instance and is selected from H, D, F, Cl, Br, I, C(═O)R³, CN, Si(R³)₃, P(═O)(R³)₂, OR³, S(═O)R³, S(═O)₂R³, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R³ radicals; where one or more CH₂ groups in the alkyl, alkoxy, alkenyl and alkynyl groups mentioned may be replaced by —R³C═CR³—, Si(R³)₂, C═O, C═NR³, —C(═O)O—, —C(═O)NR³—, NR³, P(═O)(R³), —O—, —S—, SO or SO₂; and where two or more adjacent Ar² groups together may form a mono- or polycyclic, aliphatic or aromatic ring system;
R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂, and
n is 0 or 1.

31. Compound according to claim 30, characterized in that exactly one Y is selected from O, S and NAr⁰, and two Y are CAr¹.

32. Compound according to claim 30, characterized in that exactly one Y is selected from O and S, and two Y are CAr¹.

33. Compound according to claim 30, characterized in that the Z groups are CR¹.

34. Compound according to claim 30, characterized in that Ar¹ is the same or different at each instance and is selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, triazinyl-substituted phenyl, where the groups mentioned are each substituted by R² radicals, and H.

35. Compound according to claim 30, characterized in that R¹ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals.

36. Compound according to claim 30, characterized in that Ar^{L} is the same or different at each instance and is selected from divalent groups derived from benzene, biphenyl, terphenyl, naphthalene, fluorene, indenofluorene, indenocarbazole, spirobifluorene, dibenzofuran and dibenzothiophene, each of which are substituted by R² radicals.

37. Compound according to claim 30, characterized in that the ET group is the same or different at each instance and is selected from heteroaryl groups which have 5 to 40 aromatic ring atoms and are substituted by Ar² groups.

38. Compound according to claim 30, characterized in that the ET group is the same or different at each instance and is selected from the groups of the formulae (ET-1) to (11C): 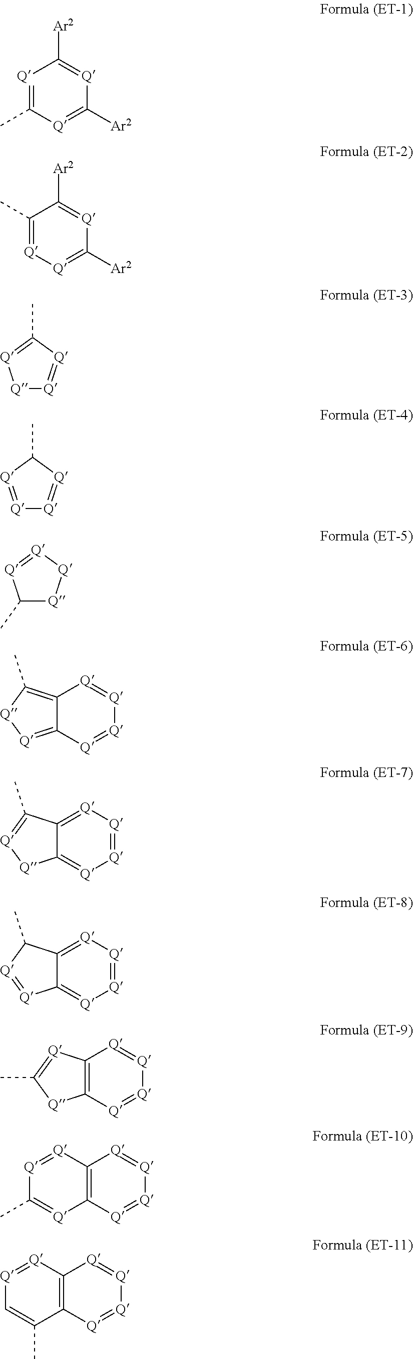 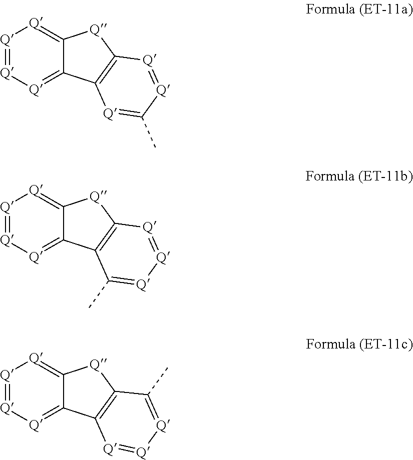
where
Q′ is the same or different at each instance and is selected from CAr² or N;
Q″ is the same or different at each instance and is selected from NR², O or S;
the dotted line in each case marks the attachment position to the rest of the formula (I), and R² and Ar² are as defined in claim 30; and
where at least one Q′ is N.

39. Compound according to claim 30, characterized in that the ET group is the same or different at each instance and is selected from the groups of the formulae (ET-12) to (ET-35) 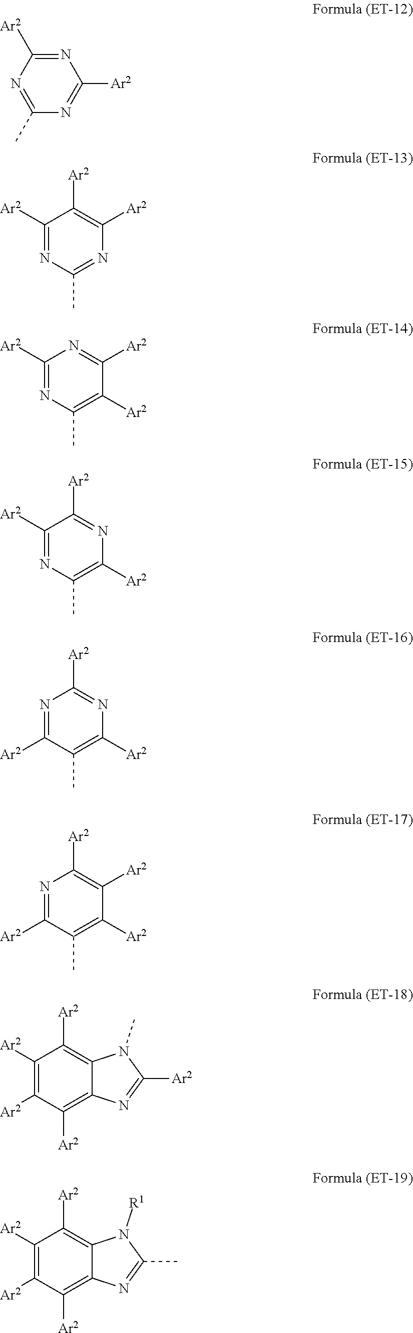 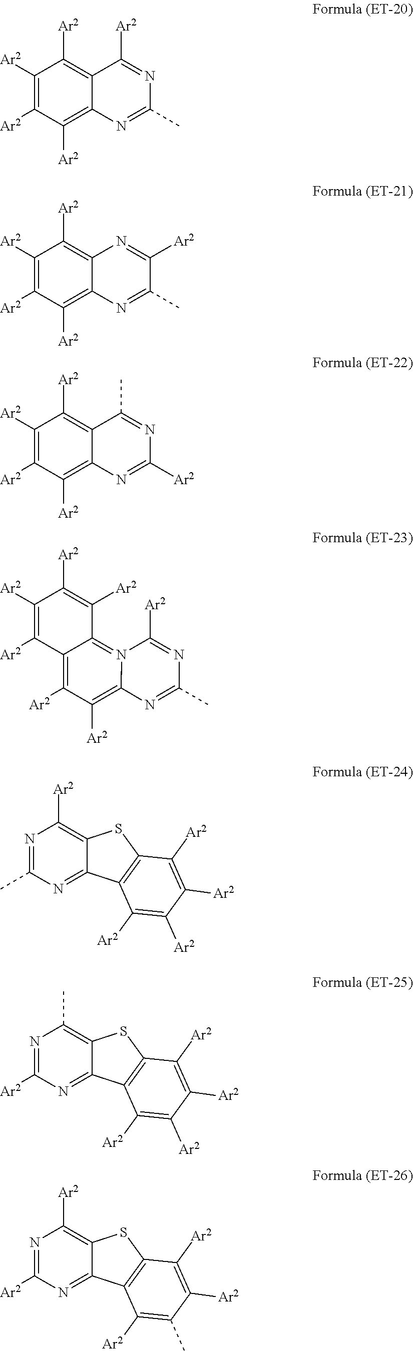 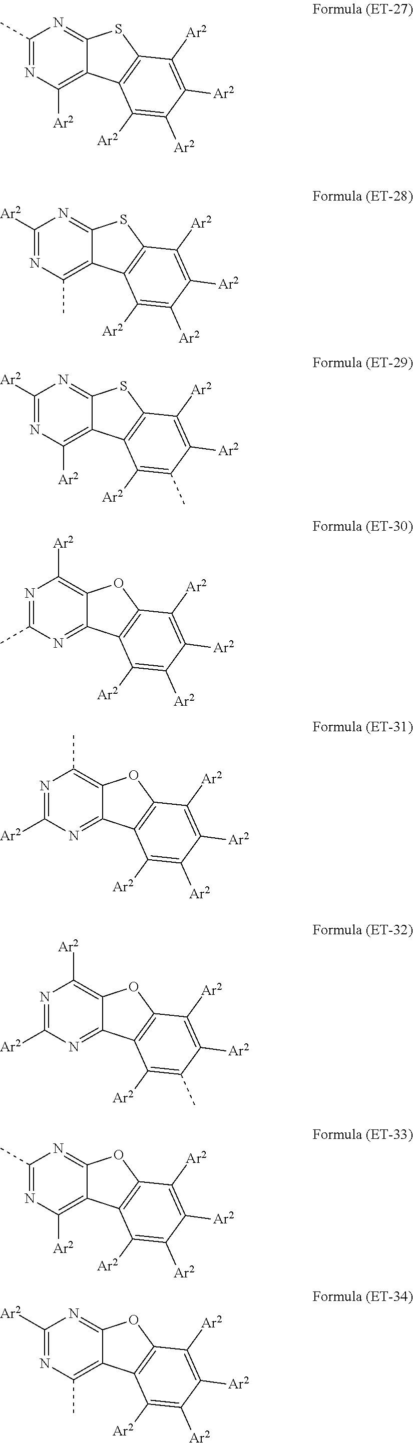 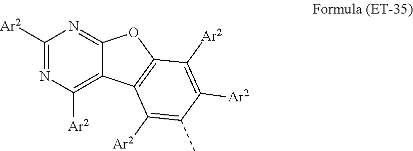
where the dotted lines represent the bonds to the rest of the formula (I), and Ar² is as defined in claim 30.

40. Compound according to claim 30, characterized in that Ar² is the same or different at each instance and is selected from H, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to aromatic ring atoms, where the alkyl groups, alkoxy groups, aromatic ring systems and heteroaromatic ring systems may each be substituted by one or more R³ radicals, and where two or more adjacent Ar² groups together may form a mono- or polycyclic, aliphatic or aromatic ring system.

41. Compound according to claim 30, characterized in that it conforms to the following formula (I-A): 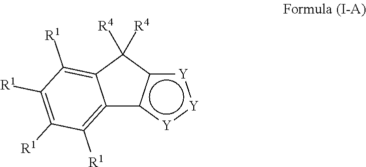
where the variables are as defined in claim 30 and at least one A group is present per formula; and
R⁴ is the same or different at each instance and is selected from H, D, F, Cl, Br, I, C(═O)R¹, CN, Si(R¹)₃, N(R¹)₂, P(═O)(R¹)₂, OR¹, S(═O)R¹, S(═O)₂R¹, straight-chain alkyl, alkoxy or thioalkyl groups having 1 to 20 carbon atoms, branched or cyclic alkyl, alkoxy or thioalkyl groups having 3 to 20 carbon atoms, alkenyl or alkynyl groups having 2 to 20 carbon atoms, aromatic ring systems having 6 to 40 aromatic ring atoms, heteroaromatic ring systems having 5 to 40 aromatic ring atoms, aryloxy or heteroaryloxy groups having 5 to 40 aromatic ring atoms, and aralkyl groups having 5 to 40 aromatic ring atoms; with exclusion of joining of the two R⁴ radicals to one another to form an aliphatic or heteroaliphatic ring; where the alkyl, alkoxy, thioalkyl, alkenyl, alkynyl, aryloxy, heteroaryloxy and aralkyl groups mentioned and the aromatic ring systems and heteroaromatic ring systems mentioned are each substituted by R¹ radicals; and where one or more CH₂ groups in the alkyl, alkoxy, thioalkyl, alkenyl, alkynyl, aryloxy, heteroaryloxy and aralkyl groups mentioned may be replaced by —R¹C═CR¹—, —C≡C, Si(R¹)₂, C═O, C═NR¹, —C(═O)O—, —C(═O)NR¹—, NR¹, P(═O)(R¹), —O—, —S—, SO or SO₂.

42. Compound according to claim 30, characterized in that it corresponds to one of the following formulae (II-A), (III-A) and (IV-A): 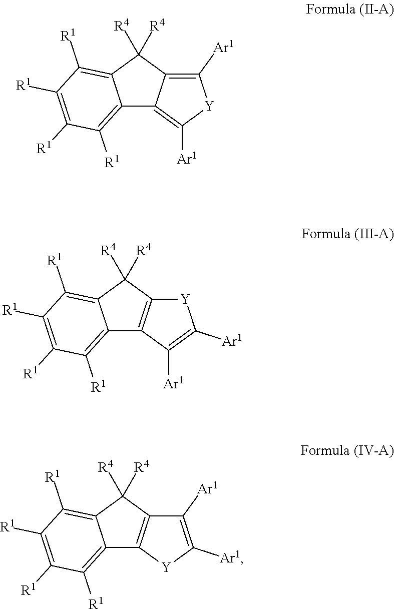
where the variables are as defined in claim 30 and at least one A group is present per formula; and
R⁴ is the same or different at each instance and is selected from F, CN, Si(R¹)₃, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where the alkyl groups mentioned and the aromatic ring systems and heteroaromatic ring systems mentioned are each substituted by R¹ radicals, and with exclusion of bonding of the two R⁴ radicals to one another to form an aliphatic or heteroaliphatic ring.

43. Compound according to claim 30, characterized in that it conforms to one of the following formulae: 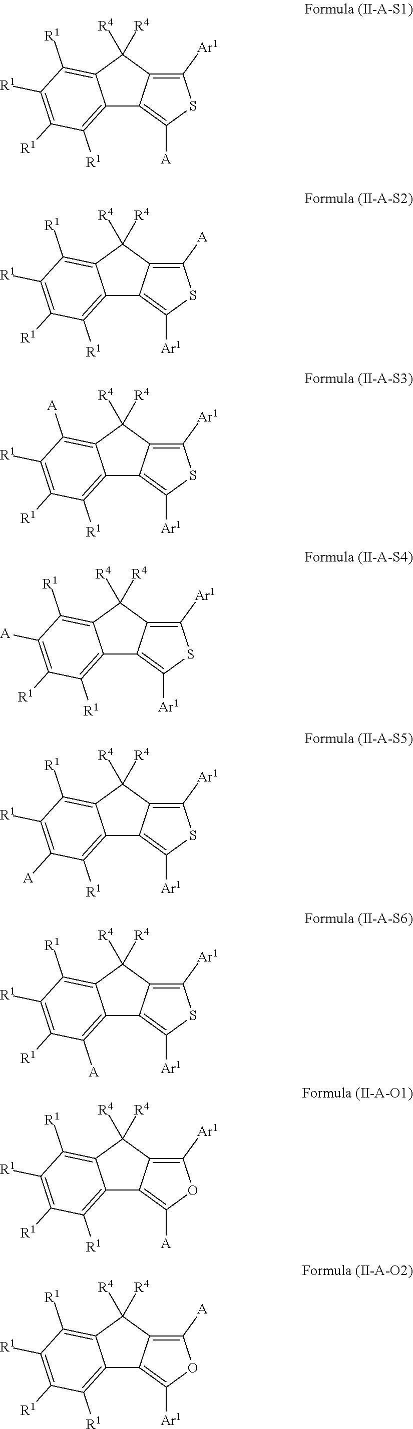 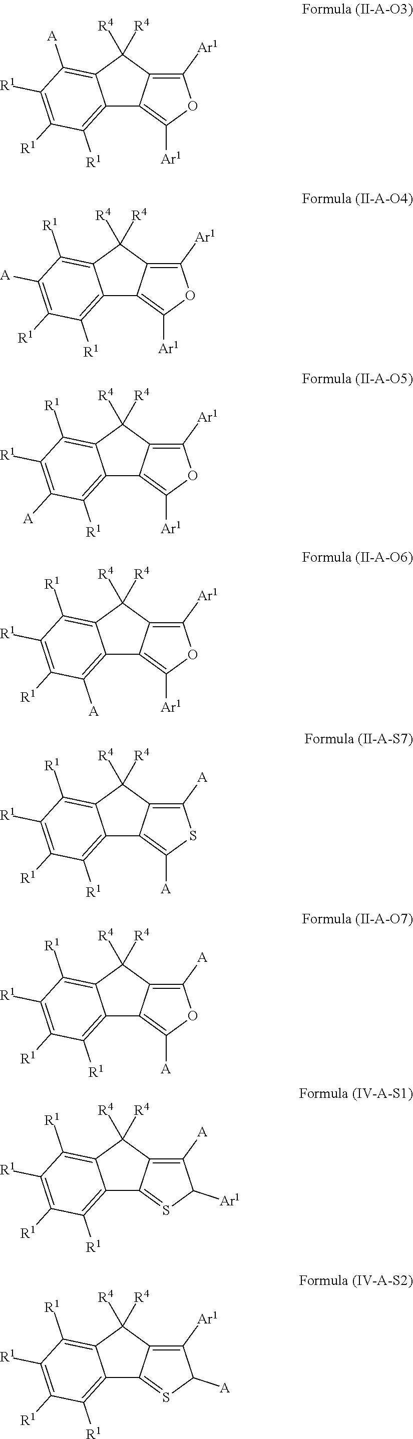 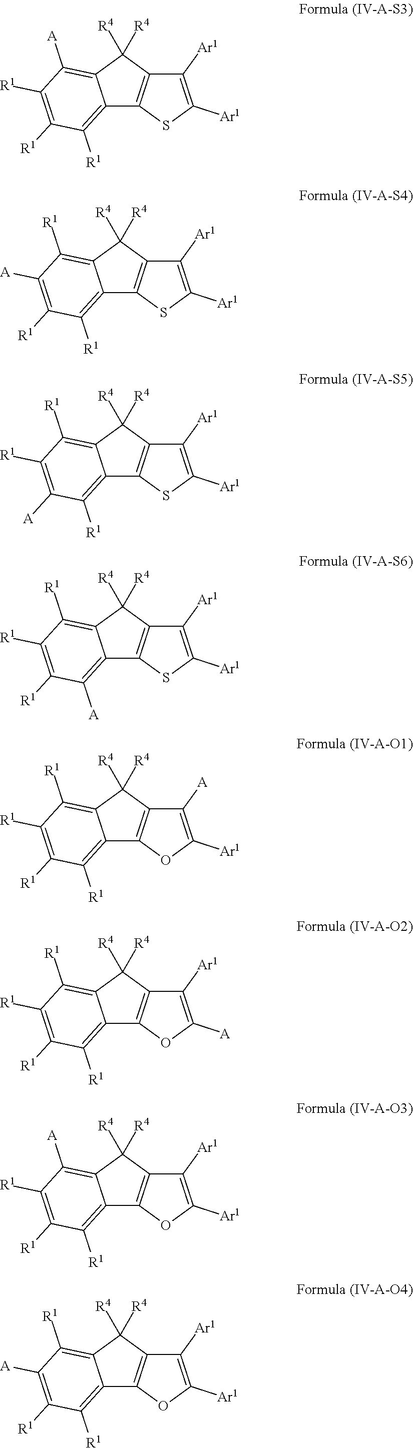 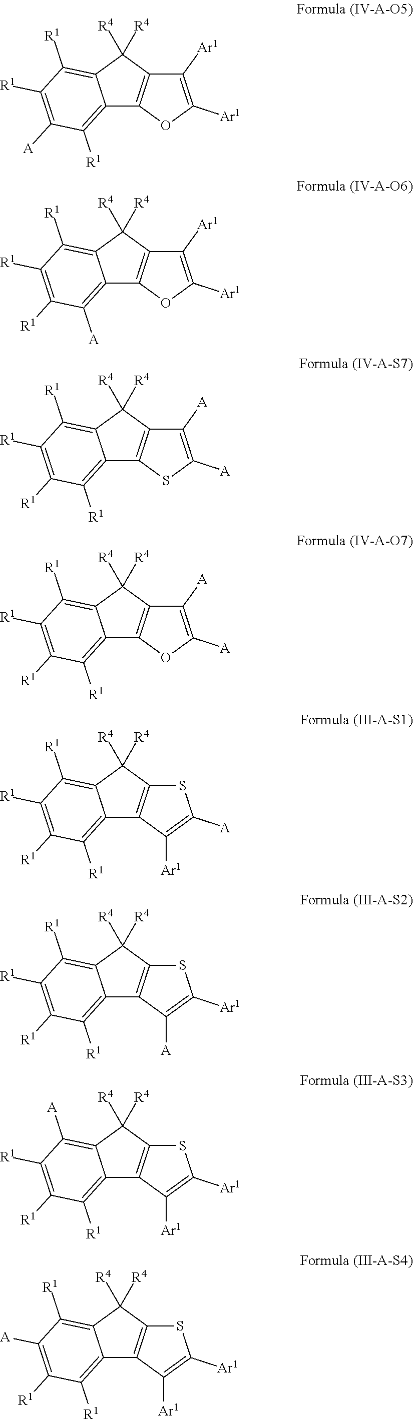 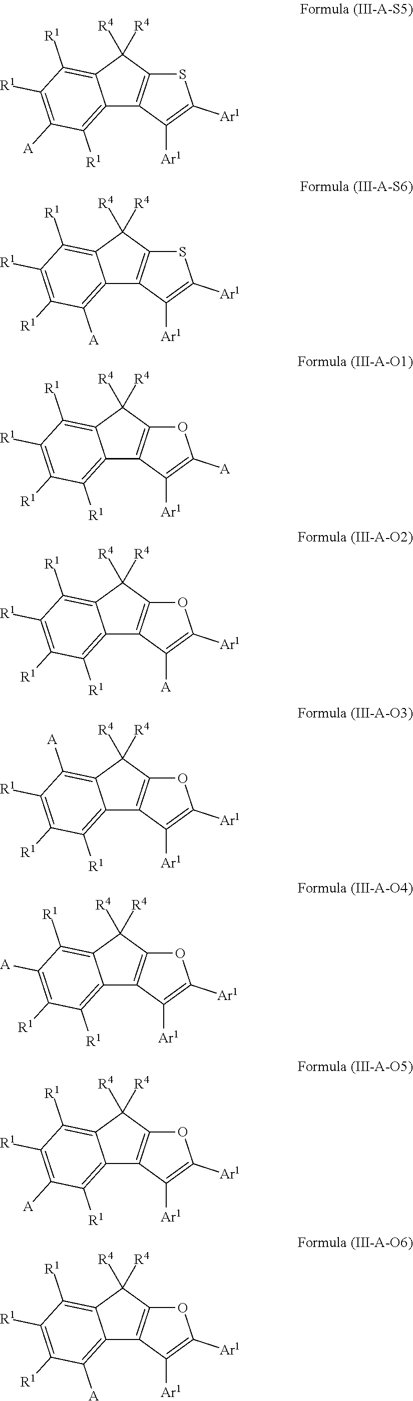 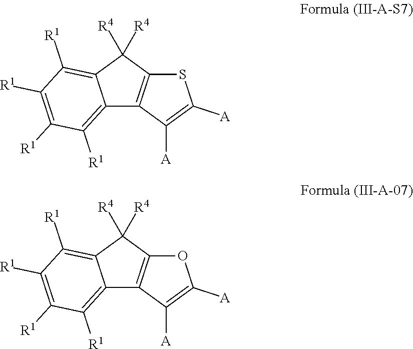
where the variables are as defined in claim 30.

44. Compound according to claim 30, characterized in that it conforms to the following formula (I-B): 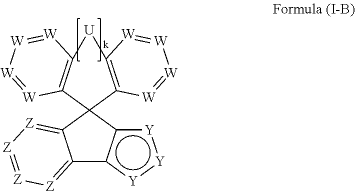
where
W is the same or different at each instance and is selected from N and CR¹;
U is selected from O and S;
k is 0 or 1, where, in the case that k=0, the relevant heteroatom U is absent and U is instead a single bond that connects the corresponding two aromatic six-membered rings; and
at least one A group is present in the formula (I-B), where the variables Z, Y and R¹ and the at least one A group are as defined in claim 30.

45. Compound according to claim 44, characterized in that the W groups are CR¹.

46. Compound according to claim 44, characterized in that it conforms to one of the following formulae (II-B) to (IV-B) and (II-C) to (IV-C): 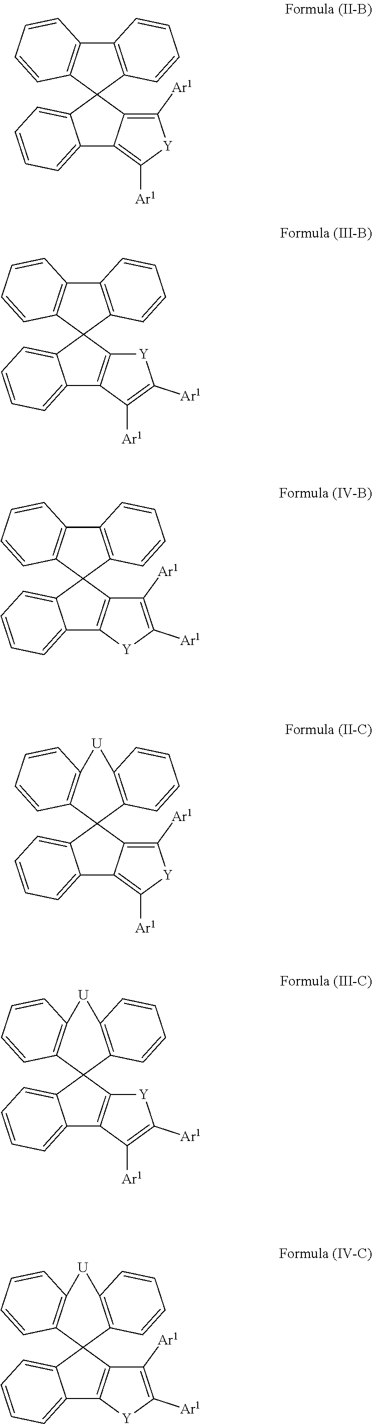
where the variables are as defined in claim 44, and the unoccupied positions on the benzene rings are each substituted by R¹ radicals, and where at least one A group is present per formula.

47. Compound according to claim 44,
characterized in that k=0.

48. Compound according to claim 44, characterized in that it conforms to one of the following formulae:

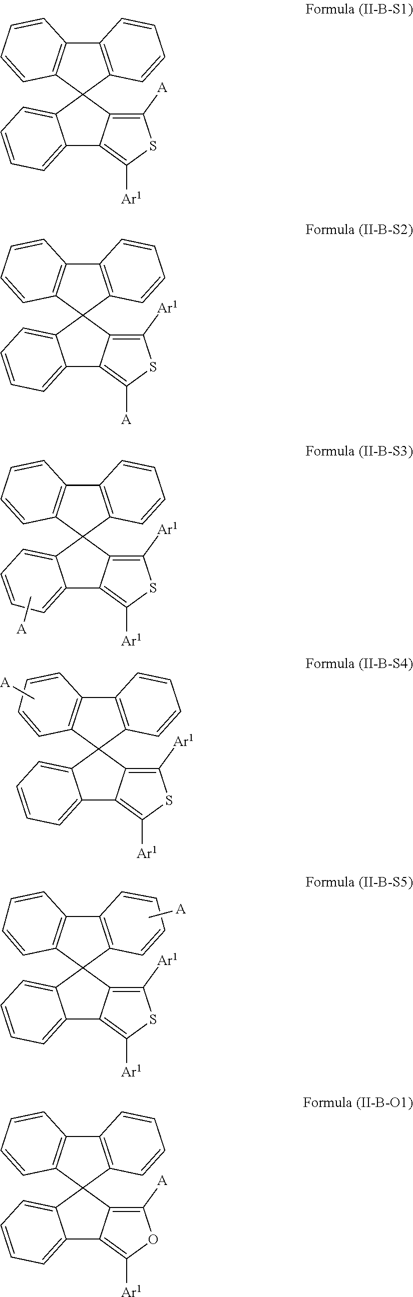

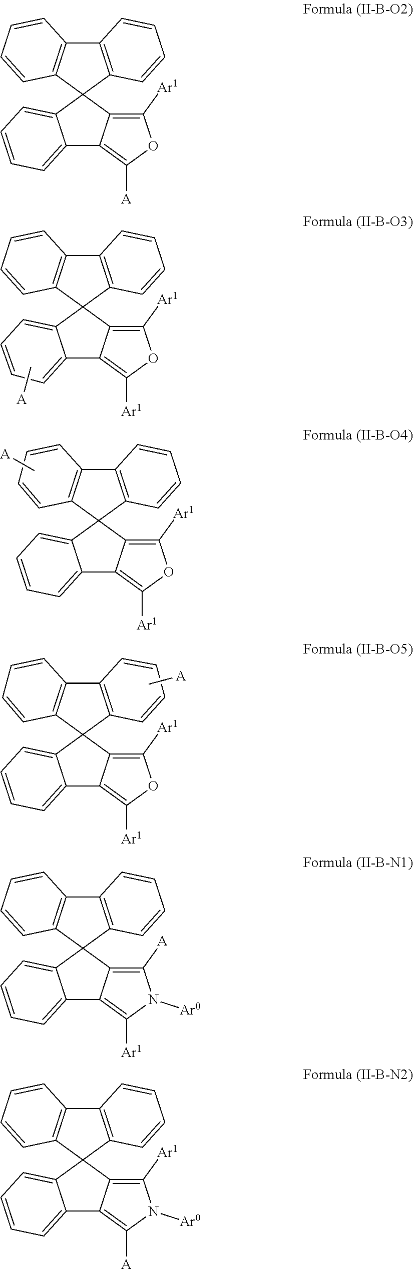

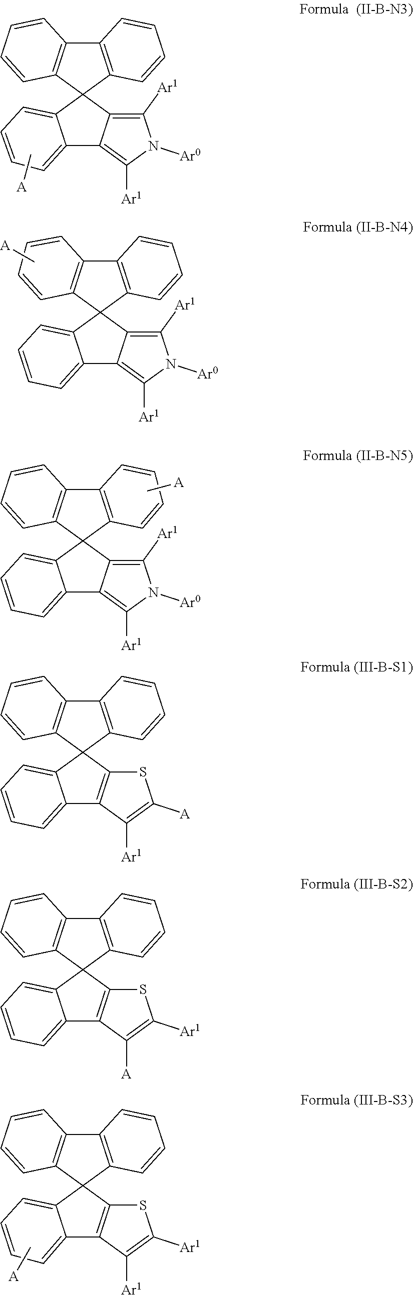

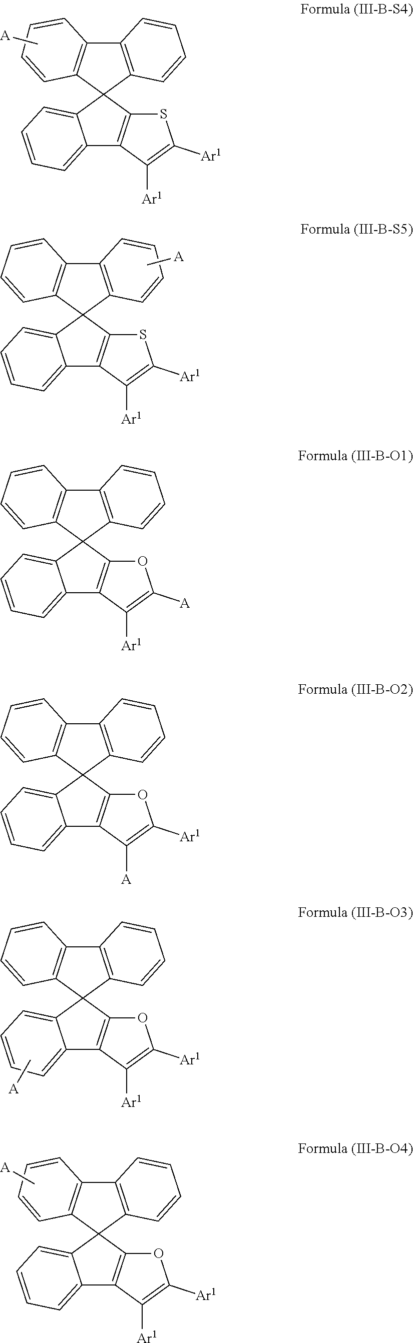

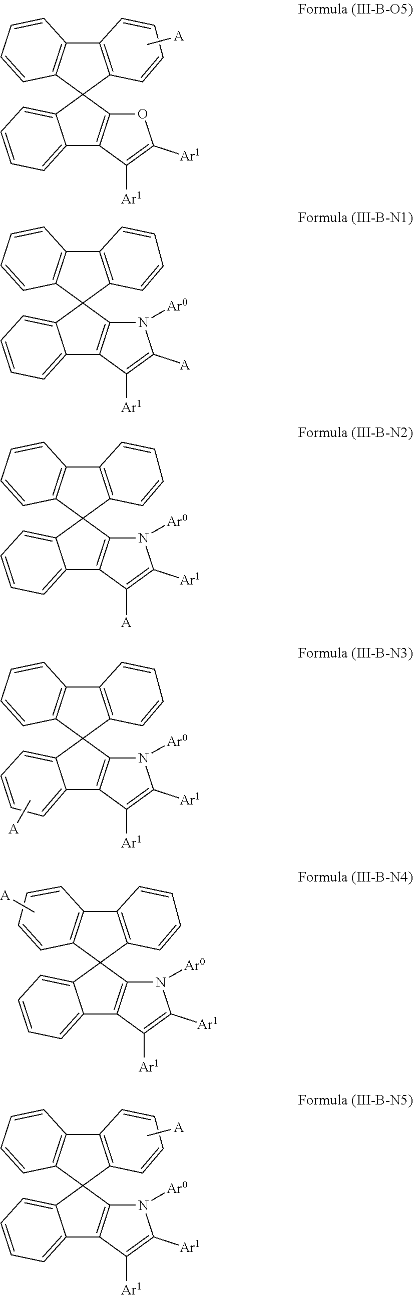

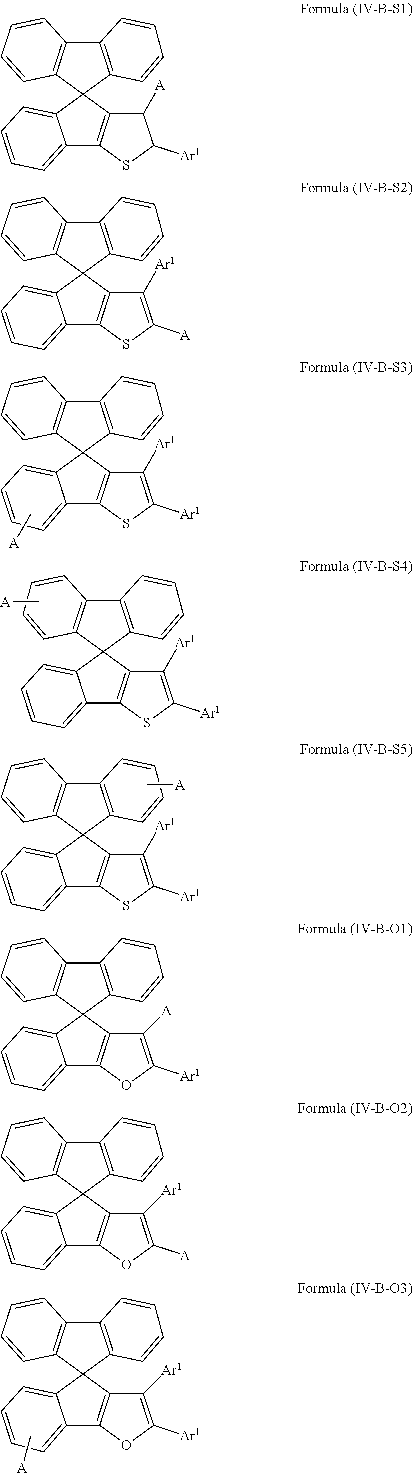

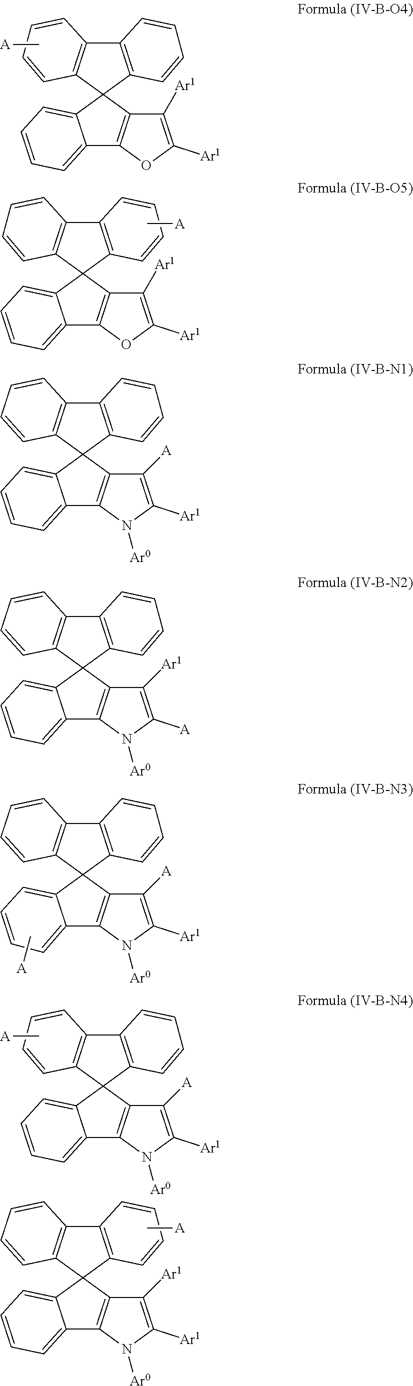

49. Process for preparing a compound according to claim 30, characterized in that
in a first step a Suzuki coupling is conducted, in which a heteroaromatic five-membered ring is coupled to a benzene ring, where the heteroaromatic five-membered ring or the benzene ring bears a carboxylic acid group;
in a second step the carboxylic acid group is cyclized by ring closure reaction under acidic conditions to form a bridging carbonyl group between the heteroaromatic five-membered ring and the benzene ring;
in a third step the carbonyl group is reduced and, with addition of an organohalogen compound or an organometallic reagent, a substituted methylene bridge is obtained between the heteroaromatic five-membered ring and the benzene ring; and
in a fourth step a Suzuki coupling with a heteroaryl compound is conducted; or
in a third step an ortho-halogenated or ortho-metallated bisaryl is added on and a further ring closure reaction is conducted; and
in a fourth step a Suzuki coupling with a heteroaryl compound is conducted to obtain a compound of the formula (I).

50. Oligomer, polymer or dendrimer containing one or more compounds according to claim 30, wherein the bond(s) to the polymer, oligomer or dendrimer may be localized at any desired positions substituted by R⁰, R¹, R² or R³ in formula (I).

51. Formulation comprising at least one compound according to claim 30, and at least one solvent.

52. Composition comprising at least one compound according to claim 30 and at least one further compound selected from hole injection materials, hole transport materials, hole blocker materials, wide bandgap materials, fluorescent emitters, phosphorescent emitters, delayed fluorescent materials, host materials, matrix materials, electron blocker materials, electron transport materials and electron injection materials, n-dopants and p-dopants.

53. Electronic device comprising at least one compound according to claim 30.

54. Electronic device according to claim 53, characterized in that it is an organic electroluminescent device and contains anode, cathode and at least one emitting layer, and in that the compound is present in an electron-transporting layer or in an emitting layer of the device.

55. Electronic device according to claim 54, characterized in that the compound is present as electron-transporting material in the electron transport layer together with one or more further electron-transporting compounds.

56. Electronic device according to claim 54, characterized in that the compound is present as electron-transporting material in the electron transport layer and one or more further electron-transporting compounds are present in the electron injection layer.

57. Electronic device according to claim 54, characterized in that the compound is present as electron-transporting material in the emitting layer together with a hole-transporting material.
